(19) United States
(12) Patent Application Publication
(10) Pub. No.: US 2026/0224688 A1
(43) Pub. Date: Aug. 6, 2026

(54) HELPER LIPIDS FOR NUCLEIC ACID DELIVERY

(71) Applicant: SANOFI PASTEUR INC., Swiftwater, PA (US)

(72) Inventors: Hongfeng DENG, Cambridge, MA (US); Frank DEROSA, Cambridge, MA (US); Shrirang KARVE, Cambridge, MA (US); Neha KAUSHAL, Cambridge, MA (US)

(21) Appl. No.: 19/431,286

(22) Filed: Dec. 23, 2025

Related U.S. Application Data
(63) Continuation of application No. PCT/IB2024/000345, filed on Jun. 28, 2024.

(30) Foreign Application Priority Data
Jun. 28, 2023 (EP) 23306048.2

(51) Int. Cl. A61K 39/145; A61K 9/00; A61K 9/1272; A61K 9/51; A61K 31/7105; A61K 39/00; A61P 31/16; C07F 9/09; C12N 7/00

(52) U.S. Cl. CPC A61K 39/145; A61K 9/0019; A61K 9/1272; A61K 9/5123; A61K 31/7105; A61P 31/16; C07F 9/09; C12N 7/00; A61K 2039/544; A61K 2039/55555; C12N 2760/16134

(57) ABSTRACT

Provided herein are a class of helper lipid compounds of use in the in vivo delivery of therapeutic agents, such as nucleic acids.

### RELATED APPLICATIONS

This application is a continuation of International Patent Application No. PCT/IB2024/000345, filed Jun. 28, 2024, which claims priority to European Patent Application No. 23306048.2, filed Jun. 28, 2023, the entire disclosures of which are hereby incorporated by reference.

### BACKGROUND

Delivery of nucleic acids has been explored extensively as a potential therapeutic option for certain disease states. In particular, messenger RNA (mRNA) therapy has become an increasingly important option for the prevention and treatment of various diseases (e.g., in the use of vaccines).

Efficient delivery of liposome-encapsulated nucleic acids remains an active area of research. Liposome-encapsulated nucleic acids can be administered intramuscularly (IM).

The helper lipid component of a liposome plays an important role in improving the efficacy of nucleic acid transfection. Various helper lipids suitable for in vivo use have been discovered. However, there remains a need to identify helper lipids that are effective for intramuscular delivery of mRNA (e.g., in vaccines, such as for Flu or Respiratory Syncytial virus (RSV)). There also remains a need to identify helper lipids that can be synthesized efficiently and cheaply without the formation of potentially toxic by-products.

### SUMMARY OF THE INVENTION

The present invention provides, among other things, a novel class of helper lipid compounds for improved in vivo delivery of therapeutic agents, such as nucleic acids. Helper lipids may add stability, rigidity, and/or fluidity within lipid bilayers/nanoparticles and facilitate cell fusion and endosomal escape. The inventors of the present invention have surprisingly found that lipid nanoparticles comprising helper lipids of the present invention are very effective for the intramuscular delivery of mRNA encapsulated in said lipid nanoparticles. Indeed, lipid nanoparticles comprising the helper lipids of the present invention have demonstrated high levels of peptide or protein expression when delivering mRNA encoding for said peptide or protein by intramuscular delivery.

It is contemplated that lipid nanoparticles comprising these helper lipid compounds are capable of highly effective in vivo intramuscular delivery of therapeutic agents and vaccines (e.g., for Flu or Respiratory Syncytial virus (RSV)).

In an aspect, provided herein are helper lipids having a structure according to Formula (I):

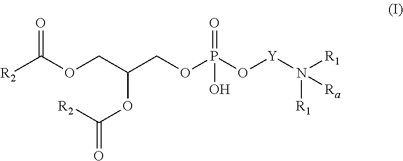

or a pharmaceutically acceptable salt thereof, wherein:
Y is selected from C₂₋₆ alkylene or C₄₋₆ alkenylene;
Rₐ is absent, or Rₐ is optionally substituted C₁-C₁₀ alkyl, wherein, when Rₐ is present, the nitrogen to which Rₐ is bonded bears a positive charge;
each R₁ is independently selected from hydrogen or optionally substituted C₁-C₁₀ alkyl;
each R₂ is independently selected from:
(i) optionally substituted C₄-C₂₄alkyl, optionally substituted C₄-C₂₄alkenyl, and optionally substituted C₄-C₂₄alkynyl;

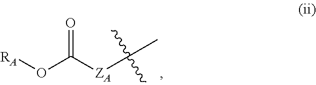

wherein each R_{A} is independently selected from optionally substituted C₁-C₃₁ alkyl, optionally substituted C₂-C₃₁ alkenyl, and optionally substituted C₂-C₃₁ alkynyl; and
   wherein each Z_{A} is independently selected from optionally substituted C₁-C₁₀ alkylene and optionally substituted C₂-C₁₀ alkenylene;

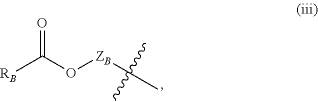

wherein each R_{B} is independently selected from optionally substituted C₁-C₃₁ alkyl, optionally substituted C₂-C₃₁alkenyl, and optionally substituted C₂-C₃₁ alkynyl; and
   wherein each Z_{B} is independently selected from optionally substituted C₁-C₁₀ alkylene and optionally substituted C₂-C₁₀ alkenylene.

In an aspect, provided herein are helper lipids that are pharmaceutically acceptable salts of Formula (I).

In an aspect, provided herein are compositions comprising one or more helper lipids of the present invention or a pharmaceutically acceptable salt thereof, and further comprising:
(i) one or more cationic lipids;
(ii) one or more sterol-based lipids; and
(iii) one or more PEG-modified lipid.

In an aspect, the composition is a lipid nanoparticle, optionally a liposome.

In an aspect, the compositions comprising a cationic lipid and one or more helper lipids of the present invention may be used in therapy, for example, to treat, prevent or ameliorate Flu or Respiratory Syncytial virus (RSV).

### DETAILED DESCRIPTION OF CERTAIN EMBODIMENTS

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an I-amino acid. “Standard amino acid” refers to any of the twenty standard I-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Cationic lipid: As used herein, the term “cationic lipid” refers to lipid species that have a net positive charge at a selected pH, such as physiological pH. Several cationic lipids have been described in the literature, many of which are commercially available. Suitable additional cationic lipids for use in the compositions include the cationic lipids as described in the literature

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery”).

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalents thereof, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Helper lipid: The term “helper lipid” as used herein refers to any neutral or zwitterionic lipid material. Without wishing to be held to a particular theory, helper lipids may add stability, rigidity, and/or fluidity within lipid bilayers/nanoparticles.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains one or more helper lipids of the present invention, a cationic lipid, and optionally further comprises:
(i) one or more additional cationic lipids;
(ii) one or more non-cationic lipids;
(iii) one or more sterol-based lipids; and/or
(iv) one or more PEG-modified lipids.

messenger RNA (mRNA): As used herein, the term “messenger RNA (mRNA)” or “mRNA” refers to a polynucleotide that encodes at least one polypeptide. mRNA as used herein encompasses both modified and unmodified RNA. The term “modified mRNA” related to mRNA comprising at least one chemically modified nucleotide. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C5-propynyl-cytidine, C5-propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. In some embodiments, “nucleic acid” encompasses ribonucleic acids (RNA), including but not limited to any one or more of interference RNAs (RNAi), small interfering RNA (siRNA), short hairpin RNA (shRNA), antisense RNA (aRNA), messenger RNA (mRNA), modified messenger RNA (mmRNA), long non-coding RNA (lncRNA), micro-RNA (miRNA) multimeric coding nucleic acid (MCNA), polymeric coding nucleic acid (PCNA), guide RNA (gRNA) and CRISPR RNA (crRNA). In some embodiments, “nucleic acid” encompasses deoxyribonucleic acid (DNA), including but not limited to any one or more of single-stranded DNA (ssDNA), double-stranded DNA (dsDNA) and complementary DNA (cDNA). In some embodiments, “nucleic acid” encompasses both RNA and DNA. In embodiments, DNA may be in the form of antisense DNA, plasmid DNA, parts of a plasmid DNA, pre-condensed DNA, a product of a polymerase chain reaction (PCR), vectors (e.g., P1, PAC, BAC, YAC, artificial chromosomes), expression cassettes, chimeric sequences, chromosomal DNA, or derivatives of these groups. In embodiments, RNA may be in the form of messenger RNA (mRNA), ribosomal RNA (rRNA), signal recognition particle RNA (7 SL RNA or SRP RNA), transfer RNA (tRNA), transfer-messenger RNA (tmRNA), small nuclear RNA (snRNA), small nucleolar RNA (snoRNA), SmY RNA, small Cajal body-specific RNA (scaRNA), guide RNA (gRNA), ribonuclease P (RNase P), Y RNA, telomerase RNA component (TERC), spliced leader RNA (SL RNA), antisense RNA (aRNA or asRNA), cis-natural antisense transcript (cis-NAT), CRISPR RNA (crRNA), long noncoding RNA (lncRNA), micro-RNA (miRNA), piwi-interacting RNA (piRNA), small interfering RNA (siRNA), transacting siRNA (tasiRNA), repeat associated siRNA (rasiRNA), 73K RNA, retrotransposons, a viral genome, a viroid, satellite RNA, or derivatives of these groups. In some embodiments, a nucleic acid is a mRNA encoding a protein such as an enzyme.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, non-toxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N*(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Systemic distribution or delivery: As used herein, the terms “systemic distribution” or “systemic delivery,” or grammatical equivalents thereof, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Chemical Definitions

Acyl: As used herein, the term “acyl” refers to R^{Z}—(C═O)—, wherein R^{Z} is, for example, any alkyl, alkenyl, alkynyl, heteroalkyl or heteroalkylene.

Aliphatic: As used herein, the term aliphatic refers to (C₁-C₅₀) hydrocarbons and includes both saturated and unsaturated hydrocarbons. An aliphatic may be linear, branched, or cyclic. For example, (C₁-C₂₀)aliphatics can include (C₁-C₂₀)alkyls (e.g., linear or branched (C₁-C₂₀) saturated alkyls), (C₂-C₂₀) alkenyls (e.g., linear or branched (C₄-C₂₀) dienyls, linear or branched (C₆-C₂₀) trienyls, and the like), and (C₂-C₂₀) alkynyls (e.g., linear or branched (C₂-C₂₀) alkynyls). (C₁-C₂₀) aliphatics can include (C₃-C₂₀) cyclic aliphatics (e.g., (C₃-C₂₀) cycloalkyls, (C₄-C₂₀) cycloalkenyls, or (C₃-C₂₀) cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the aliphatic is unsubstituted. In embodiments, the aliphatic does not include any heteroatoms. Alkyl: As used herein, the term “alkyl” means acyclic linear and branched hydrocarbon groups, e.g. “(C₁-C₃₀) alkyl” refers to alkyl groups having 1-30 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkyl is substituted (e.g., with 1, 2, 3, 4, 5, or 6 substituent groups as described herein). In embodiments, an alkyl group is substituted with a —OH group and may also be referred to herein as a “hydroxyalkyl” group, where the prefix denotes the —OH group and “alkyl” is as described herein.

As used herein, “alkyl” also refers to a radical of a straight-chain or branched saturated hydrocarbon group having from 1 to 50 carbon atoms (“(C₁-C₅₀) alkyl”). In some embodiments, an alkyl group has 1 to 40 carbon atoms (“(C₁-C₄₀) alkyl”). In some embodiments, an alkyl group has 1 to 30 carbon atoms (“(C₁-C₃₀) alkyl”). In some embodiments, an alkyl group has 1 to 20 carbon atoms (“(C₁-C₂₀) alkyl”). In some embodiments, an alkyl group has 1 to 10 carbon atoms (“(C₁-C₁₀) alkyl”). In some embodiments, an alkyl group has 1 to 9 carbon atoms (“(C₁-C₉) alkyl”). In some embodiments, an alkyl group has 1 to 8 carbon atoms (“(C₁-C₈) alkyl”). In some embodiments, an alkyl group has 1 to 7 carbon atoms (“(C₁-C₇) alkyl”). In some embodiments, an alkyl group has 1 to 6 carbon atoms (“(C₁-C₆) alkyl”). In some embodiments, an alkyl group has 1 to 5 carbon atoms (“(C₁-C₅) alkyl”). In some embodiments, an alkyl group has 1 to 4 carbon atoms (“(C₁-C₄) alkyl”). In some embodiments, an alkyl group has 1 to 3 carbon atoms (“(C₁-C₃) alkyl”). In some embodiments, an alkyl group has 1 to 2 carbon atoms (“(C₁-C₂) alkyl”). In some embodiments, an alkyl group has 1 carbon atom (“C₁ alkyl”). In some embodiments, an alkyl group has 2 to 6 carbon atoms (“(C₂-C₆) alkyl”). Examples of (C₁-C₆) alkyl groups include, without limitation, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted (C₁-C₅₀) alkyl. In certain embodiments, the alkyl group is a substituted (C₁-C₅₀) alkyl.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In certain embodiments, an alkylene, alkenylene, or alkynylene is unsubstituted. In certain embodiments, an alkylene, alkenylene, or alkynylene does not include any heteroatoms. Alkenyl: As used herein, “alkenyl” means any linear or branched hydrocarbon chains having one or more unsaturated carbon-carbon double bonds that may occur in any stable point along the chain, e.g. “(C₂-C₃₀) alkenyl” refers to an alkenyl group having 2-30 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a-OH group and may also be referred to herein as a “hydroxyalkenyl” group, where the prefix denotes the —OH group and “alkenyl” is as described herein.

As used herein, “alkenyl” also refers to a radical of a straight-chain or branched hydrocarbon group having from 2 to 50 carbon atoms and one or more carbon-carbon double bonds (e.g., 1, 2, 3, or 4 double bonds) (“(C₂-C₅₀) alkenyl”). In some embodiments, an alkenyl group has 2 to 40 carbon atoms (“(C₂-C₄₀) alkenyl”). In some embodiments, an alkenyl group has 2 to 30 carbon atoms (“(C₂-C₃₀) alkenyl”). In some embodiments, an alkenyl group has 2 to 20 carbon atoms (“(C₂-C₂₀) alkenyl”). In some embodiments, an alkenyl group has 2 to 10 carbon atoms (“(C₂-C₁₀) alkenyl”). In some embodiments, an alkenyl group has 2 to 9 carbon atoms (“(C₂-C₉) alkenyl”). In some embodiments, an alkenyl group has 2 to 8 carbon atoms (“(C₂-C₃) alkenyl”). In some embodiments, an alkenyl group has 2 to 7 carbon atoms (“(C₂-C₇) alkenyl”). In some embodiments, an alkenyl group has 2 to 6 carbon atoms (“(C₂-C₆) alkenyl”). In some embodiments, an alkenyl group has 2 to 5 carbon atoms (“(C₂-C₅) alkenyl”). In some embodiments, an alkenyl group has 2 to 4 carbon atoms (“(C₂-C₄) alkenyl”). In some embodiments, an alkenyl group has 2 to 3 carbon atoms (“(C₂-C₃) alkenyl”). In some embodiments, an alkenyl group has 2 carbon atoms (“(C₂) alkenyl”). The one or more carbon-carbon double bonds can be internal (such as in 2-butenyl) or terminal (such as in 1-butenyl). Examples of (C₂-C₄) alkenyl groups include, without limitation, ethenyl (C₂), 1-propenyl (C₃), 2-propenyl (C₃), 1-butenyl (C₄), 2-butenyl (C₄), butadienyl (C₄), and the like. Examples of (C₂-C₆) alkenyl groups include the aforementioned (C₂-C₄) alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted (C₂-C₅₀) alkenyl. In certain embodiments, the alkenyl group is a substituted (C₂-C₅₀) alkenyl.

Alkynyl: As used herein, “alkynyl” means any hydrocarbon chain of either linear or branched configuration, having one or more carbon-carbon triple bonds occurring in any stable point along the chain, e.g., “(C₂-C₃₀) alkynyl”, refers to an alkynyl group having 2-30 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkynyl is unsubstituted. In embodiments, the alkynyl is substituted (e.g., with 1, 2, 3, 4, 5, or 6 substituent groups as described herein).

As used herein, “alkynyl” also refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“(C₂-C₅₀) alkynyl”). An alkynyl group that has one or more triple bonds, and one or more double bonds is also referred to as an “ene-yne”. In some embodiments, an alkynyl group has 2 to 40 carbon atoms (“(C₂-C₄₀) alkynyl”). In some embodiments, an alkynyl group has 2 to 30 carbon atoms (“(C₂-C₃₀) alkynyl”). In some embodiments, an alkynyl group has 2 to 20 carbon atoms (“(C₂-C₂₀) alkynyl”). In some embodiments, an alkynyl group has 2 to 10 carbon atoms (“(C₂-C₁₀) alkynyl”). In some embodiments, an alkynyl group has 2 to 9 carbon atoms (“(C₂-C₉) alkynyl”). In some embodiments, an alkynyl group has 2 to 8 carbon atoms (“(C₂-C₃) alkynyl”). In some embodiments, an alkynyl group has 2 to 7 carbon atoms (“(C₂-C₇) alkynyl”). In some embodiments, an alkynyl group has 2 to 6 carbon atoms (“(C₂-C₆) alkynyl”). In some embodiments, an alkynyl group has 2 to 5 carbon atoms (“(C₂-C₅) alkynyl”). In some embodiments, an alkynyl group has 2 to 4 carbon atoms (“(C₂-C₄) alkynyl”). In some embodiments, an alkynyl group has 2 to 3 carbon atoms (“(C₂-C₃) alkynyl”). In some embodiments, an alkynyl group has 2 carbon atoms (“(C₂) alkynyl”). The one or more carbon-carbon triple bonds can be internal (such as in 2-butynyl) or terminal (such as in 1-butynyl). Examples of (C₂-C₄) alkynyl groups include, without limitation, ethynyl (C₂), 1-propynyl (C₃), 2-propynyl (C₃), 1-butynyl (C₄), 2-butynyl (C₄), and the like. Examples of (C₂-C₆) alkenyl groups include the aforementioned (C₂-C₄) alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted (C₂-C₅₀) alkynyl. In certain embodiments, the alkynyl group is a substituted (C₂-C₅₀) alkynyl.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“(C₆) aryl,” e.g., phenyl). In some embodiments, an aryl group has 10 ring carbon atoms (“(C₁₀) aryl,” e.g., naphthyl such as 1-naphthyl and 2-naphthyl). In some embodiments, an aryl group has 14 ring carbon atoms (“(C₁₄) aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

As used herein, “aryl” also refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“(C₆-C₁₄) aryl”). In some embodiments, an aryl group has 6 ring carbon atoms (“(C₆) aryl”; e.g., phenyl). In some embodiments, an aryl group has 10 ring carbon atoms (“(C₁₀) aryl”; e.g., naphthyl such as 1-naphthyl and 2-naphthyl). In some embodiments, an aryl group has 14 ring carbon atoms (“(C₁₄) aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted (C₆-C₁₄) aryl. In certain embodiments, the aryl group is a substituted (C₆-C₁₄) aryl.

Arylene: The term “arylene” as used herein refers to an aryl group that is divalent (that is, having two points of attachment to the molecule). Exemplary arylenes include phenylene (e.g., unsubstituted phenylene or substituted phenylene).

Carbocyclyl: As used herein, “carbocyclyl” or “carbocyclic” refers to a radical of a non-aromatic cyclic hydrocarbon group having from 3 to 10 ring carbon atoms (“(C₃-C₁₀) carbocyclyl”) and zero heteroatoms in the non-aromatic ring system. In some embodiments, a carbocyclyl group has 3 to 8 ring carbon atoms (“(C₃-C₈) carbocyclyl”). In some embodiments, a carbocyclyl group has 3 to 7 ring carbon atoms (“(C₃-C₇) carbocyclyl”). In some embodiments, a carbocyclyl group has 3 to 6 ring carbon atoms (“(C₃-C₆) carbocyclyl”). In some embodiments, a carbocyclyl group has 4 to 6 ring carbon atoms (“(C₄-C₆) carbocyclyl”). In some embodiments, a carbocyclyl group has 5 to 6 ring carbon atoms (“(C₅-C₆) carbocyclyl”). In some embodiments, a carbocyclyl group has 5 to 10 ring carbon atoms (“(C₅-C₁₀) carbocyclyl”). Exemplary (C₃-C₆) carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary (C₃-C₃) carbocyclyl groups include, without limitation, the aforementioned (C₃-C₆) carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary (C₃-C₁₀) carbocyclyl groups include, without limitation, the aforementioned (C₃-C₃) carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-C₁₀ carbocyclyl. In certain embodiments, the carbocyclyl group is a substituted (C₃-C₁₀) carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“(C₃-C₁₀) cycloalkyl”). In some embodiments, a cycloalkyl group has 3 to 8 ring carbon atoms (“(C₃-C₈) cycloalkyl”). In some embodiments, a cycloalkyl group has 3 to 6 ring carbon atoms (“(C₃-C₆), cycloalkyl”). In some embodiments, a cycloalkyl group has 4 to 6 ring carbon atoms (“(C₄-C₆) cycloalkyl”). In some embodiments, a cycloalkyl group has 5 to 6 ring carbon atoms (“(C₅-C₆) cycloalkyl”). In some embodiments, a cycloalkyl group has 5 to 10 ring carbon atoms (“(C₅-C₁₀) cycloalkyl”). Examples of (C₅-C₆) cycloalkyl groups include cyclopentyl (C₅) and cyclohexyl (C₅). Examples of (C₃-C₆) cycloalkyl groups include the aforementioned (C₅-C₆) cycloalkyl groups as well as cyclopropyl (C₃) and cyclobutyl (C₄). Examples of (C₃-C₃) cycloalkyl groups include the aforementioned (C₃-C₆) cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted (C₃-C₁₀) cycloalkyl. In certain embodiments, the cycloalkyl group is a substituted (C₃-C₁₀) cycloalkyl.

Halogen: As used herein, the term “halogen” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, is fully unsaturated heteroatom-containing ring wherein at least one ring atom is a heteroatom such as, but not limited to, nitrogen and oxygen.

As used herein, “heteroaryl” also refers to a radical of a 5-14 membered monocyclic or polycyclic (e.g., bicyclic or tricyclic) 4n+2 aromatic ring system (e.g., having 6, 10, or 14 π electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)) and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno [3,2-b]pyridinyl, 1,2,3,4-tetrahydro-1,6-naphthyridinyl, and the like.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

As understood from the above, alkyl, alkenyl, alkynyl, acyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valences of the heteroatoms and results in the formation of a stable moiety.

Exemplary carbon atom substituents include, but are not limited to, halogen, —CN, —NO₂, —N₃, —SO₂, —SO₃H, —OH, —OR^{aa}, —ON(R^{bb})₂, —N(R^{bb})₂, —N(R^{bb})₃+X⁻, —N(OR^{cc})R^{bb}, —SeH, —SeR^{aa}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃—OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), (C₁-C₅₀) alkyl, (C₂-C₅₀) alkenyl, (C₂-C₅₀) alkynyl, (C₃-C₁₄) carbocyclyl, 3-14 membered heterocyclyl, (C₆-C₁₄) aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, =NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{bb} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{cc} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl and 5-10 membered heteroaryl, or two R^{ff} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —O(C₁-C₅₀) alkyl, —ON((C₁-C₅₀) alkyl)₂, —N((C₁-C₅₀) alkyl)₂, —N((C₁-C₅₀) alkyl)₃+X⁻, —NH((C₁-C₅₀) alkyl)₂+X⁻, —NH₂((C₁-C₅₀) alkyl)+X⁻, —NH₃+X⁻, —N(O(C₁-C₅₀) alkyl)((C₁-C₅₀) alkyl), —N(OH)((C₁-C₅₀) alkyl), —NH(OH), —SH, —S(C₁-C₅₀) alkyl, —SS((C₁-C₅₀) alkyl), —C(═O)((C₁-C₅₀) alkyl), —CO₂H, —CO₂((C₁-C₅₀) alkyl), —OC(═O)((C₁-C₅₀) alkyl), —OCO₂((C₁-C₅₀) alkyl), —C(═O)NH₂, —C(═O)N((C₁-C₅₀) alkyl)₂, —OC(═O)NH((C₁-C₅₀) alkyl), —NHC(═O)((C₁-C₅₀) alkyl), —N((C₁-C₅₀) alkyl)C(═O)((C₁-C₅₀) alkyl), —NHCO₂((C₁-C₅₀) alkyl), —NHC(═O)N((C₁-C₅₀) alkyl)₂, —NHC(═O)NH((C₁-C₅₀) alkyl), —NHC(═O)NH₂, —C(═NH)O((C₁-C₅₀) alkyl), —OC(═NH)((C1-C₅₀) alkyl), —OC(═NH)O(C₁-C₅₀) alkyl, —C(═NH)N((C₁-C₅₀) alkyl)₂, —C(═NH)NH((C₁-C₅₀) alkyl), —C(═NH)NH₂, —OC(═NH)N((C1-C₅₀)alkyl)₂, —OC(NH)NH((C₁-C₅₀) alkyl), —OC(NH)NH₂, —NHC(NH)N((C₁-C₅₀) alkyl)₂, —NHC(═NH)NH₂, —NHSO₂((C₁-C₅₀) alkyl), —SO₂N((C₁-C₅₀) alkyl)₂, —SO₂NH((C₁-C₅₀) alkyl), —SO₂NH₂, —SO₂((C₁-C₅₀) alkyl), —SO₂O((C₁-C₅₀) alkyl), —OSO₂((C₁-C₆) alkyl), —SO((C₁-C₆) alkyl), —Si((C1-C₅₀) alkyl)₃, —OSi((C1-C₆) alkyl)₃, —C(═S)N((C₁-C₅₀) alkyl)₂, C(═S)NH((C₁-C₅₀) alkyl), C(═S)NH₂, —C(═O)S((C₁-C₆) alkyl), —C(═S)S((C₁-C₆) alkyl), —SC(═S)S((C₁-C₆) alkyl), —P(═O)₂((C₁-C₅₀) alkyl), —P(═O)((C₁-C₅₀) alkyl)₂, —OP(═O)((C₁-C₅₀) alkyl)₂, —OP(═O)(O(C₁-C₅₀) alkyl)₂, (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, 5-10 membered heteroaryl; or two geminal R⁹⁹ substituents can be joined to form ═O or =S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb}) R^{aa}, —C(═NR^{cc})OR^{aa}, —C(═NRR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{cc} groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary sulfur protecting groups include, but are not limited to, alkyl, benzyl, p-methoxybenzyl, 2,4,6-trimethylbenzyl, 2,4,6-trimethoxybenzyl, o-hydroxybenzyl, p-hydroxybenzyl, o-acetoxybenzyl, p-acetoxybenzyl, p-nitrobenzyl, 4-picolyl, 2-quinolinylmethyl, 2-picolyl N-oxido, 9-anthrylmethyl, 9-fluorenylmethyl, xanthenyl, ferrocenylmethyl, diphenylmethyl, bis(4-methoxyphenyl)methyl, 5-dibenzosuberyl, triphenylmethyl, diphenyl-4-pyridylmethyl, phenyl, 2,4-dinitrophenyl, t-butyl, 1-adamantyl, methoxymethyl (MOM), isobutoxymethyl, benzyloxymethyl, 2-tetrahydropyranyl, benzylthiomethyl, phenylthiomethyl, thiazolidino, acetamidomethyl, trimethylacetamidomethyl, benzamidomethyl, allyloxycarbonylaminomethyl, phenylacetamidomethyl, phthalimidomethyl, acetylmethyl, carboxymethyl, cyanomethyl, (2-nitro-1-phenyl)ethyl, 2-(2,4-dinitrophenyl)ethyl, 2-cyanoethyl, 2-(Trimethylsilyl)ethyl, 2,2-bis(carboethoxy)ethyl, (1-m-nitrophenyl-2-benzoyl)othyl, 2-phenylsulfonylethyl, 2-(4-methylphenylsulfonyl)-2-methylprop-2-yl, acetyl, benzoyl, trifluoroacetyl, N-[[(p-biphenylyl)isopropoxy]carbonyl]-N-methyl]-γ-aminothiobutyrate, 2,2,2-trichloroethoxycarbonyl, t-butoxycarbonyl, benzyloxycarbonyl, p-methoxybenzyloxycarbonyl, N-ethyl, N-methoxymethyl, sulfonate, sulfenylthiocarbonate, 3-nitro-2-pyridinesulfenyl sulfide, oxathiolone.

### Compounds of the Invention

Liposomal-based vehicles are considered as an attractive carrier for therapeutic agents and remain subject to continued development efforts. While liposomal-based vehicles that comprise certain lipid components have shown promising results with regard to encapsulation, stability and site localization, there remains a great need for improvement of liposomal-based delivery systems. For example, a significant drawback of liposomal delivery systems relates to the construction of liposomes that have sufficient cell culture or in vivo stability to reach desired target cells and/or intracellular compartments, and the ability of such liposomal delivery systems to efficiently release their encapsulated materials to such target cells.

In particular, there remains a need for improved helper lipids that are effective for intramuscular delivery of mRNA (e.g. for treating Flu or Respiratory Syncytial virus (RSV)). There also remains a need for improved helper lipid compounds that demonstrate improved pharmacokinetic properties, and which are capable of improving the delivery efficiency of macromolecules, such as nucleic acids, to a wide variety cell types and tissues. There also remains a particular need for novel helper lipid compounds that are characterized as having improved safety profiles and are capable of improving the delivery efficiency of encapsulated nucleic acids and polynucleotides to targeted cells, tissues and organs.

Described herein is a novel class of helper lipid compounds for improved in vivo delivery of therapeutic agents, such as nucleic acids (e.g., for Flu or Respiratory Syncytial virus (RSV)). In particular, a helper lipid described herein may be used in combination with a cationic lipid and optionally with other lipids to formulate a lipid-based nanoparticle (e.g., liposome) for encapsulating therapeutic agents, such as nucleic acids (e.g., DNA, siRNA, mRNA, microRNA) for therapeutic use, such as disease treatment and prevention (vaccine e.g., for Flu or Respiratory Syncytial virus (RSV)) purposes.

In embodiments, compounds of the invention as described herein can provide one or more desired characteristics or properties. That is, in certain embodiments, compounds of the invention as described herein can be characterized as having one or more properties that afford such compounds advantages relative to other similarly classified lipids. For example, compounds disclosed herein can allow for the control and tailoring of the properties of liposomal compositions (e.g., lipid nanoparticles) of which they are a component. In particular, compounds disclosed herein can be characterized by enhanced transfection efficiencies and their ability to provoke specific biological outcomes. Such outcomes can include, for example enhanced cellular uptake, endosomal/lysosomal disruption capabilities and/or promoting the release of encapsulated materials (e.g., polynucleotides) intracellularly. Lipid nanoparticles comprising one or more of the helper lipids of the present invention can also be characterized by achieving high levels of peptide or protein expression when delivering mRNA encoding for said peptide or protein by intravenous, intrathecal, intramuscular, intranasal, sublingual, or by pulmonary delivery, optionally through nebulization. Additionally, the compounds disclosed herein have advantageous pharmacokinetic properties, biodistribution, and efficiency.

The present application demonstrates that not only are the helper lipids of the present invention synthetically tractable from readily available starting materials, and that lipid nanoparticles comprising one or more of the helper lipids of the present invention have certain other advantages.

Additionally, the helper lipids of the present invention have cleavable groups such as ester groups. These cleavable groups (e.g., esters, thioesters, disulphides, carbonates, carbamates and thiocarbamates) are contemplated to improve biodegradability and thus contribute to the lipids' favorable safety profiles. It is contemplated that lipid nanoparticles comprising one or more of the helper lipids of the present invention are capable of highly effective in vivo intramuscular delivery of the therapeutic agents and vaccines (e.g., for Flu or Respiratory Syncytial virus (RSV)). It is also contemplated that lipid nanoparticles comprising the helper lipids of the present invention are capable of highly effective endosomal escape. It is also contemplated that lipid nanoparticles comprising the helper lipids of the present invention are capable of highly effective in vivo delivery while maintaining a favorable safety profile. It is also contemplated that lipid nanoparticles comprising the helper lipids of the present invention may exhibit beneficial degradation in vivo. It is also contemplated that the novel helper lipids of the present invention are capable of providing improved stability to the compositions disclosed herein.

Provided herein are compounds which are helper lipids. In embodiments, the helper lipids of the present invention include compounds having a structure according to Formula (I):

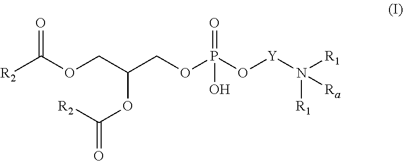

or a pharmaceutically acceptable salt thereof, wherein:
Y is selected from optionally substituted C₂-C₆ alkylene or optionally substituted C₄-C₆ alkenylene;
Rₐ is absent, or Rₐ is optionally substituted C₁-C₁₀ alkyl, wherein, when Rₐ is present, the nitrogen to which Rₐ is bonded bears a positive charge;
each R₁ is independently selected from hydrogen or optionally substituted C₁-C₁₀ alkyl;
each R₂ is independently selected from:
(i) optionally substituted C₄-C₂₄alkyl, optionally substituted C₄-C₂₄alkenyl, and optionally substituted C₄-C₂₄alkynyl;

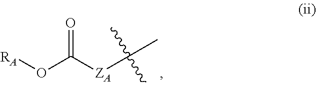

wherein each R_{A} is independently selected from optionally substituted C₁-C₃₁ alkyl, optionally substituted C₂-C₃₁ alkenyl, and optionally substituted C₂-C₃₁ alkynyl; and
   wherein each Z_{A} is independently selected from optionally substituted C₁-C₁₀ alkylene and optionally substituted C₂-C₁₀ alkenylene;

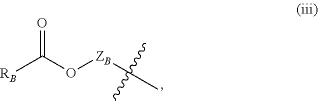

wherein each R_{B} is independently selected from optionally substituted C₁-C₃₁ alkyl, optionally substituted C₂-C₃₁alkenyl, and optionally substituted C₂-C₃₁ alkynyl; and
   wherein each Z_{B} is independently selected from optionally substituted C₁-C₁₀ alkylene and optionally substituted C₂-C₁₀ alkenylene.

In embodiments, the helper lipids of the present invention include compounds having a structure according to Formula (II):

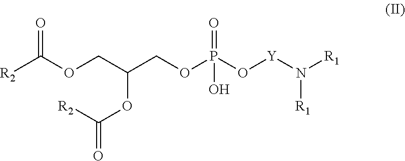

or a pharmaceutically acceptable salt thereof.

In embodiments, the helper lipids of the present invention include compounds having a structure according to Formula (IIa):

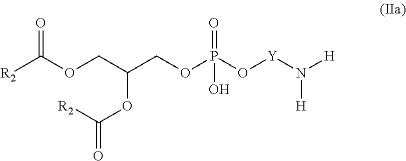

or a pharmaceutically acceptable salt thereof.

In embodiments, the helper lipids of the present invention include compounds having a structure according to Formula (IIb):

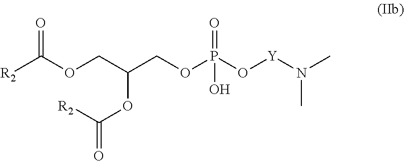

or a pharmaceutically acceptable salt thereof.

In embodiments, the helper lipids of the present invention include compounds having a structure according to Formula (IIc):

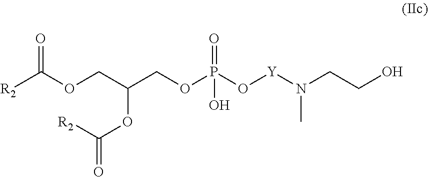

or a pharmaceutically acceptable salt thereof.

In embodiments, the helper lipids of the present invention include compounds having a structure according to Formula (IId):

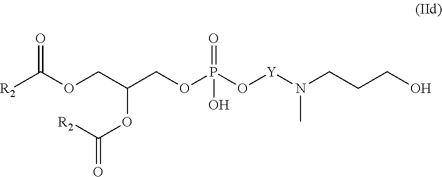

or a pharmaceutically acceptable salt thereof.

In embodiments, the helper lipids of the present invention include compounds having a structure according to Formula (III):

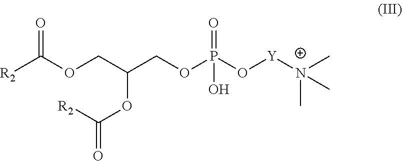

or a pharmaceutically acceptable salt thereof.

In embodiments, Y is selected from optionally substituted C₂₋₆ alkylene. In embodiments, Y is optionally substituted C₂ alkylene. In embodiments, Y is optionally substituted C₃ alkylene. In embodiments, Y is optionally substituted C₄ alkylene. In embodiments, Y is optionally substituted C₅ alkylene. In embodiments, Y is optionally substituted C₆ alkylene.

In a preferred embodiment, Y is —CH₂CH₂—.

In embodiments, Y is selected from optionally substituted C₄₋₆ alkenylene. In embodiments, Y is optionally substituted C₄ alkenylene. In embodiments, Y is optionally substituted C₅ alkenylene. In embodiments, Y is optionally substituted C₆ alkenylene.

In embodiments, the helper lipids of the present invention include compounds having a structure according to Formula (IV):

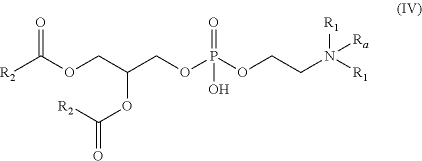

or a pharmaceutically acceptable salt thereof.

In embodiments, the helper lipids of the present invention include compounds having a structure according to Formula (V):

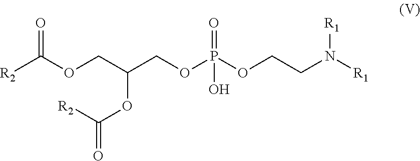

or a pharmaceutically acceptable salt thereof.

In embodiments, the helper lipids of the present invention include compounds having a structure according to Formula (Va):

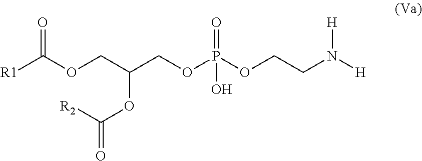

or a pharmaceutically acceptable salt thereof.

In embodiments, the helper lipids of the present invention include compounds having a structure according to Formula (Vb):

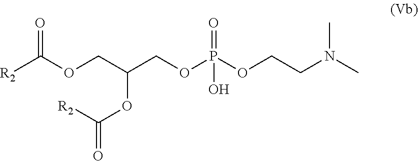

or a pharmaceutically acceptable salt thereof.

In embodiments, the helper lipids of the present invention include compounds having a structure according to Formula (Vc):

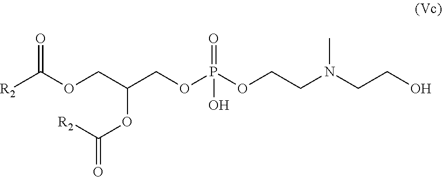

or a pharmaceutically acceptable salt thereof.

In embodiments, the helper lipids of the present invention include compounds having a structure according to Formula (Vd):

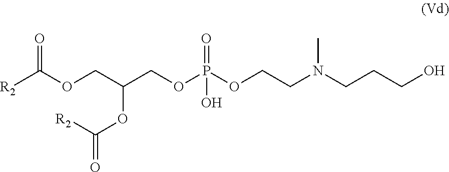

or a pharmaceutically acceptable salt thereof.

In embodiments, the stereochemistry of the carbon atom situated between R₂C(═O)O—CH₂— and —CH₂—OP(═O)(OH)—O— is as depicted in the following structure:

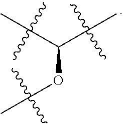

In embodiments, the stereochemistry of the carbon atom situated between R₂C(═O)O—CH₂— and —CH₂—OP(═O)(OH)—O— is as depicted in the following structure:

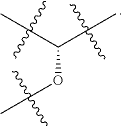

In embodiments, each R₂ is the same. In embodiments, each R₂ is different.

In embodiments, Rₐ is absent. In embodiments, each R₁ is independently selected from optionally substituted C₁-C₁₀ alkyl. In embodiments, each R₁ is methyl. In embodiments, each R₁ is ethyl. In embodiments, each R₁ is propyl. In embodiments, each R₁ is butyl. In embodiments, each R₁ is pentyl. In embodiments, Rₐ is absent and each R₁ is independently selected from optionally substituted C₁-C₁₀ alkyl. In embodiments, Rₐ is absent and each R₁ is methyl. In embodiments, Rₐ is absent and each R₁ is ethyl. In embodiments, Rₐ is absent and each R₁ is propyl. In embodiments, Rₐ is absent and each R₁ is butyl. In embodiments, Rₐ is absent and each R₁ is pentyl.

In embodiments, one R₁ is independently selected from optionally substituted C₁-C₁₀ alkyl and the other R₁ is independently selected from C₁-C₁₀ alkyl substituted with a hydroxyl group. In embodiments, one R₁ is methyl and the other Riis C₁-C₁₀ alkyl terminally substituted with a hydroxyl group. In embodiments, one R₁ is methyl and the other R₁ is hydroxymethyl. In embodiments, one R₁ is methyl and the other R₁ is hydroxyethyl, for example 2-hydroxyethyl. In embodiments, one R₁ is methyl and the other Riis hydroxypropyl, for example 3-hydroxypropyl. In embodiments, Rₐ is absent, one R₁ is independently selected from optionally substituted C₁-C₁₀ alkyl and the other Riis independently selected from C₁-C₁₀ alkyl substituted with a hydroxyl group. In embodiments, Rₐ is absent, one R₁ is methyl and the other Riis C₁-C₁₀ alkyl terminally substituted with a hydroxyl group. In embodiments, Rₐ is absent, one R₁ is methyl and the other R₁ is hydroxymethyl. In embodiments, Rₐ is absent, one R₁ is methyl and the other R₁ is hydroxyethyl, for example 2-hydroxyethyl. In embodiments, Rₐ is absent, one R₁ is methyl and the other R₁ is hydroxypropyl, for example 3-hydroxypropyl.

In embodiments, Rₐ is present. In embodiments, Rₐ is present and each R₁ is independently selected from optionally substituted C₁-C₁₀ alkyl. In embodiments, Rₐ is present and each R₁ is methyl. In embodiments, Rₐ is present and each R₁ is ethyl. In embodiments, Rₐ is present and each R₁ is propyl. In embodiments, Rₐ is present and each R₁ is butyl. In embodiments, Rₐ is present and each R₁ is pentyl.

In embodiments, each R₂ is independently selected from optionally substituted C₄-C₂₄alkyl, optionally substituted C₄-C₂₄alkenyl, and optionally substituted C₄-C₂₄alkynyl. In embodiments, each R₂ is independently selected from optionally substituted C₅-C₂₀ alkyl, optionally substituted C₅-C₂₀ alkenyl, and optionally substituted C₅-C₂₀ alkynyl. In embodiments, each R₂ is independently selected from optionally substituted C₆-C₁₈ alkyl, optionally substituted C₆-C₁₈ alkenyl, and optionally substituted C₆-C₁₈ alkynyl. In embodiments, each R₂ is independently selected from optionally substituted C₇-C₁₅ alkyl, optionally substituted C₇-C₁₅ alkenyl, and optionally substituted C₇-C₁₅ alkynyl.

In embodiments, each R₂ is independently selected from optionally substituted C₄-C₂₄alkyl, for example a branched C₄-C₂₄ alkyl. In embodiments, each R₂ is independently selected from optionally substituted C₅-C₂₀ alkyl, for example a branched C₅-C₂₀ alkyl. In embodiments, each R₂ is independently selected from optionally substituted C₆-C₁₈ alkyl, for example a branched C₆-C₁₈ alkyl. In embodiments, each R₂ is independently selected from optionally substituted C₇-C₁₅ alkyl, for example a branched C₇-C₁₅ alkyl.

In embodiments, each R₂ is independently selected from

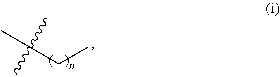

wherein each n is independently selected from 3, 4, 5, 6, 7, 8, 9, 10, 11, 12, 13, 14, 15, 16 17, 18, 19, 20, 21, 22, and 23,

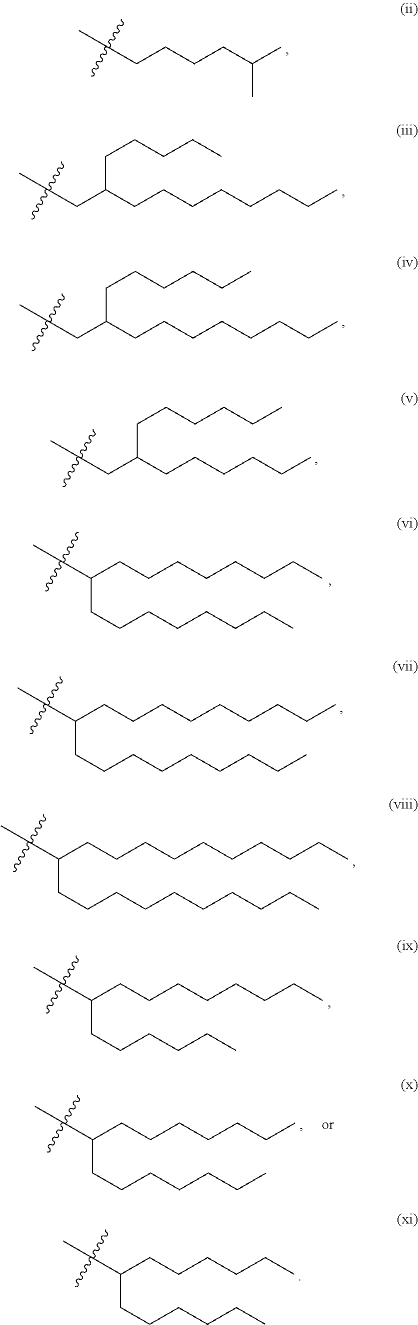

In embodiments, each R₂ is independently selected from:

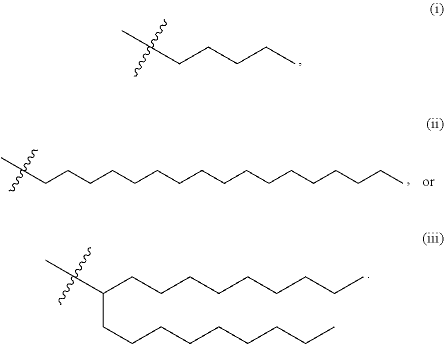

In embodiments, each R₂ is independently selected from

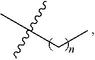

wherein each n is independently selected from 3, 4, 5, 6, 7, 8, 9, 10, 11, 12, 13, 14, 15, 16 17, 18, 19, 20, 21, 22, and 23. In embodiments, each n is 3. In embodiments, each n is 4. In embodiments, each n is 5. In embodiments, each n is 6. In embodiments, each n is 7. In embodiments, each n is 8. In embodiments, each n is 9. In embodiments, each n is 10. In embodiments, each n is 11. In embodiments, each n is 12. In embodiments, each n is 13. In embodiments, each n is 14. In embodiments, each n is 15. In embodiments, each n is 16. In embodiments, each n is 17. In embodiments, each n is 18. In embodiments, each n is 19. In embodiments, each n is 20. In embodiments, each n is 21. In embodiments, each n is 22. In embodiments, each n is 23.

In embodiments, each R₂ is independently selected from

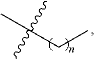

wherein each n is independently selected from 3-23. In embodiments, each R₂ is independently selected from

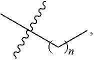

wherein each n is independently selected from 5-21. In embodiments, each R₂ is independently selected from

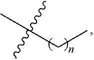

wherein each n is independently selected from 7-19. In embodiments, each R₂ is independently selected from

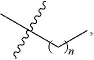

wherein each n is independently selected from 9-17. In embodiments, each R₂ is independently selected from

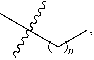

wherein each n is independently selected from 11-15.

In embodiments, each R₂ is

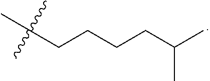 [SMILES: *CCCCC(C)C]

In embodiments, each R₂ is

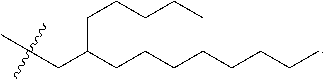 [SMILES: *CC(CCCCC)CCCCCCCC]

In embodiments, each R₂ is

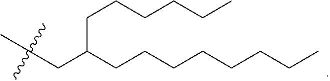 [SMILES: *CC(CCCCCC)CCCCCCCC]

In embodiments, each R₂ is

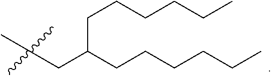 [SMILES: *CC(CCCCCC)CCCCCC]

In embodiments, each R₂ is

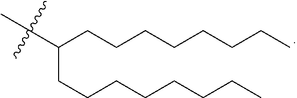 [SMILES: *C(CCCCCCCC)CCCCCCCC]

In embodiments, each R₂ is

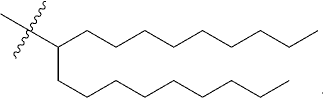 [SMILES: *C(CCCCCCCCC)CCCCCCCCC]

In embodiments, each R₂ is

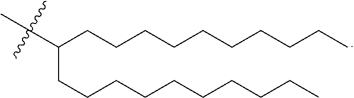 [SMILES: *C(CCCCCCCCCC)CCCCCCCCCC]

In embodiments, each R₂ is

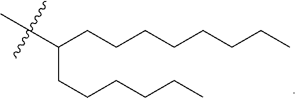 [SMILES: *C(CCCCCC)CCCCCCCC]

In embodiments, each R₂ is

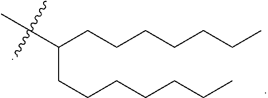

In embodiments, each R₂ is

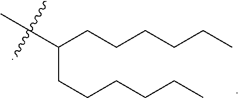

In embodiments, each R₂ is independently selected from optionally substituted C₄-C₂₄ alkenyl.

In embodiments, R₂ is independently selected from:

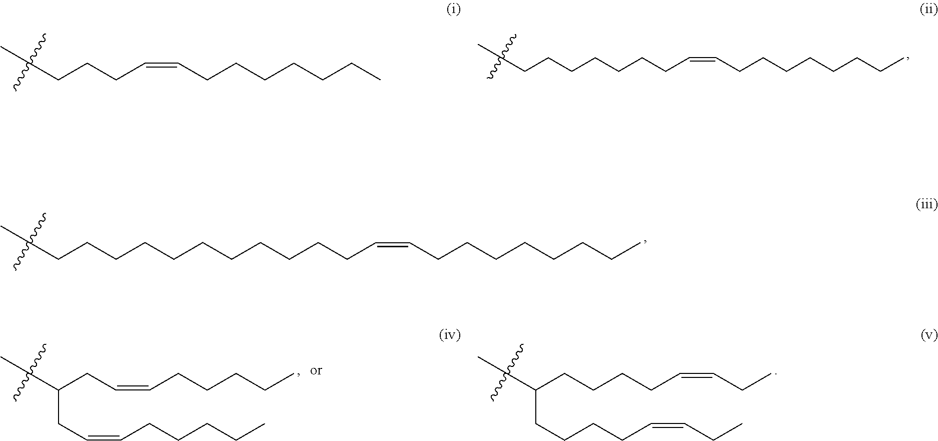 [SMILES: *CCC/C=C\CCCCCCC ; *CCCCCCC/C=C\CCCCCCCC ; *CCCCCCCCCCC/C=C\CCCCCCCC ; *C(C/C=C\CCCCC)C/C=C\CCCCC ; *C(CCCC/C=C\CC)CCCC/C=C\CC]

In embodiments, each R₂ is

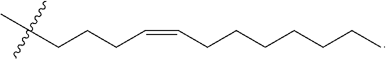 [SMILES: *CCC/C=C\CCCCCCC]

In embodiments, each R₂ is

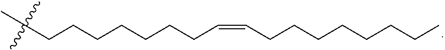 [SMILES: *CCCCCCC/C=C\CCCCCCCC]

In embodiments, each R₂ is

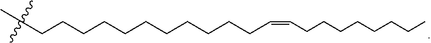 [SMILES: *CCCCCCCCCCC/C=C\CCCCCCCC]

In embodiments, each R₂ is

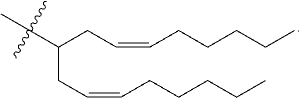 [SMILES: *C(C/C=C\CCCCC)C/C=C\CCCCC]

In embodiments, each R₂ is

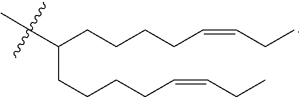 [SMILES: *C(CCCC/C=C\CC)CCCC/C=C\CC]

In embodiments, each R₂ is independently selected from

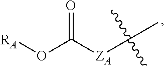

wherein each R_{A} is independently selected from optionally substituted C₁-C₃₁ alkyl, optionally substituted C₂-C₃₁alkenyl, and optionally substituted C₂-C₃₁ alkynyl; and wherein each Z_{A} is independently selected from optionally substituted C₁-C₁₀ alkylene and optionally substituted C₂-C₁₀ alkenylene.

In embodiments, each R_{A} is independently selected from optionally substituted C₁-C₃₁alkyl, for example optionally substituted C₄-C₂₄ alkyl, for example a branched C₄-C₂₄ alkyl. In some embodiments each R_{A} is independently selected from optionally substituted C₅-C₂₀ alkyl, each R_{A} is independently selected from optionally substituted C₃-C₁₅ alkyl.

In embodiments, each R_{A} is independently selected from a branched C₁₇ alkyl. In embodiments, each R_{A} is

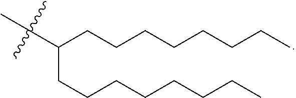 [SMILES: *C(CCCCCCCC)CCCCCCCC]

In embodiments, each R_{A} is independently selected from a branched C₁₅ alkyl. In embodiments, each R_{A} is

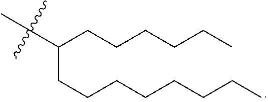 [SMILES: *C(CCCCCC)CCCCCCCC]

In embodiments, each R_{A} is independently selected from a branched C₁₃ alkyl. In embodiments, each R_{A} is

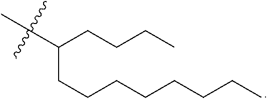 [SMILES: *C(CCCC)CCCCCCCC]

In embodiments, each R_{A} is independently selected from:

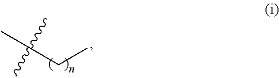

wherein each n is independently selected from 0, 1, 2, 3, 4, 5, 6, 7, 8, 9, 10, 11, 12, 13, 14, 15, 16 17, 18, 19, 20, 21, 22, 23, 24, 25, 26, 27, 28, 29, and 30,

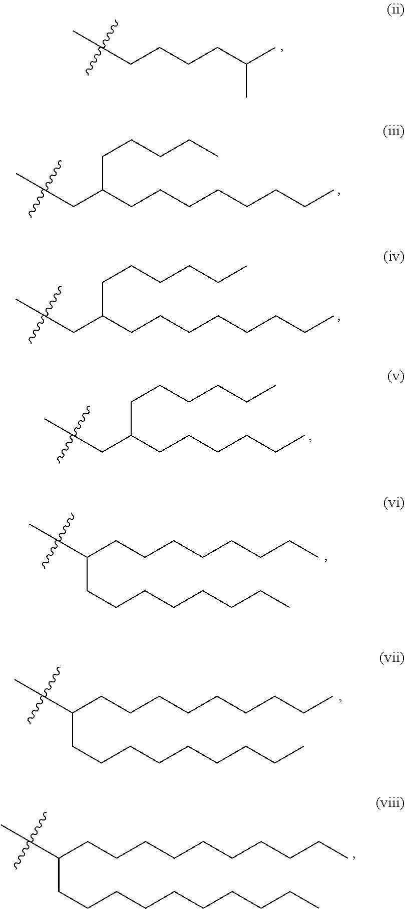

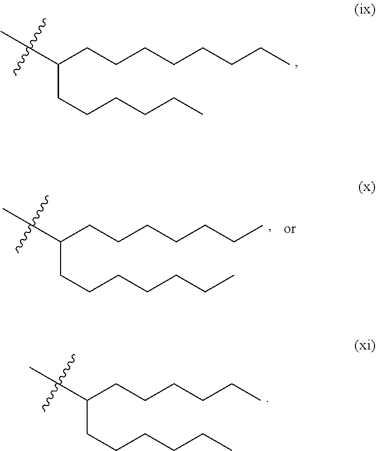 [SMILES: *C(CCCCCC)CCCCCCCC ; *C(CCCCCCC)CCCCCCC ; *C(CCCCCC)CCCCCC]

In embodiments, each R_{A} is independently selected from

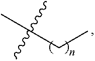

wherein each n is independently selected from 0, 1, 2, 3, 4, 5, 6, 7, 8, 9, 10, 11, 12, 13, 14, 15, 16 17, 18, 19, 20, 21, 22, 23, 24, 25, 26, 27, 28, 29, and 30. In embodiments, each n is 0. In embodiments, each n is 1. In embodiments, each n is 2. In embodiments, each n is 3. In embodiments, each n is 4. In embodiments, each n is 5. In embodiments, each n is 6. In embodiments, each n is 7. In embodiments, each n is 8. In embodiments, each n is 9. In embodiments, each n is 10. In embodiments, each n is 11. In embodiments, each n is 12. In embodiments, each n is 13. In embodiments, each n is 14. In embodiments, each n is 15. In embodiments, each n is 16. In embodiments, each n is 17. In embodiments, each n is 18. In embodiments, each n is 19. In embodiments, each n is 20. In embodiments, each n is 21. In embodiments, each n is 22. In embodiments, each n is 23. In embodiments, each n is 24. In embodiments, each n is 25. In embodiments, each n is 26. In embodiments, each n is 27. In embodiments, each n is 28. In embodiments, each n is 29. In embodiments, each n is 30.

In embodiments, each R_{A} is independently selected from

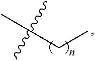

wherein each n is independently selected from 1-29. In embodiments, each R_{A} is independently selected from

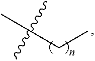

wherein each n is independently selected from 3-25. In embodiments, each R_{A} is independently selected from

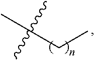

wherein each n is independently selected from 5-21. In embodiments, each R_{A} is independently selected from

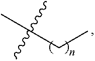

wherein each n is independently selected from 7-19.

In embodiments, each R_{A} is

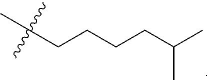 [SMILES: *CCCCC(C)C]

In embodiments, each R_{A} is

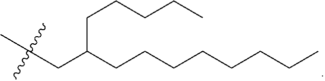 [SMILES: *CC(CCCCC)CCCCCCCC]

In embodiments, each R_{A} is

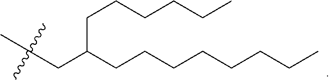 [SMILES: *CC(CCCCCC)CCCCCCCC]

In embodiments, each R_{A} is

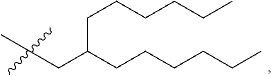 [SMILES: *CC(CCCCCC)CCCCCC]

In embodiments, each R_{A} is

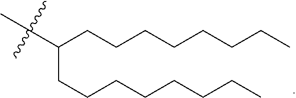 [SMILES: *C(CCCCCCCC)CCCCCCCC]

In embodiments, each R_{A} is

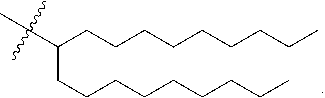 [SMILES: *C(CCCCCCCCC)CCCCCCCCC]

In embodiments, each R_{A} is

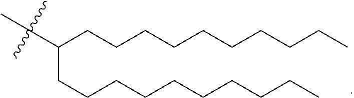 [SMILES: *C(CCCCCCCCCC)CCCCCCCCCC]

In embodiments, each R_{A} is

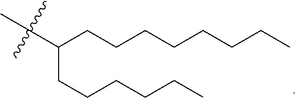 [SMILES: *C(CCCCCC)CCCCCCCC]

In embodiments, each R_{A} is

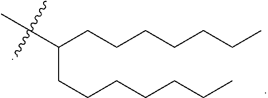

In embodiments, each R_{A} is

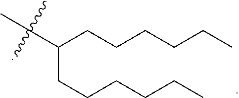

In embodiments, each R_{A} is independently selected from optionally substituted C₂-C₃₁ alkenyl, for example optionally substituted C₄-C₂₄ alkenyl.

In embodiments, each R_{A} is independently selected from:

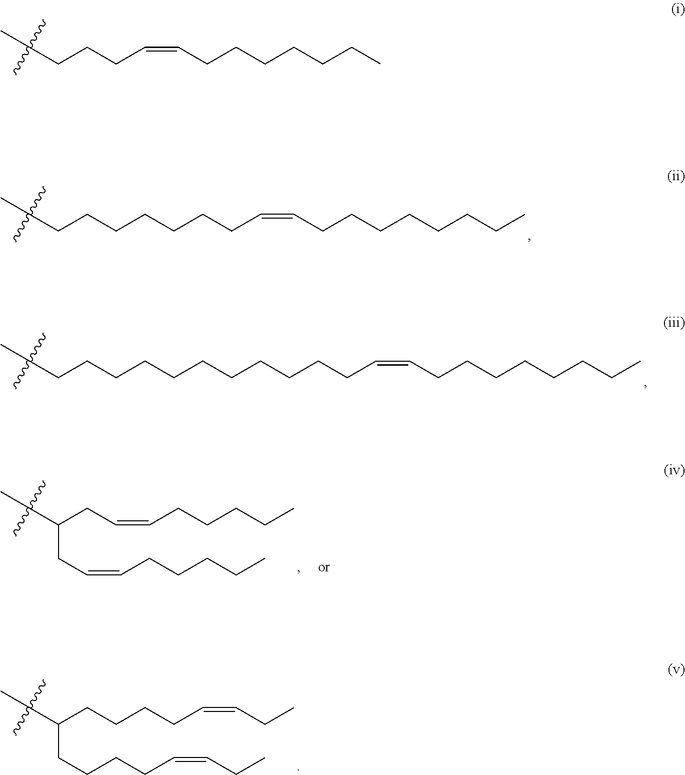 [SMILES: *CCC/C=C\CCCCCCC ; *CCCCCCC/C=C\CCCCCCCC ; *CCCCCCCCCCC/C=C\CCCCCCCC ; *C(C/C=C\CCCCC)C/C=C\CCCCC ; *C(CCCC/C=C\CC)CCCC/C=C\CC]

In embodiments, each R_{A} is

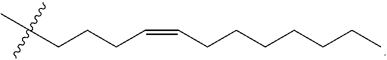 [SMILES: *CCC/C=C\CCCCCCC]

In embodiments, each R_{A} is

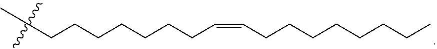 [SMILES: *CCCCCCC/C=C\CCCCCCCC]

In embodiments, each R_{A} is

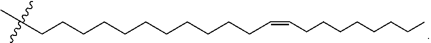 [SMILES: *CCCCCCCCCCC/C=C\CCCCCCCC]

In embodiments, each R_{A} is

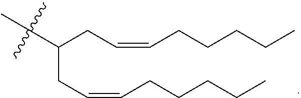 [SMILES: *C(C/C=C\CCCCC)C/C=C\CCCCC]

In embodiments, each R_{A} is

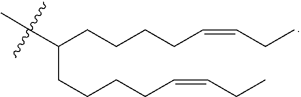 [SMILES: *C(CCCC/C=C\CC)CCCC/C=C\CC]

In embodiments, each Z_{A} is independently selected from optionally substituted C₁-C₁₀ alkylene, for example optionally substituted C₂-C₇ alkylene.

In embodiments, each Z_{A} is independently selected from optionally substituted C₆ alkylene. In embodiments, each Z_{A} is independently selected from unsubstituted C₆ alkylene. In embodiments, each Z_{A} is independently selected from unsubstituted straight-chain C₆ alkylene.

In embodiments, each Z_{A} is optionally substituted C₁ alkylene. In embodiments, each Z_{A} is optionally substituted C₂ alkylene. In embodiments, each Z_{A} is optionally substituted C₃ alkylene. In embodiments, each Z_{A} is optionally substituted C₄alkylene. In embodiments, each Z_{A} is optionally substituted C₅ alkylene. In embodiments, each Z_{A} is optionally substituted C₆alkylene. In embodiments, each Z_{A} is optionally substituted C₇alkylene. In embodiments, each Z_{A} is optionally substituted C₃ alkylene. In embodiments, each Z_{A} is optionally substituted C₉ alkylene. In embodiments, each Z_{A} is optionally substituted C₁₀ alkylene.

In embodiments, each Z_{A} is independently selected from optionally substituted C₂-C₁₀ alkenylene, for example optionally substituted C₂-C₇ alkenylene.

In embodiments, each Z_{A} is optionally substituted C₂ alkenylene. In embodiments, each Z_{A} is optionally substituted C₃ alkenylene. In embodiments, each Z_{A} is optionally substituted C₄ alkenylene. In embodiments, each Z_{A} is optionally substituted C₅ alkenylene. In embodiments, each Z_{A} is optionally substituted C₆ alkenylene. In embodiments, each Z_{A} is optionally substituted C₇ alkenylene. In embodiments, each Z_{A} is optionally substituted C₈ alkenylene. In embodiments, each Z_{A} is optionally substituted C₉ alkenylene. In embodiments, each Z_{A} is optionally substituted C₁₀ alkenylene.

In embodiments, Y if present is —CH₂CH₂—, each R_{A} is independently selected from optionally substituted C₁-C₃₁alkyl, and each Z_{A} is independently selected from optionally substituted C₁-C₁₀ alkylene.

In embodiments, each R_{A} is independently selected from optionally substituted C₁-C₃₁alkyl and each Z_{A} is C₁ alkylene. In embodiments, each R_{A} is independently selected from optionally substituted C₁-C₃₁alkyl and each Z_{A} is C₂ alkylene. In embodiments, each R_{A} is independently selected from optionally substituted C₁-C₃₁alkyl and each Z_{A} is C₃ alkylene. In embodiments, each R_{A} is independently selected from optionally substituted C₁-C₃₁ alkyl and each Z_{A} is C₄ alkylene. In embodiments, each R_{A} is independently selected from optionally substituted C₁-C₃₁alkyl and each Z_{A} is C₅ alkylene. In embodiments, each R_{A} is independently selected from optionally substituted C₁-C₃₁alkyl and each Z_{A} is C₆ alkylene. In embodiments, each R_{A} is independently selected from optionally substituted C₁-C₃₁alkyl and each Z_{A} is C₇ alkylene. In embodiments, each R_{A} is independently selected from optionally substituted C₁-C₃₁alkyl and each Z_{A} is C₃ alkylene. In embodiments, each R_{A} is independently selected from optionally substituted C₁-C₃₁alkyl and each Z_{A} is C₉ alkylene. In embodiments, each R_{A} is independently selected from optionally substituted C₁-C₃₁alkyl and each Z_{A} is C₁₀ alkylene.

In embodiments, each R_{A} is independently selected from optionally substituted C₄-C₂₄alkyl, and each Z_{A} is independently selected from optionally substituted C₂-C₇ alkylene.

In embodiments, each R_{A} is independently selected from optionally substituted C₄-C₂₄alkyl and each Z_{A} is C₂ alkylene. In embodiments, each R_{A} is independently selected from optionally substituted C₄-C₂₄alkyl and each Z_{A} is C₃ alkylene. In embodiments, each R_{A} is independently selected from optionally substituted C₄-C₂₄alkyl and each Z_{A} is C₄ alkylene. In embodiments, each R_{A} is independently selected from optionally substituted C₄-C₂₄alkyl and each Z_{A} is C₅ alkylene. In embodiments, each R_{A} is independently selected from optionally substituted C₄-C₂₄alkyl and each Z_{A} is C₆ alkylene. In embodiments, each R_{A} is independently selected from optionally substituted C₄-C₂₄alkyl and each Z_{A} is C₇ alkylene.

In embodiments, each R_{A} is

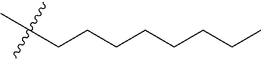 [SMILES: *CCCCCCCC]

and each Z_{A} is C₇ alkylene.

In embodiments, each R_{A} is

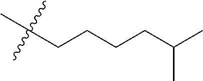 [SMILES: *CCCCC(C)C]

and each Z_{A} is C₆ alkylene.

In embodiments, each R_{A} is

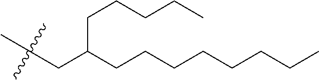 [SMILES: *CC(CCCCC)CCCCCCCC]

and each Z_{A} is C₂ alkylene.

In embodiments, each R_{A} is

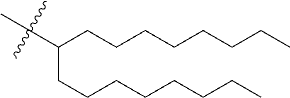 [SMILES: *C(CCCCCCCC)CCCCCCCC]

and each Z_{A} is independently selected from C₅-C₇ alkylene, for example wherein each Z_{A} is C₆ alkylene.

In embodiments, each R_{A} is

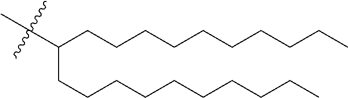 [SMILES: *C(CCCCCCCCCC)CCCCCCCCCC]

and each Z_{A} is C₆ alkylene.

In embodiments, Y if present is —CH₂CH₂—, each R_{A} is independently selected from optionally substituted C₁-C₃₁alkyl, and each Z_{A} is independently selected from optionally substituted C₂-C₁₀ alkenylene.

In embodiments, each R_{A} is independently selected from optionally substituted C₁-C₃₁alkyl and each Z_{A} is C₂ alkenylene. In embodiments, each R_{A} is independently selected from optionally substituted C₁-C₃₁alkyl and each Z_{A} is C₃ alkenylene. In embodiments, each R_{A} is independently selected from optionally substituted C₁-C₃₁alkyl and each Z_{A} is C₄ alkenylene. In embodiments, each R_{A} is independently selected from optionally substituted C₁-C₃₁alkyl and each Z_{A} is C₅ alkenylene. In embodiments, each R_{A} is independently selected from optionally substituted C₁-C₃₁alkyl and each Z_{A} is C₆ alkenylene. In embodiments, each R_{A} is independently selected from optionally substituted C₁-C₃₁alkyl and each Z_{A} is C₇ alkenylene.

In embodiments, each R_{A} is independently selected from optionally substituted C₄-C₂₄alkyl, and each Z_{A} is independently selected from optionally substituted C₂-C₇ alkenylene.

In embodiments, each R_{A} is independently selected from optionally substituted C₄-C₂₄alkyl and each Z_{A} is C₂ alkenylene. In embodiments, each R_{A} is independently selected from optionally substituted C₄-C₂₄alkyl and each Z_{A} is C₃ alkenylene. In embodiments, each R_{A} is independently selected from optionally substituted C₄-C₂₄alkyl and each Z_{A} is C₄ alkenylene. In embodiments, each R_{A} is independently selected from optionally substituted C₄-C₂₄alkyl and each Z_{A} is C₅ alkenylene. In embodiments, each R_{A} is independently selected from optionally substituted C₄-C₂₄alkyl and each Z_{A} is C₆ alkenylene. In embodiments, each R_{A} is independently selected from optionally substituted C₄-C₂₄alkyl and each Z_{A} is C₇ alkenylene.

In embodiments, each R_{A} is

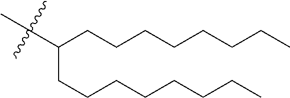 [SMILES: *C(CCCCCCCC)CCCCCCCC]

and each Z_{A} is C₆ alkenylene.

In embodiments, Y if present is —CH₂CH₂—, each R_{A} is independently selected from optionally substituted C₁-C₃₁alkenyl, and each Z_{A} is independently selected from optionally substituted C₁-C₁₀ alkylene.

In embodiments, each R_{A} is independently selected from optionally substituted C₁-C₃₁ alkenyl, and each Z_{A} is C₁ alkylene. In embodiments, each R_{A} is independently selected from optionally substituted C₁-C₃₁alkenyl, and each Z_{A} is C₂alkylene. In embodiments, each R_{A} is independently selected from optionally substituted C₁-C₃₁alkenyl, and each Z_{A} is C₃ alkylene. In embodiments, each R_{A} is independently selected from optionally substituted C₁-C₃₁alkenyl, and each Z_{A} is C₄alkylene. In embodiments, each R_{A} is independently selected from optionally substituted C₁-C₃₁alkenyl, and each Z_{A} is C₅ alkylene. In embodiments, each R_{A} is independently selected from optionally substituted C₁-C₃₁alkenyl, and each Z_{A} is C₆alkylene. In embodiments, each R_{A} is independently selected from optionally substituted C₁-C₃₁alkenyl, and each Z_{A} is C₇ alkylene. In embodiments, each R_{A} is independently selected from optionally substituted C₁-C₃₁ alkenyl, and each Z_{A} is C₈ alkylene. In embodiments, each R_{A} is independently selected from optionally substituted C₁-C₃₁alkenyl, and each Z_{A} is C₉alkylene. In embodiments, each R_{A} is independently selected from optionally substituted C₁-C₃₁alkenyl, and each Z_{A} is C₁₀ alkylene.

In embodiments, each R_{A} is independently selected from optionally substituted C₄-C₂₄ alkenyl, and each Z_{A} is independently selected from optionally substituted C₂-C₇ alkylene.

In embodiments, each R_{A} is independently selected from optionally substituted C₄-C₂₄ alkenyl, and each Z_{A} is C₂ alkylene. In embodiments, each R_{A} is independently selected from optionally substituted C₄-C₂₄alkenyl, and each Z_{A} is C₃ alkylene. In embodiments, each R_{A} is independently selected from optionally substituted C₄-C₂₄alkenyl, and each Z_{A} is C₄alkylene. In embodiments, each R_{A} is independently selected from optionally substituted C₄-C₂₄alkenyl, and each Z_{A} is C₅ alkylene. In embodiments, each R_{A} is independently selected from optionally substituted C₄-C₂₄alkenyl, and each Z_{A} is C₆alkylene. In embodiments, each R_{A} is independently selected from optionally substituted C₄-C₂₄alkenyl, and each Z_{A} is C₇alkylene.

In embodiments, each R_{A} is

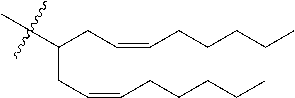 [SMILES: *C(C/C=C\CCCCC)C/C=C\CCCCC]

and each Z_{A} is C₆ alkylene.

In embodiments, each R_{A} is

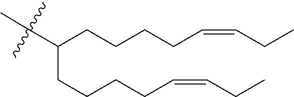 [SMILES: *C(CCCC/C=C\CC)CCCC/C=C\CC]

and each Z_{A} is C₆ alkylene.

In embodiments, each R₂ is independently selected from

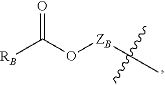

wherein each R_{B} is independently selected from optionally substituted C₁-C₃₁ alkyl, optionally substituted C₁-C₃₁alkenyl, and optionally substituted C₁-C₃₁ alkynyl; and wherein each Z_{B} is independently selected from optionally substituted C₁-C₁₀alkylene and optionally substituted C₂-C₁₀ alkenylene.

In embodiments, each R_{B} is independently selected from optionally substituted C₁-C₃₁ alkyl, for example optionally substituted C₄-C₂₄ alkyl, for example a branched C₄-C₂₄ alkyl.

In embodiments, each R_{B} is independently selected from a branched C₁₇ alkyl. In embodiments, each R_{B} is

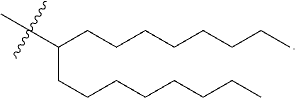 [SMILES: *C(CCCCCCCC)CCCCCCCC]

In embodiments, each R_{B} is independently selected from a branched C₁₅ alkyl. In embodiments, each R_{B} is

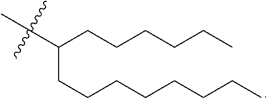 [SMILES: *C(CCCCCC)CCCCCCCC]

In embodiments, each R_{B} is independently selected from a branched C₁₃ alkyl. In embodiments, each R_{B} is

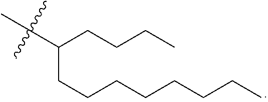 [SMILES: *C(CCCC)CCCCCCCC]

In embodiments, each R_{B} is independently selected from:

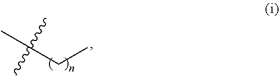

(i) n, wherein each n is independently selected from 0, 1, 2, 3, 4, 5, 6, 7, 8, 9, 10, 11, 12, 13, 14, 15, 16 17, 18, 19, 20, 21, 22, 23, 24, 25, 26, 27, 28, 29, and 30,

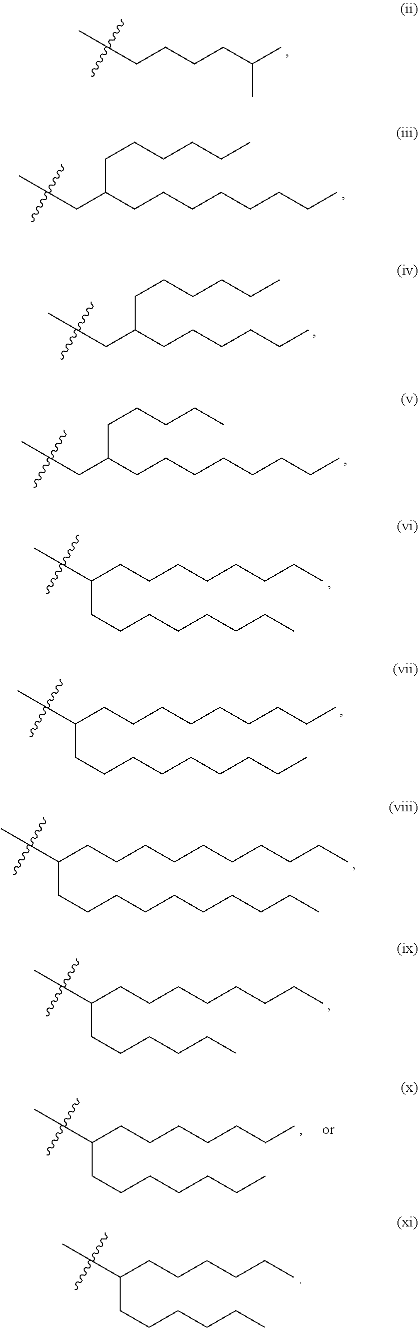

In embodiments, each R_{B} is independently selected from

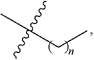

wherein each n is independently selected from 0, 1, 2, 3, 4, 5, 6, 7, 8, 9, 10, 11, 12, 13, 14, 15, 16 17, 18, 19, 20, 21, 22, 23, 24, 25, 26, 27, 28, 29, and 30. In embodiments, each n is 0. In embodiments, each n is 1. In embodiments, each n is 2. In embodiments, each n is 3. In embodiments, each n is 4. In embodiments, each n is 5. In embodiments, each n is 6. In embodiments, each n is 7. In embodiments, each n is 8. In embodiments, each n is 9. In embodiments, each n is 10. In embodiments, each n is 11. In embodiments, each n is 12. In embodiments, each n is 13. In embodiments, each n is 14. In embodiments, each n is 15. In embodiments, each n is 16. In embodiments, each n is 17. In embodiments, each n is 18. In embodiments, each n is 19. In embodiments, each n is 20. In embodiments, each n is 21. In embodiments, each n is 22. In embodiments, each n is 23. In embodiments, each n is 24. In embodiments, each n is 25. In embodiments, each n is 26. In embodiments, each n is 27. In embodiments, each n is 28. In embodiments, each n is 29. In embodiments, each n is 30.

In embodiments, each R_{B} is independently selected from

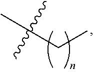

wherein each n is independently selected from 1-29. In embodiments, each R_{B} is independently selected from

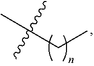

wherein each n is independently selected from 3-25. In embodiments, each R_{B} is independently selected from

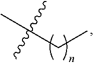

wherein each n is independently selected from 5-21. In embodiments, each R_{B} is independently selected from

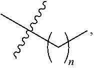

wherein each n is independently selected from 7-19

In embodiments, each R_{B} is

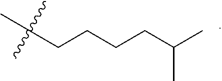 [SMILES: *CCCCC(C)C]

In embodiments each R_{B} is

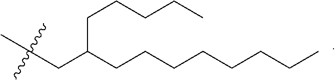 [SMILES: *CC(CCCCC)CCCCCCCC]

In embodiments, each R_{B} is

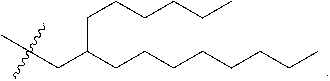 [SMILES: *CC(CCCCCC)CCCCCCCC]

In embodiments, each R_{B} is

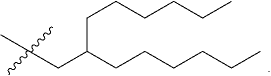 [SMILES: *CC(CCCCCC)CCCCCC]

In embodiments, each R_{B} is

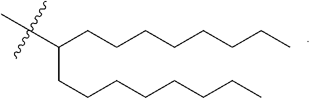 [SMILES: *C(CCCCCCCC)CCCCCCCC]

In embodiments, each R_{B} is

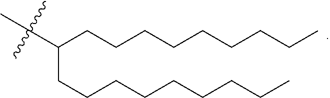 [SMILES: *C(CCCCCCCCC)CCCCCCCCC]

In embodiments, each R_{B} is

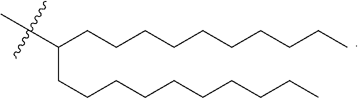 [SMILES: *C(CCCCCCCCCC)CCCCCCCCCC]

In embodiments, each R_{B} is

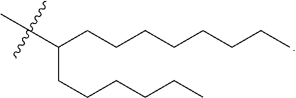 [SMILES: *C(CCCCCC)CCCCCCCC]

In embodiments, each R_{B} is

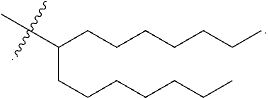

In embodiments, each R_{B} is

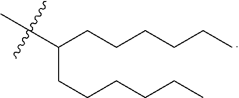 [SMILES: *C(CCCCCC)CCCCCC]

In embodiments, each R_{B} is independently selected from optionally substituted C₂-C₃₁ alkenyl, for example optionally substituted C₄-C₂₄ alkenyl.

In embodiments, each R_{B} is independently selected from:

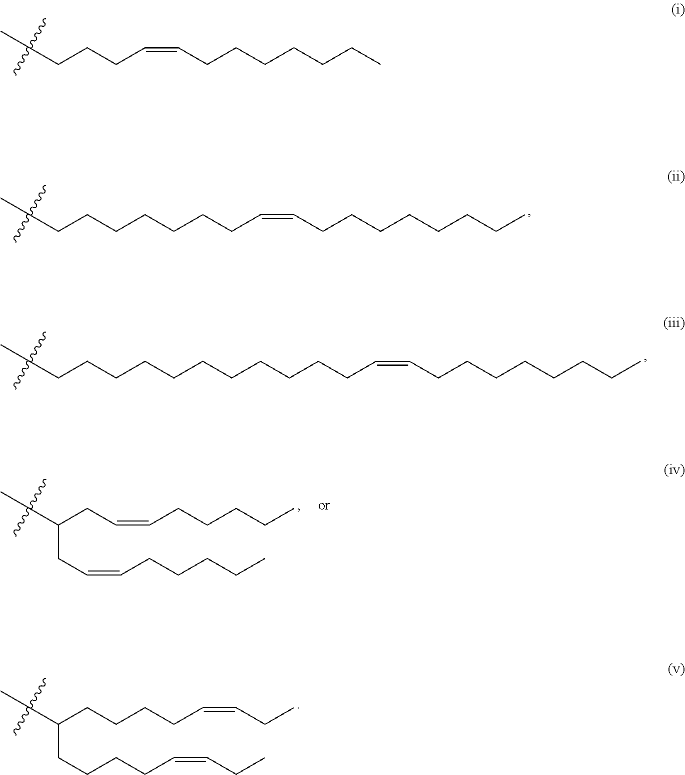 [SMILES: *CCC/C=C\CCCCCCC ; *CCCCCCC/C=C\CCCCCCCC ; *CCCCCCCCCCC/C=C\CCCCCCCC ; *C(C/C=C\CCCCC)C/C=C\CCCCC ; *C(CCCC/C=C\CC)CCCC/C=C\CC]

In embodiments, each R_{B} is

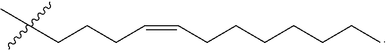 [SMILES: *CCC/C=C\CCCCCCC]

In embodiments, each R_{B} is

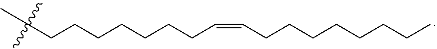 [SMILES: *CCCCCCC/C=C\CCCCCCCC]

In embodiments, each R_{B} is

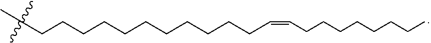 [SMILES: *CCCCCCCCCCC/C=C\CCCCCCCC]

In embodiments, each R_{B} is

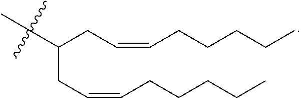 [SMILES: *C(C/C=C\CCCCC)C/C=C\CCCCC]

In embodiments, each R_{B} is

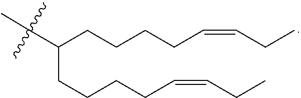 [SMILES: *C(CCCC/C=C\CC)CCCC/C=C\CC]

In embodiments, each Z_{B} is independently selected from optionally substituted C₁-C₁₀ alkylene, for example optionally substituted C₂-C₇ alkylene.

In embodiments, each Z_{B} is independently selected from optionally substituted C₆ alkylene. In embodiments, each Z_{B} is independently selected from unsubstituted C₆ alkylene. In embodiments, each Z_{B} is independently selected from unsubstituted straight-chain C₆ alkylene. In embodiments, each Z_{B} is optionally substituted C₁ alkylene. In embodiments, each Z_{B} is optionally substituted C₂ alkylene. In embodiments, each Z_{B} is optionally substituted C₃ alkylene. In embodiments, each Z_{B} is optionally substituted C₄alkylene. In embodiments, each Z_{B} is optionally substituted C₈ alkylene. In embodiments, each Z_{B} is optionally substituted C₆alkylene. In embodiments, each Z_{B} is optionally substituted C₇ alkylene. In embodiments, each Z_{B} is optionally substituted C₅ alkylene. In embodiments, each Z_{B} is optionally substituted C₉ alkylene. In embodiments, each Z_{B} is optionally substituted C₁₀ alkylene.

In embodiments, each Z_{B} is independently selected from optionally substituted C₂-C₁₀ alkenylene, for example optionally substituted C₂-C₇ alkenylene.

In embodiments, each Z_{B} is optionally substituted C₂ alkenylene. In embodiments, each Z_{B} is optionally substituted C₃ alkenylene. In embodiments, each Z_{B} is optionally substituted C₄ alkenylene. In embodiments, each Z_{B} is optionally substituted C₅ alkenylene. In embodiments, each Z_{B} is optionally substituted C₆ alkenylene. In embodiments, each Z_{B} is optionally substituted C₇ alkenylene. In embodiments, each Z_{B} is optionally substituted C₈ alkenylene. In embodiments, each Z_{B} is optionally substituted C₉ alkenylene. In embodiments, each Z_{B} is optionally substituted C₁₀ alkenylene.

In embodiments, Y if present is —CH₂CH₂—, each R_{B} is independently selected from optionally substituted C₁-C₃₁alkyl, and each Z_{B} is independently selected from optionally substituted C₁-C₁₀ alkylene.

In embodiments, each R_{B} is independently selected from optionally substituted C₁-C₃₁ alkyl and each Z_{B} is C₁ alkylene. In embodiments, each R_{B} is independently selected from optionally substituted C₁-C₃₁alkyl and each Z_{B} is C₂ alkylene. In embodiments, each R_{B} is independently selected from optionally substituted C₁-C₃₁alkyl and each Z_{B} is C₃ alkylene. In embodiments, each R_{B} is independently selected from optionally substituted C₁-C₃₁alkyl and each Z_{B} is C₄ alkylene. In embodiments, each R_{B} is independently selected from optionally substituted C₁-C₃₁alkyl and each Z_{B} is C₅ alkylene. In embodiments, each R_{B} is independently selected from optionally substituted C₁-C₃₁alkyl and each Z_{B} is C₆ alkylene. In embodiments, each R_{B} is independently selected from optionally substituted C₁-C₃₁alkyl and each Z_{B} is C₇ alkylene. In embodiments, each R_{B} is independently selected from optionally substituted C₁-C₃₁alkyl and each Z_{B} is C₈ alkylene. In embodiments, each R_{B} is independently selected from optionally substituted C₁-C₃₁alkyl and each Z_{B} is C₉ alkylene. In embodiments, each R_{B} is independently selected from optionally substituted C₁-C₃₁alkyl and each Z_{B} is C₁₀ alkylene.

In embodiments, each R_{B} is independently selected from optionally substituted C₄-C₂₄alkyl, and each Z_{B} is independently selected from optionally substituted C₂-C₇ alkylene.

In embodiments, each R_{B} is independently selected from optionally substituted C₄-C₂₄alkyl and each Z_{B} is C₂ alkylene. In embodiments, each R_{B} is independently selected from optionally substituted C₄-C₂₄alkyl and each Z_{B} is C₃ alkylene. In embodiments, each R_{B} is independently selected from optionally substituted C₄-C₂₄alkyl and each Z_{B} is C₄ alkylene. In embodiments, each R_{B} is independently selected from optionally substituted C₄-C₂₄alkyl and each Z_{B} is C₅ alkylene. In embodiments, each R_{B} is independently selected from optionally substituted C₄-C₂₄alkyl and each Z_{B} is C₆ alkylene. In embodiments, each R_{B} is independently selected from optionally substituted C₄-C₂₄alkyl and each Z_{B} is C₇ alkylene.

In embodiments, each R_{B} is independently selected from:

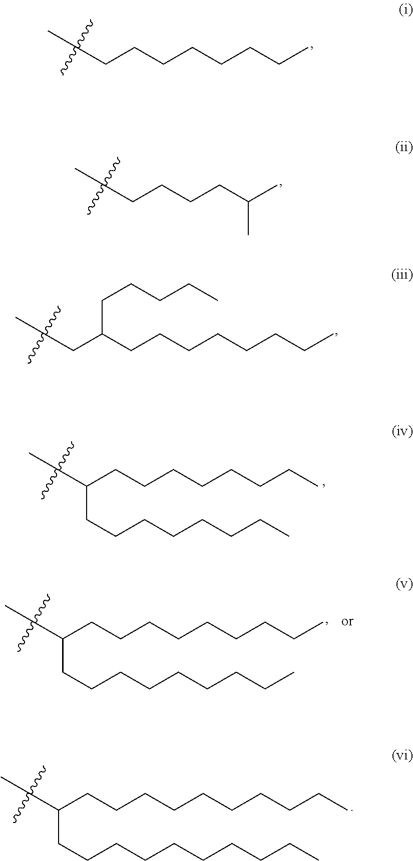

In embodiments, each R_{B} is

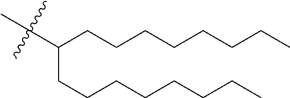 [SMILES: *C(CCCCCCCC)CCCCCCCC]

and each Z_{B} is C₅ alkylene.

In embodiments, each R_{B} is

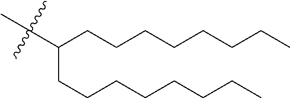 [SMILES: *C(CCCCCCCC)CCCCCCCC]

and each Z_{B} is C₆ alkylene.

In embodiments, one R₂ is optionally substituted C₄-C₂₄alkyl, optionally substituted C₄-C₂₄ alkenyl, or optionally substituted C₄-C₂₄alkynyl, and the other R₂ is

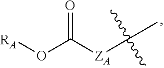

wherein each R_{A} is independently selected from optionally substituted C₁-C₃₁ alkyl, optionally substituted C₂-C₃₁alkenyl, and optionally substituted C₂-C₃₁ alkynyl; and wherein each Z_{A} is independently selected from optionally substituted C₁-C₁₀ alkylene and optionally substituted C₂-C₁₀ alkenylene.

In embodiments, one R₂ is optionally substituted C₄-C₂₄alkyl, optionally substituted C₄-C₂₄ alkenyl, or optionally substituted C₄-C₂₄alkynyl, and the other R₂ is

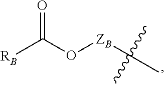

wherein each R_{B} is independently selected from optionally substituted C₁-C₃₁ alkyl, optionally substituted C₁-C₃₁ alkenyl, and optionally substituted C₁-C₃₁ alkynyl; and wherein each Z_{B} is independently selected from optionally substituted C₁-C₁₀ alkylene and optionally substituted C₂-C₁₀ alkenylene.

In embodiments, each R_{A}, when present, is the same. In embodiments, each R_{A}, when present, is different.

In embodiments, each Z_{A}, when present, is the same. In embodiments, each Z_{A}, when present, is different.

In embodiments, each R_{B}, when present, is the same. In embodiments, each R_{B}, when present, is different.

In embodiments, each Z_{B}, when present, is the same. In embodiments, each Z_{B}, when present, is different.

In embodiments, each R_{A}, when present, is independently selected from linear C₁-C₃₁ alkyl. In embodiments, each R_{A}, when present, is independently selected from branched C₁-C₃₁ alkyl. In embodiments, each R_{A}, when present, is independently selected from linear C₄-C₂₄ alkyl. In embodiments, each R_{A}, when present, is independently selected from branched C₄-C₂₄ alkyl. In embodiments, one R_{A} is linear C₁-C₃₁ alkyl and the other R_{A} is branched C₁-C₃₁ alkyl. In embodiments, one R_{A} is linear C₄-C₂₄ alkyl and the other R_{A} is branched C₄-C₂₄ alkyl.

In embodiments, one R_{A} is

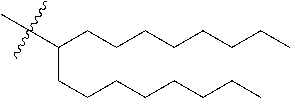 [SMILES: *C(CCCCCCCC)CCCCCCCC]

and the other R_{A} is C₁-C₃₁ alkyl. In embodiments, one R_{A} is

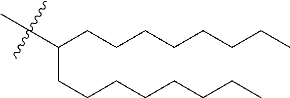 [SMILES: *C(CCCCCCCC)CCCCCCCC]

and the other R_{A} is C₄-C₂₄ alkyl. In embodiments, one R_{A} is

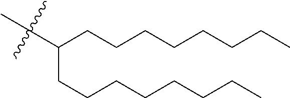 [SMILES: *C(CCCCCCCC)CCCCCCCC]

and the other R_{A} is C₃ alkyl.

In embodiments, each R_{B}, when present, is independently selected from linear C₁-C₃₁ alkyl. In embodiments, each R_{B}, when present, is independently selected from branched C₁-C₃₁ alkyl. In embodiments, each R_{B}, when present, is independently selected from linear C₄-C₂₄ alkyl. In embodiments, each R_{B}, when present, is independently selected from branched C₄-C₂₄ alkyl.

In embodiments, one R_{A} is linear C1-C₃₁ alkyl and the other R_{A} is branched C1-C₃₁ alkyl. In embodiments, one R_{A} is linear C₄-C₂₄ alkyl and the other R_{A} is branched C₄-C₂₄ alkyl.

In embodiments, the helper lipids of the present invention have any one of the structures in Table A, or a pharmaceutically acceptable salt thereof.

In embodiments, the helper lipid is

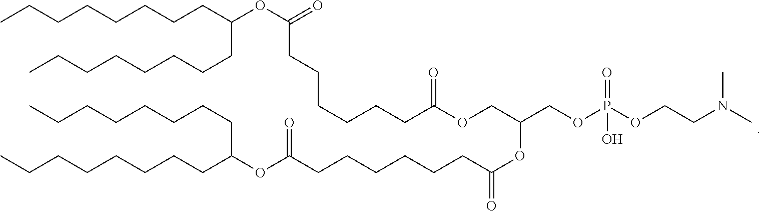 [SMILES: CCCCCCCCC(CCCCCCCC)OC(=O)CCCCCCC(=O)OCC(COP(=O)(O)OCCN(C)C)OC(=O)CCCCCCC(=O)OC(CCCCCCCC)CCCCCCCC]

In embodiments, the helper lipid is

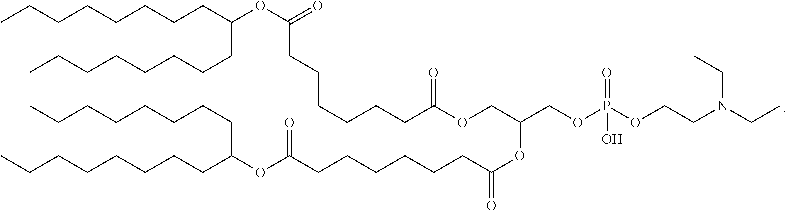 [SMILES: CCCCCCCCC(CCCCCCCC)OC(=O)CCCCCCC(=O)OCC(COP(=O)(O)OCCN(CC)CC)OC(=O)CCCCCCC(=O)OC(CCCCCCCC)CCCCCCCC]

In embodiments, the helper lipid is

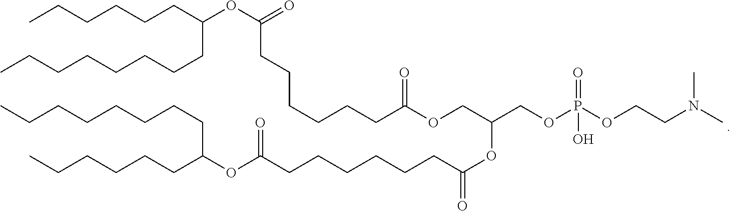 [SMILES: CCCCCCCCC(CCCCCC)OC(=O)CCCCCCC(=O)OCC(COP(=O)(O)OCCN(C)C)OC(=O)CCCCCCC(=O)OC(CCCCCC)CCCCCCCC]

In embodiments, the helper lipid is

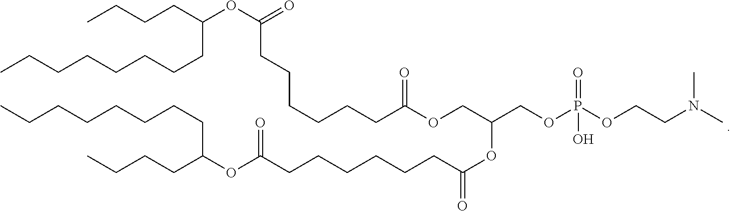 [SMILES: CCCCCCCCC(CCCC)OC(=O)CCCCCCC(=O)OCC(COP(=O)(O)OCCN(C)C)OC(=O)CCCCCCC(=O)OC(CCCC)CCCCCCCC]

In embodiments, the helper lipid is

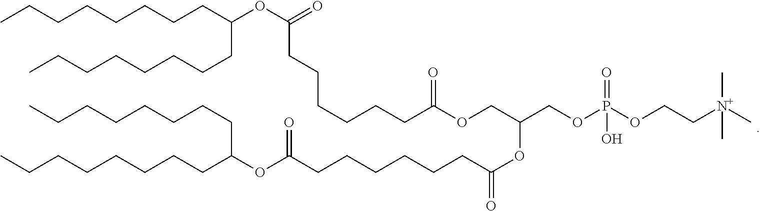 [SMILES: CCCCCCCCC(CCCCCCCC)OC(=O)CCCCCCC(=O)OCC(COP(=O)(O)OCC[N+](C)(C)C)OC(=O)CCCCCCC(=O)OC(CCCCCCCC)CCCCCCCC]

In embodiments, the helper lipid is

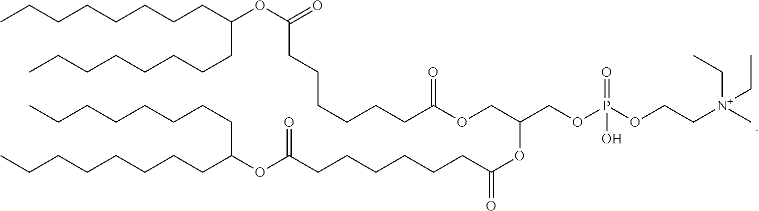 [SMILES: CCCCCCCCC(CCCCCCCC)OC(=O)CCCCCCC(=O)OCC(COP(=O)(O)OCC[N+](C)(CC)CC)OC(=O)CCCCCCC(=O)OC(CCCCCCCC)CCCCCCCC]

In embodiments, the helper lipid is

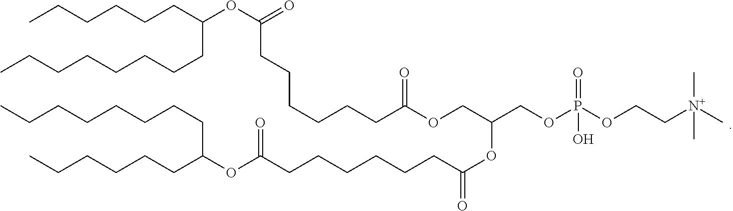 [SMILES: CCCCCCCCC(CCCCCC)OC(=O)CCCCCCC(=O)OCC(COP(=O)(O)OCC[N+](C)(C)C)OC(=O)CCCCCCC(=O)OC(CCCCCC)CCCCCCCC]

In embodiments, the helper lipid is

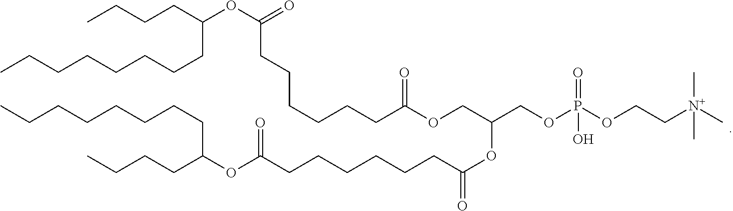 [SMILES: CCCCCCCCC(CCCC)OC(=O)CCCCCCC(=O)OCC(COP(=O)(O)OCC[N+](C)(C)C)OC(=O)CCCCCCC(=O)OC(CCCC)CCCCCCCC]

In embodiments, the helper lipid is

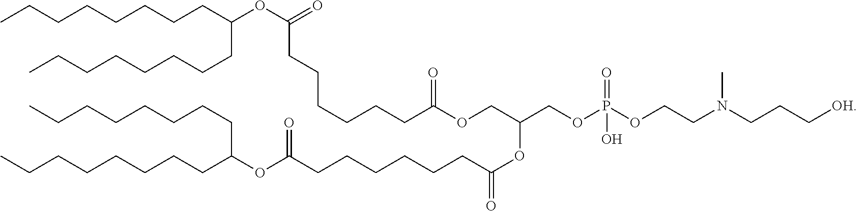 [SMILES: CCCCCCCCC(CCCCCCCC)OC(=O)CCCCCCC(=O)OCC(COP(=O)(O)OCCN(C)CCCO)OC(=O)CCCCCCC(=O)OC(CCCCCCCC)CCCCCCCC]

In embodiments, provided herein is a composition comprising one or more helper lipid(s) of the present invention or a pharmaceutically acceptable salt thereof, and further comprising:
(i) one or more cationic lipids,
(ii) one or more sterol-based lipids, and
(iii) one or more PEG-modified lipids.

In embodiments, the one or more sterol-based lipids is a cholesterol-based lipid, for example cholesterol.

In embodiments, the composition is a lipid nanoparticle, optionally a liposome. In embodiments, the one or more cationic lipid(s) constitute(s) about 20 mol % to about 60 mol % of the lipid nanoparticle. In embodiments, the one or more lipid(s) of the invention constitute(s) about 10 mol % to about 50 mol % of the lipid nanoparticle. In embodiments, the one or more PEG-modified lipid(s) constitute(s) about 1 mol % to about 4 mol % of the lipid nanoparticle. In embodiments, the one or more sterol-based lipids constitute(s) about 10 mol % to about 50 mol % of the lipid nanoparticle.

In embodiments, the lipid nanoparticle encapsulates a nucleic acid, optionally an mRNA encoding a peptide or protein. In embodiments, the lipid nanoparticle encapsulates an mRNA encoding a peptide or protein, optionally for use in a vaccine. In embodiments, the peptide is an antigen. As used herein, the phrase “encapsulation percentage” refers to the fraction of therapeutic agent (e.g. mRNA) that is effectively encapsulated within a liposomal-based vehicle (e.g. a lipid nanoparticle) relative to the initial fraction of therapeutic agent present in the lipid phase. In embodiments, the lipid nanoparticles have an encapsulation percentage for mRNA of at least 50%. In embodiments, the lipid nanoparticles have an encapsulation percentage for mRNA of at least 55%. In embodiments, the lipid nanoparticles have an encapsulation percentage for mRNA of at least 60%. In embodiments, the lipid nanoparticles have an encapsulation percentage for mRNA of at least 65%. In embodiments, the lipid nanoparticles have an encapsulation percentage for mRNA of at least 70%. In embodiments, the lipid nanoparticles have an encapsulation percentage for mRNA of at least 75%. In embodiments, the lipid nanoparticles have an encapsulation percentage for mRNA of at least 80%. In embodiments, the lipid nanoparticles have an encapsulation percentage for mRNA of at least 85%. In embodiments, the lipid nanoparticles have an encapsulation percentage for mRNA of at least 90%. In embodiments, the lipid nanoparticles have an encapsulation percentage for mRNA of at least 95%. In embodiments, the encapsulation percentage is calculated by performing the Ribogreen assay (Invitrogen) with and without the presence of 0.1% Triton-X 100.

In embodiments, the composition of the present invention is for use in therapy.

In embodiments, the composition of the present invention is for use in a method of treating or preventing a disease amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

In embodiments, a method for treating or preventing a disease is provided, wherein said method comprises administering to a subject in need thereof a composition of the present invention and wherein the disease is amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

In embodiments, the composition is administered intravenously, intrathecally, or intramuscularly, or by pulmonary delivery, optionally through nebulization. In embodiments, the composition is administered intramuscularly.

### Exemplary Compounds

In embodiments, the helper lipids of the present invention include compounds selected from those depicted in Table A, or a pharmaceutically acceptable salt thereof.

Exemplary compounds include those described in Table A, or a pharmaceutically acceptable salt thereof.

TABLE A

| Compound | |
|---|---|
| # | Compound structure |
| 1 | 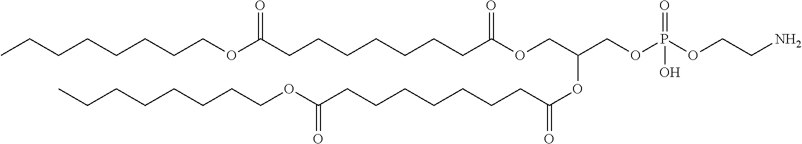 [SMILES: CCCCCCCCOC(=O)CCCCCCCC(=O)OCC(COP(=O)(O)OCCN)OC(=O)CCCCCCCC(=O)OCCCCCCCC] |
| 2 | 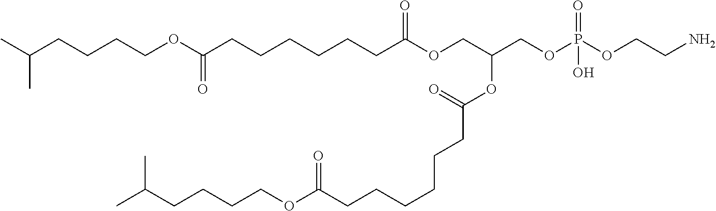 [SMILES: CC(C)CCCCOC(=O)CCCCCCC(=O)OCC(COP(=O)(O)OCCN)OC(=O)CCCCCCC(=O)OCCCCC(C)C] |
| 3 | 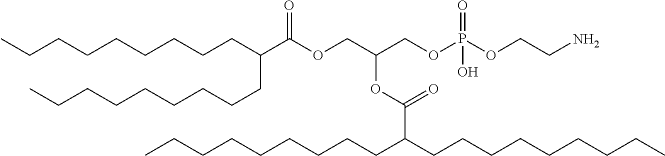 [SMILES: CCCCCCCCCC(CCCCCCCCC)C(=O)OCC(COP(=O)(O)OCCN)OC(=O)C(CCCCCCCCC)CCCCCCCCC] |
| 4 | 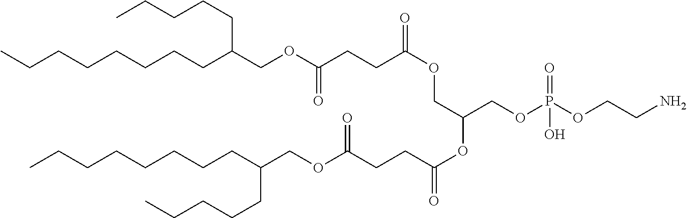 [SMILES: CCCCCCCCC(CCCCC)COC(=O)CCC(=O)OCC(COP(=O)(O)OCCN)OC(=O)CCC(=O)OCC(CCCCC)CCCCCCCC] |
| 5 | 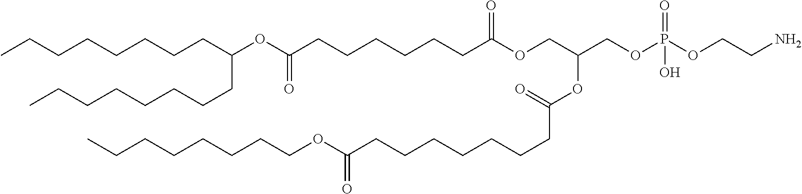 [SMILES: CCCCCCCCOC(=O)CCCCCCCC(=O)OC(COC(=O)CCCCCCC(=O)OC(CCCCCCCC)CCCCCCCC)COP(=O)(O)OCCN] |
| 6 | 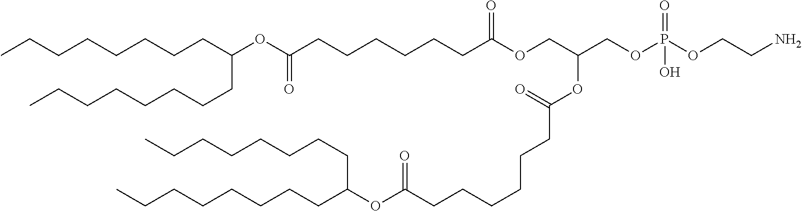 [SMILES: CCCCCCCCC(CCCCCCCC)OC(=O)CCCCCCC(=O)OCC(COP(=O)(O)OCCN)OC(=O)CCCCCCC(=O)OC(CCCCCCCC)CCCCCCCC] |
| 7 | 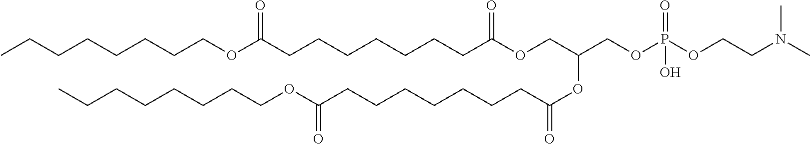 [SMILES: CCCCCCCCOC(=O)CCCCCCCC(=O)OCC(COP(=O)(O)OCCN(C)C)OC(=O)CCCCCCCC(=O)OCCCCCCCC] |
| 8 | 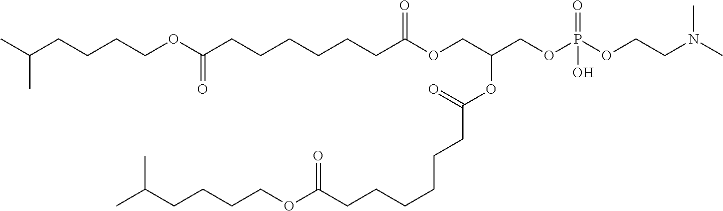 [SMILES: CC(C)CCCCOC(=O)CCCCCCC(=O)OCC(COP(=O)(O)OCCN(C)C)OC(=O)CCCCCCC(=O)OCCCCC(C)C] |
| 9 | 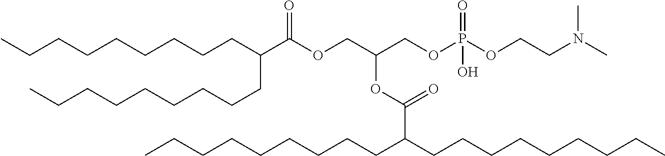 [SMILES: CCCCCCCCCC(CCCCCCCCC)C(=O)OCC(COP(=O)(O)OCCN(C)C)OC(=O)C(CCCCCCCCC)CCCCCCCCC] |
| 10 | 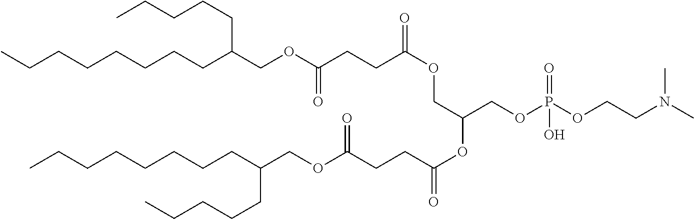 [SMILES: CCCCCCCCC(CCCCC)COC(=O)CCC(=O)OCC(COP(=O)(O)OCCN(C)C)OC(=O)CCC(=O)OCC(CCCCC)CCCCCCCC] |
| 11 | 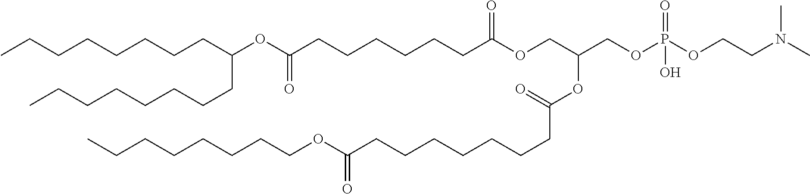 [SMILES: CCCCCCCCOC(=O)CCCCCCCC(=O)OC(COC(=O)CCCCCCC(=O)OC(CCCCCCCC)CCCCCCCC)COP(=O)(O)OCCN(C)C] |
| 12 | 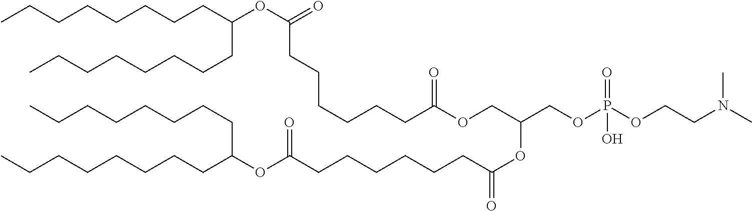 [SMILES: CCCCCCCCC(CCCCCCCC)OC(=O)CCCCCCC(=O)OCC(COP(=O)(O)OCCN(C)C)OC(=O)CCCCCCC(=O)OC(CCCCCCCC)CCCCCCCC] |
| 13 | 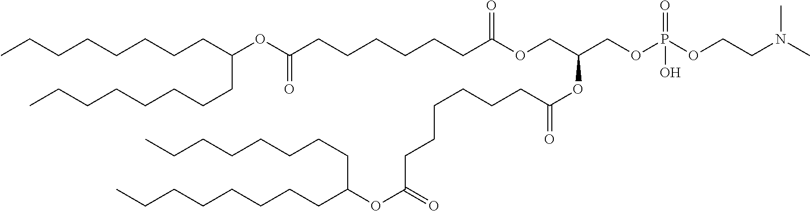 [SMILES: CCCCCCCCC(CCCCCCCC)OC(=O)CCCCCCC(=O)OC[C@@H](COP(=O)(O)OCCN(C)C)OC(=O)CCCCCCC(=O)OC(CCCCCCCC)CCCCCCCC] |
| 14 | 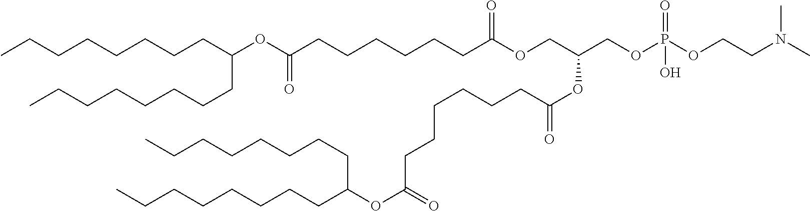 [SMILES: CCCCCCCCC(CCCCCCCC)OC(=O)CCCCCCC(=O)OC[C@H](COP(=O)(O)OCCN(C)C)OC(=O)CCCCCCC(=O)OC(CCCCCCCC)CCCCCCCC] |
| 15 | 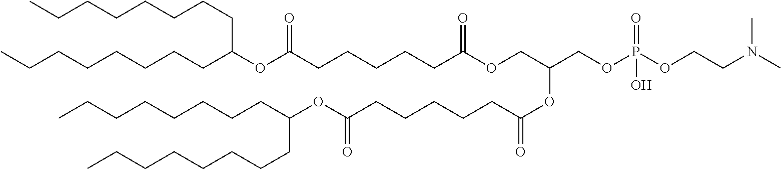 [SMILES: CCCCCCCCC(CCCCCCCC)OC(=O)CCCCCC(=O)OCC(COP(=O)(O)OCCN(C)C)OC(=O)CCCCCC(=O)OC(CCCCCCCC)CCCCCCCC] |
| 16 | 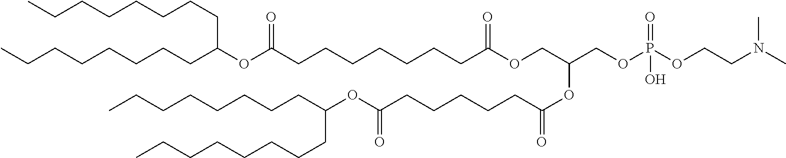 [SMILES: CCCCCCCCC(CCCCCCCC)OC(=O)CCCCCCCC(=O)OCC(COP(=O)(O)OCCN(C)C)OC(=O)CCCCCC(=O)OC(CCCCCCCC)CCCCCCCC] |
| 17 | 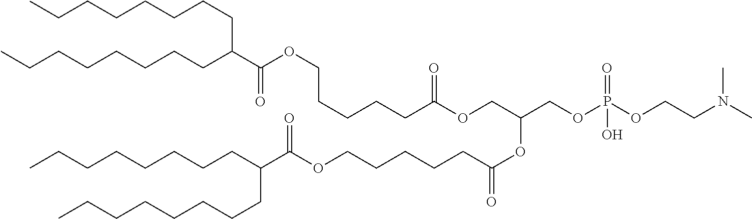 [SMILES: CCCCCCCCC(CCCCCCCC)C(=O)OCCCCCC(=O)OCC(COP(=O)(O)OCCN(C)C)OC(=O)CCCCCOC(=O)C(CCCCCCCC)CCCCCCCC] |
| 18 | 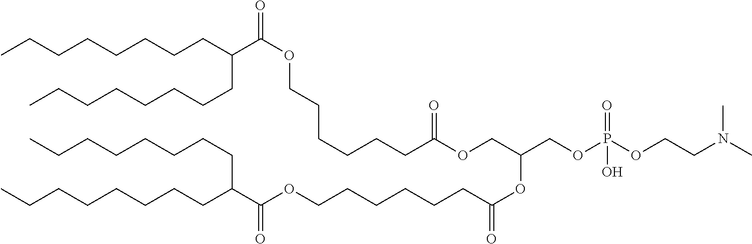 [SMILES: CCCCCCCCC(CCCCCCCC)C(=O)OCCCCCCC(=O)OCC(COP(=O)(O)OCCN(C)C)OC(=O)CCCCCCOC(=O)C(CCCCCCCC)CCCCCCCC] |
| 19 | 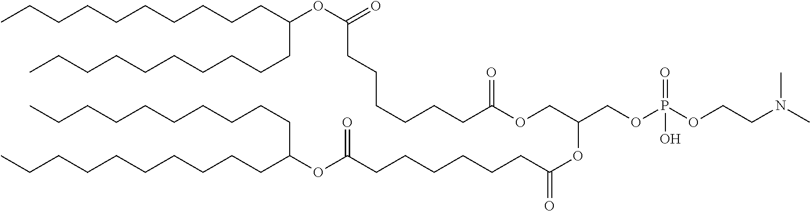 [SMILES: CCCCCCCCCCC(CCCCCCCCCC)OC(=O)CCCCCCC(=O)OCC(COP(=O)(O)OCCN(C)C)OC(=O)CCCCCCC(=O)OC(CCCCCCCCCC)CCCCCCCCCC] |
| 20 | 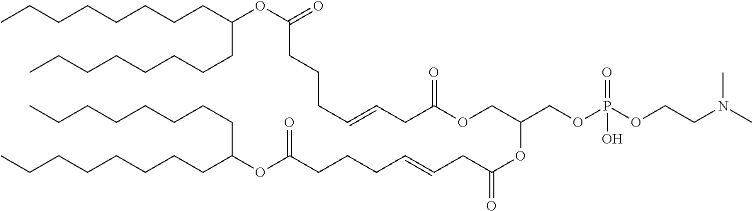 [SMILES: CCCCCCCCC(CCCCCCCC)OC(=O)CCC/C=C/CC(=O)OCC(COP(=O)(O)OCCN(C)C)OC(=O)C/C=C/CCCC(=O)OC(CCCCCCCC)CCCCCCCC] |
| 21 | 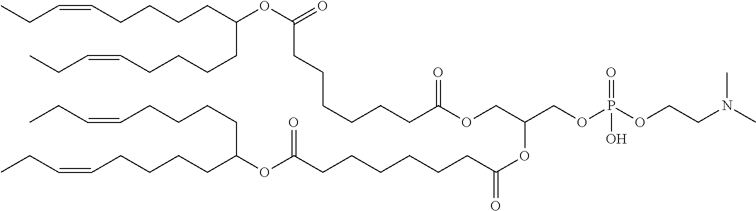 [SMILES: CC/C=C\CCCCC(CCCC/C=C\CC)OC(=O)CCCCCCC(=O)OCC(COP(=O)(O)OCCN(C)C)OC(=O)CCCCCCC(=O)OC(CCCC/C=C\CC)CCCC/C=C\CC] |
| 22 | 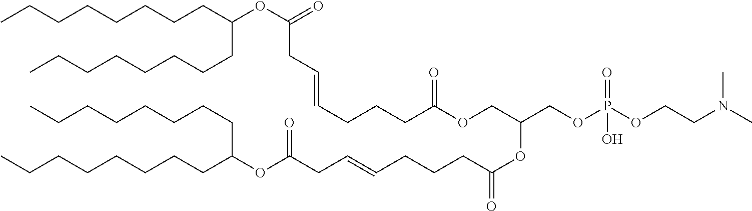 [SMILES: CCCCCCCCC(CCCCCCCC)OC(=O)C/C=C/CCCC(=O)OCC(COP(=O)(O)OCCN(C)C)OC(=O)CCC/C=C/CC(=O)OC(CCCCCCCC)CCCCCCCC] |
| 23 | 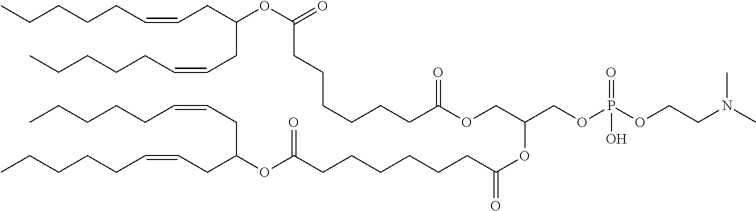 [SMILES: CCCCC/C=C\CC(C/C=C\CCCCC)OC(=O)CCCCCCC(=O)OCC(COP(=O)(O)OCCN(C)C)OC(=O)CCCCCCC(=O)OC(C/C=C\CCCCC)C/C=C\CCCCC] |
| 24 | 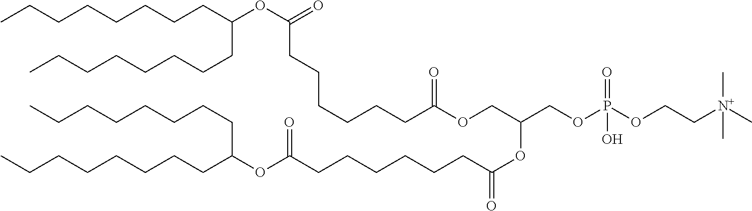 [SMILES: CCCCCCCCC(CCCCCCCC)OC(=O)CCCCCCC(=O)OCC(COP(=O)(O)OCC[N+](C)(C)C)OC(=O)CCCCCCC(=O)OC(CCCCCCCC)CCCCCCCC] |
| 25 | 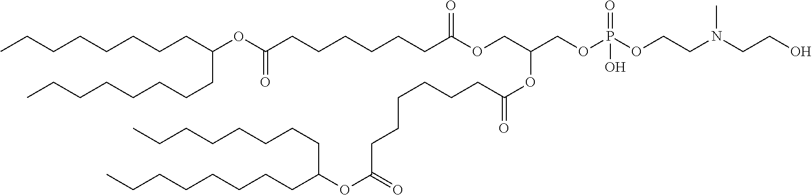 [SMILES: CCCCCCCCC(CCCCCCCC)OC(=O)CCCCCCC(=O)OCC(COP(=O)(O)OCCN(C)CCO)OC(=O)CCCCCCC(=O)OC(CCCCCCCC)CCCCCCCC] |
| 26 | 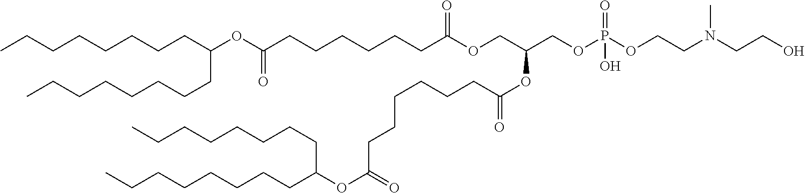 [SMILES: CCCCCCCCC(CCCCCCCC)OC(=O)CCCCCCC(=O)OC[C@@H](COP(=O)(O)OCCN(C)CCO)OC(=O)CCCCCCC(=O)OC(CCCCCCCC)CCCCCCCC] |
| 27 | 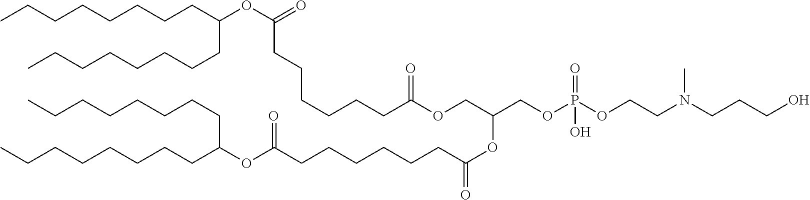 [SMILES: CCCCCCCCC(CCCCCCCC)OC(=O)CCCCCCC(=O)OCC(COP(=O)(O)OCCN(C)CCCO)OC(=O)CCCCCCC(=O)OC(CCCCCCCC)CCCCCCCC] |
| 28 | 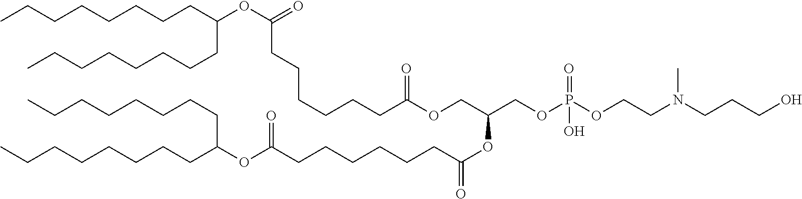 [SMILES: CCCCCCCCC(CCCCCCCC)OC(=O)CCCCCCC(=O)OC[C@@H](COP(=O)(O)OCCN(C)CCCO)OC(=O)CCCCCCC(=O)OC(CCCCCCCC)CCCCCCCC] |
| 29 | 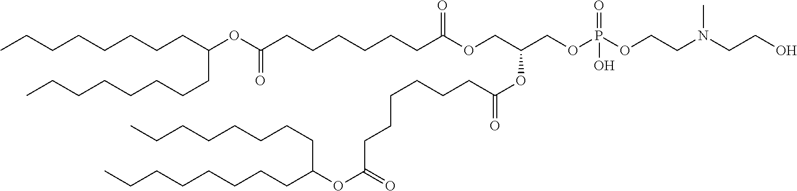 [SMILES: CCCCCCCCC(CCCCCCCC)OC(=O)CCCCCCC(=O)OC[C@H](COP(=O)(O)OCCN(C)CCO)OC(=O)CCCCCCC(=O)OC(CCCCCCCC)CCCCCCCC] |
| 30 | 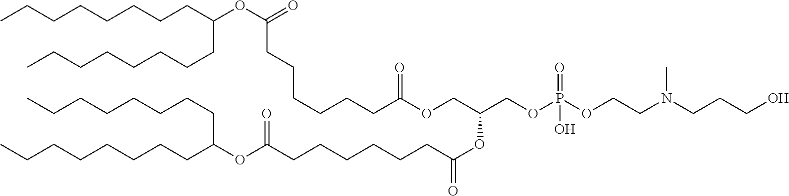 [SMILES: CCCCCCCCC(CCCCCCCC)OC(=O)CCCCCCC(=O)OC[C@H](COP(=O)(O)OCCN(C)CCCO)OC(=O)CCCCCCC(=O)OC(CCCCCCCC)CCCCCCCC] |
| 31 | 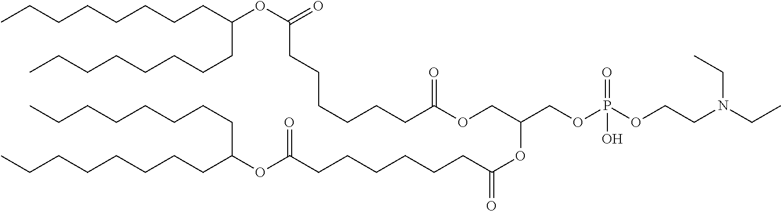 [SMILES: CCCCCCCCC(CCCCCCCC)OC(=O)CCCCCCC(=O)OCC(COP(=O)(O)OCCN(CC)CC)OC(=O)CCCCCCC(=O)OC(CCCCCCCC)CCCCCCCC] |
| 32 | 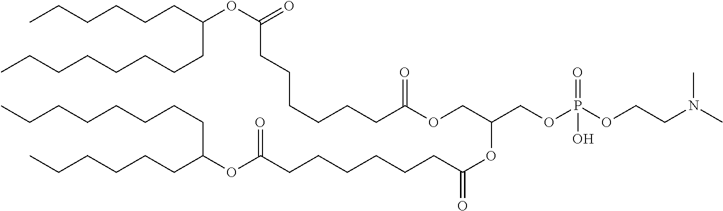 [SMILES: CCCCCCCCC(CCCCCC)OC(=O)CCCCCCC(=O)OCC(COP(=O)(O)OCCN(C)C)OC(=O)CCCCCCC(=O)OC(CCCCCC)CCCCCCCC] |
| 33 | 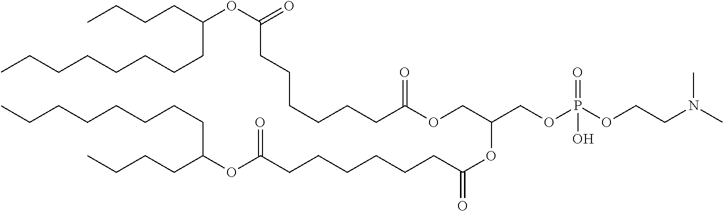 [SMILES: CCCCCCCCC(CCCC)OC(=O)CCCCCCC(=O)OCC(COP(=O)(O)OCCN(C)C)OC(=O)CCCCCCC(=O)OC(CCCC)CCCCCCCC] |
| 34 | 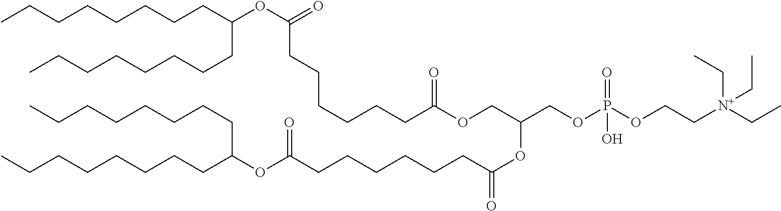 [SMILES: CCCCCCCCC(CCCCCCCC)OC(=O)CCCCCCC(=O)OCC(COP(=O)(O)OCC[N+](CC)(CC)CC)OC(=O)CCCCCCC(=O)OC(CCCCCCCC)CCCCCCCC] |
| 35 | 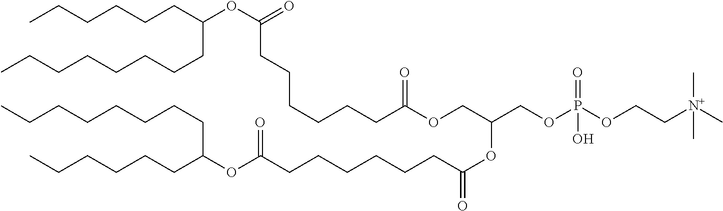 [SMILES: CCCCCCCCC(CCCCCC)OC(=O)CCCCCCC(=O)OCC(COP(=O)(O)OCC[N+](C)(C)C)OC(=O)CCCCCCC(=O)OC(CCCCCC)CCCCCCCC] |
| 36 | 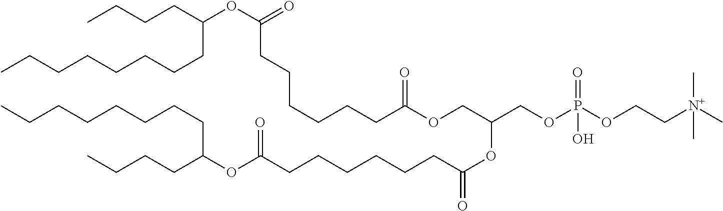 [SMILES: CCCCCCCCC(CCCC)OC(=O)CCCCCCC(=O)OCC(COP(=O)(O)OCC[N+](C)(C)C)OC(=O)CCCCCCC(=O)OC(CCCC)CCCCCCCC] |
| 37 | 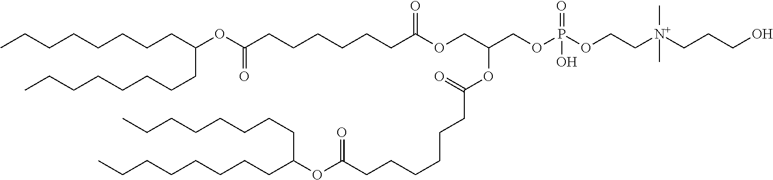 [SMILES: CCCCCCCCC(CCCCCCCC)OC(=O)CCCCCCC(=O)OCC(COP(=O)(O)OCC[N+](C)(C)CCCO)OC(=O)CCCCCCC(=O)OC(CCCCCCCC)CCCCCCCC] |
| 38 | 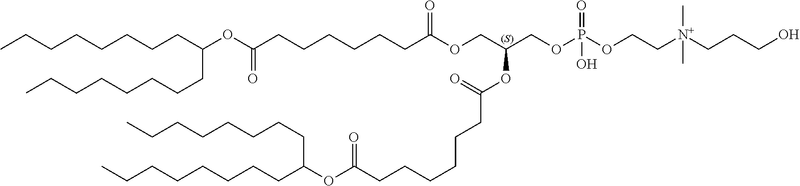 |
| 39 | 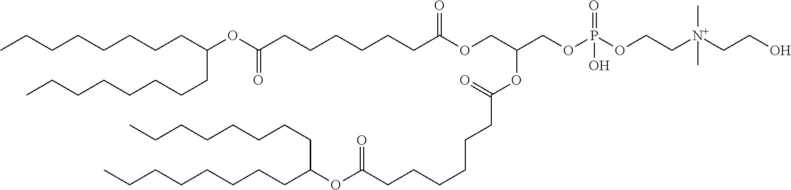 [SMILES: CCCCCCCCC(CCCCCCCC)OC(=O)CCCCCCC(=O)OCC(COP(=O)(O)OCC[N+](C)(C)CCO)OC(=O)CCCCCCC(=O)OC(CCCCCCCC)CCCCCCCC] |
| 40 | 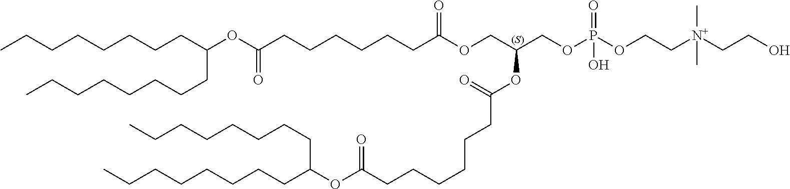 |
| 41 | 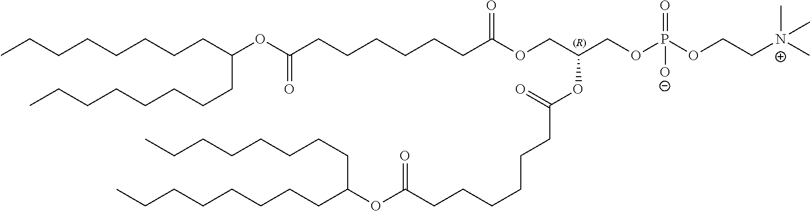 |
| 42 | 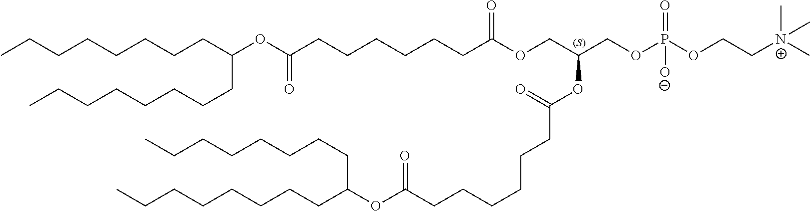 |
| 43 | 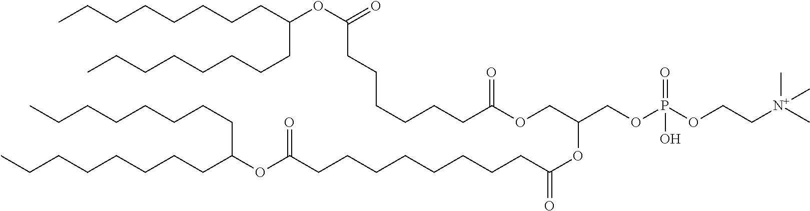 [SMILES: CCCCCCCCC(CCCCCCCC)OC(=O)CCCCCCCCC(=O)OC(COC(=O)CCCCCCC(=O)OC(CCCCCCCC)CCCCCCCC)COP(=O)(O)OCC[N+](C)(C)C] |
| 44 | 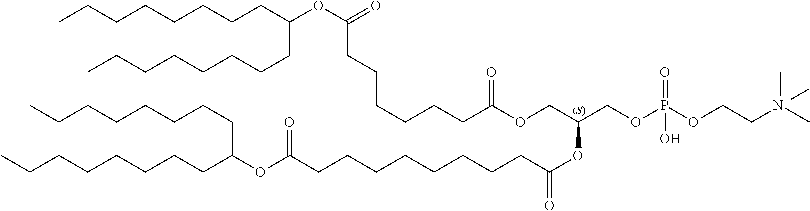 |
| 45 | 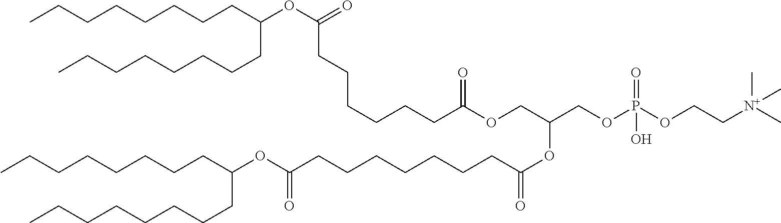 [SMILES: CCCCCCCCC(CCCCCCCC)OC(=O)CCCCCCCC(=O)OC(COC(=O)CCCCCCC(=O)OC(CCCCCCCC)CCCCCCCC)COP(=O)(O)OCC[N+](C)(C)C] |
| 46 | 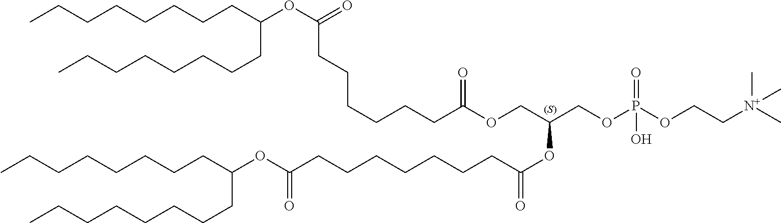 |
| 47 | 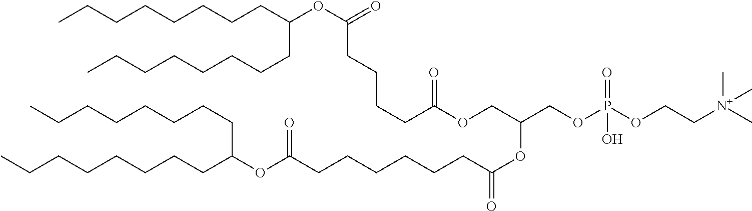 [SMILES: CCCCCCCCC(CCCCCCCC)OC(=O)CCCCCCC(=O)OC(COC(=O)CCCCC(=O)OC(CCCCCCCC)CCCCCCCC)COP(=O)(O)OCC[N+](C)(C)C] |
| 48 | 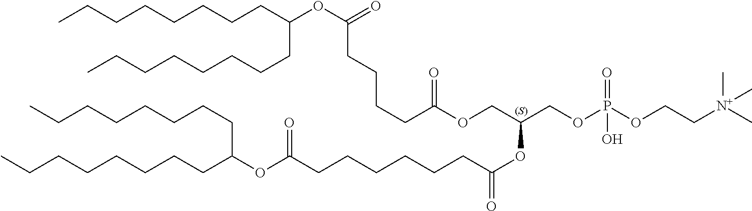 |
| 49 | 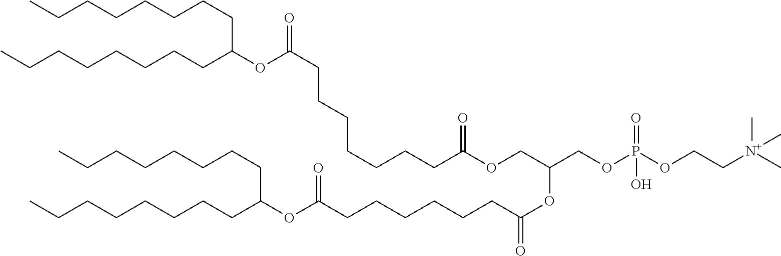 [SMILES: CCCCCCCCC(CCCCCCCC)OC(=O)CCCCCCCC(=O)OCC(COP(=O)(O)OCC[N+](C)(C)C)OC(=O)CCCCCCC(=O)OC(CCCCCCCC)CCCCCCCC] |
| 50 | 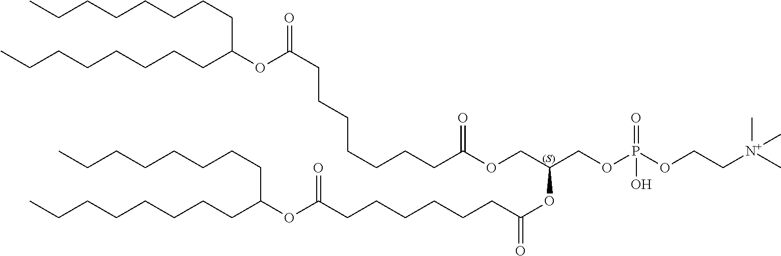 |
| 51 | 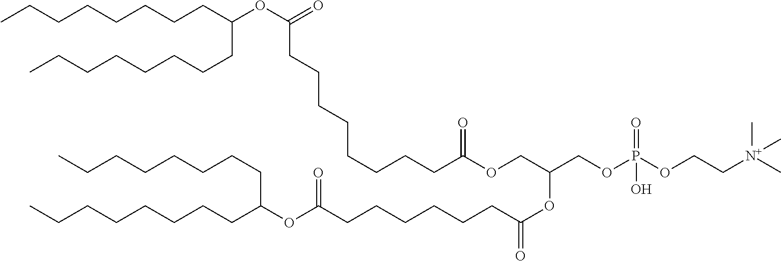 [SMILES: CCCCCCCCC(CCCCCCCC)OC(=O)CCCCCCCCC(=O)OCC(COP(=O)(O)OCC[N+](C)(C)C)OC(=O)CCCCCCC(=O)OC(CCCCCCCC)CCCCCCCC] |
| 52 | 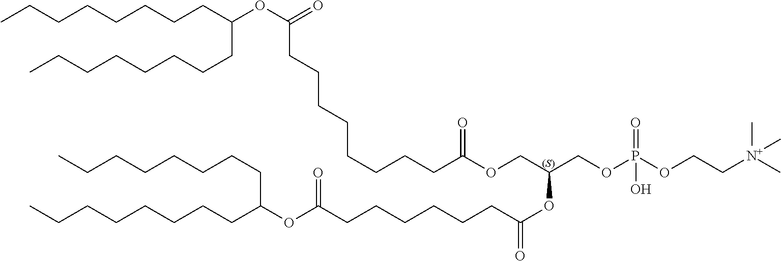 |
| 53 | 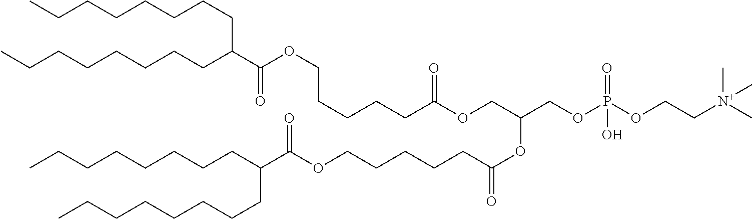 [SMILES: CCCCCCCCC(CCCCCCCC)C(=O)OCCCCCC(=O)OCC(COP(=O)(O)OCC[N+](C)(C)C)OC(=O)CCCCCOC(=O)C(CCCCCCCC)CCCCCCCC] |
| 54 | 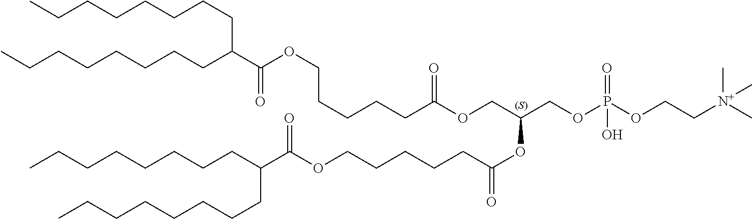 |
| 55 | 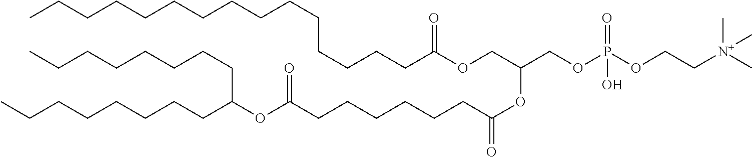 [SMILES: CCCCCCCCCCCCCCCC(=O)OCC(COP(=O)(O)OCC[N+](C)(C)C)OC(=O)CCCCCCC(=O)OC(CCCCCCCC)CCCCCCCC] |
| 56 | 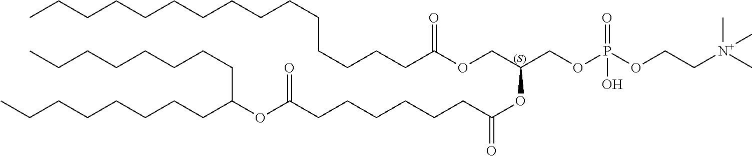 |
| 57 | 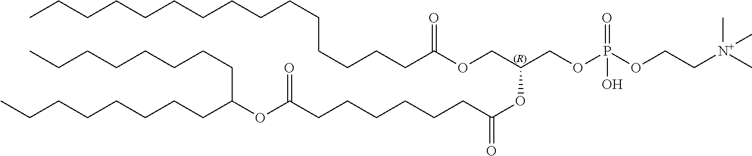 |
| 58 | 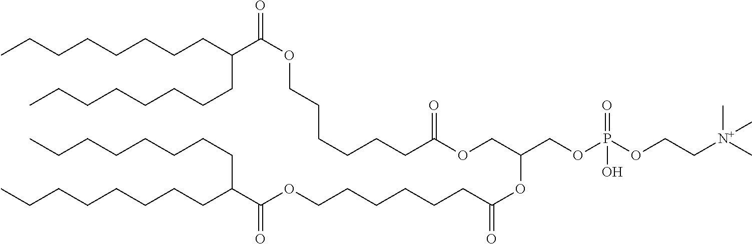 [SMILES: CCCCCCCCC(CCCCCCCC)C(=O)OCCCCCCC(=O)OCC(COP(=O)(O)OCC[N+](C)(C)C)OC(=O)CCCCCCOC(=O)C(CCCCCCCC)CCCCCCCC] |
| 59 | 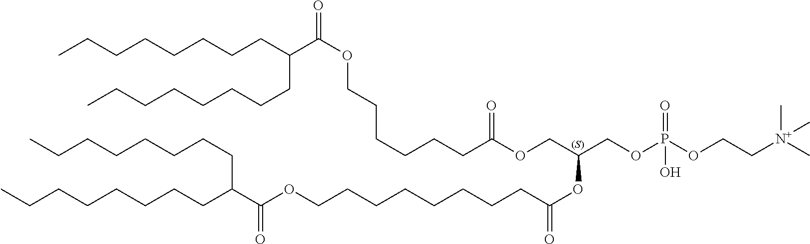 |
| 60 | 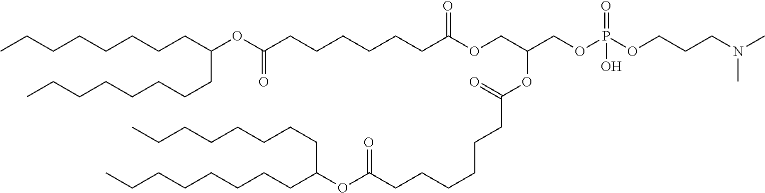 [SMILES: CCCCCCCCC(CCCCCCCC)OC(=O)CCCCCCC(=O)OCC(COP(=O)(O)OCCCN(C)C)OC(=O)CCCCCCC(=O)OC(CCCCCCCC)CCCCCCCC] |
| 61 | 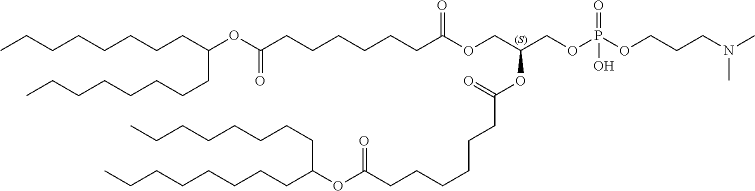 |
| 62 | 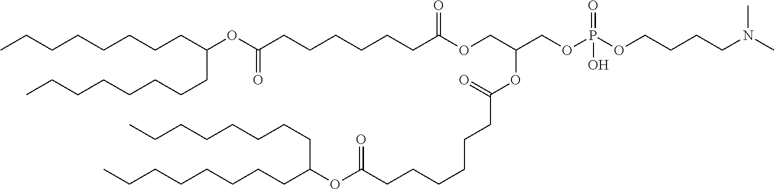 [SMILES: CCCCCCCCC(CCCCCCCC)OC(=O)CCCCCCC(=O)OCC(COP(=O)(O)OCCCCN(C)C)OC(=O)CCCCCCC(=O)OC(CCCCCCCC)CCCCCCCC] |
| 63 | 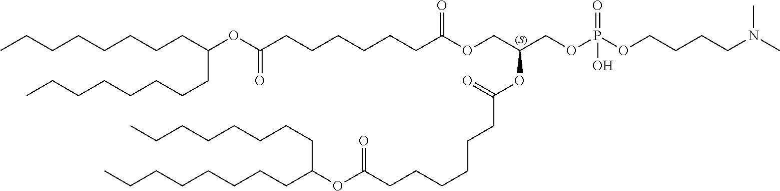 |
| 64 | 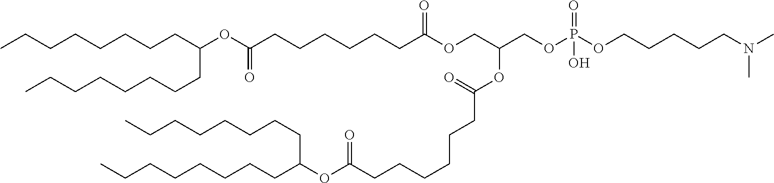 [SMILES: CCCCCCCCC(CCCCCCCC)OC(=O)CCCCCCC(=O)OCC(COP(=O)(O)OCCCCCN(C)C)OC(=O)CCCCCCC(=O)OC(CCCCCCCC)CCCCCCCC] |
| 65 | 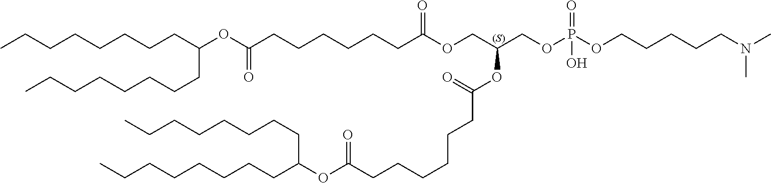 |

Any of the compounds (1-65) identified in Table A above may be provided in the form of a pharmaceutically acceptable salt and such salts are intended to be encompassed by the present invention.

The compounds of the invention as described herein can be prepared according to methods known in the art, including the exemplary syntheses of the Examples provided herein.

### Nucleic Acids

The compounds of the invention as described herein can be used to prepare compositions useful for the delivery of nucleic acids.

### Synthesis of Nucleic Acids

Nucleic acids according to the present invention may be synthesized according to any known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, mutated T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7, mutated T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

Modified mRNA

In some embodiments, mRNA according to the present invention may be synthesized as unmodified or modified mRNA. Modified mRNA comprises nucleotide modifications in the RNA. A modified mRNA according to the invention can thus include nucleotide modification that are, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g., 1-methyl-adenine, 2-methyl-adenine, 2-methylthio-N-6-isopentenyl-adenine, N-6-methyl-adenine, N-6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queuosine, beta-D-mannosyl-queuosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

### Pharmaceutical Formulations Comprising Compounds of the Invention

In certain embodiments, pharmaceutical and liposomal compositions comprising compounds of the invention as described herein can be used in formulations to facilitate the delivery of encapsulated materials (e.g., one or more polynucleotides such as mRNA) to, and subsequent transfection of one or more target cells.

According to the present invention, a nucleic acid, e.g., mRNA encoding a protein (e.g., a full length, fragment or portion of a protein) as described herein may be delivered via a delivery vehicle comprising a compound of the invention as described herein.

As used herein, the terms “delivery vehicle,” “transfer vehicle,” “nanoparticle,” or grammatical equivalents thereof, are used interchangeably.

For example, the present invention provides a composition (e.g., a pharmaceutical composition) comprising one or more compounds described herein and one or more polynucleotides. A composition (e.g., a pharmaceutical composition) may further comprise
(i) one or more cationic lipids,
(ii) one or more non-cationic lipids,
(iii) one or more sterol-based lipids and/or
(iv) one or more PEG-modified lipids.

In certain embodiments a composition exhibits an enhanced (e.g., increased) ability to transfect one or more target cells. Accordingly, also provided herein are methods of transfecting one or more target cells. Such methods generally comprise the step of contacting the one or more target cells with the pharmaceutical compositions disclosed herein (e.g., a liposomal formulation comprising a compound described herein encapsulating one or more polynucleotides) such that the one or more target cells are transfected with the materials encapsulated therein (e.g., one or more polynucleotides). As used herein, the terms “transfect” or “transfection” refer to the intracellular introduction of one or more encapsulated materials (e.g., nucleic acids and/or polynucleotides) into a cell (e.g., into a target cell). The introduced polynucleotide may be stably or transiently maintained in the target cell. The term “transfection efficiency” refers to the relative amount of such encapsulated material (e.g., polynucleotides) up-taken by, introduced into, and/or expressed by the target cell which is subject to transfection. In practice, transfection efficiency may be estimated by the amount of a reporter polynucleotide product produced by the target cells following transfection. In certain embodiments, the compounds and pharmaceutical compositions described herein demonstrate high transfection efficiencies thereby improving the likelihood that appropriate dosages of the encapsulated materials (e.g., one or more polynucleotides) will be delivered to the site of pathology and subsequently expressed, while at the same time minimizing potential systemic adverse effects or toxicity associated with the compound or their encapsulated contents.

Following transfection of one or more target cells by, for example, the polynucleotides encapsulated in the one or more lipid nanoparticles comprising the pharmaceutical or liposomal compositions disclosed herein, the production of the product (e.g., a polypeptide or protein) encoded by such polynucleotide may be stimulated and the capability of such target cells to express the polynucleotide and produce, for example, a polypeptide or protein of interest is enhanced. For example, transfection of a target cell by one or more compounds or pharmaceutical compositions encapsulating mRNA will enhance (i.e., increase) the production of the protein or enzyme encoded by such mRNA.

Further, delivery vehicles described herein (e.g., liposomal delivery vehicles) may be prepared to preferentially distribute to other target tissues, cells or organs, such as the heart, lungs, kidneys, spleen. In embodiments, the delivery vehicles described herein (e.g., liposomal delivery vehicles) may be prepared to preferentially distribute to the lungs. In embodiments, the lipid nanoparticles of the present invention may be prepared to achieve enhanced delivery to the target cells and tissues. For example, polynucleotides (e.g., mRNA) encapsulated in one or more of the pharmaceutical and liposomal compositions described herein can be delivered to and/or transfect targeted cells or tissues. In some embodiments, the encapsulated polynucleotides (e.g., mRNA) are capable of being expressed and functional polypeptide products produced (and in some instances excreted) by the target cell, thereby conferring a beneficial property to, for example the target cells or tissues. Such encapsulated polynucleotides (e.g., mRNA) may encode, for example, an antigen hormone, enzyme, receptor, polypeptide, peptide or other protein of interest.

### Liposomal Delivery Vehicles

In some embodiments, a composition is a suitable delivery vehicle. In embodiments, a composition is a liposomal delivery vehicle, e.g., a lipid nanoparticle.

The terms “liposomal delivery vehicle” and “liposomal composition” are used interchangeably.

Enriching liposomal compositions with one or more of the helper lipids disclosed herein may be used as a means of improving the safety profile or otherwise conferring one or more desired properties to such enriched liposomal composition (e.g., improved stability, improved endosomal escape, improved delivery of the encapsulated polynucleotides to one or more target cells, and/or reduced in vivo toxicity of a liposomal composition). Accordingly, also contemplated are pharmaceutical compositions, and in particular liposomal compositions, that comprise one or more of the lipids disclosed herein.

Thus, in certain embodiments, the compounds of the invention as described herein may be used as a component of a liposomal composition to facilitate or enhance the delivery and release of encapsulated materials (e.g., one or more therapeutic agents) to one or more target cells (e.g., by permeating or fusing with the lipid membranes of such target cells).

As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphophilic polymers and surfactants (e.g., polymerosomes, niosomes, etc.). In the context of the present invention, a liposomal delivery vehicle typically serves to transport a desired mRNA to a target cell or tissue.

In certain embodiments, such compositions (e.g., liposomal compositions) are loaded with or otherwise encapsulate materials, such as for example, one or more biologically-active polynucleotides (e.g., mRNA).

In embodiments, a composition (e.g., a pharmaceutical composition) comprises an mRNA encoding a peptide or protein, encapsulated within a liposome. In embodiments, a liposome comprises:
(i) one or more cationic lipids,
(ii) one or more non-cationic lipids,
(iii) one or more sterol-based lipids and
(iv) one or more PEG-modified lipids,
   wherein at least one lipid is a compound of the invention as described herein.

In embodiments, a composition comprises an mRNA encoding for a peptide or protein (e.g., any peptide or protein described herein). In embodiments, a composition comprises an mRNA encoding for a peptide (e.g., any peptide described herein). In embodiments, a composition comprises an mRNA encoding for a protein (e.g., any protein described herein).

In embodiments, a composition (e.g., a pharmaceutical composition) comprises a nucleic acid encapsulated within a liposome, wherein the liposome comprises a compound described herein.

In embodiments, a nucleic acid is an mRNA encoding a peptide or protein. In embodiments, an mRNA encodes a peptide or protein for use in the delivery to or treatment of the lung of a subject or a lung cell. In embodiments, an mRNA encodes a peptide or protein for use in the delivery to or treatment of the liver of a subject or a liver cell. In embodiments, an mRNA encodes a peptide or protein for use in the delivery to or treatment of a muscle cell. In embodiments, an mRNA encodes a peptide or protein for use in the delivery to or treatment of an immune cell. Still other exemplary mRNAs are described herein.

In embodiments, a liposomal delivery vehicle (e.g., a lipid nanoparticle) can have a net positive charge.

In embodiments, a liposomal delivery vehicle (e.g., a lipid nanoparticle) can have a net negative charge.

In embodiments, a liposomal delivery vehicle (e.g., a lipid nanoparticle) can have a net neutral charge.

In embodiments, a lipid nanoparticle that encapsulates a nucleic acid (e.g., mRNA encoding a peptide or protein) comprises one or more compounds of the invention as described herein.

The amount of a compound of the invention as described herein in a composition also can be described as a percentage (“mol %”) of the combined molar amounts of total lipids of a composition (e.g., the combined molar amounts of all lipids present in a liposomal delivery vehicle, for example the lipid nanoparticle).

In embodiments of pharmaceutical compositions described herein, a compound of the invention as described herein is present in an amount that is about 5 mol % to about 60 mol %, for example about 10 mol % to about 50 mol % of the combined molar amounts of all lipids present in a composition such as a liposomal delivery vehicle, for example a lipid nanoparticle.

In embodiments, a compound of the invention as described herein is present in an amount that is about 5 mol % to about 15 mol %, about 10 mol % to about 20 mol %, about 15 mol % to about 30 mol %, about 20 mol % to about 35 mol %, about 25 mol % to about 40 mol %, about 30 mol % to about 45 mol %, about 35 mol % to about 50 mol %, about 40 mol % to about 55 mol %, or about 45 mol % to about 60 mol % of the combined molar amounts of all lipids present in a composition such as a liposomal delivery vehicle, e.g. lipid nanoparticle.

In embodiments, a compound of the invention as described herein is present in an amount that is about 5 mol % to about 60 mol %, 5 mol % to about 50 mol %, 5 mol % to about 40 mol %, 5 mol % to about 30 mol %, about 5 mol % to about 20 mol %, about 5 mol % to about 15 mol %, about 5 mol % to about 10 mol %, about 5 mol % to about 55 mol %, about 5 mol % to about 45 mol %, about 5 mol % to about 35 mol % or about 5 mol % to about 25 mol % of the combined molar amounts of all lipids present in a composition such as a liposomal delivery vehicle.

In certain embodiments, a compound of the invention as described herein can comprise from about 10 mol % to about 50 mol %, or from 15 mol % to about 50 mol %, or from about 20 mol % to about 50 mol %, or from about 25 mol % to about 50 mol %, or from about 30 mol % to about 50 mol %, or from about 35 mol % to about 50 mol %, or from about 40 mol % to about 50 mol %, or from about 45 mol % to about 50 mol %, of the total amount of lipids in a composition (e.g., a liposomal delivery vehicle, such as a lipid nanoparticle).

In certain embodiments, a compound of the invention as described herein can comprise greater than about 5 mol %, or greater than about 10 mol %, or greater than about 15 mol %, greater than about 20 mol %, greater than about 25 mol %, greater than about 30 mol %, greater than about 35 mol %, or greater than about 40 mol % of the total amount of lipids in the lipid nanoparticle.

In certain embodiments, a compound as described can comprise less than about 60 mol %, or less than about 55 mol %, or less than about 50 mol %, or less than about 45 mol %, or less than about 40 mol %, or less than about 35 mol %, less than about 30 mol %, or less than about 25 mol %, or less than about 10 mol % of the total amount of lipids in a composition (e.g., a liposomal delivery vehicle, such as a lipid nanoparticle).

In embodiments, the amount of a compound of the invention as described herein is present in an amount that is about 5 mol %, about 10 mol %, about 15 mol %, about 20 mol %, about 25 mol %, about 30 mol %, about 35 mol %, about 40 mol %, about 45 mol %, about 50 mol %, about 55 mol %, or about 60 mol % of the combined molar amounts of total lipids in a composition (e.g., a liposomal composition such as a lipid nanoparticle).

In embodiments, the amount of a compound of the invention as described herein is present in an amount that is no more than about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol % of the combined molar amounts of total lipids in a composition (e.g., a liposomal composition such as a lipid nanoparticle).

In embodiments, the percentage results in an improved beneficial effect (e.g., improved delivery to targeted tissues such as the liver, the lung or muscle).

In a typical embodiment, a composition of the invention (e.g., a liposomal composition) comprises:
(i) one or more cationic lipids,
(ii) one or more non-cationic lipids,
(iii) one or more sterol-based lipids, and
(iv) one or more PEG-modified lipids,
   wherein at least one lipid is a compound of the invention as described herein.

For example, a composition suitable for practicing the invention has four lipid components comprising a compound of the invention as described herein, and further comprising:
(i) a cationic lipid;
(ii) a sterol-based lipid; and
(iii) a PEG-modified lipid.

The non-cationic lipid may be a helper lipid of the present invention. Cationic lipids suitable for use in compositions of the invention are well known in the art. For example, the cationic lipid may be OF-02 ((3R,6S)-3,6-bis(4-(bis((9Z,12Z)-2-hydroxyoctadeca-9,12-dien-1-yl)amino)butyl)piperazine-2,5-dione) (Fenton et al., doi: 10.1002/adma.201505822). For example, the cationic lipid may be cKK-E10 ((3R,6S)-3,6-bis(4-(bis(2-hydroxydecyl)amino)butyl)piperazine-2,5-dione) (Dong et al., doi: 10.1073/pnas.1322937111). For example, the cationic lipid may be 2-(4-(2-((4-(bis(2-hydroxydecyl)amino)butyl)disulfaneyl)ethyl)piperazin-1-yl)ethyl 4-(bis(2-hydroxydodecyl)amino)butanoate. For example, the cationic lipid may be SM-102. For example, the cationic lipid may be ALC-0315. For example, the cationic lipid may be D-Lin-MC3-DMA. For example, the cationic lipid may be DOTMA (dioleoyl-3-trimethylammonium propane) or DOTAP (1,2-dioleoyl-3-trimethylammonium propane).

The sterol-based lipid may be cholesterol. The PEG-modified lipid may be DMG-PEG2K.

In further embodiments, pharmaceutical (e.g., liposomal) compositions comprise one or more of a cationic lipid, PEG-modified lipid, a non-cationic lipid, and a sterol lipid, wherein at least one lipid present in the composition is a helper lipid of the present invention.

In other embodiments, such pharmaceutical (e.g., liposomal) compositions comprise: one or more cationic lipids; one or more PEG-modified lipids; one or more non-cationic lipids; and one or more sterol lipids, wherein at least one lipid present in the composition is a helper lipid of the present invention.

In embodiments, a composition (e.g., lipid nanoparticle) that encapsulates a nucleic acid (e.g., mRNA encoding a peptide or protein) comprises one or more compounds of the invention as described herein, one or more cationic lipids, and one or more lipids selected from the group consisting of non-cationic lipids and PEGylated lipids.

In embodiments, a composition (e.g., lipid nanoparticle) that encapsulates a nucleic acid (e.g., mRNA encoding a peptide or protein) comprises one or more compounds of the invention as described herein; one or more lipids selected from the group consisting of a cationic lipid, a non-cationic lipid, and a PEGylated lipid; and further comprises a sterol-based lipid. Typically, such a composition has four lipid components comprising a compound of the invention as described herein, a cationic lipid, and further comprising:
(i) a sterol-based lipid (e.g., cholesterol); and
(ii) a PEG-modified lipid (e.g., DMG-PEG2K).

In embodiments, a lipid nanoparticle that encapsulates a nucleic acid (e.g., mRNA encoding a peptide or protein) comprises one or more compounds of the invention as described herein, as well as one or more lipids selected from the group consisting of:
(i) a cationic lipid,
(ii) a non-cationic lipid,
(iii) a PEGylated lipid, and
(iv) a sterol-based lipid.

In embodiments, the cationic lipid is any one of the cationic lipids disclosed in the literature. In embodiments, the cationic lipid is (3R,6S)-3,6-bis(4-(bis((9Z,12Z)-2-hydroxyoctadeca-9,12-dien-1-yl)amino)butyl)piperazine-2,5-dione. In embodiments, the cationic lipid is (3R,6S)-3,6-bis(4-(bis(2-hydroxydecyl)amino)butyl)piperazine-2,5-dione. In embodiments, the cationic lipid is 2-(4-(2-((4-(bis(2-hydroxydecyl)amino)butyl)disulfaneyl)ethyl)piperazin-1-yl)ethyl 4-(bis(2-hydroxydodecyl)amino)butanoate.

According to various embodiments, the selection of lipids of the present invention, cationic lipids, and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly.

### Cationic Lipids

A composition described herein, e.g. a lipid nanoparticle may comprise one or more cationic lipids.

In some embodiments, liposomes may comprise one or more cationic lipids. As used herein, the phrase “cationic lipid” refers to any of a number of lipid species that have a net positive charge at a selected pH, such as physiological pH. Several cationic lipids have been described in the literature, many of which are commercially available.

Suitable cationic lipids for use in the compositions include the cationic lipids as described in the literature. For example, the cationic lipid may be (3R,6S)-3,6-bis(4-(bis((9Z,12Z)-2-hydroxyoctadeca-9,12-dien-1-yl)amino)butyl)piperazine-2,5-dione. For example, the cationic lipid may be (3R,6S)-3,6-bis(4-(bis(2-hydroxydecyl)amino)butyl)piperazine-2,5-dione. For example, the cationic lipid may be 2-(4-(2-((4-(bis(2-hydroxydecyl)amino)butyl)disulfaneyl)ethyl)piperazin-1-yl)ethyl 4-(bis(2-hydroxydodecyl)amino)butanoate. For example, the cationic lipid may be SM-102. For example, the cationic lipid may be ALC-0315. For example, the cationic lipid may be D-Lin-MC3-DMA. For example, the cationic lipid may be DOTMA (dioleoyl-3-trimethylammonium propane) or DOTAP (1,2-dioleoyl-3-trimethylammonium propane).

### Non-Cationic Lipids

Compositions (e.g., liposomal compositions) may also comprise one or more non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 1,2-Dierucoyl-sn-glycero-3-phosphoethanolamine (DEPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. A non-cationic or helper lipid suitable for practicing the invention is dioleoylphosphatidylethanolamine (DOPE). Alternatively, 1,2-Dierucoyl-sn-glycero-3-phosphoethanolamine (DEPE) can be used as a non-cationic or helper lipid.

In some embodiments, a non-cationic lipid is a neutral lipid, i.e., a lipid that does not carry a net charge in the conditions under which the composition is formulated and/or administered.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

### Sterol-Based Lipids

In some embodiments, a composition (e.g., a liposomal composition) comprising a helper lipid of the present invention further comprises one or more sterol-based lipids. For example, a suitable sterol-based lipid known in the literature. The sterol-based lipid may be a cholesterol-based lipid such as cholesterol. Other suitable cholesterol-based lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), beta-sitosterol, or imidazole cholesterol ester (ICE), which has the following structure,

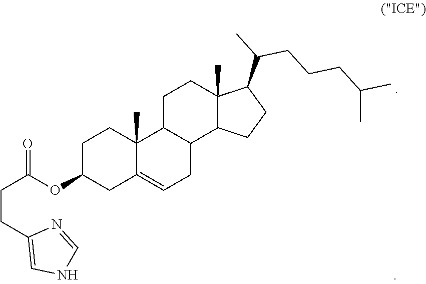 [SMILES: CC(C)CCCC(C)[C@H]1CCC2C3CC=C4C[C@@H](OC(=O)CCc5c[nH]cn5)CC[C@]4(C)C3CC[C@@]21C]

In some embodiments, a sterol-based lipid may be present in a molar ratio (mol %) of about 1% to about 30%, or about 5% to about 20% of the total lipids present in a liposome. In some embodiments, a sterol-based lipid may be present in a molar ratio (mol %) of about 10% to about 50%, or about 40% to about 50% of the total lipids present in a liposome. In some embodiments, the percentage of sterol-based lipid in the lipid nanoparticle may be greater than about 5 mol %, greater than about 10 mol %, greater than about 20 mol %, greater than about 30 mol %, or greater than about 40 mol %. In some embodiments, the percentage of sterol-based lipid in the lipid nanoparticle may be no more than about 5 mol %, no more than about 10 mol %, no more than about 20 mol %, no more than about 30 mol %, or no more than about 40 mol %.

### PEGylated Lipids

In some embodiments, a composition (e.g., a liposomal composition) comprises one or more further PEGylated lipids. A suitable PEG-modified or PEGylated lipid for practicing the invention is 1,2-dimyristoyl-rac-glycero-3-methoxypolyethylene glycol-2000 (DMG-PEG2K).

For example, the use of polyethylene glycol (PEG)-modified phospholipids and derivatized lipids such as derivatized ceramides (PEG-CER), including N-octanoyl-sphingosine-1-[succinyl(methoxy polyethylene glycol)-2000](C8 PEG-2000 ceramide) is also contemplated by the present invention in combination with one or more of compounds of the invention as described herein and, in some embodiments, other lipids together which comprise the liposome. In some embodiments, particularly useful exchangeable lipids are PEG-ceramides having shorter acyl chains (e.g., (C₁₄) or (C₁₈)).

Contemplated further PEG-modified lipids (also referred to herein as a PEGylated lipid, which term is interchangeable with PEG-modified lipid) include, but are not limited to, a polyethylene glycol chain of up to 5 kDa in length covalently attached to a lipid with alkyl chain(s) of (C₆-C₂₀) length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

Further PEG-modified phospholipid and derivatized lipids of the present invention may be present in a molar ratio (mol %) from about 0% to about 10%, about 0.5% to about 10%, about 1% to about 10%, about 2% to about 10%, about 3% to about 5%, about 1% to about 5%, or about 1.5% to about 3% of the total lipid present in the composition (e.g., a liposomal composition).

### Pharmaceutical Formulations and Therapeutic Uses

Compounds of the invention as described herein may be used in the preparation of compositions (e.g., to construct liposomal compositions) that facilitate or enhance the delivery and release of encapsulated materials (e.g., one or more therapeutic polynucleotides) to one or more target cells (e.g., by permeating or fusing with the lipid membranes of such target cells).

For example, when a liposomal composition (e.g., a lipid nanoparticle) comprises or is otherwise enriched with one or more of the compounds disclosed herein, the phase transition in the lipid bilayer of the one or more target cells may facilitate the delivery of the encapsulated materials (e.g., one or more therapeutic polynucleotides encapsulated in a lipid nanoparticle) into the one or more target cells.

Similarly, in certain embodiments compounds of the invention as described herein may be used to prepare liposomal vehicles that are characterized by their reduced toxicity in vivo. In certain embodiments, the reduced toxicity is a function of the high transfection efficiencies associated with the compositions disclosed herein, such that a reduced quantity of such composition may be administered to the subject to achieve a desired therapeutic response or outcome.

In certain embodiments, compounds of the invention as described herein may be used to prepare liposomal vehicles that are characterized by effective intranasal delivery of mRNA. In certain embodiments, compounds of the invention as described herein may be used to prepare liposomal vehicles that are characterized by effective pulmonary delivery of mRNA. In certain embodiments, compounds of the invention as described herein may be used to prepare liposomal vehicles that are characterized by achieving high levels of peptide or protein expression when delivering mRNA encoding for said peptide or protein by intravenous, intrathecal, intramuscular, intranasal, sublingual, or by pulmonary delivery, optionally through nebulization. In certain embodiments, compounds of the invention as described herein may be used to prepare liposomal vehicles that are characterized by achieving high levels of peptide or protein expression when delivering mRNA encoding for said peptide or protein by intramuscular delivery.

Thus, pharmaceutical formulations comprising a compound described and nucleic acids provided by the present invention may be used for various therapeutic disease and/or disease prevention purposes. To facilitate delivery of nucleic acids in vivo, a compound described herein and nucleic acids can be formulated in combination with one or more additional pharmaceutical carriers, targeting ligands or stabilizing reagents. In some embodiments, a compound described herein can be formulated via pre-mixed lipid solution. In other embodiments, a composition comprising a compound described herein can be formulated using post-insertion techniques into the lipid membrane of the nanoparticles. Techniques for formulation and administration of drugs may be found in “Remington's Pharmaceutical Sciences,” Mack Publishing Co., Easton, Pa., latest edition.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In embodiments, the route of administration is selected from intravenous, intrathecal, intramuscular, intranasal, sublingual, or by pulmonary delivery, optionally through nebulization. In embodiments, the route of administration is intramuscular. In particular embodiments, the intramuscular administration is to a muscle selected from the group consisting of skeletal muscle, smooth muscle and cardiac muscle. In some embodiments the administration results in delivery of the nucleic acids to a muscle cell. In some embodiments the administration results in delivery of the nucleic acids to a hepatocyte (i.e., liver cell).

A common route for administering a liposomal composition of the invention may be intravenous delivery, in particular when treating metabolic disorders, especially those affecting the liver (e.g., ornithine transcarbamylase (OTC) deficiency). Alternatively, depending on the disease or disorder to be treated, the liposomal composition may be administered via pulmonary delivery (e.g., for the treatment of cystic fibrosis). For vaccination, a liposomal composition of the invention is typically administered intramuscularly. Alternatively, a liposomal composition of the invention may be administered intranasally for vaccination. Diseases or disorders affecting the eye may be treated by administering a liposomal composition of the invention intravitreally.

Alternatively or additionally, pharmaceutical formulations of the invention may be administered in a local rather than systemic manner, for example, via injection of the pharmaceutical formulation directly into a targeted tissue (e.g., in a sustained release formulation). Local delivery can be affected in various ways, depending on the tissue to be targeted. Exemplary tissues in which mRNA may be delivered and/or expressed include, but are not limited to the liver, kidney, heart, spleen, serum, brain, skeletal muscle, lymph nodes, skin, and/or cerebrospinal fluid. In embodiments, the tissue to be targeted in the liver. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection.

Alternatively or additionally, pharmaceutical formulations of the invention may be administered intranasally. For example, the pharmaceutical formulations of the invention may be administered via nasal spray. Exemplary tissues in which mRNA may be delivered and/or expressed include, but are not limited to the lungs, heart, liver, spleen and muscle. In embodiments, the tissue to be targeted is in the lungs. In embodiments, the tissue to be targeted is in muscle.

Alternatively or additionally, pharmaceutical formulations of the invention may be administered by pulmonary delivery, optionally through nebulization or dry powder inhalation. In embodiments, the pharmaceutical formulations of the invention are administered by pulmonary delivery through nebulization. In embodiments, the pharmaceutical formulations of the invention are administered by pulmonary delivery through dry powder inhalation. Exemplary tissues in which mRNA may be delivered and/or expressed include, but are not limited to the lungs, heart, liver, spleen and muscle. In embodiments, the tissue to be targeted is in the lungs. In embodiments, the tissue to be targeted is in muscle.

Compositions described herein can comprise mRNA encoding peptides including those described herein (e.g., a polypeptide such as a protein).

In embodiments, a mRNA encodes a polypeptide. In embodiments, a mRNA encodes a peptide. In embodiments, the peptide is an antigen. In embodiments, a mRNA encodes a peptide for treating Flu. In embodiments, a mRNA encodes a peptide for treating Respiratory Syncytial virus (RSV).

In embodiments, a mRNA encodes a protein. In embodiments, a mRNA encodes a protein for treating Flu. In embodiments, a mRNA encodes a protein for treating Respiratory Syncytial virus (RSV).

The present invention provides methods for delivering a composition having full-length mRNA molecules encoding a peptide or protein of interest for use in the treatment of a subject, e.g., a human subject or a cell of a human subject or a cell that is treated and delivered to a human subject.

### Delivery Methods

The route of delivery used in the methods of the invention allows for non-invasive, self-administration of the compounds of the invention. In some embodiments, the methods involve intranasal, intratracheal or pulmonary administration by aerosolization, nebulization, or instillation of a composition comprising mRNA encoding a therapeutic peptide or protein in a suitable transfection or lipid carrier vehicles as described above. In some embodiments, the methods involve intranasal, intratracheal or pulmonary administration by intravenous, intrathecal, intramuscular, intranasal, sublingual, or by pulmonary delivery, optionally through nebulization of a composition comprising mRNA encoding a therapeutic peptide or protein in a suitable transfection or lipid carrier vehicles as described above. In some embodiments, the peptide or protein is encapsulated with a liposome. In some embodiments, the liposome comprises a lipid, which is a compound of the invention. As used herein below, administration of a compound of the invention includes administration of a composition comprising a compound of the invention.

Although the local cells and tissues of the lung represent a potential target capable of functioning as a biological depot or reservoir for production and secretion of the protein encoded by the mRNA, applicants have discovered that administration of the compounds of the invention to the lung via aerosolization, nebulization, or instillation results in the distribution of even non-secreted proteins outside the lung cells. Without wishing to be bound by any particular theory, it is contemplated that nanoparticle compositions of the invention pass, through the lung airway-blood barrier, resulting in translation of the intact nanoparticle to non-lung cells and tissues, such as, e.g., the heart, the liver, the spleen, where it results in the production of the encoded peptide or protein in these non-lung tissues. Thus, the utility of the compounds of the invention and methods of the invention extend beyond production of therapeutic protein in lung cells and tissues of the lung and can be used to delivery to non-lung target cells and/or tissues. They are useful in the management and treatment of a large number of diseases. In certain embodiments, the compounds of the invention, used in the methods of the invention result in the distribution of the mRNA encapsulated nanoparticles and production of the encoded peptide or protein in the liver, spleen, heart, and/or other non-lung cells. For example, administration of the compounds of the invention, by aerosolization, nebulization, or instillation to the lung will result in the composition itself and its peptide or protein product (e.g., an antigen or functional protein) will be detectable in both the local cells and tissues of the lung, as well as in peripheral target cells, tissues and organs as a result of translocation of the mRNA and delivery vehicle to non-lung cells.

In certain embodiments, the compounds of the invention may be employed in the methods of the invention to specifically target peripheral cells or tissues. Following the pulmonary delivery, it is contemplated the compounds of the invention cross the lung airway-blood barrier and distribute into cells other than the local lung cells. Accordingly, the compounds disclosed herein may be administered to a subject by way of the pulmonary route of administration, using a variety of approach known by those skilled in the art (e.g., by inhalation), and distribute to both the local target cells and tissues of the lung, as well as in peripheral non-lung cells and tissues (e.g., cells of the liver, spleen, kidneys, heart, skeletal muscle, lymph nodes, brain, cerebrospinal fluid, and plasma). As a result, both the local cells of the lung and the peripheral non-lung cells can serve as biological reservoirs or depots capable of producing and/or secreting a translation product encoded by one or more polynucleotides. Accordingly, the present invention is not limited to the treatment of lung diseases or conditions, but rather can be used as a non-invasive means of facilitating the delivery of polynucleotides, or the production of peptides or proteins encoded thereby, in peripheral organs, tissues and cells (e.g., hepatocytes) which would otherwise be achieved only by systemic administration. Exemplary peripheral non-lung cells include, but are not limited to, hepatocytes, epithelial cells, hematopoietic cells, epithelial cells, endothelial cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Following administration of the composition to the subject, the peptide or protein product encoded by the mRNA (e.g., a functional protein or enzyme) is detectable in the peripheral target tissues for at least about one to seven days or longer following administration of the compound to the subject. The amount of peptide or protein product necessary to achieve a therapeutic effect will vary depending on the condition being treated, the peptide or protein encoded, and the condition of the patient. For example, the peptide or protein product may be detectable in the peripheral target tissues at a concentration (e.g., a therapeutic concentration) of at least 0.025-1.5 μg/ml (e.g., at least 0.050 μg/ml, at least 0.075 μg/ml, at least 0.1 μg/ml, at least 0.2 μg/ml, at least 0.3 μg/ml, at least 0.4 μg/ml, at least 0.5 μg/ml, at least 0.6 μg/ml, at least 0.7 μg/ml, at least 0.8 μg/ml, at least 0.9 μg/ml, at least 1.0 μg/ml, at least 1.1 μg/ml, at least 1.2 μg/ml, at least 1.3 μg/ml, at least 1.4 μg/ml, or at least 1.5 μg/ml), for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the compound to the subject.

It has been demonstrated that nucleic acids can be delivered to the lungs by intratracheal administration of a liquid suspension of the compound and inhalation of an aerosol mist produced by a liquid nebulizer or the use of a dry powder apparatus such as that described in U.S. Pat. No. 5,780,014, incorporated herein by reference.

In certain embodiments, the compounds of the invention may be formulated such that they may be aerosolized or otherwise delivered as a particulate liquid or solid prior to or upon administration to the subject. Such compounds may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.5 mg/kg of mRNA per dose) to the subject. For example, in certain embodiments, the compounds of the invention are administered to a subject using a metered dose inhaler containing a suspension or solution comprising the compound and a suitable propellant. In certain embodiments, the compounds of the invention may be formulated as a particulate powder (e.g., respirable dry particles) intended for inhalation. In certain embodiments, compositions of the invention formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 μm or smaller).

In yet other embodiments, the compositions of the invention are formulated to include one or more pulmonary surfactants (e.g., lamellar bodies). In some embodiments, the compositions of the invention are administered to a subject such that a concentration of at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compositions of the invention are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg mRNA is administered in one or more doses.

### EXAMPLES

While certain compounds, compositions and methods of the present invention have been described with specificity in accordance with certain embodiments, the following examples serve only to illustrate the compounds of the invention and are not intended to limit the same.

### List of Abbreviations

DMAP: 4-Dimethylaminopyridine
DMF: Dimethylformamide
EDCI: 1-Ethyl-3-(3-dimethylaminopropyl)carbodiimide
EtOAc: Ethyl acetate
LDA: Lithium diisopropylamide
THF: Tetrahydrofuran
MS: Mass spectrometry
APCI-MS: Atmospheric-pressure chemical ionization mass spectrometry
ESI-MS: Electrospray ionization mass spectrometry
TLC: Thin Layer Chromatography

### Example 1: Synthesis of Compounds of the Present Invention

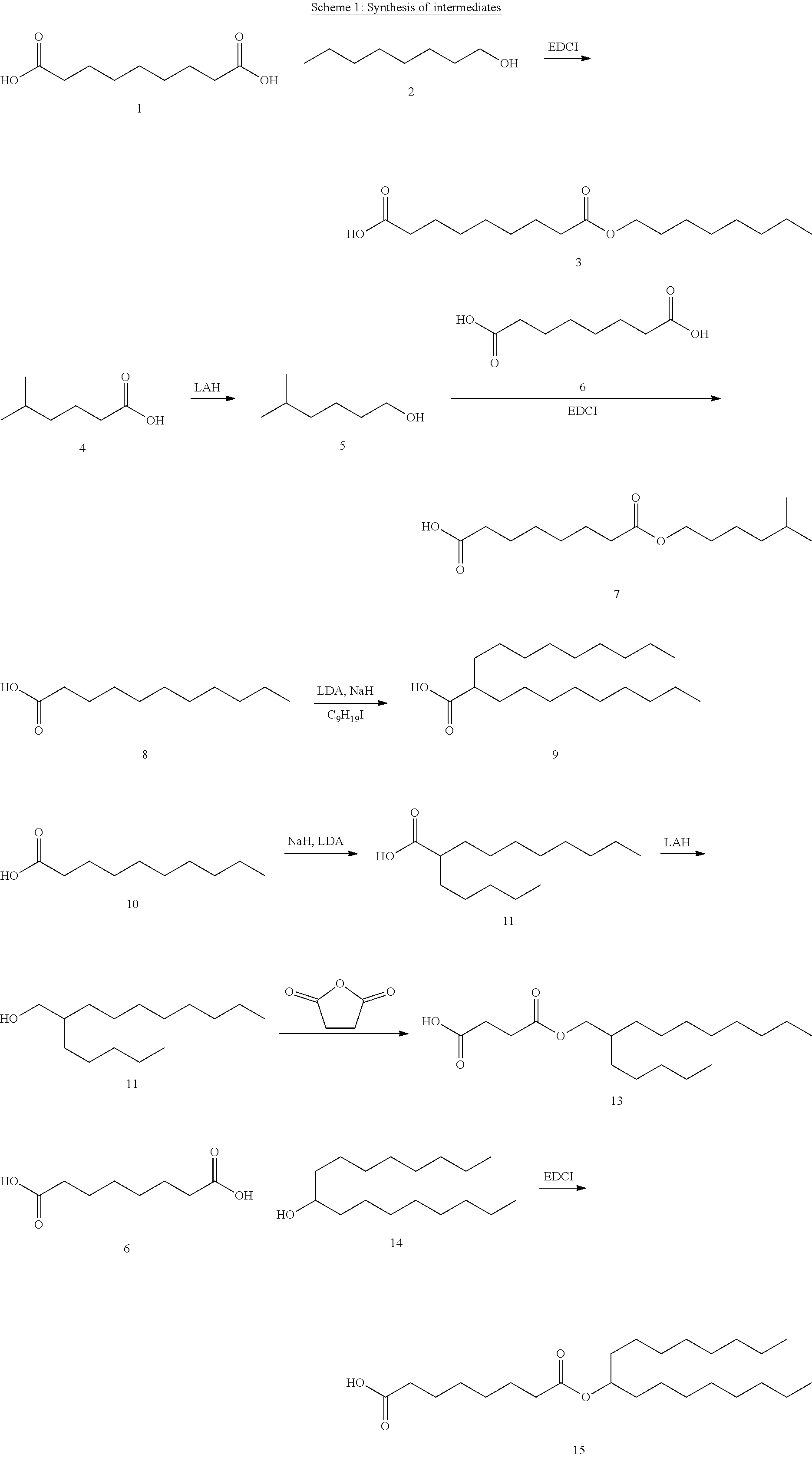

### Synthesis of 9-(Octyloxy)-9-oxononanoic acid (3)

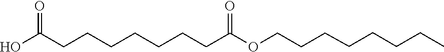 [SMILES: CCCCCCCCOC(=O)CCCCCCCC(=O)O]

A solution of nonanedioic acid 1 (25 g, 0.133 mole) octan-1-ol 2 (21 g, 0.133 mole), EDCI (25.5 g, 0.133 mole) and DMAP (3.25 g, 26.5 mmol) in 300 mL dichloromethane was stirred at room temperature overnight. The reaction mixture was diluted with water, extracted with dichloromethane (2×500 mL), and the combined organic layer was washed with brine. After dried over sodium sulfate, the solvent was evaporated under vacuum, and the crude was purified by flash column chromatography (SiO₂: 0 to 10% methanol in dichloromethane) to give 9-(octyloxy)-9-oxononanoic acid as colorless oil (15 g, 37%).

### Synthesis of 8-((5-Methylhexyl)oxy)-8-oxooctanoic acid (7)

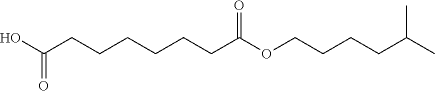 [SMILES: CC(C)CCCCOC(=O)CCCCCCC(=O)O]

### Step 1

To a cold suspension of LiAlH₄ (21.4 g, 0.564 mole) in 200 mL THF, was slowly added a solution of 5-methylhexanoic acid 4 (21.8 g, 0.167 mole) in 90 mL THF. After addition, the resulting mixture was stirred at room temperature overnight. The reaction was carefully quenched by adding 21 mL water, 21 mL 15% NaOH and 60 mL water. The resulting mixture was diluted with 400 mL ether and stirred for 1 h. After filtration through celite, the filtrate was dried over magnesium sulfate, and the solvent was removed under vacuum to get 5-methyl-1-hexanol as colorless oil (19 g, 98%).

### Step 2

A solution of suberic acid 6 (30.3 g, 0.174 mole) and 5-methyl-1-hexanol 5 (19 g, 0.163 mole), EDCI (33.25 g, 0.174 mole) and DMAP (4.25 g, 34.8 mmol) in 300 mL dichloromethane was stirred at room temperature overnight. The reaction mixture was diluted with water, extracted with dichloromethane (2×500 mL), and the combined organic layer was washed with brine. After dried over sodium sulfate, the solvent was evaporated under vacuum, and the crude was purified by flash column chromatography (SiO₂: 0 to 10% methanol in dichloromethane) to give 9-(octyloxy)-9-oxononanoic acid as colorless oil (14 g, 29%).

### Synthetic of 2-Nonylundecanoic acid (9)

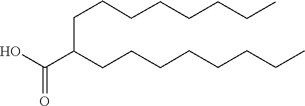 [SMILES: CCCCCCCCC(CCCCCCCC)C(=O)O]

To a suspension of NaH (60%, 2.32 g, 58 mmol) in 50 mL THF, was added a solution of nonanoic acid 8 (10 g, 52.5 mmol) in 30 mL THF dropwise. After addition, the reaction mixture was cooled to 0° C., a solution of LDA (2.0 M in THF/heptane/ethylbenzene, 31.7 mL, 63.5 mmol) was added slowly, and then the mixture was stirred at room temperature for 30 minutes. Octyl iodide (33 g, 63.5 mmol) was added, and the reaction was heated at 45° C. for 16 h. After cooled to room temperature, the reaction was quenched with 1N HCl to pH 2. After separation, the organic layer was dried over magnesium sulfate and concentrated, and the residue was purified by flash column chromatography (SiO₂: 0 to 20% ethyl acetate in hexanes) to give 2-nonylundecanoic acid as white solid (8 g, 50%).

### Synthesis of 4-Oxo-4-((2-pentyldecyl)oxy)butanoic acid (13)

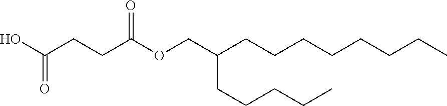 [SMILES: CCCCCCCCC(CCCCC)COC(=O)CCC(=O)O]

### Step 1

To a suspension of NaH (60%, 5.11 g, 0.128 mole) in 30 mL THF, was added a solution of decanoic acid 10 (20 g, 0.116 mole) in 20 mL THF dropwise. After addition, the reaction mixture was cooled to 0° C., a solution of LDA (2.0 M in THF/heptane/ethylbenzene, 69.8 mL, 0.14 mole) was added slowly, and then the mixture was stirred at room temperature for 30 minutes. Pentyl iodide (26.6 g, 0.14 mole) was added, and the reaction was heated at 45° C. for 16 h. After cooled to room temperature, the reaction was quenched with 1N HCl to pH 2. After separation, the organic layer was dried over magnesium sulfate and concentrated, and the residue was purified by flash column chromatography (SiO₂: 0 to 20% ethyl acetate in hexanes) to give 2-pentyldecanoic acid as white solid (14 g, 50%).

### Step 2

To a cold suspension of LiAlH₄ (7.4 g, 0.197 mole) in 100 mL THF, was slowly added a solution of 2-pentyldecanoic acid 11 (14 g, 57.9 mmol) in 60 mL THF. After addition, the resulting mixture was stirred at room temperature overnight. The reaction was carefully quenched by adding 7.4 mL water, 7.4 mL 15% NaOH and 24 mL water, then the resulting mixture was diluted with 200 mL ether and stirred for 1 h. After filtration through celite, the filtrate was dried over magnesium sulfate, and the solvent was removed under vacuum to get 2-pentyldecan-1-ol as colorless oil (13 g, 98%).

### Step 3

A mixture of 2-pentyldecan-1-ol 12 (13 g, 57 mmol), succinic anhydride (11.4 g, 0.114 mole) and DMAP (17.4 g, 0.142 mole) in 200 mL dichloromethane was stirred at room temperature overnight. The reaction mixture was diluted with water, extracted with dichloromethane (2×500 mL), and the combined organic layer was washed with brine. After dried over sodium sulfate and concentration, the crude purified by flash column chromatography (SiO₂: 0 to 10% methanol in dichloromethane) to give 4-oxo-4-((2-pentyldecyl)oxy)butanoic acid as colorless oil (15 g, 80%).

### Synthesis of 8-(heptadecane-9-yloxy)-8-oxooctanoic acid (15)

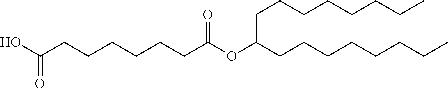 [SMILES: CCCCCCCCC(CCCCCCCC)OC(=O)CCCCCCC(=O)O]

A mixture of suberic acid 6 (19 g, 0.109 mole), 1-octylnonanol 14 (20 g, 78.1 mmol), EDCI (14.9 g, 78.1 mmol) and DMAP (1.9 g, 15.6 mmol) in 300 mL dichloromethane was stirred at room temperature overnight. The reaction mixture was diluted with water, extracted with dichloromethane (2×500 mL), and the combined organic layer was washed with brine. After dried over sodium sulfate and concentrated, the residue was purified by flash column chromatography (SiO₂: 0 to 10% methanol in dichloromethane) to give 8-(heptadecane-9-yloxy)-8-oxooctanoic acid as colorless oil (13 g, 26%).

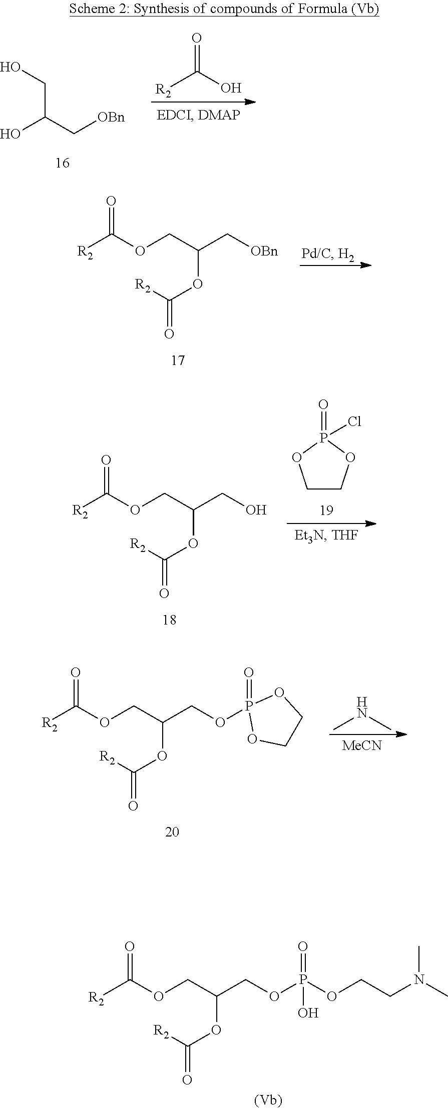

### Synthetic Procedure for Compound 7

### Step 1: Synthesis of O′¹,O¹-(3-(Benzyloxy)propane-1,2-diyl) 9-dioctyl di(nonanedioate) (17)

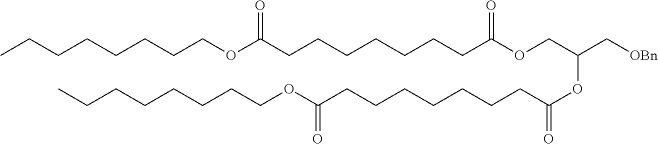 [SMILES: CCCCCCCCOC(=O)CCCCCCCC(=O)OCC(COCc1ccccc1)OC(=O)CCCCCCCC(=O)OCCCCCCCC]

A solution of 9-(octyloxy)-9-oxononanoic acid 3 (9.43 g, 31.4 mmol), 3-(benzyloxy)propane-1,2-diol 16 (2.86 g, 15.7 mmol), EDCI (6.6 g, 34.5 mmol) and DMAP (0.38 g, 3.1 mmol) in 60 mL dichloromethane was stirred at room temperature overnight. The reaction mixture was diluted with water, extracted with dichloromethane (2×150 mL), and the combined organic layer was washed with brine. After dried over sodium sulfate and concentrated, the crude was purified by flash column chromatography (SiO₂: 0 to 20% ethyl acetate in hexanes) to give O′¹,O¹-(3-(benzyloxy)propane-1,2-diyl) 9-dioctyl di(nonanedioate) as colorless oil (6.44 g, 55%).

### Step 2: Synthesis of O′¹,O¹-(3-Hydroxypropane-1,2-diyl) 9-dioctyl di(nonanedioate) (18)

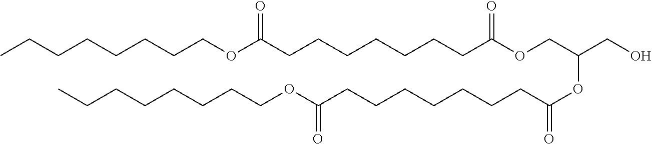 [SMILES: CCCCCCCCOC(=O)CCCCCCCC(=O)OCC(CO)OC(=O)CCCCCCCC(=O)OCCCCCCCC]

A mixture of O′¹,O¹-(3-(benzyloxy)propane-1,2-diyl) 9-dioctyl di(nonanedioate) 17 (4 g, 5.35 mmol) and 5% Pd/C (200 mg) in 30 mL ethyl acetate was purged with nitrogen and hydrogen 3 times respectively, and then the reaction was stirred at room temperature with hydrogen balloon for 16 h. After filtration and concentration, O′¹,O¹-(3-hydroxypropane-1,2-diyl) 9-dioctyl di(nonanedioate) was obtained as colorless oil (3.2 g, 91%).

### Step 3: Synthesis of 9-Dioctyl O′¹,O¹-(3-((2-oxido-1,3,2-dioxaphospholan-2-yl)oxy)propane-1,2-diyl) di(nonanedioate) (20)

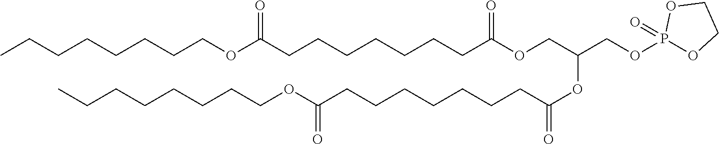 [SMILES: CCCCCCCCOC(=O)CCCCCCCC(=O)OCC(COP1(=O)OCCO1)OC(=O)CCCCCCCC(=O)OCCCCCCCC]

A solution of O′¹,O¹-(3-hydroxypropane-1,2-diyl) 9-dioctyl di(nonanedioate) 18 (1.5 g, 2.28 mmol) and triethylamine (0.23 g, 2.28 mmol) in 10 mL THF was cooled to 0° C., and then a solution of 2-chloro-2-oxo-1,3,2-dioxaphospholane 19 (0.32 g, 2.28 mol) in 4 mL THF was added dropwise over 30 min. The reaction mixture was stirred at room temperature overnight. After filtration, the filtrate was concentrated under vacuum, and the residue was used for next step without further purification.

### Step 4: Synthesis of O′¹,O¹-(3-(((2-(Dimethylamino)ethoxy)(hydroxy)phosphoryl)oxy)-propane-1,2-diyl) 9-dioctyl di(nonanedioate) (Compound 7)

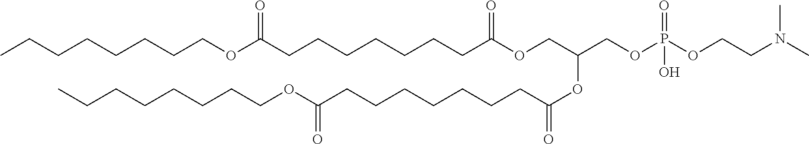 [SMILES: CCCCCCCCOC(=O)CCCCCCCC(=O)OCC(COP(=O)(O)OCCN(C)C)OC(=O)CCCCCCCC(=O)OCCCCCCCC]

In a seal tube, a solution of 9-dioctyl O′¹,O¹-(3-((2-oxido-1,3,2-dioxaphospholan-2-yl)oxy)propane-1,2-diyl) di(nonanedioate) 20 (0.5 g, 0.66 mmol) in 10 mL acetonitrile was cooled in dry ice bath, and a solution of dimethylamine (2 M in THF, 1.65 mL, 3.3 mmol) was added. The bottle was sealed and heated at 65° C. for 16 h. After cooled to room temperature, the volatiles were evaporated in vacuo, and the crude was purified by column chromatography (Gold column: CHCl₃/MeOH/H₂O 65:25:4) to give the desired product as white solid (0.11 g, 20%).

¹H NMR (300 MHz, CDCl₃) δ 5.26-5.19 (m, 1H), 4.37 (dd, 1H), 4.27-4.21 (m, 2H), 4.14 (dd, 1H), 4.02 (m, 6H), 3.17 (m, 2H), 2.86 (s, 6H), 2.31-2.25 (m, 8H), 1.65-1.51 (m, 12H), 1.29 (m, 32H), 0.86 (t, 6H). ESI-MS analysis: Calculated C41H78NO12P [M+H]=808.5, Observed=808.7.

### O′¹,O¹-(3-(((2-(Dimethylamino)ethoxy)(hydroxy)phosphoryl)-oxy)propane-1,2-diyl) 8-bis(5-methylhexyl) di(octanedioate) (Compound 8)

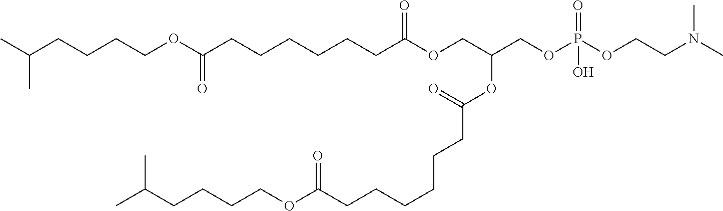 [SMILES: CC(C)CCCCOC(=O)CCCCCCC(=O)OCC(COP(=O)(O)OCCN(C)C)OC(=O)CCCCCCC(=O)OCCCCC(C)C]

The title compound was prepared analogously to compound 7. ¹H NMR (300 MHz, CDCl₃) δ 5.23-5.13 (1H), 4.35 (dd, 1H), 4.23 (m, 2H), 4.12 (dd, 1H), 4.04-3.97 (m, 6H), 3.17 (m, 2H), 2.85 (s, 6H), 2.26 (t, 8H), 1.65-1.45 (m, 10H), 1.26 (m, 28H), 0.85 (t, 6H).

ESI-MS analysis: Calculated C37H70NO12P [M+H]=752.5, Observed=752.6.

### O,O′-(3-(((2-(Dimethylamino)ethoxy)(hydroxy)phosphoryl)-oxy)propane-1,2-diyl) bis(2-pentyldecyl) disuccinate) (Compound 10)

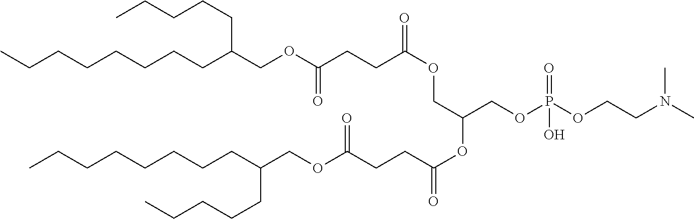 [SMILES: CCCCCCCCC(CCCCC)COC(=O)CCC(=O)OCC(COP(=O)(O)OCCN(C)C)OC(=O)CCC(=O)OCC(CCCCC)CCCCCCCC]

The title compound was prepared analogously to compound 7. ¹H NMR (300 MHz, CDCl₃) δ 5.30-5.22 (m, 1H), 4.38 (dd, 1H), 4.26-4.18 (m, 4H), 4.10-4.04 (m, 2H), 3.98-3.93 (m, 4H), 3.21-3.16 (m, 2H), 2.85 (s, 6H), 2.64-2.62 (m, 8H), 1.70-1.55 (m, 4H), 1.26 (m, 40H), 0.87 (t, 12H).

ESI-MS analysis: Calculated C45H86NO12P [M+H]=864.5, Observed=864.6.

### 1-(1-(((2-(Dimethylamino)ethoxy)(hydroxy)phosphoryl)oxy)-3-((8-(heptadecan-9-yloxy)-8-oxooctanoyl)oxy)propan-2-yl) 9-octyl nonanedioate (Compound 11)

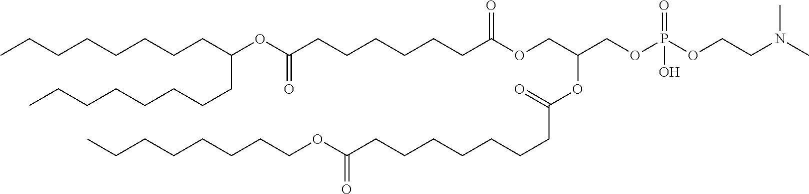 [SMILES: CCCCCCCCOC(=O)CCCCCCCC(=O)OC(COC(=O)CCCCCCC(=O)OC(CCCCCCCC)CCCCCCCC)COP(=O)(O)OCCN(C)C]

The title compound was prepared analogously to compound 7. ¹H NMR (300 MHz, CDCl₃) δ 5.28-5.20 (m, 1H), 4.84 (quint, 1H), 4.37 (dd, 1H), 4.26-4.19 (m, 2H), 4.15 (dd, 1H), 4.04 (t, 4H), 3.20-3.15 (m, 2H), 2.85 (s, 6H), 2.32-2.24 (m, 8H), 1.66-1.44 (m, 12H), 1.36-1.20 (m, 48H), 0.87 (t, 9H).

ESI-MS analysis: Calculated C49H94NO12P [M+H]=920.6, Observed=920.1.

### O′¹,O¹-(3-(((2-(Dimethylamino)ethoxy)(hydroxy)phosphoryl)-oxy)propane-1,2-diyl) 8-di(heptadecan-9-yl) di(octanedioate) (Compound 12)

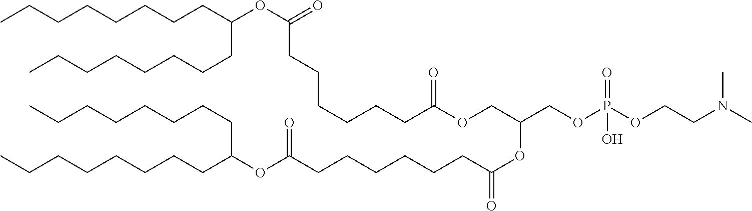 [SMILES: CCCCCCCCC(CCCCCCCC)OC(=O)CCCCCCC(=O)OCC(COP(=O)(O)OCCN(C)C)OC(=O)CCCCCCC(=O)OC(CCCCCCCC)CCCCCCCC]

The title compound was prepared analogously to compound 7. ¹H NMR (300 MHz, CDCl₃) δ 5.28-5.21 (m, 1H), 4.85 (quint, 2H), 4.39 (dd, 1H), 4.27-4.20 (m, 2H), 4.16 (dd, 1H), 4.08-4.02 (m, 2H), 3.22-3.17 (m, 2H), 2.86 (s, 6H), 2.34-2.24 (m, 8H), 1.68-1.42 (m, 12H), 1.25 (m, 60H), 0.88 (t, 12H).

ESI-MS analysis: Calculated C57H110NO12P [M+H]=1032.8, Observed=1032.9.

### O′¹,O¹-(3-(((2-(Dimethylamino)ethoxy)(hydroxy)phosphoryl)oxy)propane-1,2-diyl) 7-di(heptadecan-9-yl) di(heptanedioate) (Compound 15)

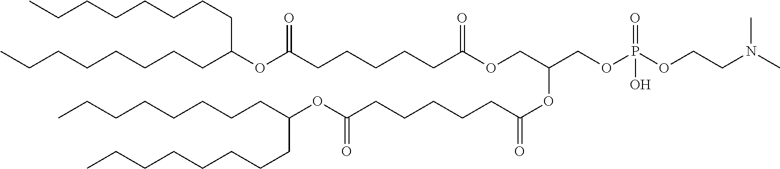 [SMILES: CCCCCCCCC(CCCCCCCC)OC(=O)CCCCCC(=O)OCC(COP(=O)(O)OCCN(C)C)OC(=O)CCCCCC(=O)OC(CCCCCCCC)CCCCCCCC]

The title compound was prepared analogously to compound 7. ¹H NMR (300 MHz, CDCl₃) δ 5.20 (m, 1H), 4.82 (quint, 2H), 4.38 (dd, 1H), 4.21 (m, 2H), 4.12 (dd, 1H), 3.97 (t, 2H), 3.21 (m, 2H), 2.85 (s, 6H), 2.32-2.21 (m, 8H), 1.64-1.42 (m, 12H), 1.22 (m, 56H), 0.84 (t, 12H).

APCI-MS analysis: Calculated C55H106NO12P [M+H]=1004.7, Observed=1004.8.

### O′¹,O¹-(3-(((2-(Dimethylamino)ethoxy)(hydroxy)phosphoryl)oxy)propane-1,2-diyl) 9-di(heptadecan-9-yl) di(nonanedioate) (Compound 16)

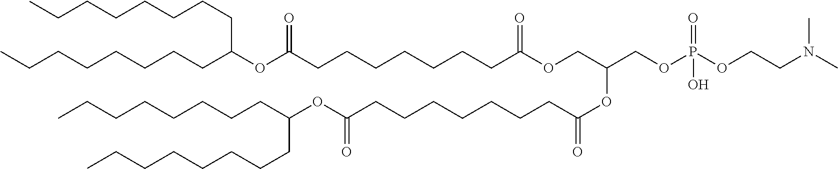 [SMILES: CCCCCCCCC(CCCCCCCC)OC(=O)CCCCCCCC(=O)OCC(COP(=O)(O)OCCN(C)C)OC(=O)CCCCCCCC(=O)OC(CCCCCCCC)CCCCCCCC]

The title compound was prepared analogously to compound 7. ¹H NMR (300 MHz, CDCl₃) δ 5.21 (m, 1H), 4.84 (quint, 2H), 4.38 (dd, 1H), 4.23 (m, 2H), 4.12 (dd, 1H), 3.99 (t, 2H), 3.24 (m, 2H), 2.85 (s, 6H), 2.30-2.21 (m, 8H), 1.64-1.42 (m, 12H), 1.23 (m, 64H), 0.86 (t, 12H).

APCI-MS analysis: Calculated C59H114NO12P [M+H]=1060.5, Observed=1060.8.

### ((3-(((2-(Dimethylamino)ethoxy)(hydroxy)phosphoryl)oxy)propane-1,2-diyl)bis(oxy))bis(6-oxohexane-6,1-diyl) bis(2-octyldecanoate) (Compound 17)

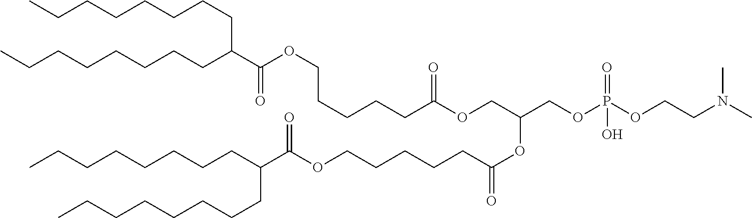 [SMILES: CCCCCCCCC(CCCCCCCC)C(=O)OCCCCCC(=O)OCC(COP(=O)(O)OCCN(C)C)OC(=O)CCCCCOC(=O)C(CCCCCCCC)CCCCCCCC]

The title compound was prepared analogously to compound 7. ¹H NMR (300 MHz, CDCl₃) δ 5.23 (m, 1H), 4.38 (dd, 1H), 4.22 (m, 2H), 4.13 (dd, 1H), 4.04 (t, 4H), 4.00-3.93 (m, 2H), 3.23 (m, 2H), 2.87 (s, 6H), 2.36-2.24 (m, 6H), 1.68-1.52 (m, 8H), 1.45-1.34 (m, 4H), 1.24 (m, 56H), 0.86 (t, 12H).

APCI-MS analysis: Calculated C55H106NO12P [M+H]=1004.7, Observed=1004.7.

### ((3-(((2-(Dimethylamino)ethoxy)(hydroxy)phosphoryl)oxy)propane-1,2-diyl)bis(oxy))bis(7-oxoheptane-7,1-diyl) bis(2-octyldecanoate) (Compound 18)

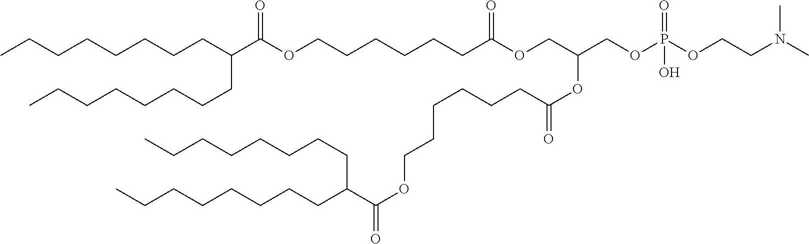 [SMILES: CCCCCCCCC(CCCCCCCC)C(=O)OCCCCCCC(=O)OCC(COP(=O)(O)OCCN(C)C)OC(=O)CCCCCCOC(=O)C(CCCCCCCC)CCCCCCCC]

The title compound was prepared analogously to compound 7. ¹H NMR (300 MHz, CDCl₃) δ 5.26 (m, 1H), 4.38 (dd, 1H), 4.22 (m, 2H), 4.17 (dd, 1H), 4.04 (t, 4H), 4.00-3.93 (m, 2H), 3.22 (m, 2H), 2.86 (s, 6H), 2.36-2.24 (m, 6H), 1.68-1.52 (m, 8H), 1.45-1.34 (m, 4H), 1.24 (m, 60H), 0.86 (t, 12H).

APCI-MS analysis: Calculated C57H110NO12P [M+H]=1032.7, Observed=1032.7.

### O′1,O1-(3-(((2-(dimethylamino)ethoxy)(hydroxy)phosphoryl)oxy)propane-1,2-diyl) 8-di(henicosan-11-yl) di(octanedioate) (Compound 19)

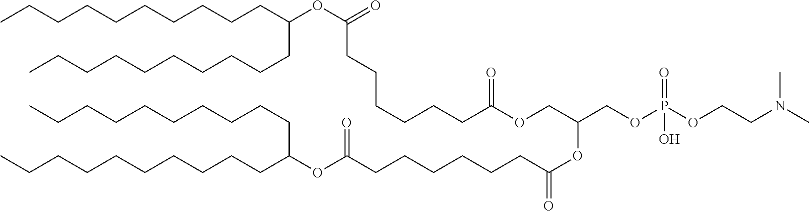 [SMILES: CCCCCCCCCCC(CCCCCCCCCC)OC(=O)CCCCCCC(=O)OCC(COP(=O)(O)OCCN(C)C)OC(=O)CCCCCCC(=O)OC(CCCCCCCCCC)CCCCCCCCCC]

The title compound was prepared analogously to compound 7. ¹H NMR (300 MHz, CDCl₃) δ 5.23 (m, 1H), 4.84 (quint, 2H), 4.38 (dd, 1H), 4.23 (m, 2H), 4.15 (dd, 1H), 4.01 (t, 2H), 3.24 (m, 2H), 2.86 (s, 6H), 2.34-2.21 (m, 8H), 1.64-1.42 (m, 16H), 1.24 (m, 72H), 0.86 (t, 12H).

APCI-MS analysis: Calculated C65H126NO12P [M+H]=1144.9, Observed=1144.1.

### Synthetic Procedure for Compound 9

### Step 1: Synthesis of 3-(Benzyloxy)propane-1,2-diyl bis(2-nonylundecanoate) (17c)

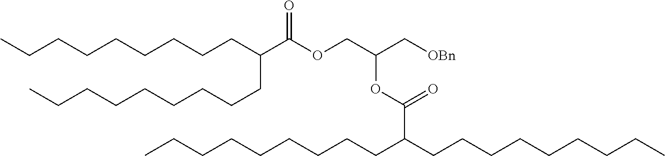 [SMILES: CCCCCCCCCC(CCCCCCCCC)C(=O)OCC(COCc1ccccc1)OC(=O)C(CCCCCCCCC)CCCCCCCCC]

A mixture of 2-nonylundecanoic acid 9 (6.27 g, 20 mmol) and 1,1′-carbonyldiimidazole (3.57 g, 22 mmol) in 40 mL THF was stirred for 40 min. A solution of 3-(benzyloxy)propane-1,2-diol 16 (911 mg, 5 mmol) in 50 mL DMF was cooled to 0° C., sodium hydride (60% in mineral oil, 880 mg, 22 mmol) was added, and the resulting mixture was stirred at this temperature for 15 min. The aliquot of 1-(1H-imidazol-1-yl)-2-nonylundecan-1-one was added slowly, and the mixture was stirred at room temperature for 2 h. TLC showed complete reaction. The reaction mixture was poured into 1 N HCl and extracted with ethyl acetate, and the combined organic layer was washed by water and brine. After dried over sodium sulfate, the solvent was concentrated, and the crude was purified by column chromatography (SiO₂: EtOAc/hexanes 0 to 10%) to get mixture, which was further purified by column (SiO₂: ether/hexanes 0 to 20%) to get pure product as wax (2.27 g, 57%).

### Step 2: Synthesis of 3-Hydroxypropane-1,2-diyl bis(2-nonylundecanoate) (18c)

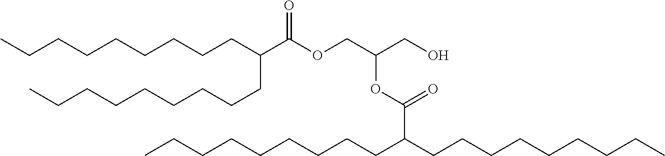 [SMILES: CCCCCCCCCC(CCCCCCCCC)C(=O)OCC(CO)OC(=O)C(CCCCCCCCC)CCCCCCCCC]

A mixture of 3-(benzyloxy)propane-1,2-diyl bis(2-nonylundecanoate) 17c (2.27 g, 2.94 mmol) and 5% Pd/C (300 mg) in 30 mL ethyl acetate was purged with nitrogen and hydrogen 3 times respectively, and then the reaction was proceed at room temperature in parr hydrogenator for 16 h. After filtration and concentration, 3-hydroxypropane-1,2-diyl bis(2-nonylundecanoate) was obtained as colorless oil (1.94 g, 97%).

### Step 3: Synthesis of 3-((2-Oxido-1,3,2-dioxaphospholan-2-yl)oxy)propane-1,2-diyl bis(2-nonylundecanoate) (20c)

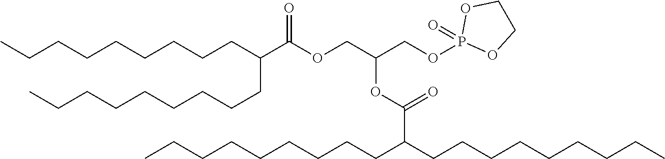 [SMILES: CCCCCCCCCC(CCCCCCCCC)C(=O)OCC(COP1(=O)OCCO1)OC(=O)C(CCCCCCCCC)CCCCCCCCC]

A solution of 3-hydroxypropane-1,2-diyl bis(2-nonylundecanoate) 18c (1.0 g, 1.47 mmol) and triethylamine (0.20 mL, 1.47 mmol) in 10 mL THF was cooled to 0° C., and then a solution of 2-chloro-2-oxo-1,3,2-dioxaphospholane 19 (0.21 g, 1.47 mol) in 4 mL THF was added dropwise over 30 min. The reaction mixture was stirred at room temperature overnight. After filtration, the filtrate was concentrated under vacuum, and the residue was used for next step without further purification.

### Step 4: 3-(((2 (Dimethylamino)ethoxy)(hydroxy)phosphoryl)oxy)-propane-1,2-diyl bis(2-nonylundecanoate) (Compound 9)

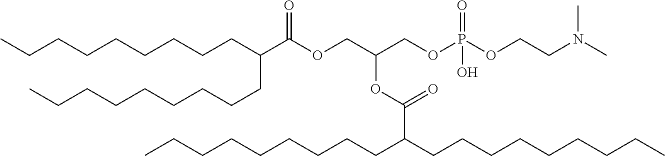 [SMILES: CCCCCCCCCC(CCCCCCCCC)C(=O)OCC(COP(=O)(O)OCCN(C)C)OC(=O)C(CCCCCCCCC)CCCCCCCCC]

In a seal tube, a solution of 3-((2-oxido-1,3,2-dioxaphospholan-2-yl)oxy)propane-1,2-diyl bis(2-nonylundecanoate) 20c (1.26 g, 1.47 mmol) in 10 mL acetonitrile was cooled in dry ice bath, and a solution of dimethylamine (2 M in THF, 3.65 mL, 7.3 mmol) was added. The bottle was sealed and heated at 65° C. for 16 h. After cooled to room temperature, the volatiles were evaporated in vacuo, and the crude was purified by column chromatography (Gold column: CHCl₃/MeOH/H₂O 65:25:4) to give the desired product as white solid (110 mg, 9%).

¹H NMR (300 MHz, CDCl₃) δ 5.28-5.16 (1H), 4.50 (dd, 1H), 4.24 (m, 2H), 4.10 (dd, 1H), 4.05-3.90 (m, 4H), 3.20 (m, 2H), 2.86 (s, 6H), 2.35-2.21 (m, 2H), 1.65-1.36 (m, 6H), 1.24 (m, 56H), 0.87 (t, 12H).

APCI-MS analysis: Calculated C47H94NO8P [M+H]=832.6, Observed=832.2.

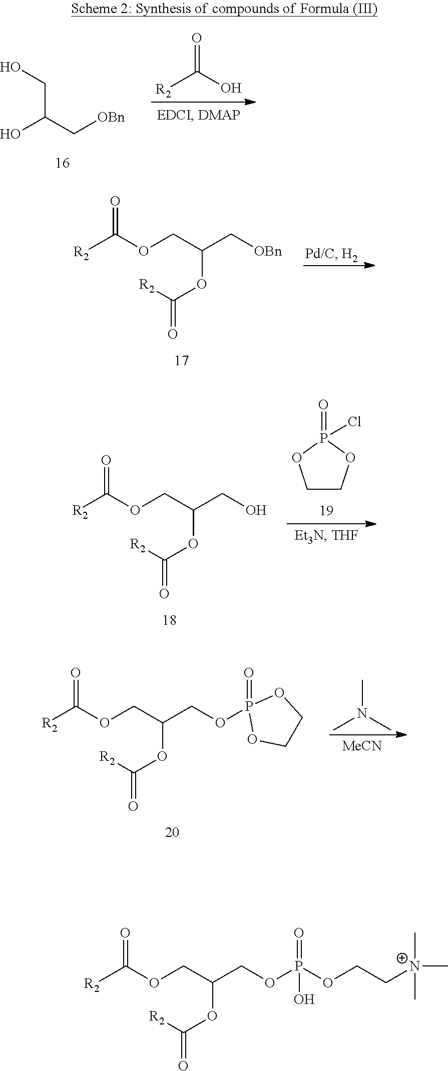

### Synthesis of 2-(((2,3-Bis((8-(heptadecan-9-yloxy)-8-oxooctanoyl)oxy)propoxy)(hydroxy)-phosphoryl)oxy)-N,N,N-trimethylethan-1-aminium (Compound 24)

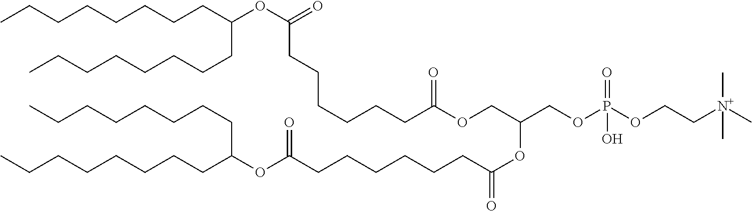 [SMILES: CCCCCCCCC(CCCCCCCC)OC(=O)CCCCCCC(=O)OCC(COP(=O)(O)OCC[N+](C)(C)C)OC(=O)CCCCCCC(=O)OC(CCCCCCCC)CCCCCCCC]

In a seal tube, a solution of 9-dioctyl O′¹,O¹-(3-((2-oxido-1,3,2-dioxaphospholan-2-yl)oxy)propane-1,2-diyl) di(nonanedioate) 20 (900 mg, 1.0 mmol) in 10 mL acetonitrile was cooled in dry ice bath, and a solution of trimethylamine (2 M in THF, 2.5 mL, 5 mmol) was added. The bottle was sealed and heated at 65° C. for 3 days. After cooled to room temperature, the volatiles were evaporated in vacuo, and the crude was purified by column chromatography (Gold column: CHCl₃/MeOH/H₂O 65:25:4) to give the desired product as white solid (560 mg, 54%).

¹H NMR (300 MHz, CDCl₃) δ 5.17 (m, 1H), 4.83 (quint, 2H), 4.38 (dd, 1H), 4.30 (m, 2H), 4.09 (dd, 1H), 3.91 (m, 2H), 3.78 (m, 2H), 3.35 (s, 9H), 2.32-2.21 (m, 8H), 1.64-1.42 (m, 16H), 1.24 (m, 56H), 0.86 (t, 12H).

APCI-MS analysis: Calculated C58H113NO12P [M+]=1046.8, Observed=1046.8.

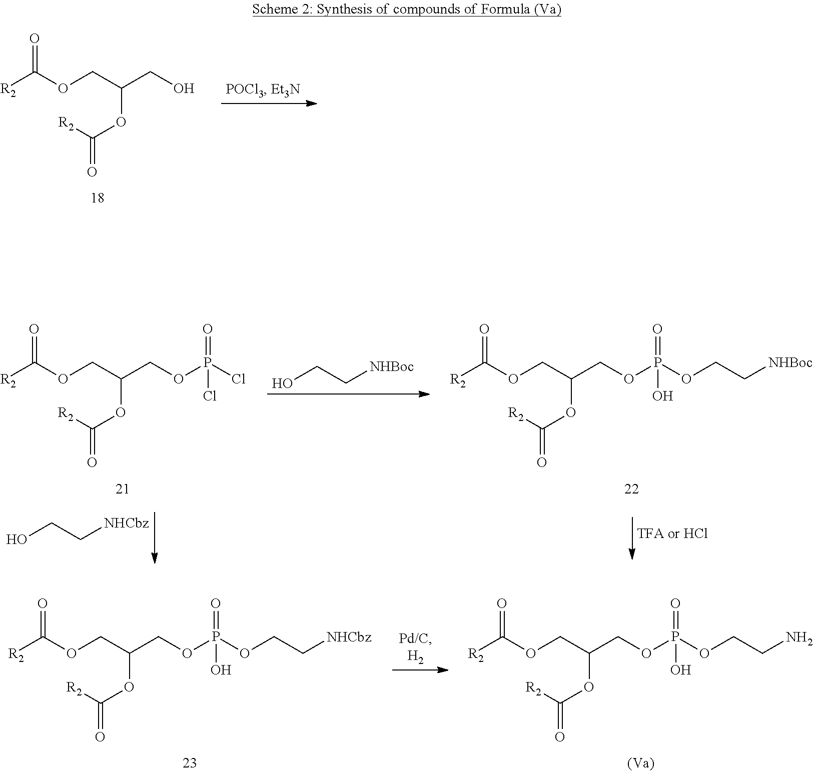

Synthetic Procedure for Compound 1:

### Step 1: Synthesis of O′¹,O¹-(3-(((2-(((Benzyloxy)carbonyl)amino)ethoxy)(hydroxy)-phosphoryl)oxy)propane-1,2-diyl) 9-dioctyl di(nonanedioate) (23)

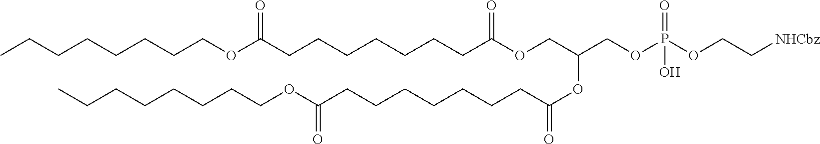 [SMILES: CCCCCCCCOC(=O)CCCCCCCC(=O)OCC(COP(=O)(O)OCCNC(=O)OCc1ccccc1)OC(=O)CCCCCCCC(=O)OCCCCCCCC]

A mixture POCl₃ (266 mg, 1.7 mmol) in 20 mL dichloromethane and 20 mL diethyl ether was cooled to 0° C., then triethylamine (193 mg, 1.9 mmol) was added under N₂ atmosphere, followed by a solution of O′¹,O¹-(3-hydroxypropane-1,2-diyl) 8-bis(5-methylhexyl) di(octanedioate) 18 (1.0 g, 1.59 mmol) in 5 mL dichloromethane, and the resulting mixture was stirred at this temperature for 2 h. TLC showed the disappearance of alcohol. The mixture was cooled to 0° C., then a solution of benzyl (2-hydroxyethyl)carbamate (341 mg, 1.75 mmol) and triethylamine (241 mg, 2.4 mmol) in 10 mL dichloromethane was added, and the mixture was stirred at room temperature overnight. 2 mL water was added, and the mixture was stirred for 2 h. After concentration, the crude was purified by column chromatography (SiO₂: 0-10% methanol in dichloromethane) to give the desired product as pale yellow oil (200 mg, 14%).

### Step 2: Synthesis of O′¹,O¹-(3-(((2-Aminoethoxy)(hydroxy)phosphoryl)oxy)propane-1,2-diyl) 8-bis(5-methylhexyl) di(octanedioate) (Compound 1)

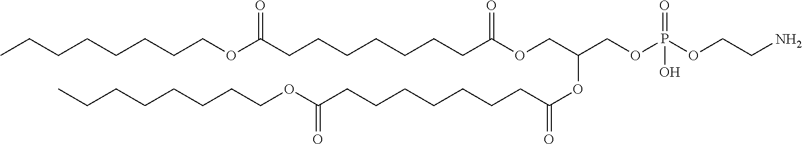 [SMILES: CCCCCCCCOC(=O)CCCCCCCC(=O)OCC(COP(=O)(O)OCCN)OC(=O)CCCCCCCC(=O)OCCCCCCCC]

To a mixture of O′¹,O¹-(3-(((2-(((benzyloxy)carbonyl)amino)ethoxy)(hydroxy)-phosphoryl)oxy)propane-1,2-diyl) 9-dioctyl di(nonanedioate) 23 (200 mg, 0.23 mmol) and 5% Pd/C (70 mg) in 50 mL CH₂Cl₂, was added 3 drops of acetic acid, and then the mixture was kept in Parr reactor under hydrogen at 38 psi overnight. The reaction mixture was filtered through Celite and concentrated, the residue was purified by column chromatography eluted with 0-30% methanol in dichloromethane to give the desired product as white wax (50 mg, 30%).

¹H NMR (300 MHz, CD₃OD) δ 5.28 (m, 1H), 4.42 (dd, 1H), 4.16 (dd, 1H), 4.12-3.94 (m, 8H), 3.17 (m, 2H), 2.40-2.27 (m, 8H), 1.70-1.53 (m, 10H), 1.33 (m, 36H), 0.97-0.86 (m, 6H).

APCI-MS analysis: Calculated C39H74NO12P [M+H]=779.5, Observed=779.5.

### O′¹,O¹-(3-(((2-Aminoethoxy)(hydroxy)phosphoryl)oxy)-propane-1,2-diyl) 8-bis(5-methylhexyl) di(octanedioate) (Compound 2)

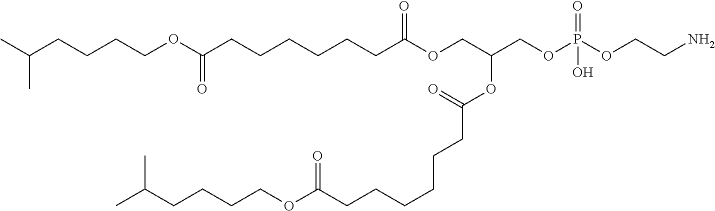 [SMILES: CC(C)CCCCOC(=O)CCCCCCC(=O)OCC(COP(=O)(O)OCCN)OC(=O)CCCCCCC(=O)OCCCCC(C)C]

The title compound was prepared analogously to compound 1. ¹H NMR (300 MHz, DMSO-d₆) δ 5.05 (m, 1H), 4.28 (dd, 1H), 4.08 (dd, 1H), 3.97 (t, 4H), 3.85-3.73 (m, 4H), 2.95 (m, 2H), 2.47 (t, 8H), 1.56-1.44 (m, 14H), 1.32-1.09 (m, 16H), 0.82 (d, 12H).

ESI-MS analysis: Calculated C35H66NO12P [M+H]=724.4, Observed=724.5.

### Synthetic Procedure for Compound 6

### Step 1: Synthesis of O′¹,O¹-(3-(((2-((tert-Butoxycarbonyl)amino)ethoxy)(hydroxy)phosphoryl)oxy)propane-1,2-diyl) 8-di(heptadecan-9-yl) di(octanedioate) (22)

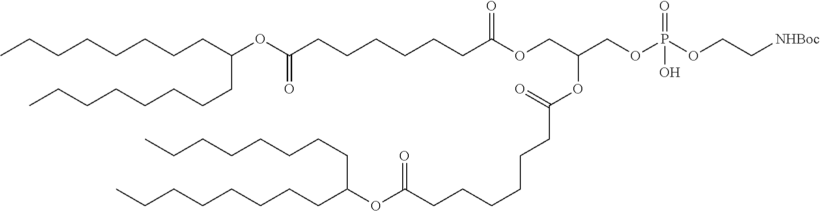 [SMILES: CCCCCCCCC(CCCCCCCC)OC(=O)CCCCCCC(=O)OCC(COP(=O)(O)OCCNC(=O)OC(C)(C)C)OC(=O)CCCCCCC(=O)OC(CCCCCCCC)CCCCCCCC]

A mixture of POCl₃ (191 mg, 1.25 mmol) and triethylamine (0.24 mL, 1.7 mmol) in 10 mL THF was cooled to 0° C., then a solution of 8-di(heptadecan-9-yl) O′¹,O¹-(3-hydroxypropane-1,2-diyl) di(octanedioate) 18 (1.0 g, 1.13 mmol) in 5 mL THF was added slowly, and the resulting mixture was stirred at this temperature for 2 h. TLC showed the disappearance of alcohol. The volatiles were evaporated under vacuum, and the residue was redissolved in 10 mL dichloromethane. After cooled to 0° C., a solution of tert-butyl (2-hydroxyethyl)carbamate (183 mg, 1.13 mmol) and triethylamine (0.24 mL, 1.7 mmol) in 5 mL dichloromethane was added, and the mixture was stirred at room temperature overnight. 2 mL water was added, and the mixture was stirred for 2 h. After concentration, the crude was purified by column chromatography (SiO₂: 0-10% methanol in dichloromethane) to give the desired product with triethylamine, which was partitioned between water and dichloromethane to give pure product as wax (400 mg, 35%).

### Step 2: Synthesis of 2-Aminoethyl (2,3-bis((8-(heptadecan-9-yloxy)-8-oxooctanoyl)oxy)propyl) phosphate (Compound 6)

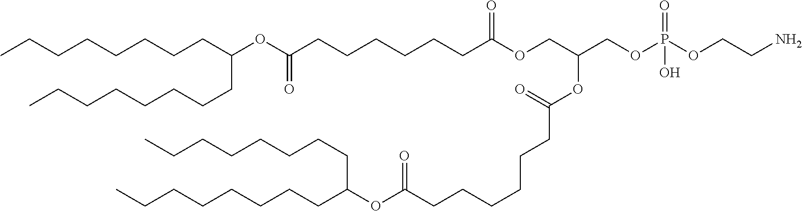 [SMILES: CCCCCCCCC(CCCCCCCC)OC(=O)CCCCCCC(=O)OCC(COP(=O)(O)OCCN)OC(=O)CCCCCCC(=O)OC(CCCCCCCC)CCCCCCCC]

To a solution of O′¹,O¹-(3-(((2-((tert-butoxycarbonyl)amino)ethoxy)(hydroxy)phosphoryl)oxy)propane-1,2-diyl) 8-di(heptadecan-9-yl) di(octanedioate) 22 (400 mg, 0.46 mmol) in 5 mL CH₂Cl₂, was added 2 mL trifluoroacetic acid, and then the mixture was stirred overnight. After concentration, the residue was purified by column chromatography eluted with chloroform/methanol/water 35:13:2 to give the desired product as white wax (250 mg, 68%).

¹H NMR (300 MHz, CDCl₃) δ 8.45 (s, 3H), 5.21 (m, 1H), 4.84 (quint, 2H), 4.36 (dd, 1H), 4.18-4.04 (m, 3H), 3.93 (m, 2H), 3.17 (m, 2H), 2.36-2.23 (m, 8H), 1.66-1.53 (m, 8H), 1.51-1.43 (m, 8H), 1.24 (m, 56H), 0.86 (t, 12H).

APCI-MS analysis: Calculated C55H106NO12P [M+H]=1004.7, Observed=1004.1.

### 3-(((2-Aminoethoxy)(hydroxy)phosphoryl)oxy)propane-1,2-diyl bis(2-nonylundecanoate) (Compound 3)

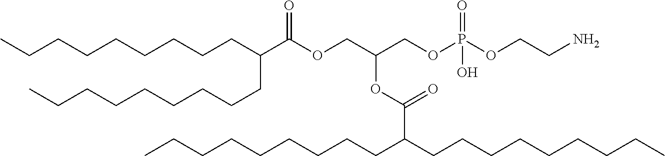 [SMILES: CCCCCCCCCC(CCCCCCCCC)C(=O)OCC(COP(=O)(O)OCCN)OC(=O)C(CCCCCCCCC)CCCCCCCCC]

The title compound was prepared analogously to compound 6. ¹H NMR (300 MHz, CDCl₃) δ 8.44 (m, 3H), 5.21 (m, 1H), 4.50-4.44 (m, 1H), 4.35-3.85 (m, 5H), 3.17 (m, 2H), 2.44-2.02 (m, 6H), 1.63-1.33 (m, 4H), 1.24 (m, 56H), 0.87 (t, 12H).

APCI-MS analysis: Calculated C45H90NO8P [M+H]=804.2, Observed=804.1.

### O,O′-(3-(((2-Aminoethoxy)(hydroxy)phosphoryl)oxy)propane-1,2-diyl) bis(2-pentyldecyl) disuccinate (Compound 4)

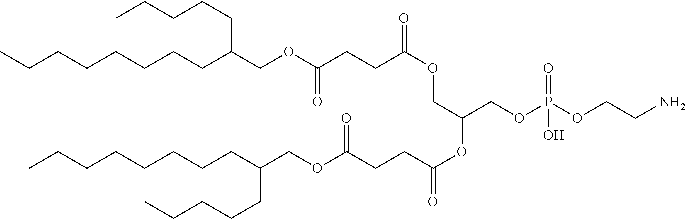 [SMILES: CCCCCCCCC(CCCCC)COC(=O)CCC(=O)OCC(COP(=O)(O)OCCN)OC(=O)CCC(=O)OCC(CCCCC)CCCCCCCC]

The title compound was prepared analogously to compound 6. ¹H NMR (300 MHz, CDCl₃) δ 8.47 (s, 3H), 5.21 (m, 1H), 4.36 (dd, 1H), 4.20 (dd, 1H), 4.10 (m, 2H), 3.96 (d, 6H), 3.17 (m, 2H), 2.61 (s, 8H), 1.61 (m, 2H), 1.25 (m, 44H), 0.87 (t, 12H).

ESI-MS analysis: Calculated C43H82NO12P [M+H]=836.5, Observed=836.0.

### O,O′-(3-(((2-Aminoethoxy)(hydroxy)phosphoryl)oxy)propane-1,2-diyl) bis(2-pentyldecyl) disuccinate (Compound 5)

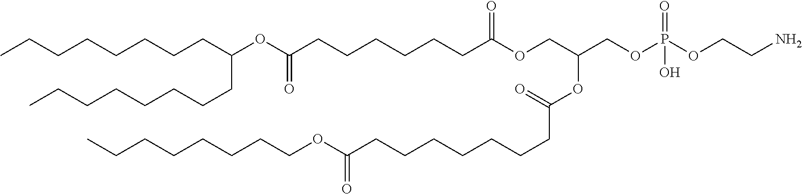 [SMILES: CCCCCCCCOC(=O)CCCCCCCC(=O)OC(COC(=O)CCCCCCC(=O)OC(CCCCCCCC)CCCCCCCC)COP(=O)(O)OCCN]

The title compound was prepared analogously to compound 6. ¹H NMR (300 MHz, CDCl₃) δ 8.47 (s, 3H), 5.21 (m, 1H), 4.36 (dd, 1H), 4.20 (dd, 1H), 4.10 (m, 2H), 3.96 (d, 6H), 3.17 (m, 2H), 2.61 (s, 8H), 1.61 (m, 2H), 1.25 (m, 44H), 0.87 (t, 12H).

ESI-MS analysis: Calculated C43H82NO12P [M+H]=836.5, Observed=836.0.

### Synthetic Scheme for Compound 34

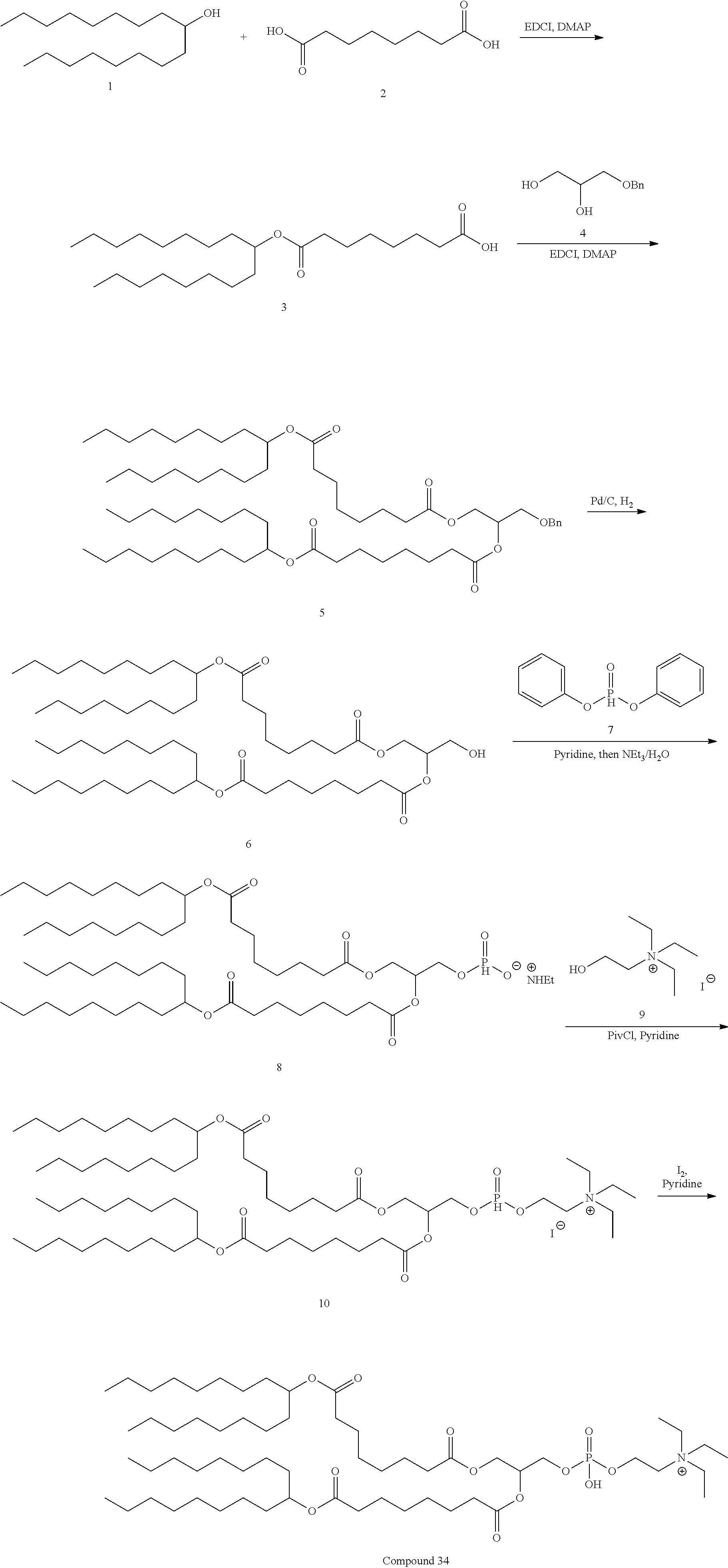

### Synthetic Procedure for Compound 34

### Step 1: Synthesis of 8-(heptadecane-9-yloxy)-8-oxooctanoic acid (3)

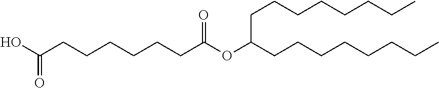 [SMILES: CCCCCCCCC(CCCCCCCC)OC(=O)CCCCCCC(=O)O]

A mixture of suberic acid 2 (22.0 g, 0.126 mole), 1-octylnonanol 1 (16.2 g, 63.1 mmol), EDCI (24.0 g, 0.126 mole) and DMAP (771 mg, 6.3 mmol) in 400 mL dichloromethane was stirred at room temperature overnight. TLC showed alcohol was still present. The reaction mixture was diluted with water, extracted with dichloromethane, and the combined organic layer was washed with brine. After dried over sodium sulfate and concentrated, the residue was purified by flash column chromatography (SiO₂: 0 to 40% EtOAc in hexane) to give 8-(heptadecane-9-yloxy)-8-oxooctanoic acid as colorless oil (13.2 g, 50%).

### Step 2: Synthesis of O′¹,O¹-(3-(Benzyloxy)propane-1,2-diyl) 8-di(heptadecan-9-yl) di(octanedioate) (5)

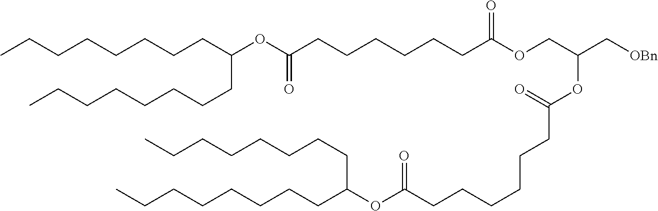 [SMILES: CCCCCCCCC(CCCCCCCC)OC(=O)CCCCCCC(=O)OCC(COCc1ccccc1)OC(=O)CCCCCCC(=O)OC(CCCCCCCC)CCCCCCCC]

A solution of 8-(heptadecane-9-yloxy)-8-oxooctanoic acid 3 (5.1 g, 12 mmol), 3-(benzyloxy)propane-1,2-diol 4 (1.0 g, 5.5 mmol), EDCI (5.5 g, 29 mmol) and DMAP (1.5 g, 12 mmol) in 50 mL dichloromethane was stirred at room temperature overnight. The solvent was removed under reduced pressure, and the residue was triturated with hexanes three times. The solution was concentrated, and the residue was purified by flash column chromatography (SiO₂: 0 to 30% ethyl acetate in hexanes) to give O′¹,O¹-(3-(benzyloxy)propane-1,2-diyl) 8-di(heptadecan-9-yl) di(octanedioate) as colorless oil (5.1 g, 97%).

### Step 3: Synthesis of 8-Di(heptadecan-9-yl) O′¹,O¹-(3-hydroxypropane-1,2-diyl) di(octanedioate) (6)

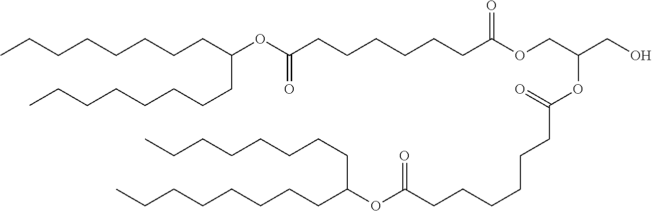 [SMILES: CCCCCCCCC(CCCCCCCC)OC(=O)CCCCCCC(=O)OCC(CO)OC(=O)CCCCCCC(=O)OC(CCCCCCCC)CCCCCCCC]

A mixture of O′¹,O¹-(3-(benzyloxy)propane-1,2-diyl) 8-di(heptadecan-9-yl) di(octanedioate) 5 (5.1 g, 5.3 mmol) and 10% Pd/C (0.7 g) in 20 mL ethyl acetate was purged with nitrogen and hydrogen 3 times respectively, and then the reaction was stirred at room temperature with hydrogen balloon for 16 h. After filtration and concentration, 8-di(heptadecan-9-yl) O′¹,O¹-(3-hydroxypropane-1,2-diyl) di(octanedioate) was obtained as colorless oil (4.7 g, quant.).

### Step 4: Synthesis of Triethylammonium 2,3-Bis((8-(heptadecan-9-yloxy)-8-oxooctanoyl)oxy)propyl phosphonate (8)

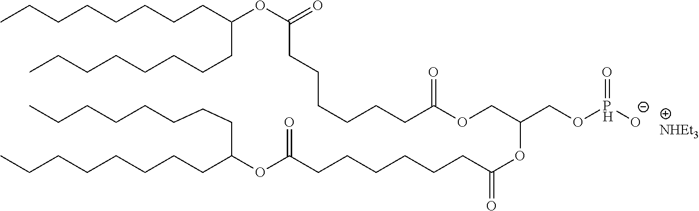

To a solution of 8-di(heptadecan-9-yl) O′¹,O¹-(3-hydroxypropane-1,2-diyl) di(octanedioate) δ (3.0 g, 3.4 mmol) in 10 mL pyridine, was added diphenyl phosphite 7 (3.9 mL, 20 mmol) dropwise at 0° C., and the resulting mixture was stirred at this temperature for 1 h. A mixture of triethylamine and water (10 mL, 1:1) was added, and the mixture was stirred at room temperature for another hour. After concentration, the crude was partitioned with dichloromethane and saturated sodium bicarbonate solution. The combined organic layer was washed with brine and dried over Na₂SO₄, and then concentrated. The residue was purified by column chromatography (SiO₂: 95:5:0.5 CHCl₃-MeOH-NEt₃) to afford triethylammonium 2,3-bis((8-(heptadecan-9-yloxy)-8-oxooctanoyl)oxy)propyl phosphonate as colorless oil (2.5 g, 70%).

¹H NMR (400 MHz, CDCl₃): δ 7.61 (s, 0.5H), 6.04 (s, 0.5H), 5.24-5.13 (m, 1H), 4.83 (quint, 2H), 4.35 (dd, 1H), 4.15 (dd, 1H), 3.98 (dd, 2H), 3.12-2.94 (m, 6H), 2.38-2.15 (m, 8H), 1.70-1.39 (m, 16H), 1.37-1.09 (m, 65H), 0.86 (t, 12H).

³¹P NMR (162 MHz, CDCl₃): δ 5.44.

ESI-MS: m/z 1046.5 (M+H).

### Step 5: Synthesis of 2-(((2,3-Bis((8-(heptadecan-9-yloxy)-8-oxooctanoyl)oxy)propoxy)-phosphoryl)oxy)-N,N,N-triethylethan-1-aminium iodide (10)

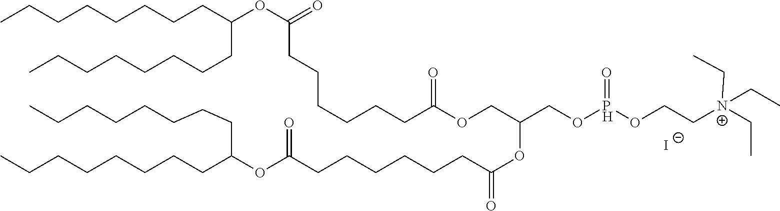

A solution of triethylammonium 2,3-bis((8-(heptadecan-9-yloxy)-8-oxooctanoyl)oxy)propyl phosphonate 8 (1.0 g, 0.96 mmol) and N,N,N-triethyl-2-hydroxyethan-1-aminium iodide 9 (0.31 g, 1.1 mmol) in 10 mL pyridine and 2 mL dichloromethane was cooled to 0° C., pivaloyl chloride (0.15 mL, 1.12 mmol) was added dropwise, and the resulting solution was stirred at this temperature for 1 h. After concentration, the residue was partitioned with dichloromethane and saturated sodium bicarbonate solution. After washed with brine and dried over sodium sulfate, the solvent was removed, and the crude was used for the next step without further purification.

ESI-MS: m/z 1072.8 (M+H).

### Step 6: Synthesis of 2,3-Bis((8-(heptadecan-9-yloxy)-8-oxooctanoyl)oxy)propyl (2-(triethylammonio)ethyl) phosphate (Compound 34)

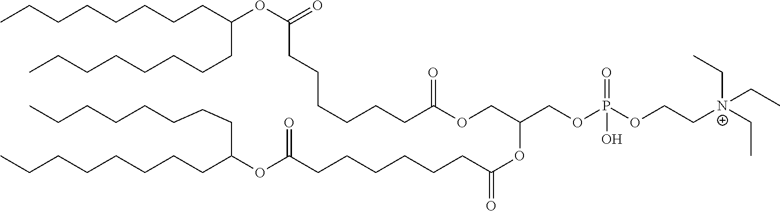 [SMILES: CCCCCCCCC(CCCCCCCC)OC(=O)CCCCCCC(=O)OCC(COP(=O)(O)OCC[N+](CC)(CC)CC)OC(=O)CCCCCCC(=O)OC(CCCCCCCC)CCCCCCCC]

A solution of 2-(((2,3-bis((8-(heptadecan-9-yloxy)-8-oxooctanoyl)oxy)propoxy)-phosphoryl)oxy)-N,N,N-triethylethan-1-aminium iodide 10 (1.0 g crude) in 4.7 mL 95% pyridine-water was cooled to 0° C., 800 mg iodine was added, and then the mixture was stirred at room temperature for 3 h. After concentration, the residue was partitioned by dichloromethane and saturated sodium thiosulfate solution, and the organic layer was washed with saturated sodium bicarbonate and brine. After dried over sodium sulfate and concentration, the crude was purified by column chromatography (SiO₂: CHCl₃/MeOH/H₂O 65:25:4) to give 2,3-bis((8-(heptadecan-9-yloxy)-8-oxooctanoyl)oxy)propyl (2-(triethylammonio)ethyl) phosphate as colorless oil (107 mg, 10%).

¹H NMR (400 MHz, CDCl₃): 5.24-5.15 (m, 1H), 4.83 (quint, 2H), 4.39 (dd, 1H), 4.33-4.24 (m, 2H), 4.10 (dd, 1H), 3.96 (t, 2H), 3.66-3.57 (m, 2H), 3.55-3.43 (m, 6H), 2.41-2.06 (m, 10H), 1.69-1.43 (m, 16H), 1.42-1.11 (m, 63H), 0.86 (t, 12H).

³¹P NMR (162 MHz, CDCl₃): δ 0.02.

APCI-MS analysis: Calculated C61H118NO12P [M+H]=1088.8, Observed=1088.8.

HPLC-ELSD: t_{R}=10.420 min (method 1).

### Synthetic Scheme for Compound 31

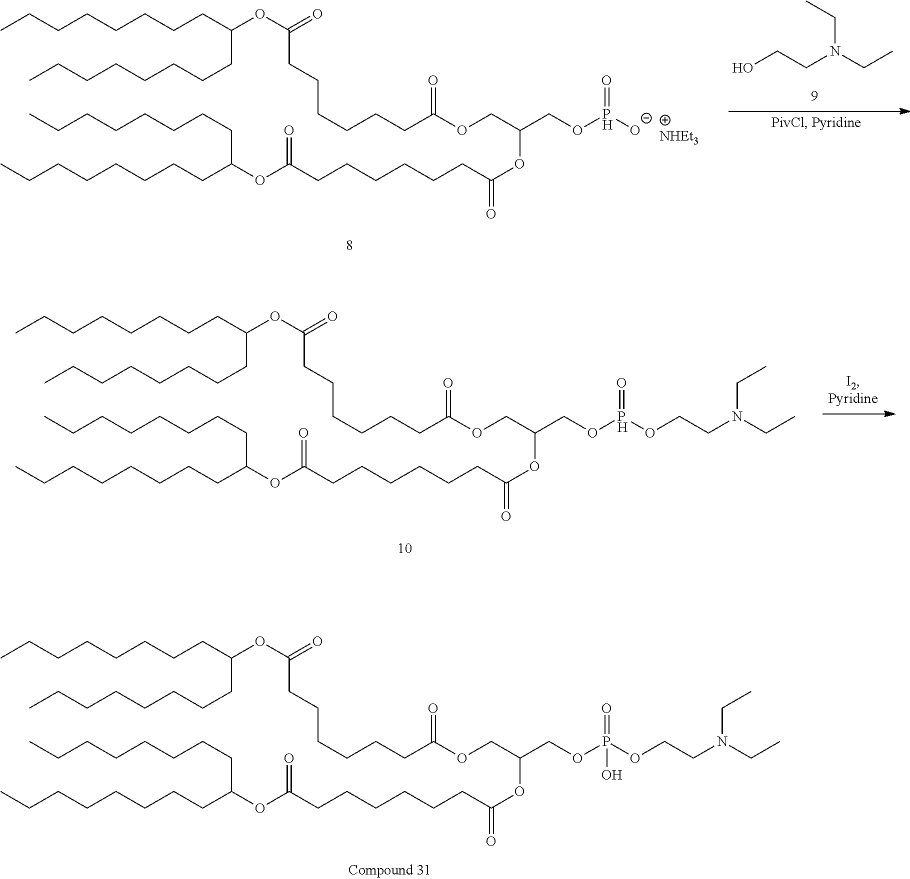

### Synthetic Procedure for Compound 31

### Step 1: Synthesis of O′¹,O¹-(3-(((2-(Diethylamino)ethoxy)phosphoryl)oxy)propane-1,2-diyl) 8-di(heptadecan-9-yl) di(octanedioate) (10)

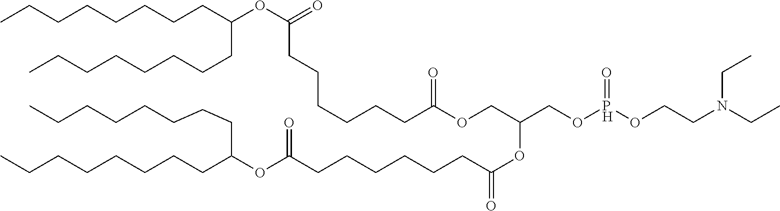

A solution of triethylammonium 2,3-bis((8-(heptadecan-9-yloxy)-8-oxooctanoyl)oxy)propyl phosphonate 8 (1.1 g, 1.1 mmol) and 2-(diethylamino)ethan-1-ol 9 (0.15 g, 1.3 mmol) in 10 mL pyridine was cooled to 0° C., pivaloyl chloride (0.77 mL, 6.3 mmol) was added dropwise, and the resulting solution was stirred at this temperature for 1 h. After concentration, the residue was partitioned with dichloromethane and saturated sodium bicarbonate solution. After washed with brine and dried over sodium sulfate, the solvent was removed, and the crude was used for the next step without further purification.

ESI-MS: m/z 1044.8 (M+H).

### Step 2: Synthesis of O′¹,O¹-(3-(((2-(Diethylamino)ethoxy)(hydroxy)phosphoryl)oxy)-propane-1,2-diyl) 8-di(heptadecan-9-yl) di(octanedioate) (Compound 31)

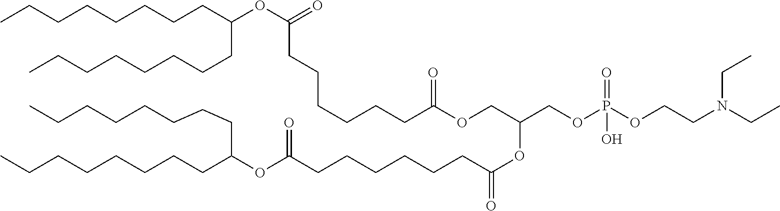 [SMILES: CCCCCCCCC(CCCCCCCC)OC(=O)CCCCCCC(=O)OCC(COP(=O)(O)OCCN(CC)CC)OC(=O)CCCCCCC(=O)OC(CCCCCCCC)CCCCCCCC]

A solution of O′¹,O¹-(3-(((2-(diethylamino)ethoxy)phosphoryl)oxy)propane-1,2-diyl) 8-di(heptadecan-9-yl) di(octanedioate) 10 (1.1 g crude) in 10 mL 95% pyridine-water was cooled to 0° C., iodine (800 mg, 3.2 mmol) was added, and then the mixture was stirred at room temperature for 3 h. After concentration, the residue was partitioned by dichloromethane and saturated sodium thiosulfate solution, and the organic layer was washed with saturated sodium bicarbonate and brine. After dried over sodium sulfate and concentration, the crude was purified by column chromatography (SiO₂: CHCl₃/MeOH/H₂O 65:25:4) to give O′¹,O¹′-(3-(((2-(diethylamino)ethoxy)(hydroxy)phosphoryl)oxy)-propane-1,2-diyl) 8-di(heptadecan-9-yl) di(octanedioate) as pale brown gum (150 mg, 13%).

¹H NMR (400 MHz, CDCl₃): 5.24-5.14 (m, 1H), 4.83 (quint, 2H), 4.34 (dd, 1H), 4.24-4.16 (m, 2H), 4.15-4.07 (m, 1H), 4.03-3.94 (m, 2H), 3.20-3.06 (m, 6H), 2.32-2.16 (m, 8H), 1.66-1.39 (m, 16H), 1.36-1.10 (m, 63H), 0.84 (t, 12H).

³¹P NMR (162 MHz, CDCl₃): δ 1.74.

APCI-MS analysis: Calculated C59H114NO12P [M+H]=1060.8, Observed=1060.8.

HPLC-ELSD: t_{R}=10.372 min (method 1).

### Synthetic Scheme for Compound 32

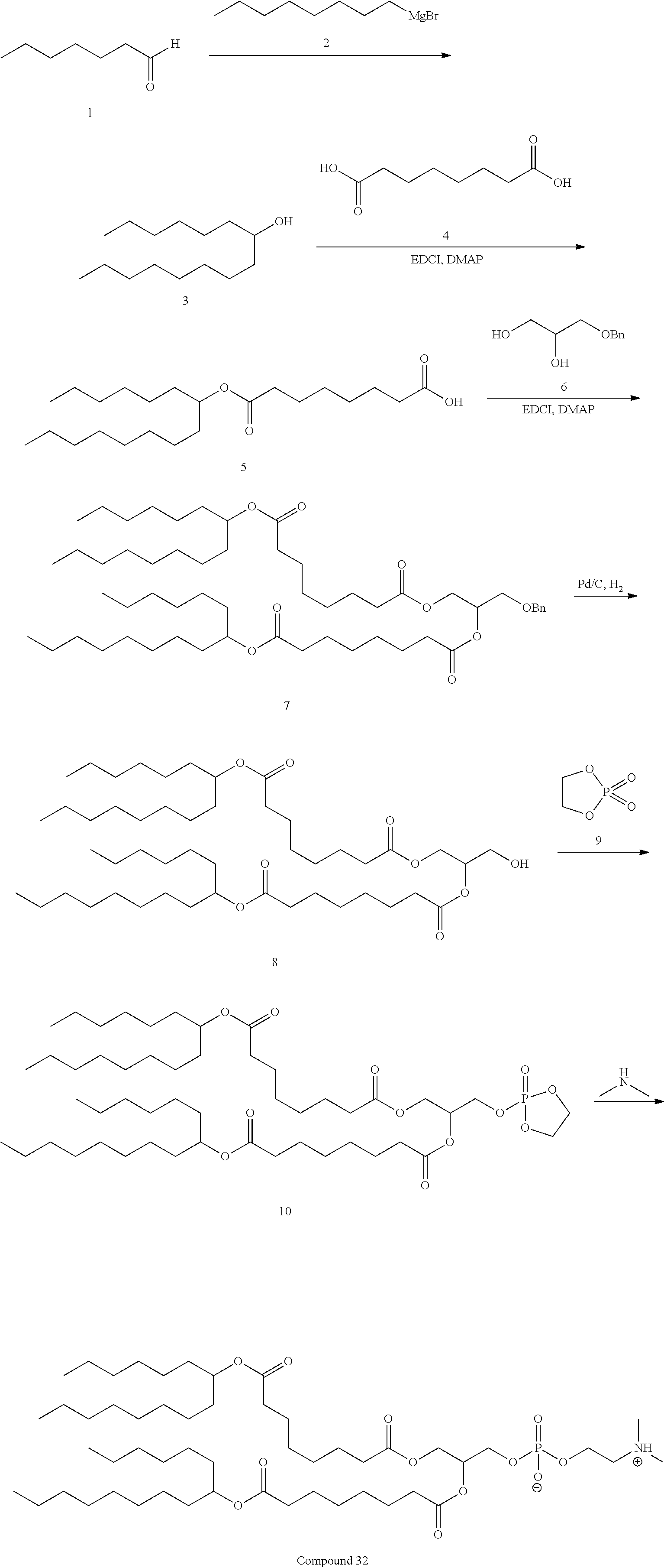

### Synthetic Procedure for Compound 32

### Step 1: Synthesis of Pentadecan-7-ol (3)

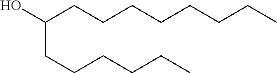 [SMILES: CCCCCCCCC(O)CCCCCC]

At 0° C., to a solution of octylmagnesium bromide 2 (2 M in ether, 52.5 mL, 0.105 mole) in 100 mL ether, was slowly added a solution of heptaldehyde 1 (12.6 mL, 90 mmol) in 40 mL ether, and the mixture was warmed up to room temperature and stirred overnight. The reaction was quenched by saturated ammonium chloride and extracted with ether; the organic layer was dried over sodium sulfate. After concentration, the crude was purified by column chromatography (SiO₂: 0-20% EtOAc in hexane) to afford the desired product as white wax (10.0 g, 50%).

### Step 2: Synthetic of 8-Oxo-8-(pentadecan-7-yloxy)octanoic acid (5)

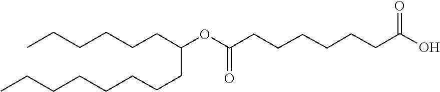 [SMILES: CCCCCCCCC(CCCCCC)OC(=O)CCCCCCC(=O)O]

To a mixture of suberic acid 4 (12.8 g, 74 mmol), EDCI (14.1 g, 74 mmol) and DMAP (3.8 g, 30.8 mmol) in a mixture of 40 mL dichloromethane and 30 mL DMF, was added a solution pentadecan-7-ol 3 (7.0 g, 30.8 mmol) of in 35 mL dichloromethane slowly, and the resulting mixture was stirred at room temperature overnight. The clear solution was concentrated, and the residue was partitioned with saturated ammonium chloride solution and hexane then ethyl acetate. The combined organic layer was concentrated under vacuum, and the crude was purified by flash column chromatography (SiO₂: 0 to 20% ethyl acetate in hexane) to give 8-oxo-8-(pentadecan-7-yloxy)octanoic acid as colorless oil (7.0 g, 59%).

### Step 3: Synthetic of O′¹,O¹-(3-(Benzyloxy)propane-1,2-diyl) 8-di(pentadecan-7-yl) di(octanedioate) (7)

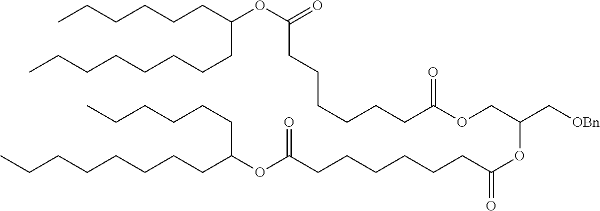 [SMILES: CCCCCCCCC(CCCCCC)OC(=O)CCCCCCC(=O)OCC(COCc1ccccc1)OC(=O)CCCCCCC(=O)OC(CCCCCC)CCCCCCCC]

A solution of 8-oxo-8-(pentadecan-7-yloxy)octanoic acid 5 (7.0 g, 18 mmol), 3-(benzyloxy)propane-1,2-diol 6 (1.5 g, 8.2 mmol), EDCI (9.5 g, 49 mmol) and DMAP (2.2 g, 18 mmol) in 50 mL dichloromethane was stirred at room temperature for 48 h. After concentration, the residue was partitioned with saturated ammonium chloride solution and ethyl acetate. The combined organic layer was concentrated under vacuum, and the crude was purified by flash column chromatography (SiO₂: 0 to 20% ethyl acetate in hexane) to give O′¹,O¹-(3-(benzyloxy)propane-1,2-diyl) 8-di(pentadecane-7-yl) di(octanedioate) as colorless oil (6.5 g, 86%).

### Step 4: Synthesis of O′¹,O¹-(3-Hydroxypropane-1,2-diyl) 8-di(pentadecan-7-yl) di(octanedioate) (8)

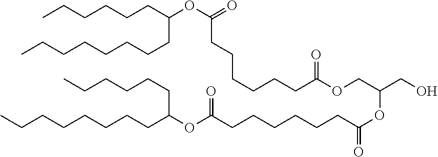 [SMILES: CCCCCCCCC(CCCCCC)OC(=O)CCCCCCC(=O)OCC(CO)OC(=O)CCCCCCC(=O)OC(CCCCCC)CCCCCCCC]

A mixture of O′¹,O¹-(3-(benzyloxy)propane-1,2-diyl) 8-di(pentadecane-7-yl) di(octanedioate) 7 (6.5 g, 7.1 mmol) and Pd/C (10 wt %, 700 mg) in 30 mL ethyl acetate was hydrogenated with balloon for 16 h. After filtration and concentration, O′¹,O¹-(3-hydroxypropane-1,2-diyl) 8-di(pentadecan-7-yl) di(octanedioate) was obtained as colorless oil (5.9 g, quant.).

¹H NMR (400 MHz, CDCl₃) δ 5.06 (quint, 1H), 4.85 (quint, 2H), 4.30 (dd, 1H), 4.21 (dd, 1H), 3.71 (t, 2H), 2.40-2.20 (m, 8H), 2.19-2.09 (m, 1H), 1.70-1.54 (m, 8H), 1.53-1.41 (m, 8H), 1.38-1.15 (m, 48H), 0.86 (t, 12H).

APCI-MS analysis: Calculated C49H9209 [M+H]=825.7, Observed=825.6, 807.6 (M+H—H₂O).

HPLC-ELSD: t_{R}=6.476 min (method 2).

### Step 5: Synthesis of O′¹,O¹-(3-((2-Oxido-1,3,2-dioxaphospholan-2-yl)oxy)propane-1,2-diyl) 8-di(pentadecan-7-yl) di(octanedioate) (10)

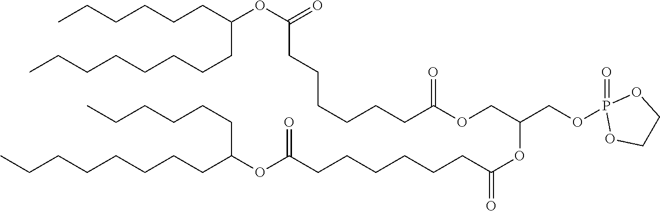 [SMILES: CCCCCCCCC(CCCCCC)OC(=O)CCCCCCC(=O)OCC(COP1(=O)OCCO1)OC(=O)CCCCCCC(=O)OC(CCCCCC)CCCCCCCC]

A solution of O′¹,O¹-(3-hydroxypropane-1,2-diyl) 8-di(pentadecan-7-yl) di(octanedioate) 8 (1.5 g, 1.8 mmol) and triethylamine (0.38 mL, 2.7 mmol) in 10 mL THF was cooled to 0° C., and then 2-chloro-2-oxo-1,3,2-dioxaphospholane (390 mg, 2.7 mmol) was added dropwise. The reaction mixture was stirred at room temperature overnight. After filtration, the filtrate was concentrated under vacuum, and the residue was used for next step without further purification.

### Step 6: Synthesis of O′¹,O¹-(3-(((2-(Dimethylamino)ethoxy)(hydroxy)phosphoryl)oxy)-propane-1,2-diyl) 8-di(pentadecan-7-yl) di(octanedioate) (Compound 32)

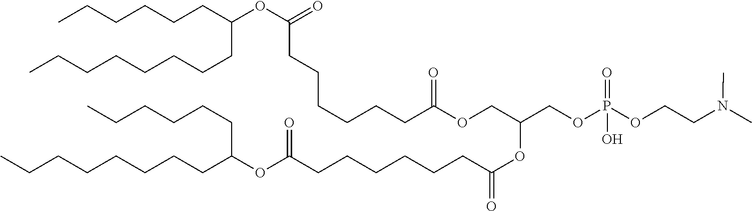 [SMILES: CCCCCCCCC(CCCCCC)OC(=O)CCCCCCC(=O)OCC(COP(=O)(O)OCCN(C)C)OC(=O)CCCCCCC(=O)OC(CCCCCC)CCCCCCCC]

In a seal tube, a solution of O,O′-(3-((2-oxido-1,3,2-dioxaphospholan-2-yl)oxy)propane-1,2-diyl) 8-di(pentadecan-7-yl) di(octanedioate) 10 (crude, 1.8 mmol) in 10 mL acetonitrile was cooled in ice bath, and a solution of dimethylamine (2 M in THF, 5.5 mL, 11 mmol) was added. The bottle was sealed and heated at 85° C. for 16 h. After cooled to room temperature, the volatiles were evaporated in vacuo, and the crude was purified by column chromatography (SiO₂: CHCl₃/MeOH/H₂O 88:13:1) to give the desired product as pale yellow wax (800 mg, 45%).

¹H NMR (400 MHz, CDCl₃) δ 5.30-5.16 (m, 1H), 4.84 (quint, 2H), 4.37 (dd, 1H), 4.27-4.19 (m, 2H), 4.13 (dd, 1H), 4.01 (t, 2H), 3.22-3.13 (m, 2H), 2.85 (s, 6H), 2.35-2.20 (m, 8H), 1.68-1.40 (m, 16H), 1.38-1.13 (m, 48H), 0.86 (t, 12H).

³¹P NMR (162 MHz, CDCl₃): δ 0.38.

APCI-MS analysis: Calculated C53H102NO12P [M+H]=976.7, Observed=976.7.

HPLC-ELSD: t_{R}=8.133 min (method 1).

### Synthesis of 2,3-Bis((8-oxo-8-(pentadecan-7-yloxy)octanoyl)oxy)propyl (2-(trimethylammonio)ethyl) phosphate (Compound 35)

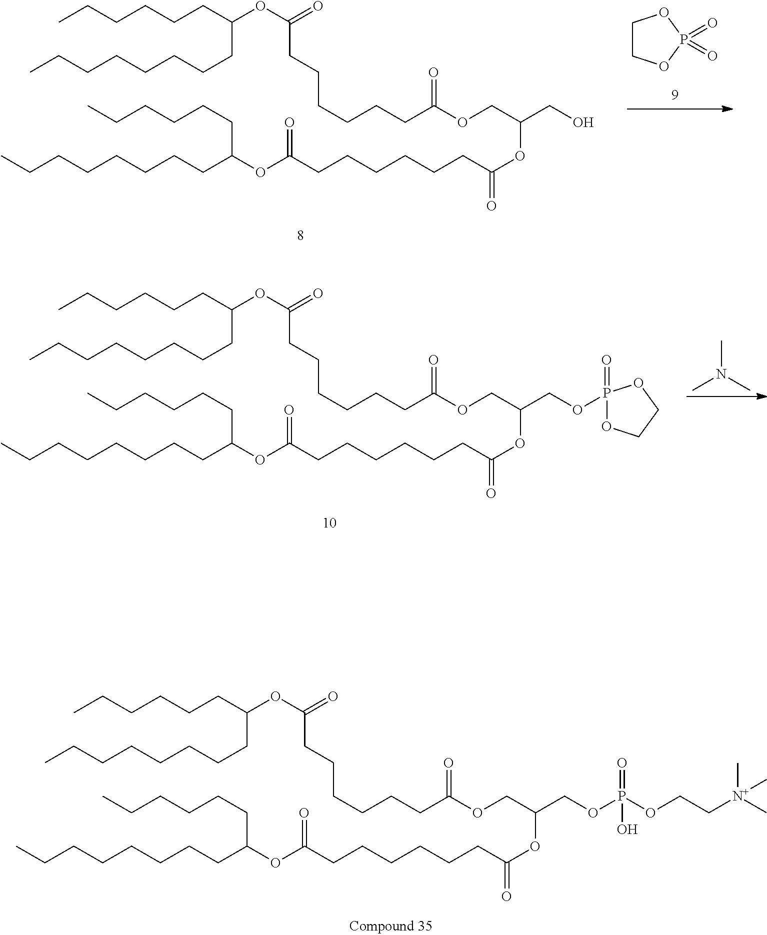

A solution of O′¹,O¹-(3-hydroxypropane-1,2-diyl) 8-di(pentadecan-7-yl) di(octanedioate) 8 (1.0 g, 1.2 mmol) and triethylamine (0.25 mL, 1.8 mmol) in 10 mL THF was cooled to 0° C., and then 2-chloro-2-oxo-1,3,2-dioxaphospholane (260 mg, 1.8 mmol) was added dropwise. The reaction mixture was stirred at room temperature overnight. After filtration, the filtrate was concentrated under vacuum, and the residue was used for next step without further purification. In a seal tube, a solution of O′¹,O¹-(3-((2-oxido-1,3,2-dioxaphospholan-2-yl)oxy)propane-1,2-diyl) 8-di(pentadecan-7-yl) di(octanedioate) 10 (crude) in 10 mL acetonitrile was cooled in ice bath, and a solution of trimethylamine (2 M in THF, 3.5 mL, 7 mmol) was added. The bottle was sealed and heated at 85° C. for 48 h. After cooled to room temperature, the volatiles were evaporated in vacuo, and the crude was purified by column chromatography (SiO₂: CHCl₃/MeOH/H₂O 88:13:1) to give the desired product as colorless wax (590 mg, 50%).

¹H NMR (400 MHz, CDCl₃) δ 5.25-5.15 (m, 1H), 4.83 (quint, 2H), 4.43-4.32 (m, 3H), 4.11 (dd, 1H), 4.01-3.87 (m, 4H), 3.40 (s, 9H), 2.36-2.16 (m, 8H), 1.70-1.42 (m, 16H), 1.37-1.14 (m, 48H), 0.86 (t, 12H).

³¹P NMR (162 MHz, CDCl₃): δ −0.24.

APCI-MS analysis: Calculated C54H104NO12P [M+H]=990.7, Observed=990.6.

HPLC-ELSD: t_{R}=7.460 min (method 1).

### Synthetic Scheme for Compound 33

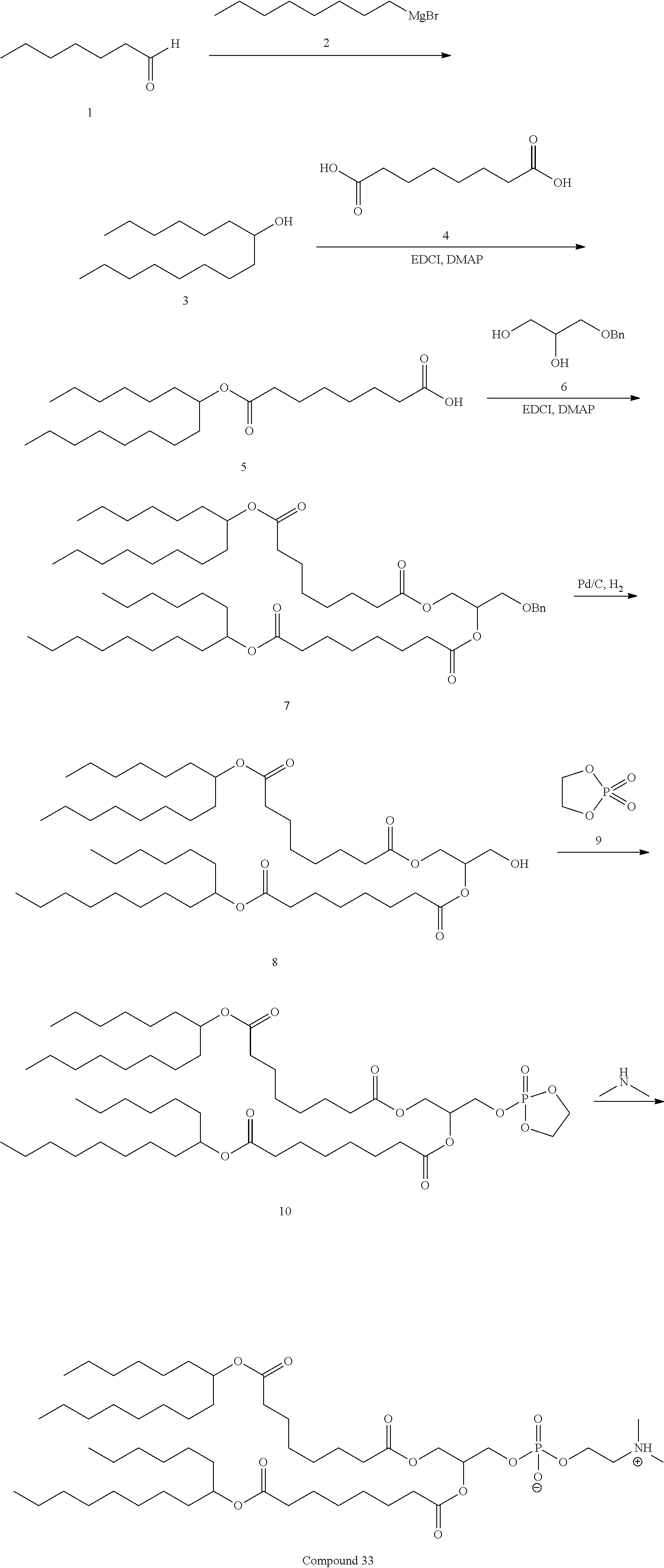

### Synthetic Procedure for Compound 33

### Step 1: Synthesis of Tridecan-5-ol (3)

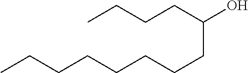

At 0° C., to a solution of nonanal 1 (23 g, 0.16 mole) in 200 mL THF, was slowly added a solution of butylmagnesium chloride 2 (2 M in ether, 100 mL, 0.2 mole), and the mixture was warmed up to room temperature and stirred overnight. The reaction was quenched by saturated ammonium chloride and extracted with ethyl acetate; the organic layer was dried over sodium sulfate. After concentration, the crude was purified by column chromatography (SiO₂: 0-20% EtOAc in hexane) to afford the desired product as white wax (5.3 g, 16%).

### Step 2: Synthetic of 8-Oxo-8-(tridecan-5-yloxy)octanoic acid (5)

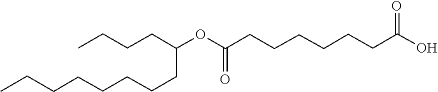 [SMILES: CCCCCCCCC(CCCC)OC(=O)CCCCCCC(=O)O]

To a mixture of suberic acid 4 (9.2 g, 53 mmol), EDCI (10 g, 53 mmol) and DMAP (3.2 g, 26 mmol) in a mixture of 20 mL dichloromethane and 20 mL DMF, was added a solution of tridecan-5-ol 3 (5.3 g, 26 mmol) in 20 mL dichloromethane slowly, and the resulting mixture was stirred at room temperature overnight. The clear solution was concentrated, and the residue was partitioned with saturated ammonium chloride solution and hexane then ethyl acetate. The combined organic layer was concentrated under vacuum, and the crude was purified by flash column chromatography (SiO₂: 0 to 50% ethyl acetate in hexane) to give 8-oxo-8-(tridecan-5-yloxy)octanoic acid as colorless oil (4.2 g, 45%).

### Step 3: Synthetic of O′¹,O¹-(3-(Benzyloxy)propane-1,2-diyl) 8-di(tridecan-5-yl) di(octanedioate) (7)

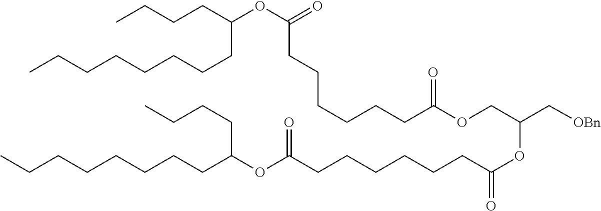 [SMILES: CCCCCCCCC(CCCC)OC(=O)CCCCCCC(=O)OCC(COCc1ccccc1)OC(=O)CCCCCCC(=O)OC(CCCC)CCCCCCCC]

A solution of 8-oxo-8-(tridecan-5-yloxy)octanoic acid 5 (4.22 g, 11.8 mmol), 3-(benzyloxy)propane-1,2-diol 6 (0.93 g, 5.1 mmol), EDCI (5.9 g, 31 mmol) and DMAP (1.2 g, 10 mmol) in 60 mL dichloromethane was stirred at room temperature for 17 h. After concentration, the residue was partitioned with saturated ammonium chloride solution and ethyl acetate. The combined organic layer was concentrated under vacuum, and the crude was purified by flash column chromatography (SiO₂: 0 to 20% ethyl acetate in hexane) to give O′¹,O¹-(3-(benzyloxy)propane-1,2-diyl) 8-di(tridecane-5-yl) di(octanedioate) as colorless oil (3.9 g, 89%).

### Step 4: Synthesis of O′¹,O¹-(3-Hydroxypropane-1,2-diyl) 8-di(tridecan-5-yl) di(octanedioate) (8)

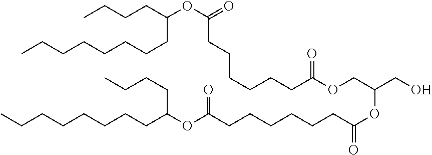 [SMILES: CCCCCCCCC(CCCC)OC(=O)CCCCCCC(=O)OCC(CO)OC(=O)CCCCCCC(=O)OC(CCCC)CCCCCCCC]

A mixture of O′¹,O¹-(3-(benzyloxy)propane-1,2-diyl) 8-di(tridecane-5-yl) di(octanedioate) 7 (3.9 g, 4.5 mmol) and Pd/C (10 wt %, 120 mg) in 20 mL ethyl acetate was hydrogenated in a Parr system (40 psi) for 16 h. After filtration and concentration, O′¹,O¹-(3-hydroxypropane-1,2-diyl) 8-di(tridecan-5-yl) di(octanedioate) was obtained as colorless oil (3.4 g, 97%).

¹H NMR (400 MHz, CDCl₃) δ 5.06 (quint, 1H), 4.85 (quint, 2H), 4.31 (dd, 1H), 4.22 (dd, 1H), 3.71 (t, 2H), 2.40-2.22 (m, 8H), 2.17-2.09 (m, 1H), 1.69-1.42 (m, 16H), 1.39-1.15 (m, 40H), 0.93-0.79 (m, 12H).

APCI-MS analysis: Calculated C45H8409 [M+H]=769.6, Observed=769.1, 751.1 (M+H—H₂O).

HPLC-ELSD: t_{R}=6.668 min (method 2).

### Step 5: Synthesis of O′¹,O¹-(3-((2-Oxido-1,3,2-dioxaphospholan-2-yl)oxy)propane-1,2-diyl) 8-di(tridecan-5-yl) di(octanedioate) (10)

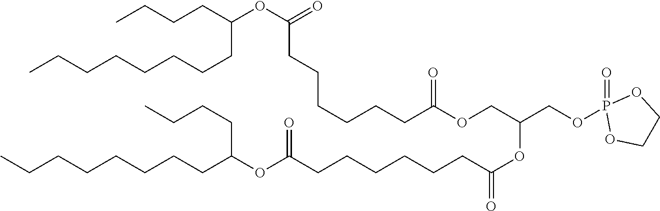 [SMILES: CCCCCCCCC(CCCC)OC(=O)CCCCCCC(=O)OCC(COP1(=O)OCCO1)OC(=O)CCCCCCC(=O)OC(CCCC)CCCCCCCC]

A solution of O′¹,O¹-(3-hydroxypropane-1,2-diyl) 8-di(tridecan-5-yl) di(octanedioate) 8 (800 mg, 1.04 mmol) and triethylamine (0.36 mL, 2.6 mmol) in 8 mL THF was cooled to 0° C., and then 2-chloro-2-oxo-1,3,2-dioxaphospholane 9 (370 mg, 2.6 mmol) was added dropwise. The reaction mixture was stirred at room temperature overnight. After filtration, the filtrate was concentrated under vacuum, and the residue was used for next step without further purification.

### Step 6: Synthesis of O′¹,O¹-(3-(((2-(Dimethylamino)ethoxy)(hydroxy)phosphoryl)oxy)-propane-1,2-diyl) 8-di(tridecan-5-yl) di(octanedioate) (Compound 33)

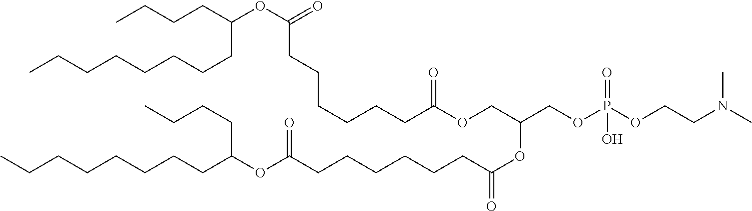 [SMILES: CCCCCCCCC(CCCC)OC(=O)CCCCCCC(=O)OCC(COP(=O)(O)OCCN(C)C)OC(=O)CCCCCCC(=O)OC(CCCC)CCCCCCCC]

In a seal tube, a solution of O,O′-(3-((2-oxido-1,3,2-dioxaphospholan-2-yl)oxy)propane-1,2-diyl) 8-di(tridecan-5-yl) di(octanedioate) 10 (crude) in 10 mL acetonitrile was cooled in ice bath, and a solution of dimethylamine (2 M in THF, 6 mL, 12 mmol) was added. The bottle was sealed and heated at 85° C. for 17 h. After cooled to room temperature, the volatiles were evaporated in vacuo, and the crude was purified by column chromatography (SiO₂: CHCl₃/MeOH/H₂O 80:15:2) to give the desired product as pale yellow wax (535 mg, 56%).

¹H NMR (400 MHz, CDCl₃) δ 5.27-5.18 (m, 1H), 4.84 (quint, 2H), 4.37 (dd, 1H), 4.27-4.18 (m, 2H), 4.14 (dd, 1H), 4.02 (t, 2H), 3.25-3.16 (m, 2H), 2.85 (s, 6H), 2.35-2.17 (m, 8H), 1.67-1.42 (m, 16H), 1.38-1.12 (m, 40H), 0.92-0.79 (m, 12H).

³¹P NMR (162 MHz, CDCl₃): δ 0.30.

ESI-MS analysis: Calculated C49H94NO12P [M+H]=920.6, Observed=920.6.

HPLC-ELSD: t_{R}=7.403 min (method 1).

### Synthesis of 2,3-Bis((8-oxo-8-(tridecan-5-yloxy)octanoyl)oxy)propyl (2-(trimethylammonio)ethyl) phosphate (Compound 36)

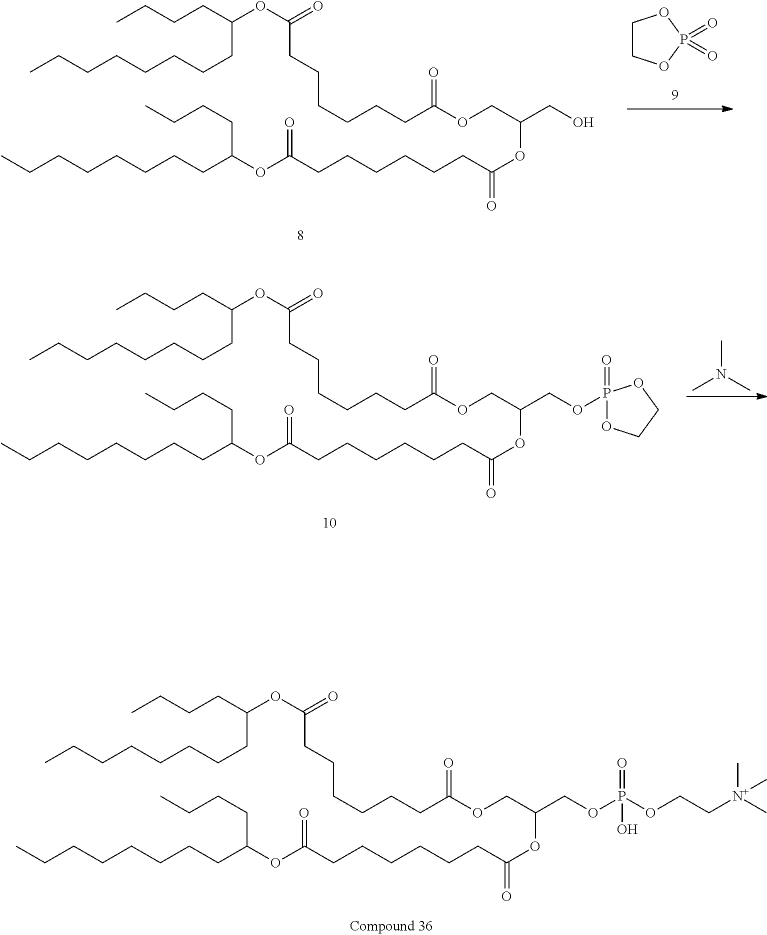

A solution of O′¹,O¹-(3-hydroxypropane-1,2-diyl) 8-di(tridecan-5-yl) di(octanedioate) 8 (700 mg, 0.91 mmol) and triethylamine (0.32 mL, 2.28 mmol) in 8 mL THF was cooled to 0° C., and then 2-chloro-2-oxo-1,3,2-dioxaphospholane 9 (324 mg, 2.28 mmol) was added dropwise. The reaction mixture was stirred at room temperature for 6 h. After filtration, the filtrate was concentrated under vacuum, and the residue was used for next step without further purification.

In a seal tube, a solution of O′¹,O¹-(3-((2-oxido-1,3,2-dioxaphospholan-2-yl)oxy)propane-1,2-diyl) 8-di(tridecan-5-yl) di(octanedioate) 10 (crude) in 10 mL acetonitrile was cooled in ice bath, and a solution of trimethylamine (2 M in THF, 6 mL, 12 mmol) was added. The bottle was sealed and heated at 85° C. for 17 h. After cooled to room temperature, the volatiles were evaporated in vacuo, and the crude was purified by column chromatography (SiO₂: CHCl₃/MeOH/H₂O 80:15:5) to give the desired product as pale yellow wax (535 mg, 56%).

¹H NMR (400 M Hz, CDCl₃) δ 5.22-5.12 (m, 1H), 4.83 (quint, 2H), 4.43-4.24 (m, 3H), 4.10 (dd, 1H), 4.02-3.70 (m, 4H), 3.37 (s, 9H), 2.37-2.16 (m, 8H), 1.68-1.39 (m, 16H), 1.39-1.11 (m, 40H), 0.94-0.76 (m, 12H).

³¹P NMR (162 MHz, CDCl₃): δ −0.42.

ESI-MS analysis: Calculated C50H96NO12P [M+H]=934.6, Observed=934.6.

HPLC-ELSD: t_{R}=7.292 min (method 1).

### Synthetic Scheme for Compound 38

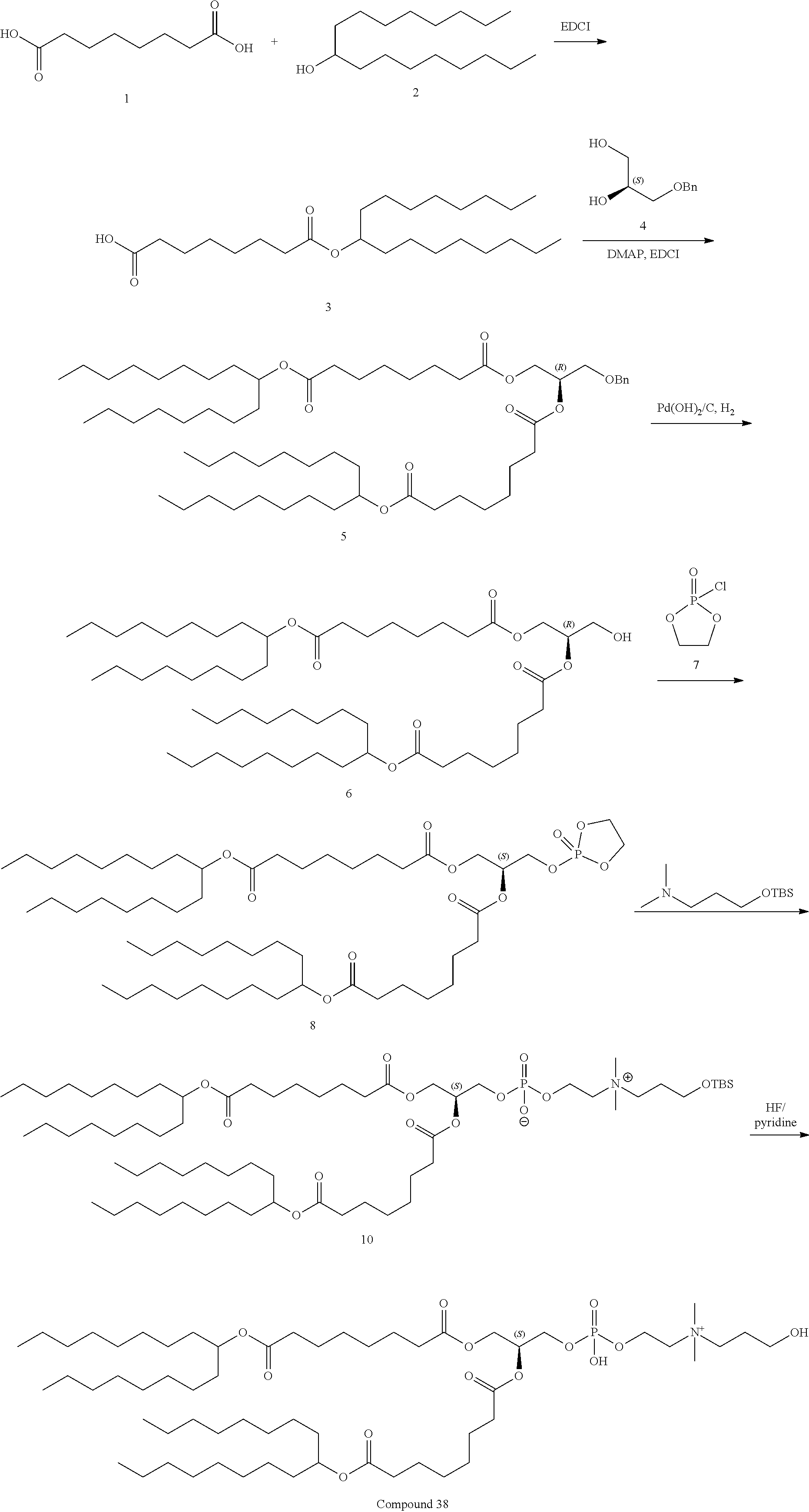

### Synthetic Procedure for Compound 38

### Step 1: Synthesis of 8-(heptadecane-9-yloxy)-8-oxooctanoic acid (3)

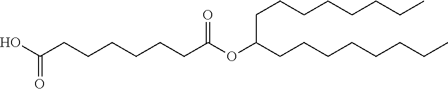 [SMILES: CCCCCCCCC(CCCCCCCC)OC(=O)CCCCCCC(=O)O]

To a stirred homogenous solution of suberic acid 1 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 2 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (R)—O′¹,O¹-(3-(Benzyloxy)propane-1,2-diyl) 8-di(heptadecan-9-yl) di(octanedioate) (5)

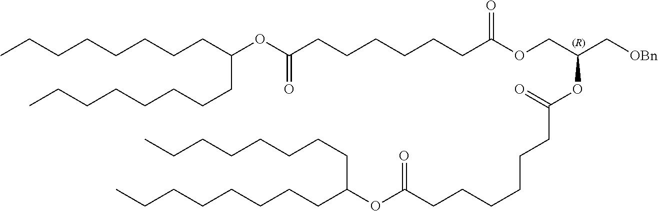

A mixture of (S)-3-(benzyloxy)propane-1,2-diol 4 (3.8 g, 20.9 mmol), 8-(heptadecane-9-yloxy)-8-oxooctanoic acid 3 (19.5 g, 47.3 mmol), EDCI (27.2 g, 142 mmol) and DMAP (5.8 g, 47.5 mmol) in 300 mL dichloromethane was stirred at room temperature for 15 h. TLC showed complete reaction. After concentration, the crude was triturated with hexane, and the solvent was removed. The crude was purified by column chromatography eluted with 0-15% ethyl acetate in hexane to yield (R)—O′¹,O¹-(3-(benzyloxy)propane-1,2-diyl) 8-di(heptadecan-9-yl) di(octanedioate) as colorless oil (18.2 g, 90%).

### Step 3: Synthesis of (R)-8-Di(heptadecan-9-yl) O′¹,O¹-(3-hydroxypropane-1,2-diyl) di(octanedioate) (6)

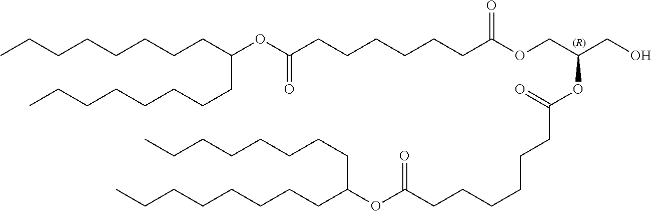

A mixture of (R)—O′¹,O¹-(3-(benzyloxy)propane-1,2-diyl) 8-di(heptadecan-9-yl) di(octanedioate) 5 (6.3 g, 6.5 mmol) and 20% Pd(OH)₂ on carbon (1 g) in 100 mL ethyl acetate was hydrogenated using Parr hydrogenator at 35-40 psi for 12 h. After filtration through silica gel and concentration, the product was used for the next step without further purification (5.6 g, 97%).

### Step 4: Synthesis of (S)-8-Di(heptadecan-9-yl) O′¹,O¹-(3-((2-oxido-1,3,2-dioxaphospholan-2-yl)oxy)propane-1,2-diyl) di(octanedioate) (8)

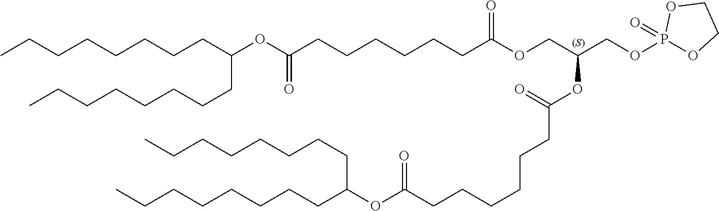

A solution of (R)-8-di(heptadecan-9-yl) O′¹,O¹-(3-hydroxypropane-1,2-diyl) di(octanedioate) δ (705 mg, 0.8 mmol) and triethylamine (0.16 mL, 1.2 mmol) in 5 mL THF was cooled to 0° C., and then a solution of 2-chloro-1,3,2-dioxaphospholane 2-oxide 7 (214 mg, 1.2 mmol) in 1 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 5: Synthesis of (S)-2,3-Bis((8-(heptadecan-9-yloxy)-8-oxooctanoyl)oxy)propyl (2-((3-hydroxypropyl)dimethylammonio)ethyl) phosphate (Compound 38)

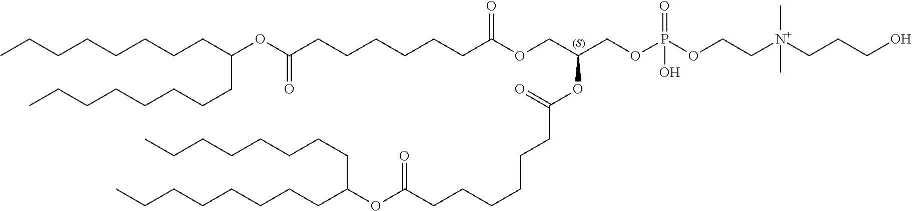

To a solution of the crude (S)-di(heptadecan-9-yl) O′¹,O¹-(3-((2-oxido-1,3,2-dioxaphospholan-2-yl)oxy)propane-1,2-diyl) di(octanedioate) 8 (0.8 mmol) in 2 mL THF and 10 mL acetonitrile, was added 3-((tert-butyldimethylsilyl)oxy)-N,N-dimethylpropan-1-amine 9 (2.5 g, 11.5 mmol), and the resulting mixture was heated at 85-90° C. in a seal tube overnight. MS showed the formation of the desired product. After cooling to room temperature and concentration, the residue was dissolved in 10 mL THF and transferred into Teflon round-bottom flask, 3 mL pyridine and 1.5 mL hydrogen fluoride pyridine complex were added, and the mixture was stirred at room temperature overnight. The reaction was quenched with saturated NaHCO₃ and extracted with EtOAc. The combined organic layer was dried over sodium sulfate and concentrated, and the crude was purified by column chromatography (SiO₂: CHCl₃/MeOH/H₂O 32:13:1) to give the desired product as pale yellow wax (55 mg, 5%).

¹H NMR (400 MHz, CDCl₃) δ 5.26-5.21 (m, 1H), 4.83 (quint, 2H), 4.43-4.23 (m, 3H), 4.15-4.03 (m, 1H), 3.99-3.82 (m, 3H), 3.79-3.60 (m, 6H), 3.35-3.17 (s, 6H), 2.36-2.19 (m, 8H), 2.09-1.94 (m, 2H), 1.69-1.42 (m, 18H), 1.41-1.15 (m, 60H), 0.86 (t, 12H).

APCI-MS analysis: Calculated C60H116NO13P [M+H]=1090.8, Observed=1090.8.

HPLC-ELSD: t_{R}=8.426 min (method 1).

### Synthetic Scheme for Compound 28

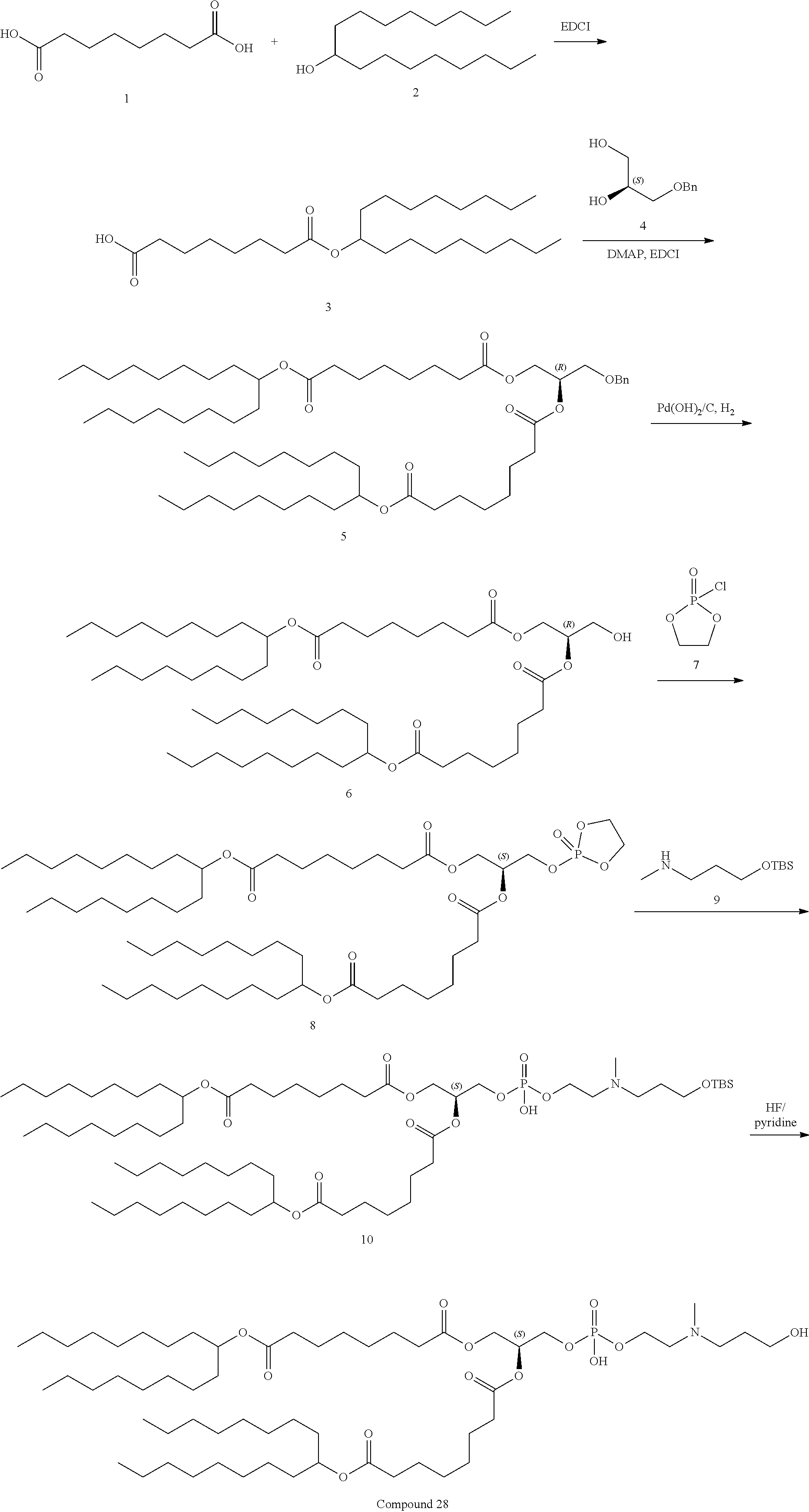

### Synthetic Procedure for Compound 28

### Step 1: Synthesis of 8-(heptadecane-9-yloxy)-8-oxooctanoic acid (3)

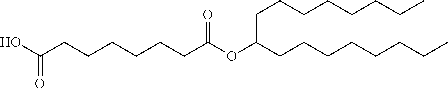 [SMILES: CCCCCCCCC(CCCCCCCC)OC(=O)CCCCCCC(=O)O]

To a stirred homogenous solution of suberic acid 1 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 2 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (R)—O′¹,O¹-(3-(Benzyloxy)propane-1,2-diyl) 8-di(heptadecan-9-yl) di(octanedioate) (5)

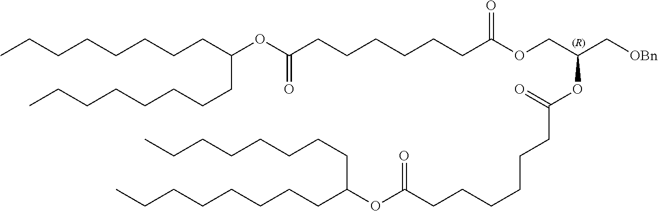

A mixture of (S)-3-(benzyloxy)propane-1,2-diol 4 (3.8 g, 20.9 mmol), 8-(heptadecane-9-yloxy)-8-oxooctanoic acid 3 (19.5 g, 47.3 mmol), EDCI (27.2 g, 142 mmol) and DMAP (5.8 g, 47.5 mmol) in 300 mL dichloromethane was stirred at room temperature for 15 h. TLC showed complete reaction. After concentration, the crude was triturated with hexane, and the solvent was removed. The crude was purified by column chromatography eluted with 0-15% ethyl acetate in hexane to yield (R)—O′¹,O¹-(3-(benzyloxy)propane-1,2-diyl) 8-di(heptadecan-9-yl) di(octanedioate) as colorless oil (18.2 g, 90%).

### Step 3: Synthesis of (R)-8-Di(heptadecan-9-yl) O′¹,O¹-(3-hydroxypropane-1,2-diyl) di(octanedioate) (6)

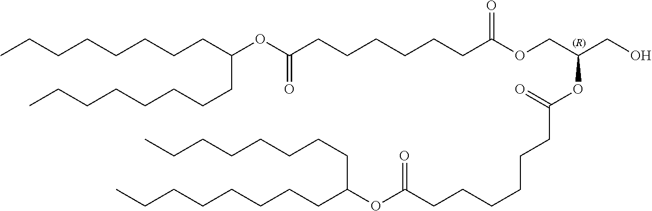

A mixture of (R)—O′¹,O¹-(3-(benzyloxy)propane-1,2-diyl) 8-di(heptadecan-9-yl) di(octanedioate) 5 (6.3 g, 6.5 mmol) and 20% Pd(OH)₂ on carbon (1 g) in 100 mL ethyl acetate was hydrogenated using Parr hydrogenator at 35-40 psi for 12 h. After filtration through silica gel and concentration, the product was used for the next step without further purification (5.6 g, 97%).

### Step 4: Synthesis of (S)-8-Di(heptadecan-9-yl) O′¹,O¹-(3-((2-oxido-1,3,2-dioxaphospholan-2-yl)oxy)propane-1,2-diyl) di(octanedioate) (8)

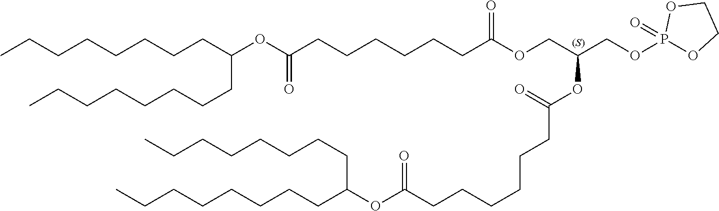

A solution of (R)-8-di(heptadecan-9-yl) O′¹,O¹-(3-hydroxypropane-1,2-diyl) di(octanedioate) δ (2.24 g, 2.5 mmol) and triethylamine (0.40 mL, 2.86 mmol) in 15 mL THF was cooled to 0° C., and then a solution of 2-chloro-1,3,2-dioxaphospholane 2-oxide 7 (408 mg, 2.86 mmol) in 5 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 5: Synthesis of 8-Di(heptadecan-9-yl) O′1,O1-((2S)-3-((hydroxy(2-((3-hydroxypropyl)(methyl)amino)ethoxy)phosphoryl)oxy)propane-1,2-diyl) di(octanedioate) (Compound 28)

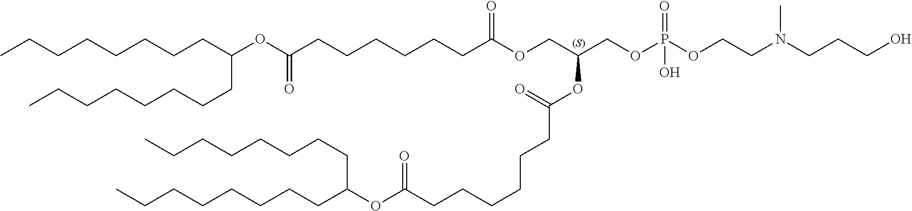

To a solution of the crude (S)-di(heptadecan-9-yl) O′¹,O¹-(3-((2-oxido-1,3,2-dioxaphospholan-2-yl)oxy)propane-1,2-diyl) di(octanedioate) 8 (2.5 mmol) in 3 mL THF and 15 mL acetonitrile, was added 3-((tert-butyldimethylsilyl)oxy)-N-methylpropan-1-amine 9 (4.1 g, 20 mmol), and the resulting mixture was heated at 85-90° C. in a seal tube overnight. MS showed the formation of the desired product. After cooling to room temperature and concentration, the residue was dissolved in 25 mL THF and transferred into Teflon round-bottom flask, 10 mL pyridine and 5 mL hydrogen fluoride pyridine complex were added, and the mixture was stirred at room temperature overnight. The reaction was quenched with saturated NaHCO₃ and extracted with EtOAc. The combined organic layer was dried over sodium sulfate and concentrated, and the crude was purified by column chromatography (SiO₂: CHCl₃/MeOH/H₂O 32:13:1) to give the desired product as off-white wax (1.03 g, 37%).

¹H NMR (400 MHz, CDCl₃) δ 5.26-5.16 (m, 1H), 4.83 (quint, 2H), 4.43-4.31 (m, 1H), 4.29-4.18 (m, 2H), 4.18-4.06 (m, 1H), 4.03-3.92 (m, 2H), 3.80-3.67 (m, 2H), 3.37-3.14 (m, 4H), 2.81 (s, 3H), 2.38-2.17 (m, 8H), 2.02-1.88 (m, 2H), 1.69-1.41 (m, 16H), 1.37-1.13 (m, 58H), 0.85 (t, 12H).

APCI-MS analysis: Calculated C59H114NO13P [M+H]=1076.8, Observed=1076.8.

### Synthesis of 8-Di(heptadecan-9-yl) O′¹,O¹-((2S)-3-((hydroxy(2-((2-hydroxyethyl)(methyl)amino)ethoxy)phosphoryl)oxy)propane-1,2-diyl) di(octanedioate) (Compound 26)

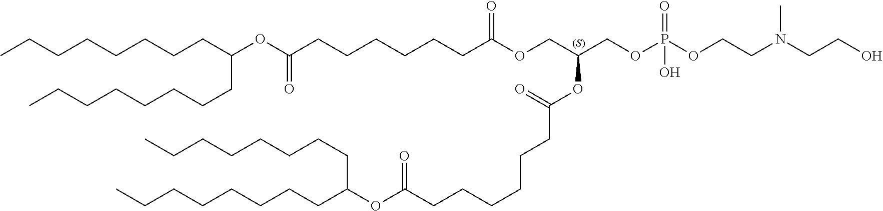

To a solution of the crude (S)-di(heptadecan-9-yl) O′¹,O¹-(3-((2-oxido-1,3,2-dioxaphospholan-2-yl)oxy)propane-1,2-diyl) di(octanedioate) 8 (3.4 mmol) in 5 mL THF and 20 mL acetonitrile, was added 2-((tert-butyldimethylsilyl)oxy)-N-methylethan-1-amine 9 (4.0 g, 21 mmol), and the resulting mixture was heated at 85-90° C. in a seal tube overnight. MS showed the formation of the desired product. After cooling to room temperature and concentration, the residue was dissolved in 50 mL THF and transferred into Teflon round-bottom flask, 8 mL pyridine and 5 mL hydrogen fluoride pyridine complex were added, and the mixture was stirred at room temperature overnight. The reaction was quenched with saturated NaHCO₃ and extracted with EtOAc. The combined organic layer was dried over sodium sulfate and concentrated, and the crude was purified by column chromatography (SiO₂: CHCl₃/MeOH/H₂O 32:13:1) to give the desired product as pale yellow wax (1.05 g, 29%).

¹H NMR (400 MHz, CDCl₃) δ 5.27-5.15 (m, 1H), 4.83 (quint, 2H), 4.45-4.06 (m, 4H), 4.04-3.82 (m, 4H), 3.43-3.07 (m, 2H), 2.88 (s, 3H), 2.37-2.16 (m, 8H), 1.69-1.40 (m, 16H), 1.38-1.12 (m, 60H), 0.85 (t, 12H).

API-MS analysis: Calculated C58H112NO13P [M+H]=1062.8, Observed=1062.9.

### Synthetic Scheme for Compound 40

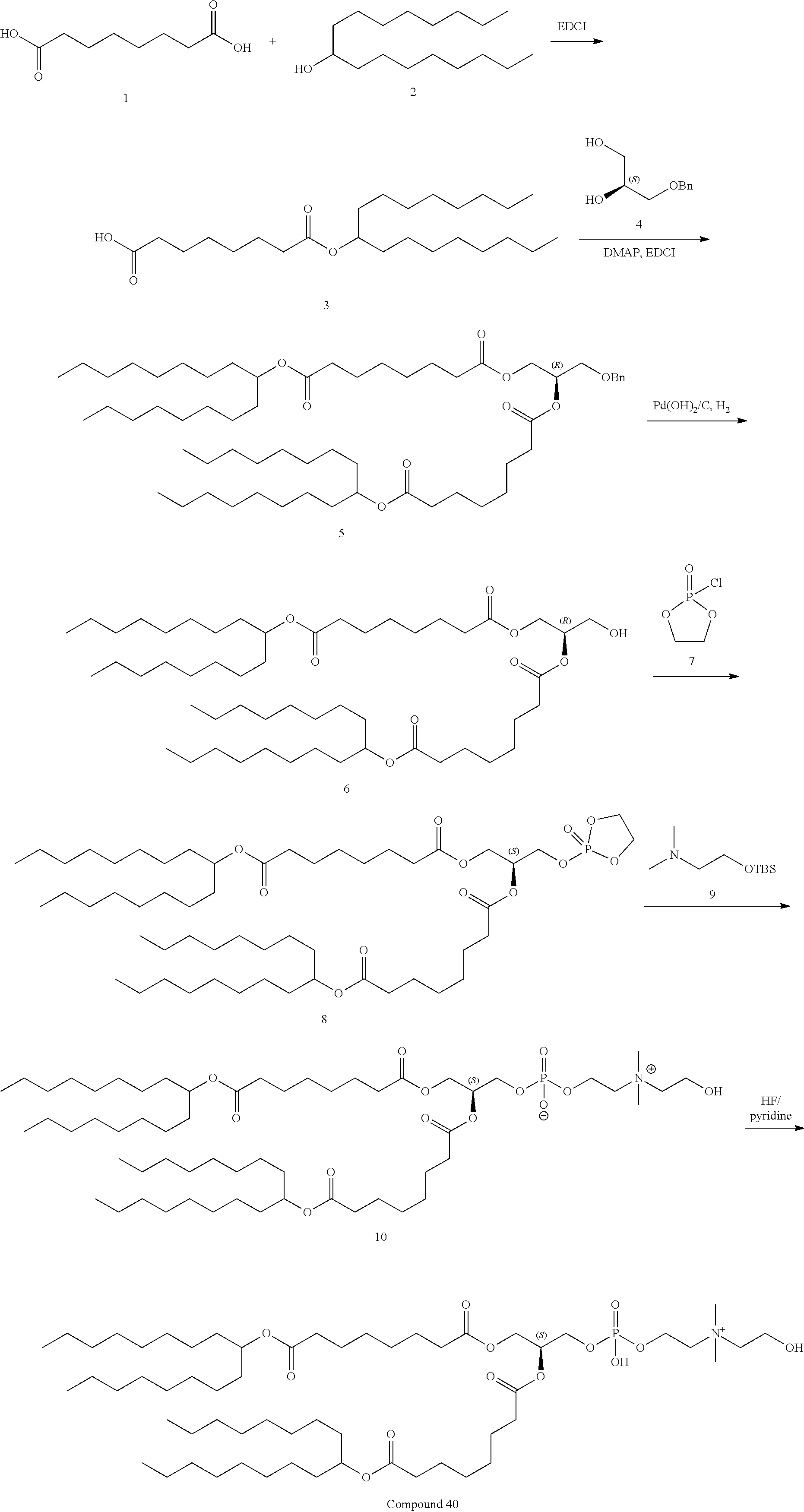

### Synthetic Procedure for Compound 40

### Step 1: Synthesis of 8-(Heptadecane-9-yloxy)-8-oxooctanoic acid (3)

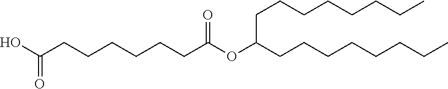 [SMILES: CCCCCCCCC(CCCCCCCC)OC(=O)CCCCCCC(=O)O]

To a stirred homogenous solution of suberic acid 1 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 2 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (R)—O′¹,O¹-(3-(Benzyloxy)propane-1,2-diyl) 8-di(heptadecan-9-yl) di(octanedioate) (5)

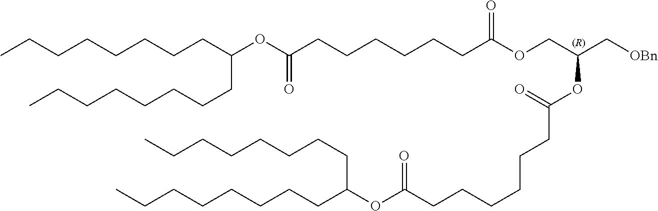

A mixture of (S)-3-(benzyloxy)propane-1,2-diol 4 (3.8 g, 20.9 mmol), 8-(heptadecane-9-yloxy)-8-oxooctanoic acid 3 (19.5 g, 47.3 mmol), EDCI (27.2 g, 142 mmol) and DMAP (5.8 g, 47.5 mmol) in 300 mL dichloromethane was stirred at room temperature for 15 h. TLC showed complete reaction. After concentration, the crude was triturated with hexane, and the solvent was removed. The crude was purified by column chromatography eluted with 0-15% ethyl acetate in hexane to yield (R)—O′¹,O¹-(3-(benzyloxy)propane-1,2-diyl) 8-di(heptadecan-9-yl) di(octanedioate) as colorless oil (18.2 g, 90%).

### Step 3: Synthesis of (R)-8-Di(heptadecan-9-yl) O′¹,O¹′-(3-hydroxypropane-1,2-diyl) di(octanedioate) (6)

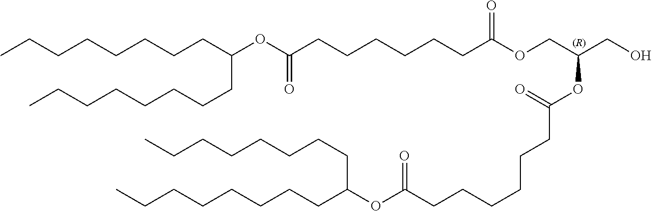

A mixture of (R)—O′¹,O¹-(3-(benzyloxy)propane-1,2-diyl) 8-di(heptadecan-9-yl) di(octanedioate) 5 (6.3 g, 6.5 mmol) and 20% Pd(OH)₂ on carbon (1 g) in 100 mL ethyl acetate was hydrogenated using Parr hydrogenator at 35-40 psi for 12 h. After filtration through silica gel and concentration, the product was used for the next step without further purification (5.6 g, 97%).

### Step 4: Synthesis of (S)-8-Di(heptadecan-9-yl) O′¹,O¹-(3-((2-oxido-1,3,2-dioxaphospholan-2-yl)oxy)propane-1,2-diyl) di(octanedioate) (8)

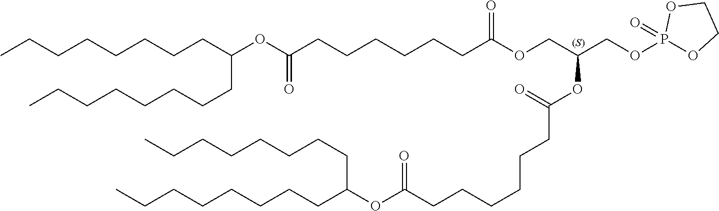

A solution of (R)-8-di(heptadecan-9-yl) O′¹,O¹-(3-hydroxypropane-1,2-diyl) di(octanedioate) δ (2.5 g, 2.84 mmol) and triethylamine (788 mg, 5.68 mmol) in 15 mL THF was cooled to 0° C., and then a solution of 2-chloro-1,3,2-dioxaphospholane 2-oxide 7 (809 mg, 5.68 mmol) in 5 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 5: Synthesis of (S)-2,3-Bis((8-(heptadecan-9-yloxy)-8-oxooctanoyl)oxy)propyl (2-((2-hydroxyethyl)dimethylammonio)ethyl) phosphate (Compound 40)

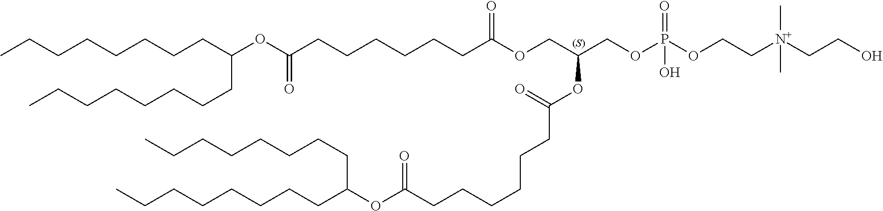

To a solution of the crude (S)-di(heptadecan-9-yl) O′¹,O¹-(3-((2-oxido-1,3,2-dioxaphospholan-2-yl)oxy)propane-1,2-diyl) di(octanedioate) 8 (2.84 mmol) in 5 mL THF and 10 mL acetonitrile, was added 2-((tert-butyldimethylsilyl)oxy)-N,N-dimethylethan-1-amine 9 (4 g, 19.7 mmol), and the resulting mixture was heated at 85-90° C. in a seal tube for 3 days. MS showed the formation of the desired product. After cooling to room temperature and concentration, the residue was dissolved in 50 mL THF and transferred into Teflon round-bottom flask, 7 mL pyridine and 5 mL hydrogen fluoride pyridine complex were added, and the mixture was stirred at room temperature overnight. The reaction was quenched with saturated NaHCO₃ and extracted with EtOAc. The combined organic layer was dried over sodium sulfate and concentrated, and the crude was purified by column chromatography (SiO₂: CHCl₃/MeOH/H₂O 32:13:1) to give the desired product as pale yellow wax (100 mg, 6%).

¹H NMR (400 MHz, CDCl₃) δ 6.68-6.33 (m, 1H), 4.82 (quint, 2H), 4.58-3.46 (m, 12H), 3.31 (s, 6H), 2.43-2.09 (m, 8H), 1.67-1.39 (m, 16H), 1.37-1.10 (m, 56H), 0.85 (t, 12H).

ESI-MS analysis: Calculated C59H114NO13P [M+H]=1076.8, Observed=1076.8, 1098.8 (M+Na).

HPLC-ELSD: t_{R}=6.539 min (method 1).

### Synthetic Scheme for Compound 41

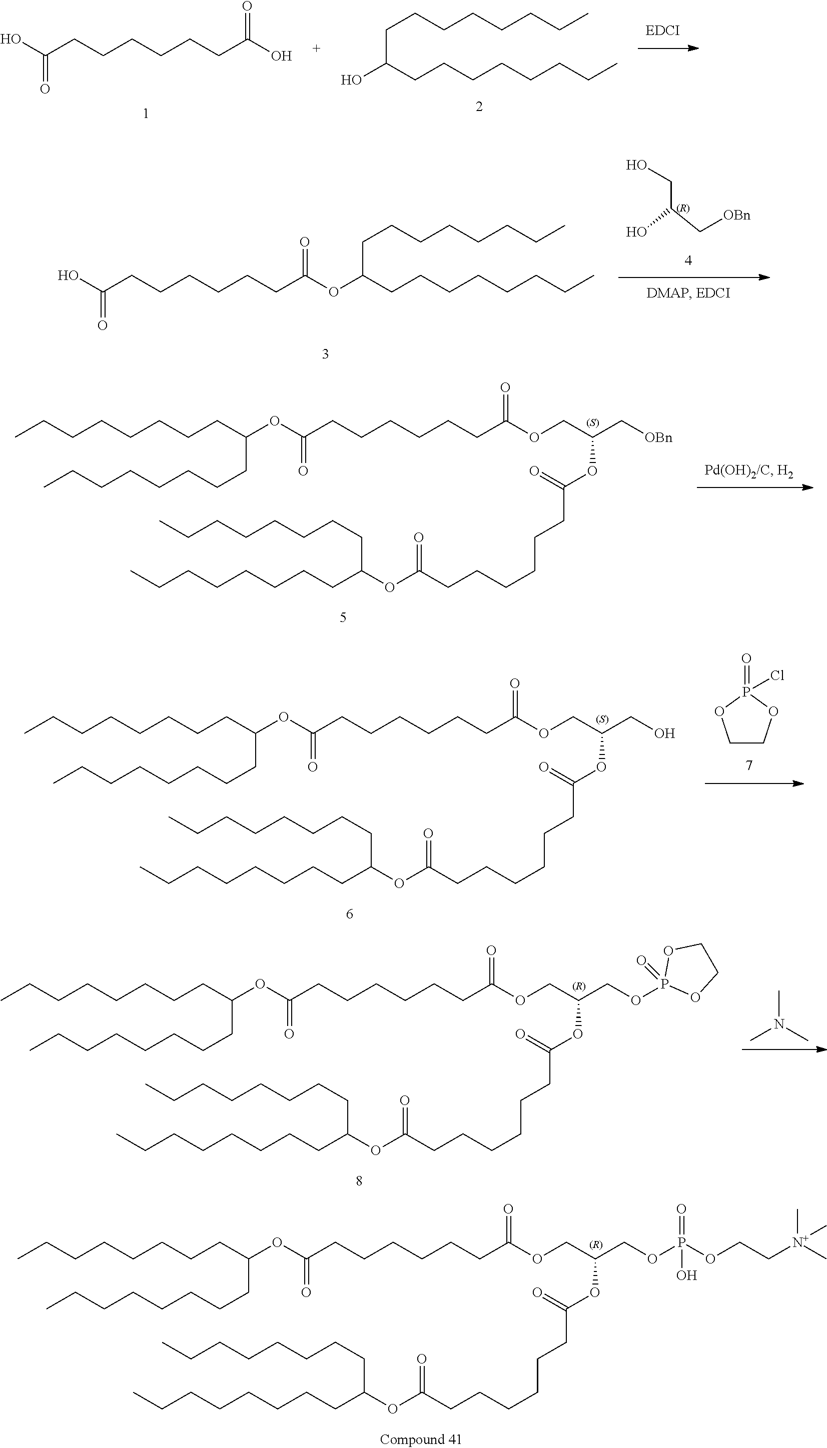

### Synthetic Procedure for Compound 41

### Step 1: Synthesis of 8-(Heptadecane-9-yloxy)-8-oxooctanoic acid (3)

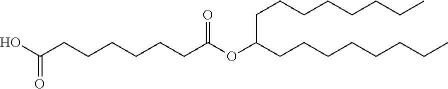 [SMILES: CCCCCCCCC(CCCCCCCC)OC(=O)CCCCCCC(=O)O]

To a stirred homogenous solution of suberic acid 1 (30 g, 172 mmol) in 200 mL of DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 2 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (S)—O′¹,O¹-(3-(Benzyloxy)propane-1,2-diyl) 8-di(heptadecan-9-yl) di(octanedioate) (5)

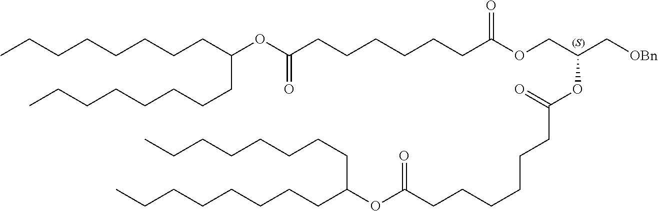

A mixture of (R)-3-(benzyloxy)propane-1,2-diol 4 (3.46 g, 19 mmol), 8-(heptadecane-9-yloxy)-8-oxooctanoic acid 3 (17.6 g, 42.75 mmol), EDCI (19.2 g, 100 mmol) and DMAP (5.2 g, 42.75 mmol) in 150 mL dichloromethane was stirred at room temperature for 2 days. TLC showed complete reaction. After concentration, the crude was triturated with hexane, and the solvent was removed. The crude was purified by column chromatography eluted with 0-15% ethyl acetate in hexane to yield (S)—O′¹,O¹-(3-(benzyloxy)propane-1,2-diyl) 8-di(heptadecan-9-yl) di(octanedioate) as colorless oil (17.4 g, 94%).

### Step 3: Synthesis of (S)-8-Di(heptadecan-9-yl) O′¹,O¹′-(3-hydroxypropane-1,2-diyl) di(octanedioate) (6)

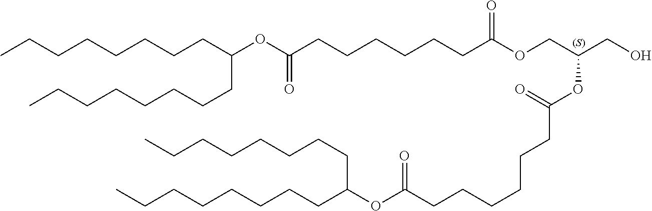

A mixture of (S)—O′1,O1-(3-(benzyloxy)propane-1,2-diyl) 8-di(heptadecan-9-yl) di(octanedioate) 5 (17.4 g, 17.8 mmol), 20% Pd(OH)₂ on carbon (4 g) in 200 mL ethyl acetate was hydrogenated using Parr hydrogenator at 35-40 psi for 12 h. After filtration through silica gel and concentration, the product was used for the next step without further purification (15.7 g, 99%).

### Step 4: Synthesis of (R)-8-Di(heptadecan-9-yl) O′¹,O¹-(3-((2-oxido-1,3,2-dioxaphospholan-2-yl)oxy)propane-1,2-diyl) di(octanedioate) (8)

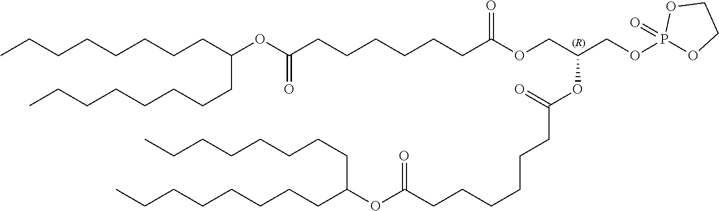

A solution of (S)-8-di(heptadecan-9-yl) O′¹,O¹-(3-hydroxypropane-1,2-diyl) di(octanedioate) δ (15.7 g, 17.8 mmol) and triethylamine (3.5 mL, 25.45 mmol) in 50 mL THF was cooled to 0° C., and then a solution of 2-chloro-1,3,2-dioxaphospholane 2-oxide 7 (3.6 g, 25.45 mmol) in 10 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 5: Synthesis of (R)-2,3-Bis((8-(heptadecan-9-yloxy)-8-oxooctanoyl)oxy)propyl (2-(trimethylammonio)ethyl) phosphate (Compound 41)

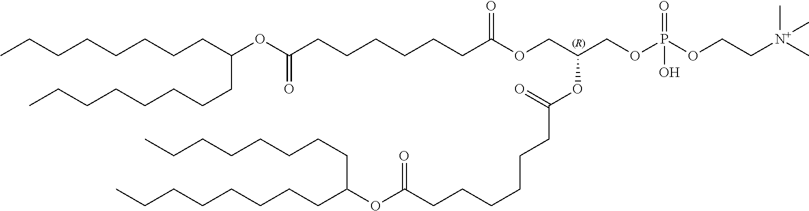

In a seal tube, a solution of (R)-8-di(heptadecan-9-yl) O′¹,O¹-(3-((2-oxido-1,3,2-dioxaphospholan-2-yl)oxy)propane-1,2-diyl) di(octanedioate) 8 (crude) in 60 mL acetonitrile was cooled in ice bath, and a solution of trimethylamine (1 M in THF, 56 mL, 56 mmol) was added. The bottle was sealed and heated at 65° C. for 3 days. After cooling to room temperature, the volatiles were evaporated under vacuum, and the crude was purified by column chromatography (SiO₂: CHCl₃/MeOH/H₂O 32:13:0.4) to give the desired product as white solid (5.5 g, 30%).

¹H NMR (400 MHz, CDCl₃) δ 5.25-5.11 (m, 1H), 4.83 (quint, 2H), 4.43-4.25 (m, 3H), 4.16-4.04 (m, 1H), 4.00-3.76 (m, 4H), 3.36 (s, 9H), 2.36-2.18 (m, 8H), 1.69-1.39 (m, 16H), 1.38-1.08 (m, 56H), 0.85 (t, 12H).

ESI-MS analysis: Calculated C58H112NO12P [M+H]=1046.8, Observed=1046.8.

HPLC-ELSD: t_{R}=6.762 min (method 1).

### Synthetic Scheme for Compound 42

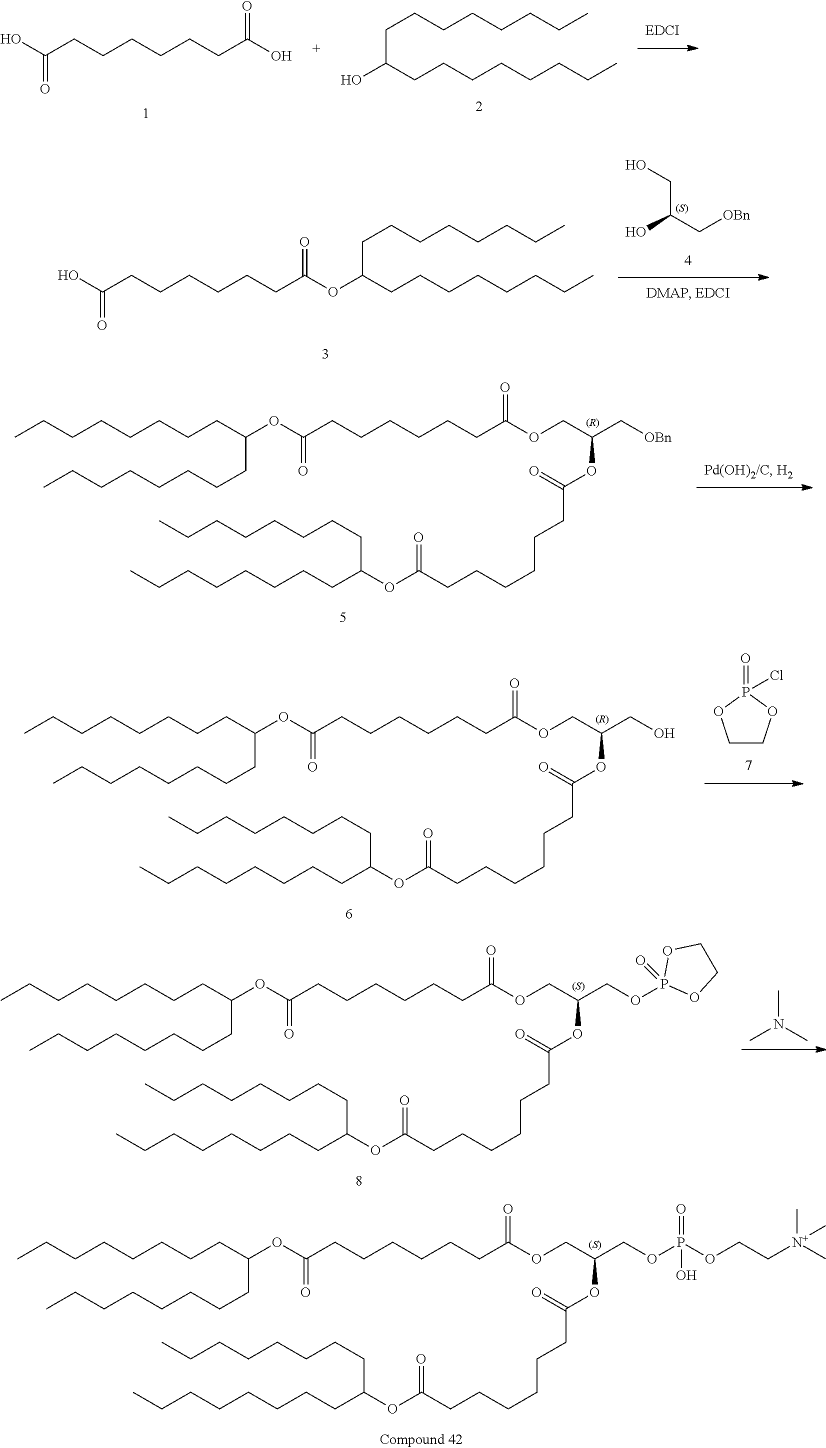

### Synthetic Procedure for Compound 42

### Step 1: Synthesis of 8-(Heptadecane-9-yloxy)-8-oxooctanoic acid (3)

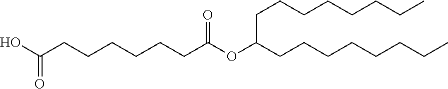 [SMILES: CCCCCCCCC(CCCCCCCC)OC(=O)CCCCCCC(=O)O]

To a stirred homogenous solution of suberic acid 1 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 2 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (R)—O′¹,O¹-(3-(Benzyloxy)propane-1,2-diyl) 8-di(heptadecan-9-yl) di(octanedioate) (5)

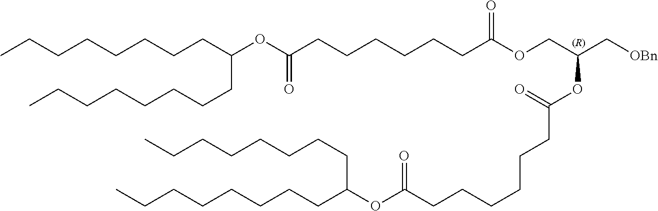

A mixture of (S)-3-(benzyloxy)propane-1,2-diol 4 (4.7 g, 25.7 mmol), 8-(heptadecane-9-yloxy)-8-oxooctanoic acid 3 (23.8 g, 58 mmol), EDCI (26 g, 135 mmol) and DMAP (7.0 g, 58 mmol) in 200 mL dichloromethane was stirred at room temperature for 15 h. TLC showed complete reaction. After concentration, the crude was triturated with hexane, and the solvent was removed. The crude was purified by column chromatography eluted with 0-15% ethyl acetate in hexane to yield (R)—O′¹,O¹-(3-(benzyloxy)propane-1,2-diyl) 8-di(heptadecan-9-yl) di(octanedioate) as colorless oil (24.5 g, 98%).

### Step 3: Synthesis of (R)-8-Di(heptadecan-9-yl) O′¹,O¹-(3-hydroxypropane-1,2-diyl) di(octanedioate) (6)

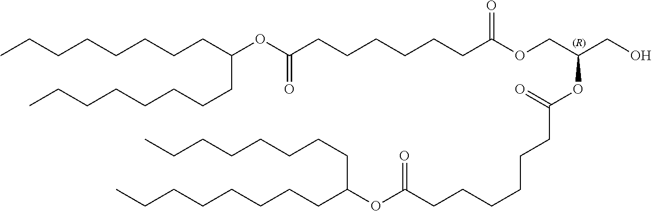

A mixture of (R)—O′¹,O¹-(3-(benzyloxy)propane-1,2-diyl) 8-di(heptadecan-9-yl) di(octanedioate) 5 (12 g, 12.3 mmol), 20% Pd(OH)₂ on carbon (4 g) in 80 mL ethyl acetate was hydrogenated using Parr hydrogenator at 35-40 psi for 12 h. After filtration through silica gel and concentration, the product was used for the next step without further purification (10.8 g, quant.).

### Step 4: Synthesis of (S)-8-Di(heptadecan-9-yl) O′¹,O¹-(3-((2-oxido-1,3,2-dioxaphospholan-2-yl)oxy)propane-1,2-diyl) di(octanedioate) (8)

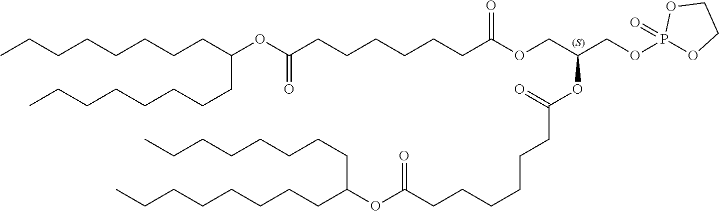

A solution of (R)-8-di(heptadecan-9-yl) O′¹,O¹-(3-hydroxypropane-1,2-diyl) di(octanedioate) δ (4.4 g, 5 mmol) and triethylamine (1.12 mL, 8 mmol) in 20 mL THF was cooled to 0° C., and then a solution of 2-chloro-1,3,2-dioxaphospholane 2-oxide 7 (1.14 mg, 8 mmol) in 5 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 5: Synthesis of (S)-2,3-Bis((8-(heptadecan-9-yloxy)-8-oxooctanoyl)oxy)propyl (2-(trimethylammonio)ethyl) phosphate (Compound 42)

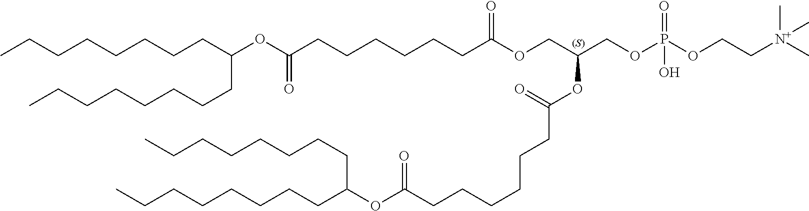

In a seal tube, a solution of (S)-8-di(heptadecan-9-yl) O′¹,O¹-(3-((2-oxido-1,3,2-dioxaphospholan-2-yl)oxy)propane-1,2-diyl) di(octanedioate) 8 (crude, 5 mmol) in 50 mL acetonitrile was cooled in ice bath, and a solution of trimethylamine (2 M in THF, 30 mL, 60 mmol) was added. The bottle was sealed and heated at 85° C. for 3 days. After cooling to room temperature, the volatiles were evaporated under vacuum, and the crude was purified by column chromatography (SiO₂: CHCl₃/MeOH/H₂O 32:13:0.4) to give the desired product as white solid (3.0 g, 57%).

¹H NMR (400 MHz, CDCl₃) δ 5.28-5.10 (m, 1H), 4.92-4.76 (m, 2H), 4.46-4.24 (m, 3H), 4.18-4.07 (m, 1H), 4.04-3.76 (m, 4H), 3.38 (s, 9H), 2.54-2.00 (m, 8H), 1.70-1.42 (m, 12H), 1.39-1.11 (m, 60H), 0.86 (t, 12H).

ESI-MS analysis: Calculated C58H112NO12P [M+H]=1046.8, Observed=1046.7, 1069.8 (M+Na).

HPLC-ELSD: t_{R}=6.780 min (method 1).

### Synthetic Scheme for Compound 44

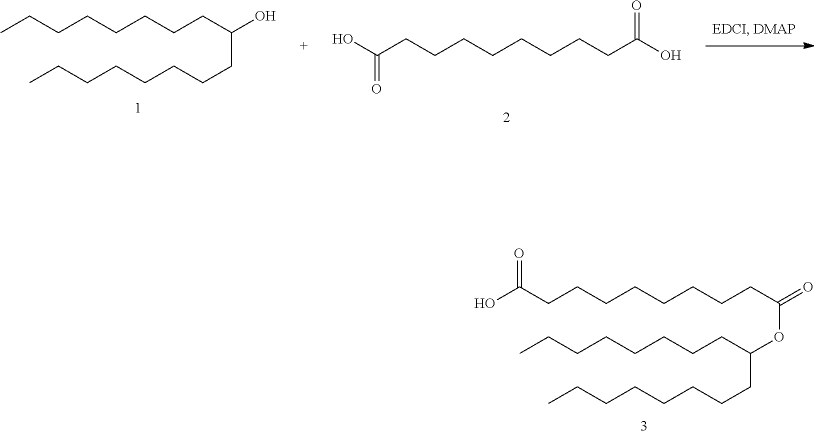 [SMILES: CCCCCCCCC(O)CCCCCCCC ; O=C(O)CCCCCCCCC(=O)O ; CCCCCCCCC(CCCCCCCC)OC(=O)CCCCCCCCC(=O)O]

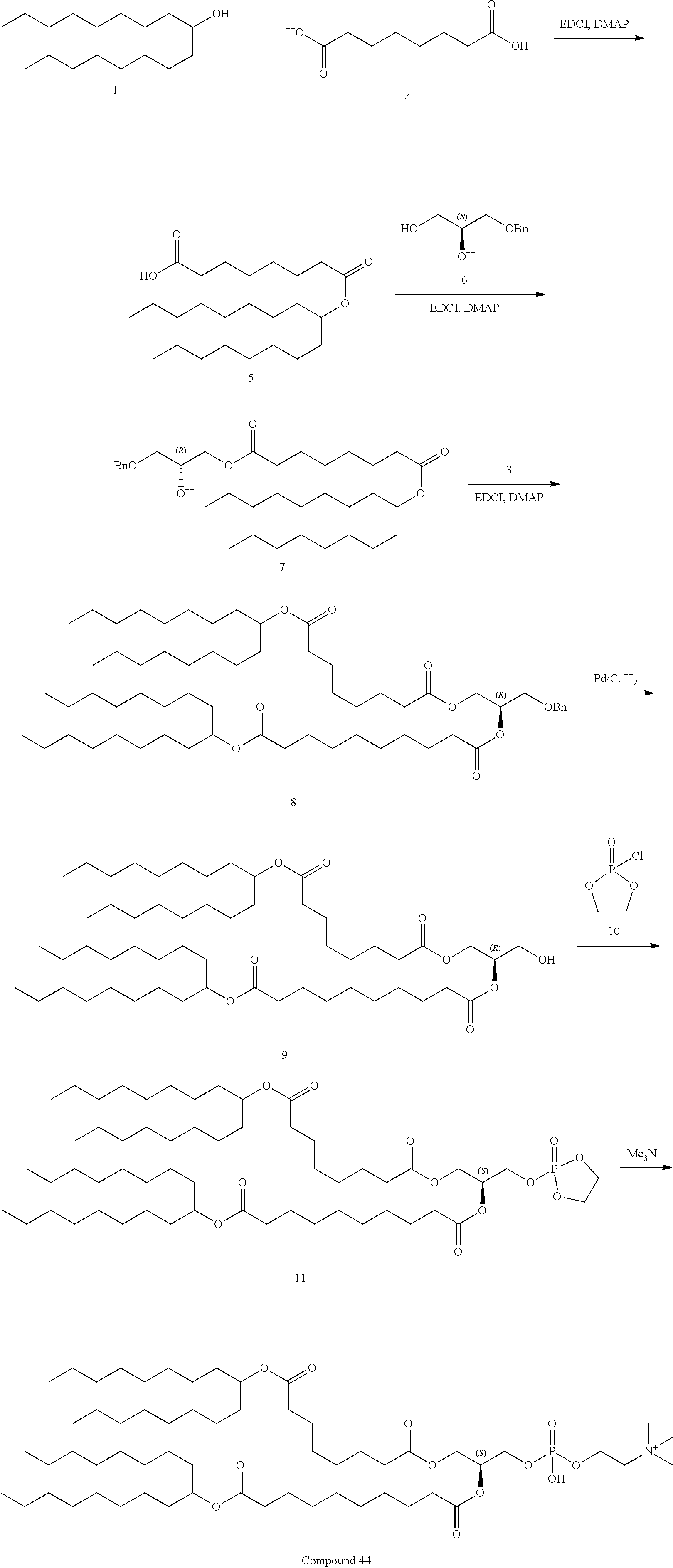

### Synthetic Procedure for Compound 44

### Step 1: Synthesis of 10-(Heptadecan-9-yloxy)-10-oxodecanoic acid (3)

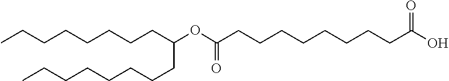 [SMILES: CCCCCCCCC(CCCCCCCC)OC(=O)CCCCCCCCC(=O)O]

To a stirred homogenous solution of sebacic acid 2 (26.3 g, 130 mmol) in 150 mL DMF: DCM (1:2, v/v), was added DMAP (5.2 g, 43.4 mmol) and EDCI (18 g, 95.5 mmol), and the mixture was stirred at room temperature for 10 minutes. Then a homogeneous solution of 9-heptadecanol 1 (11 g, 43.4 mmol) in 100 mL DMF: DCM (1:1, v/v) was added dropwise via addition funnel over a period of 8 hours. The mixture was stirred at room temperature for 10 h. After removing the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 10-(heptadecan-9-yloxy)-10-oxodecanoic acid as colorless oil (14.5 g, 76%).

### Step 2: Synthesis of 8-(Heptadecane-9-yloxy)-8-oxooctanoic acid (5)

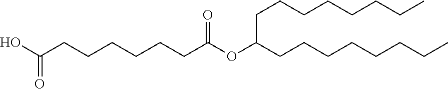 [SMILES: CCCCCCCCC(CCCCCCCC)OC(=O)CCCCCCC(=O)O]

To a stirred homogenous solution of suberic acid 4 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 1 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 3: Synthesis of (R)-1-(3-(Benzyloxy)-2-hydroxypropyl) 8-(heptadecan-9-yl) octanedioate (7)

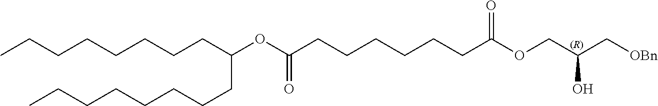

A mixture of (S)-3-(benzyloxy)propane-1,2-diol 6 (1.1 g, 6 mmol), 8-(heptadecane-9-yloxy)-8-oxooctanoic acid 5 (1.65 g, 4 mmol), EDCI (1.15 g, 6 mmol) and DMAP (488 mg, 4 mmol) in 20 mL dichloromethane was stirred at room temperature for 15 h. After concentration, the crude was triturated with hexane, and the solvent was removed. The crude was purified by column chromatography eluted with 0-40% ethyl acetate in hexane to yield (R)-1-(3-(benzyloxy)-2-hydroxypropyl) 8-(heptadecan-9-yl) octanedioate as colorless oil (635 mg, 27%).

### Step 4: Synthesis of (R)-1-(1-(Benzyloxy)-3-((8-(heptadecan-9-yloxy)-8-oxooctanoyl)oxy)propan-2-yl) 10-(heptadecan-9-yl) decanedioate (8)

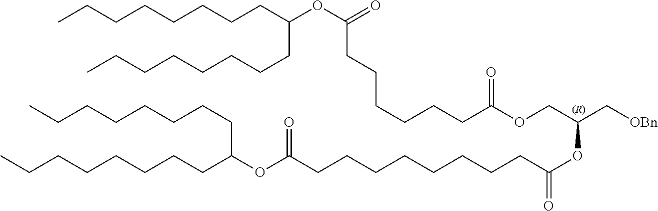

A mixture of (R)-1-(3-(benzyloxy)-2-hydroxypropyl) 8-(heptadecan-9-yl) octanedioate 7 (750 mg, 1.3 mmol), 10-(heptadecan-9-yloxy)-10-oxodecanoic acid 3 (860 mg, 1.95 mmol), EDCI (591 mg, 4 mmol) and DMAP (244 mg, 2 mmol) in 40 mL dichloromethane was stirred at room temperature for 15 h. TLC showed complete reaction. After concentration, the crude was triturated with hexane, and the solvent was removed. The crude was purified by column chromatography eluted with 0-15% ethyl acetate in hexane to yield (R)-1-(1-(benzyloxy)-3-((8-(heptadecan-9-yloxy)-8-oxooctanoyl)oxy)propan-2-yl) 10-(heptadecan-9-yl) decanedioate as colorless oil (1.2 g, 92%).

### Step 5: Synthesis of (R)-1-(Heptadecan-9-yl) 10-(1-((8-(heptadecan-9-yloxy)-8-oxooctanoyl)oxy)-3-hydroxypropan-2-yl) decanedioate (9)

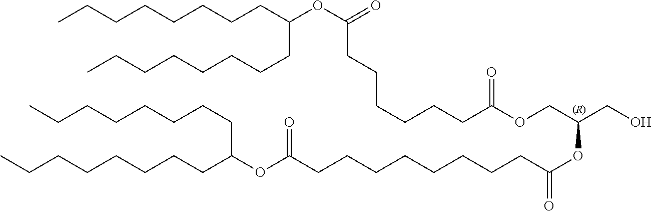

A mixture of (R)-1-(1-(benzyloxy)-3-((8-(heptadecan-9-yloxy)-8-oxooctanoyl)oxy)propan-2-yl) 10-(heptadecan-9-yl) decanedioate 8 (1.1 g, 1.1 mmol) and 10% palladium on carbon (500 mg) in 20 mL ethyl acetate was hydrogenated with balloon for 12 h. After filtration through silica gel and concentration, the product was used for the next step without further purification (1.0 g, quant.).

### Step 6: Synthesis of (S)-1-(Heptadecan-9-yl) 10-(1-((8-(heptadecan-9-yloxy)-8-oxo-octanoyl)oxy)-3-((2-oxido-1,3,2-dioxaphospholan-2-yl)oxy)propan-2-yl) decanedioate (11)

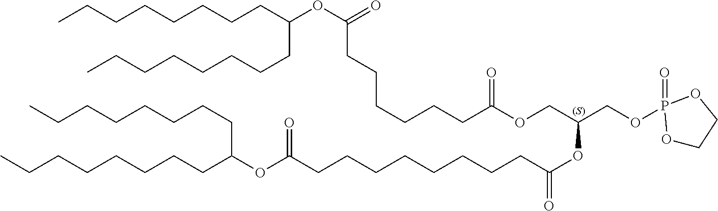

A solution of (R)-1-(heptadecan-9-yl) 10-(1-((8-(heptadecan-9-yloxy)-8-oxooctanoyl)oxy)-3-hydroxypropan-2-yl) decanedioate 9 (910 mg, 1 mmol) and triethylamine (0.21 mL, 1.5 mmol) in 15 mL THF was cooled to 0° C., and then a solution of 2-chloro-1,3,2-dioxaphospholane 2-oxide 10 (214 mg, 1.5 mmol) in 1 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 7: Synthesis of (S)-2-((10-(Heptadecan-9-yloxy)-10-oxodecanoyl)oxy)-3-((8-(heptadecan-9-yloxy)-8-oxooctanoyl)oxy)propyl (2-(trimethylammonio)ethyl) phosphate (Compound 44)

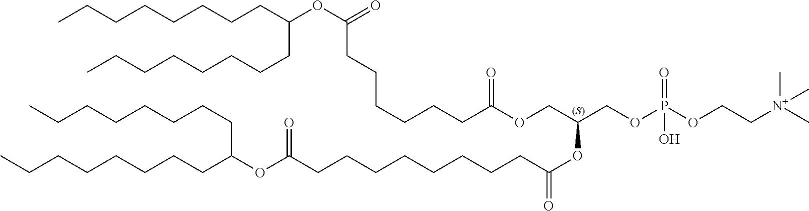

In a seal tube, a solution of (S)-8-di(heptadecan-9-yl) O′¹,O¹-(3-((2-oxido-1,3,2-dioxaphospholan-2-yl)oxy)propane-1,2-diyl) di(octanedioate) 11 (crude) in 10 mL acetonitrile was cooled in ice bath, and a solution of trimethylamine (2 M in THF, 5 mL, 10 mmol) was added. The bottle was sealed and heated at 95° C. overnight. After cooling to room temperature, the volatiles were evaporated under vacuum, and the crude was purified by column chromatography (SiO₂: CHCl₃/MeOH/H₂O 32:13:0.4) to give the desired product as pale yellow solid (320 mg, 30%).

¹H NMR (400 MHz, CDCl₃) δ 5.25-5.11 (m, 1H), 4.90-4.77 (m, 2H), 4.43-4.24 (m, 3H), 4.18-4.04 (m, 1H), 4.00-3.86 (m, 2H), 3.84-3.72 (m, 2H), 3.34 (s, 9H), 2.35-2.18 (m, 8H), 1.69-1.40 (m, 16H), 1.37-1.11 (m, 60H), 0.86 (t, 12H).

ESI-MS analysis: Calculated C60H116NO12P [M+H]=1074.8, Observed=1074.8, 1096.8 (M+Na).

HPLC-ELSD: t_{R}=6.906 min (method 1).

### Synthetic Scheme for Compound 46

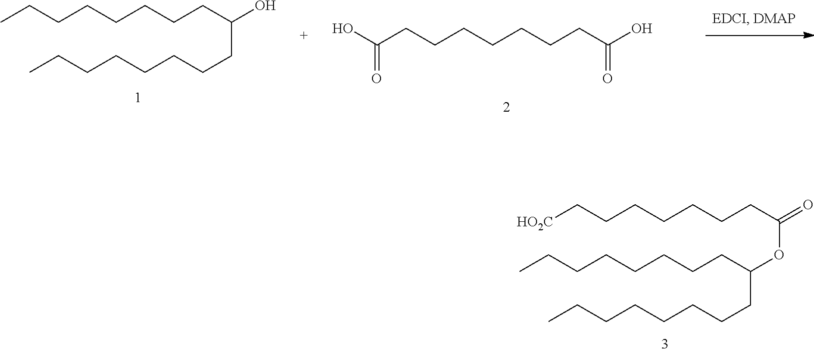 [SMILES: CCCCCCCCC(O)CCCCCCCC ; O=C(O)CCCCCCCC(=O)O ; CCCCCCCCC(CCCCCCCC)OC(=O)CCCCCCCC(=O)O]

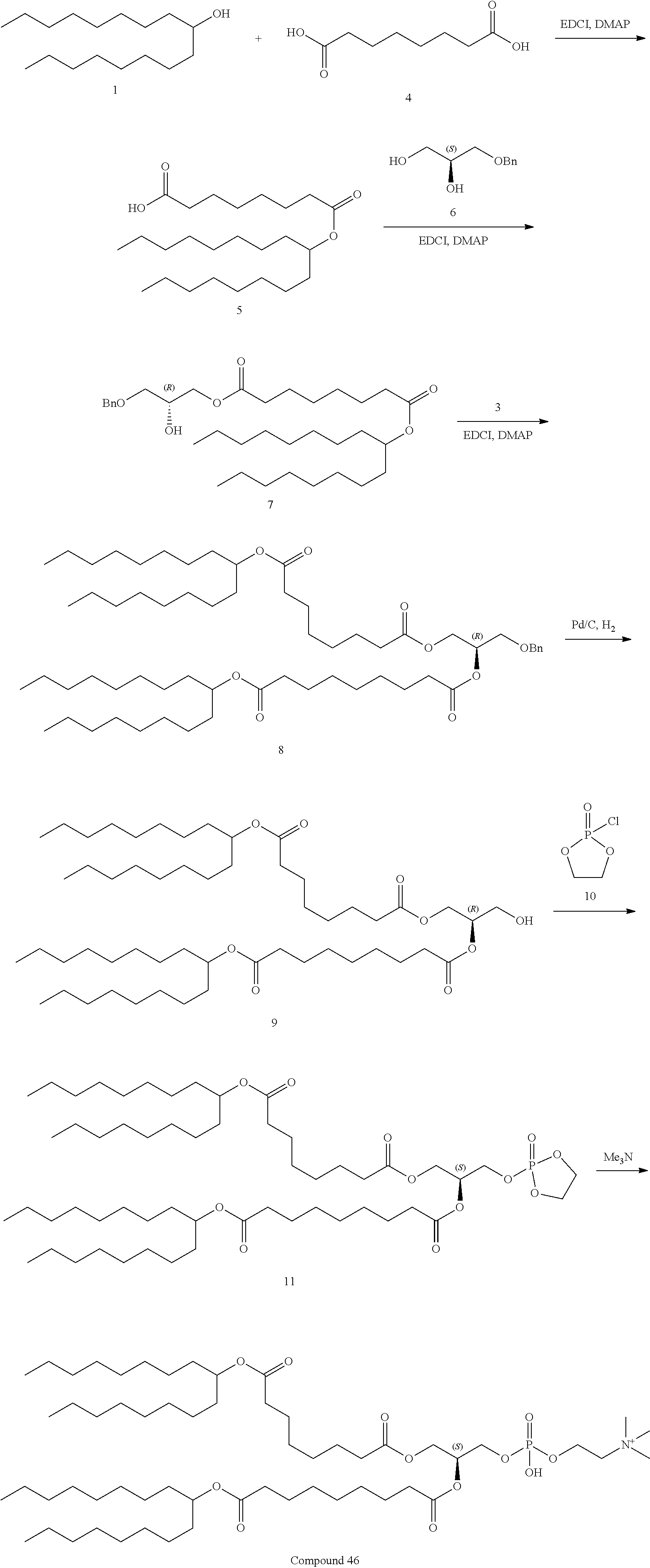

### Synthetic Procedure for Compound 46

### Step 1: Synthesis of 9-(Heptadecan-9-yloxy)-9-oxononanoic acid (3)

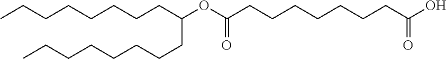 [SMILES: CCCCCCCCC(CCCCCCCC)OC(=O)CCCCCCCC(=O)O]

To a stirred homogenous solution of azelaic acid 2 (18 g, 94.4 mmol) in 100 mL DMF: DCM (1:1, v/v), was added DMAP (0.26 g, 2.15 mmol) and EDCI (9.8 g, 51.5 mmol), and the mixture was stirred at room temperature for 10 minutes. Then a homogeneous solution of 9-heptadecanol 1 (11 g, 43.4 mmol) in 50 mL DMF: DCM (1:1, v/v) was added dropwise via addition funnel over a period of 8 hours. The mixture was stirred at room temperature for 10 h. After removing the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 9-(heptadecan-9-yloxy)-9-oxononanoic acid as colorless oil (12.5 g, 70%).

### Step 2: Synthesis of 8-(Heptadecane-9-yloxy)-8-oxooctanoic acid (5)

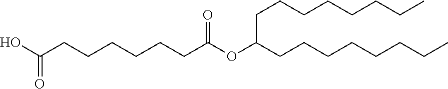 [SMILES: CCCCCCCCC(CCCCCCCC)OC(=O)CCCCCCC(=O)O]

To a stirred homogenous solution of suberic acid 4 (30 g, 172 mmol) in 200 mL of DMF: DCM (1:1, v/v), was added DMAP (7.13 g, 59 mmol) and EDCI (25 g, 130 mmol), and the mixture was stirred at room temperature for 10 minutes. Then a homogeneous solution 9-heptadecanol 1 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 3: Synthesis of (R)-1-(3-(Benzyloxy)-2-hydroxypropyl) 8-(heptadecan-9-yl) octanedioate (7)

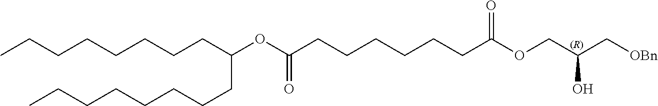

A mixture of (S)-3-(benzyloxy)propane-1,2-diol 6 (1.1 g, 6 mmol), 8-(heptadecane-9-yloxy)-8-oxooctanoic acid 5 (1.65 g, 4 mmol), EDCI (1.15 g, 6 mmol) and DMAP (488 mg, 4 mmol) in 20 mL dichloromethane was stirred at room temperature for 15 h. After concentration, the crude was triturated with hexane, and the solvent was removed. The crude was purified by column chromatography eluted with 0-40% ethyl acetate in hexane to yield (R)-1-(3-(benzyloxy)-2-hydroxypropyl) 8-(heptadecan-9-yl) octanedioate as colorless oil (635 mg, 27%).

### Step 4: Synthesis of (R)-1-(1-(Benzyloxy)-3-((8-(heptadecan-9-yloxy)-8-oxooctanoyl)oxy)propan-2-yl) 9-(heptadecan-9-yl) nonanedioate (8)

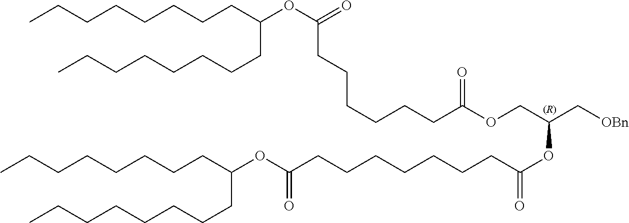

A mixture of (R)-1-(3-(benzyloxy)-2-hydroxypropyl) 8-(heptadecan-9-yl) octanedioate 7 (635 mg, 1.1 mmol), 10-(heptadecan-9-yloxy)-10-oxodecanoic acid 3 (640 mg, 1.5 mmol), EDCI (576 mg, 3 mmol) and DMAP (183 mg, 1.5 mmol) in 40 mL dichloromethane was stirred at room temperature for 2 days. TLC showed complete reaction. After concentration, the crude was triturated with hexane, and the solvent was removed. The crude was purified by column chromatography eluted with 0-15% ethyl acetate in hexane to yield (R)-1-(1-(benzyloxy)-3-((8-(heptadecan-9-yloxy)-8-oxooctanoyl)oxy)propan-2-yl) 9-(heptadecan-9-yl) nonanedioate as colorless oil (980 mg, 92%).

### Step 5: Synthesis of (R)-1-(Heptadecan-9-yl) 9-(1-((8-(heptadecan-9-yloxy)-8-oxooctanoyl)oxy)-3-hydroxypropan-2-yl) nonanedioate (9)

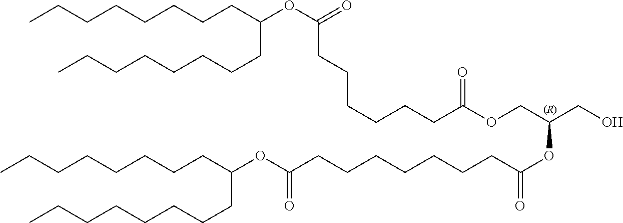

A mixture of (R)-1-(1-(benzyloxy)-3-((8-(heptadecan-9-yloxy)-8-oxooctanoyl)oxy)propan-2-yl) 9-(heptadecan-9-yl) nonanedioate 8 (900 mg, 0.91 mmol) and 10% palladium on carbon (500 mg) in 50 mL ethyl acetate was hydrogenated using Parr hydrogenator at 35-40 psi for 12 h. After filtration through silica gel and concentration, the product was used for the next step without further purification (893 mg, quant.).

### Step 6: Synthesis of (S)-1-(Heptadecan-9-yl) 9-(1-((8-(heptadecan-9-yloxy)-8-oxo-octanoyl)oxy)-3-((2-oxido-1,3,2-dioxaphospholan-2-yl)oxy)propan-2-yl) nonanedioate (11)

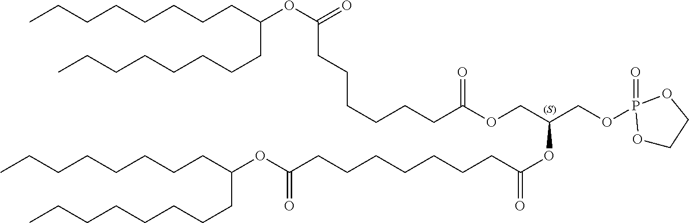

A solution of (R)-1-(heptadecan-9-yl) 9-(1-((8-(heptadecan-9-yloxy)-8-oxooctanoyl)oxy)-3-hydroxypropan-2-yl) nonanedioate 9 (893 mg, 0.91 mmol) and triethylamine (0.19 mL, 1.37 mmol) in 15 mL THF was cooled to 0° C., and then a solution of 2-chloro-1,3,2-dioxaphospholane 2-oxide 10 (194 mg, 1.37 mmol) in 5 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 7: Synthesis of (S)-3-((8-(Heptadecan-9-yloxy)-8-oxooctanoyl)oxy)-2-((9-(heptadecan-9-yloxy)-9-oxononanoyl)oxy)propyl (2-(trimethylammonio)ethyl) phosphate (Compound 46)

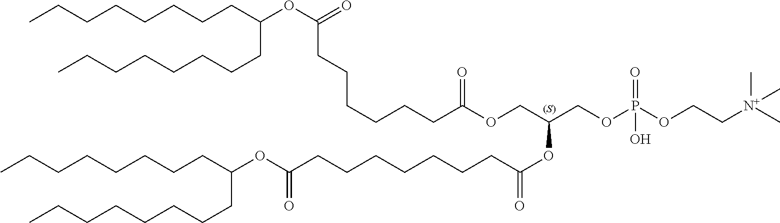

In a seal tube, a solution of (S)-1-(heptadecan-9-yl) 9-(1-((8-(heptadecan-9-yloxy)-8-oxo-octanoyl)oxy)-3-((2-oxido-1,3,2-dioxaphospholan-2-yl)oxy)propan-2-yl) nonanedioate 11 (crude) in 12 mL acetonitrile was cooled in ice bath, and a solution of trimethylamine (2 M in THF, 5.0 mL, 10 mmol) was added. The bottle was sealed and heated at 85° C. for 3 days. After cooling to room temperature, the volatiles were evaporated under vacuum, and the crude was purified by column chromatography (SiO₂: CHCl₃/MeOH/H₂O 32:13:0.4) to give the desired product as pale yellow solid (530 mg, 50%).

¹H NMR (400 MHz, CDCl₃) δ 5.25-5.10 (m, 1H), 4.90-4.75 (m, 2H), 4.45-4.22 (m, 3H), 4.18-4.04 (m, 1H), 4.00-3.84 (m, 2H), 3.83-3.70 (m, 2H), 3.34 (s, 9H), 2.34-2.16 (m, 8H), 1.70-1.41 (m, 16H), 1.39-1.08 (m, 58H), 0.85 (t, 12H).

ESI-MS analysis: Calculated C59H114NO12P [M+H]=1060.8, Observed=1060.8, 1082.8 (M+Na).

HPLC-ELSD: t_{R}=6.845 min (method 1).

### Synthetic Scheme for Compound 48

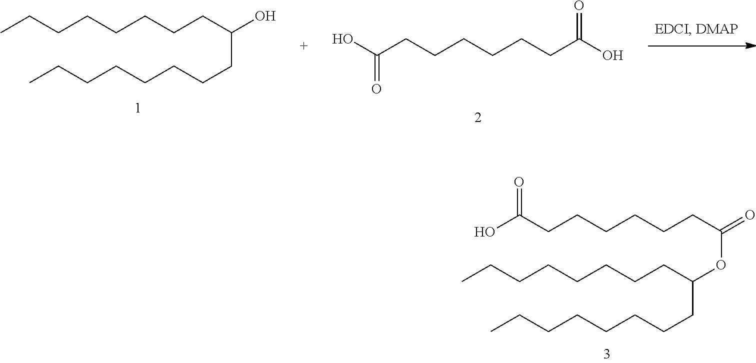 [SMILES: CCCCCCCCC(O)CCCCCCCC ; O=C(O)CCCCCCC(=O)O ; CCCCCCCCC(CCCCCCCC)OC(=O)CCCCCCC(=O)O]

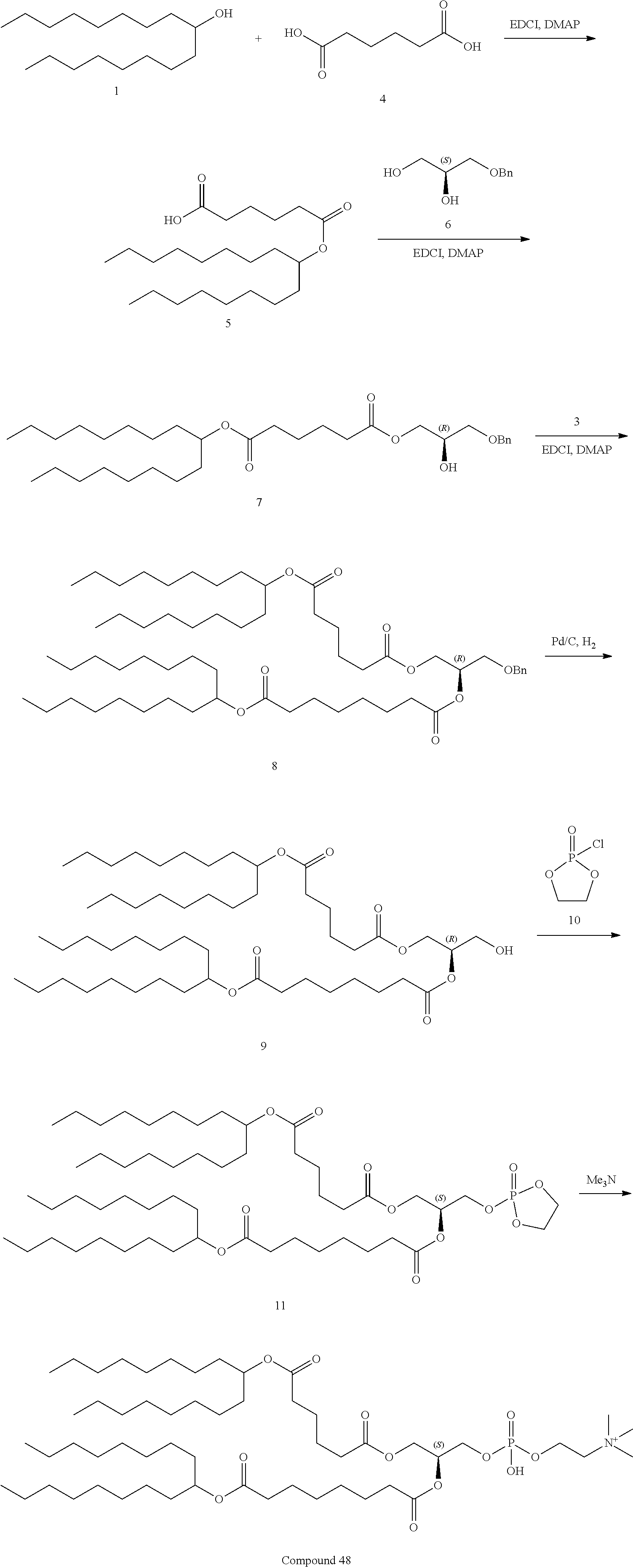

Synthetic Procedure for Compound 48:

### Step 1: Synthesis of 8-(Heptadecane-9-yloxy)-8-oxooctanoic acid (3)

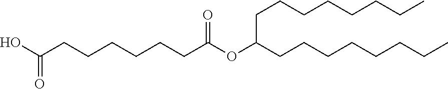 [SMILES: CCCCCCCCC(CCCCCCCC)OC(=O)CCCCCCC(=O)O]

To a stirred homogenous solution of suberic acid 2 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 1 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of 6-(Heptadecan-9-yloxy)-6-oxohexanoic acid (5)

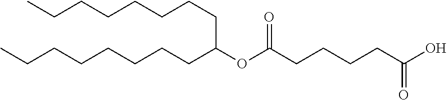 [SMILES: CCCCCCCCC(CCCCCCCC)OC(=O)CCCCC(=O)O]

To a stirred homogenous solution of adipic acid 4 (19 g, 130 mmol) in 100 mL of DMF: DCM (1:1, v/v), was added DMAP (5.2 g, 43.4 mmol) and EDCI (18 g, 95.4 mmol), and the mixture was stirred at room temperature for 10 minutes. Then a homogeneous solution of 9-heptadecanol 1 (11 g, 43.4 mmol) in 50 mL DMF: DCM (1:1, v/v) was added dropwise via addition funnel over a period of 8 hours. The mixture was stirred at room temperature for 2 days. After removing the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 6-(heptadecan-9-yloxy)-6-oxohexanoic acid as colorless oil (10.3 g, 62%).

### Step 3: Synthesis of (R)-3-(Benzyloxy)-2-hydroxypropyl heptadecan-9-yl adipate (7)

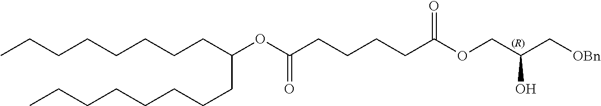

A mixture of (S)-3-(benzyloxy)propane-1,2-diol 6 (1.1 g, 6 mmol), 6-(heptadecan-9-yloxy)-6-oxohexanoic acid 5 (1.5 g, 4 mmol), EDCI (2.2 g, 12 mmol) and DMAP (488 mg, 4 mmol) in 30 mL dichloromethane was stirred at room temperature for 15 h. After concentration, the crude was triturated with hexane, and the solvent was removed. The crude was purified by column chromatography eluted with 0-40% ethyl acetate in hexane to yield (R)-3-(benzyloxy)-2-hydroxypropyl heptadecan-9-yl adipate as colorless oil (620 mg, 26%).

### Step 4: Synthesis of (R)-1-(1-(Benzyloxy)-3-((6-(heptadecan-9-yloxy)-6-oxohexanoyl)oxy)propan-2-yl) 8-(heptadecan-9-yl) octanedioate (8)

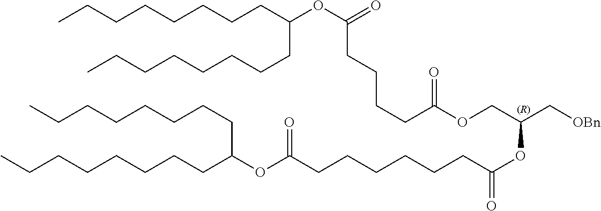

A mixture of (R)-3-(benzyloxy)-2-hydroxypropyl heptadecan-9-yl adipate 7 (620 mg, 1.13 mmol), 8-(heptadecan-9-yloxy)-8-oxooctanoic acid 3 (700 mg, 1.7 mmol), EDCI (326 mg, 1.7 mmol) and DMAP (138 mg, 1.13 mmol) in 40 mL dichloromethane was stirred at room temperature overnight. TLC showed complete reaction. After concentration, the crude was triturated with hexane, and the solvent was removed. The crude was purified by column chromatography eluted with 0-15% ethyl acetate in hexane to yield (R)-1-(1-(benzyloxy)-3-((6-(heptadecan-9-yloxy)-6-oxohexanoyl)oxy)propan-2-yl) 8-(heptadecan-9-yl) octanedioate as colorless oil (840 mg, 79%).

### Step 5: Synthesis of (R)-1-(Heptadecan-9-yl) 8-(1-((6-(heptadecan-9-yloxy)-6-oxohexanoyl)oxy)-3-hydroxypropan-2-yl) octanedioate (9)

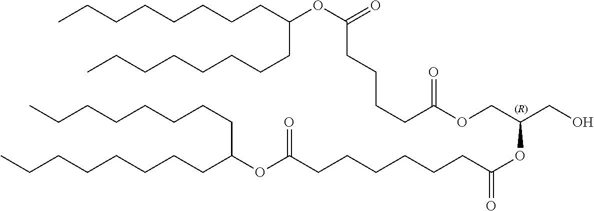

A mixture of (R)-1-(1-(benzyloxy)-3-((6-(heptadecan-9-yloxy)-6-oxohexanoyl)oxy)propan-2-yl) 8-(heptadecan-9-yl) octanedioate 8 (840 mg, 0.89 mmol) and 10% palladium on carbon (500 mg) in 40 mL ethyl acetate was hydrogenated over balloon for 12 h. After filtration through silica gel and concentration, the product was used for the next step without further purification (750 mg, 98%).

### Step 6: Synthesis of (S)-1-(Heptadecan-9-yl) 8-(1-((6-(heptadecan-9-yloxy)-6-oxo-hexanoyl)oxy)-3-((2-oxido-1,3,2-dioxaphospholan-2-yl)oxy)propan-2-yl) octanedioate (11)

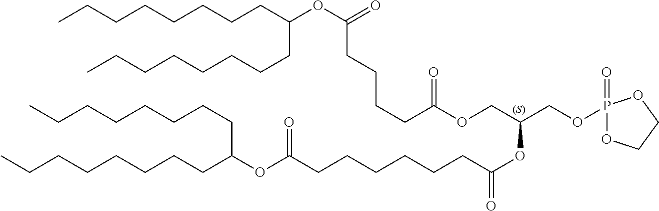

A solution of (R)-1-(heptadecan-9-yl) 8-(1-((6-(heptadecan-9-yloxy)-6-oxohexanoyl)oxy)-3-hydroxypropan-2-yl) octanedioate 9 (750 mg, 0.88 mmol) and triethylamine (0.18 mL, 1.32 mmol) in 5 mL THF was cooled to 0° C., and then a solution of 2-chloro-1,3,2-dioxaphospholane 2-oxide 10 (188 mg, 1.32 mmol) in 1 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 7: Synthesis of (S)-3-((6-(Heptadecan-9-yloxy)-6-oxohexanoyl)oxy)-2-((8-(heptadecan-9-yloxy)-8-oxooctanoyl)oxy)propyl (2-(trimethylammonio)ethyl) phosphate (Compound 48)

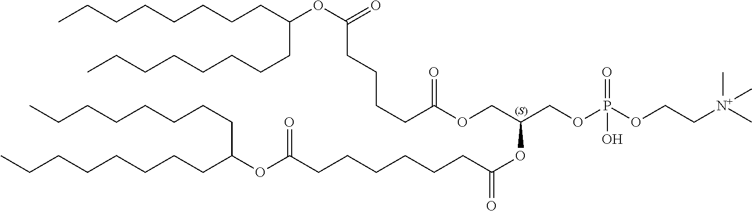

In a seal tube, a solution of (S)-1-(heptadecan-9-yl) 8-(1-((6-(heptadecan-9-yloxy)-6-oxo-hexanoyl)oxy)-3-((2-oxido-1,3,2-dioxaphospholan-2-yl)oxy)propan-2-yl) octanedioate 11 (crude) in 10 mL acetonitrile was cooled in ice bath, and a solution of trimethylamine (2 M in THF, 5.0 mL, 10 mmol) was added. The bottle was sealed and heated at 95° C. overnight. After cooling to room temperature, the volatiles were evaporated under vacuum, and the crude was purified by column chromatography (SiO₂: CHCl₃/MeOH/H₂O 32:13:0.4) to give the desired product as pale yellow solid (625 mg, 69%).

¹H NMR (400 MHz, CDCl₃) δ 5.25-5.12 (m, 1H), 4.89-4.74 (m, 2H), 4.44-4.24 (m, 3H), 4.19-4.05 (m, 1H), 4.00-3.88 (m, 2H), 3.84-3.74 (m, 2H), 3.35 (s, 9H), 2.36-2.16 (m, 8H), 1.69-1.39 (m, 16H), 1.36-1.12 (m, 52H), 0.85 (t, 12H).

ESI-MS analysis: Calculated C56H108NO12P [M+H]=1018.8, Observed=1018.8, 1040.7 (M+Na).

HPLC-ELSD: t_{R}=7.626 min (method 1).

### Synthetic Scheme for Compound 50

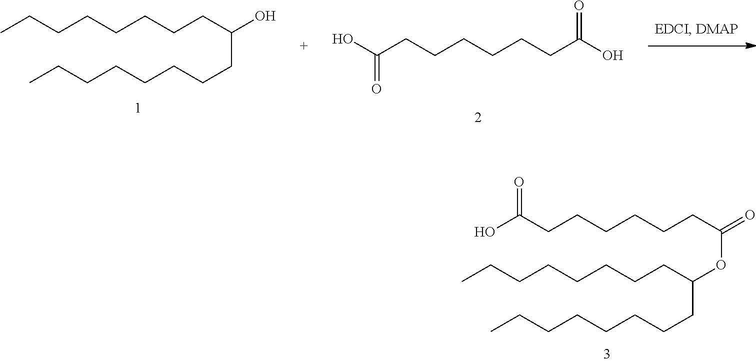 [SMILES: CCCCCCCCC(O)CCCCCCCC ; O=C(O)CCCCCCC(=O)O ; CCCCCCCCC(CCCCCCCC)OC(=O)CCCCCCC(=O)O]

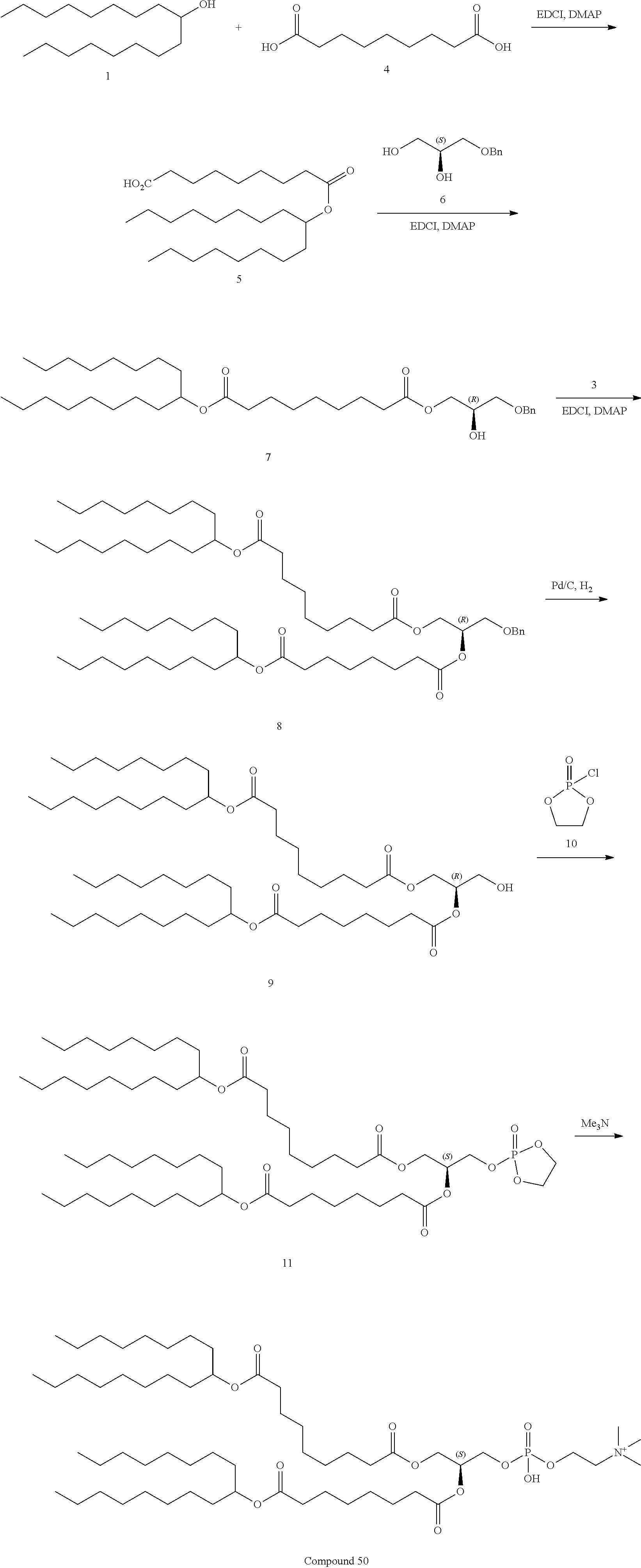

Synthetic Procedure for Compound 50:

### Step 1: Synthesis of 8-(Heptadecane-9-yloxy)-8-oxooctanoic acid (3)

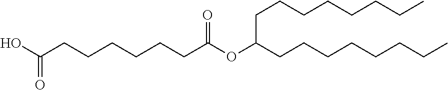 [SMILES: CCCCCCCCC(CCCCCCCC)OC(=O)CCCCCCC(=O)O]

To a stirred homogenous solution of suberic acid 2 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 1 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of 9-(Heptadecan-9-yloxy)-9-oxononanoic acid (5)

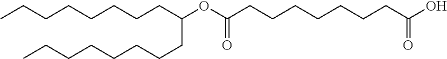 [SMILES: CCCCCCCCC(CCCCCCCC)OC(=O)CCCCCCCC(=O)O]

To a stirred homogenous solution of azelaic acid 4 (18 g, 94.4 mmol) in 100 mL DMF: DCM (1:1, v/v), was added DMAP (0.26 g, 2.15 mmol) and EDCI (9.8 g, 51.5 mmol), and the mixture was stirred at room temperature for 10 minutes. Then a homogeneous solution of 9-heptadecanol 1 (11 g, 43.4 mmol) in 50 mL DMF: DCM (1:1, v/v) was added dropwise via addition funnel over a period of 8 hours. The mixture was stirred at room temperature for 10 h. After removing the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 9-(heptadecan-9-yloxy)-9-oxononanoic acid as colorless oil (12.5 g, 70%).

### Step 3: Synthesis of (R)-1-(3-(Benzyloxy)-2-hydroxypropyl) 9-(heptadecan-9-yl) nonanedioate (7)

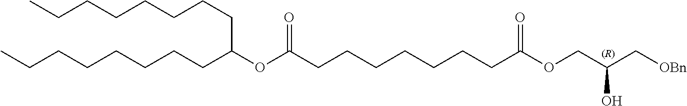

A mixture of (S)-3-(benzyloxy)propane-1,2-diol 6 (1.0 g, 6 mmol), 9-(heptadecan-9-yloxy)-9-oxononanoic acid 5 (1.7 g, 4 mmol), EDCI (2.2 g, 12 mmol) and DMAP (0.48 g, 4 mmol) in 30 mL dichloromethane was stirred at room temperature for 15 h. After concentration, the crude was triturated with hexane, and the solvent was removed. The crude was purified by column chromatography eluted with 0-40% ethyl acetate in hexane to yield (R)-1-(3-(benzyloxy)-2-hydroxypropyl) 9-(heptadecan-9-yl) nonanedioate as colorless oil (680 mg, 29%).

### Step 4: Synthesis of (R)-1-(3-(Benzyloxy)-2-((8-(heptadecan-9-yloxy)-8-oxooctanoyl)oxy)propyl) 9-(heptadecan-9-yl) nonanedioate (8)

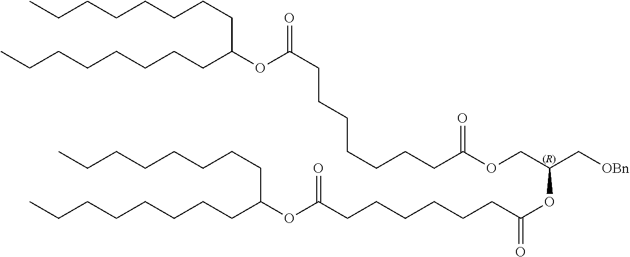

A mixture of (R)-1-(3-(benzyloxy)-2-hydroxypropyl) 9-(heptadecan-9-yl) nonanedioate 7 (680 mg, 1.15 mmol), 8-(heptadecan-9-yloxy)-8-oxooctanoic acid 3 (712 mg, 1.73 mmol), EDCI (1.2 g, 6 mmol) and DMAP (366 mg, 3 mmol) in 40 mL dichloromethane was stirred at room temperature overnight. TLC showed complete reaction. After concentration, the crude was triturated with hexane, and the solvent was removed. The crude was purified by column chromatography eluted with 0-15% ethyl acetate in hexane to yield (R)-1-(3-(benzyloxy)-2-((8-(heptadecan-9-yloxy)-8-oxooctanoyl)oxy)propyl) 9-(heptadecan-9-yl) nonanedioate as colorless oil (830 mg, 81%).

### Step 5: Synthesis of (R)-1-(Heptadecan-9-yl) 9-(2-((8-(heptadecan-9-yloxy)-8-oxooctanoyl)oxy)-3-hydroxypropyl) nonanedioate (9)

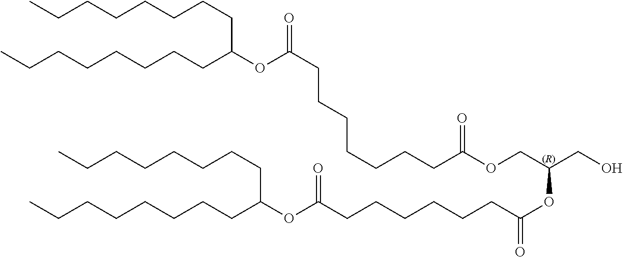

A mixture of (R)-1-(3-(benzyloxy)-2-((8-(heptadecan-9-yloxy)-8-oxooctanoyl)oxy)propyl) 9-(heptadecan-9-yl) nonanedioate 8 (830 mg, 0.87 mmol) and 10% palladium on carbon (500 mg) in 30 mL ethyl acetate was hydrogenated using balloon for 12 h. After filtration through silica gel and concentration, the product was used for the next step without further purification (750 mg, quant.).

### Step 6: Synthesis of (S)-1-(Heptadecan-9-yl) 9-(2-((8-(heptadecan-9-yloxy)-8-oxooctanoyl)oxy)-3-((2-oxido-1,3,2-dioxaphospholan-2-yl)oxy)propyl) nonanedioate (11)

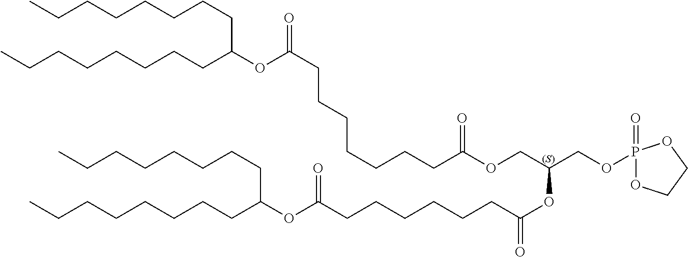

A solution of (R)-1-(heptadecan-9-yl) 9-(2-((8-(heptadecan-9-yloxy)-8-oxooctanoyl)oxy)-3-hydroxypropyl) nonanedioate 9 (750 mg, 0.87 mmol) and triethylamine (178 mg, 1.74 mmol) in 15 mL THF was cooled to 0° C., and then a solution of 2-chloro-1,3,2-dioxaphospholane 2-oxide 10 (248 mg, 1.74 mmol) in 1 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 7: Synthesis of (S)-2-((8-(Heptadecan-9-yloxy)-8-oxooctanoyl)oxy)-3-((9-(heptadecan-9-yloxy)-9-oxononanoyl)oxy)propyl (2-(trimethylammonio)ethyl) phosphate (Compound 50)

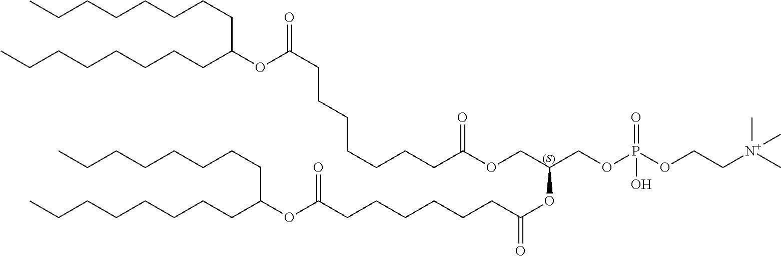

In a seal tube, a solution of (S)-1-(heptadecan-9-yl) 9-(2-((8-(heptadecan-9-yloxy)-8-oxooctanoyl)oxy)-3-((2-oxido-1,3,2-dioxaphospholan-2-yl)oxy)propyl) nonanedioate 11 (crude) in 15 mL acetonitrile was cooled in ice bath, and a solution of trimethylamine (2 M in THF, 5.0 mL, 10.0 mmol) was added. The bottle was sealed and heated at 95° C. overnight. After cooling to room temperature, the volatiles were evaporated under vacuum, and the crude was purified by column chromatography (SiO₂: CHCl₃/MeOH/H₂O 32:13:0.4) to give the desired product as pale yellow solid (60 mg, 6%).

¹H NMR (400 MHz, CDCl₃) δ 5.26-5.09 (m, 1H), 4.90-4.74 (m, 2H), 4.44-4.25 (m, 3H), 4.24-4.03 (m, 1H), 4.01-3.85 (m, 2H), 3.85-3.66 (m, 2H), 3.32 (s, 9H), 2.34-2.15 (m, 8H), 1.68-1.39 (m, 16H), 1.37-1.07 (m, 58H), 0.85 (t, 12H).

ESI-MS analysis: Calculated C59H114NO12P [M+H]=1060.8, Observed=1060.9, 1082.8 (M+Na).

HPLC-ELSD: t_{R}=6.516 min (method 1).

### Synthetic Scheme for Compound 52

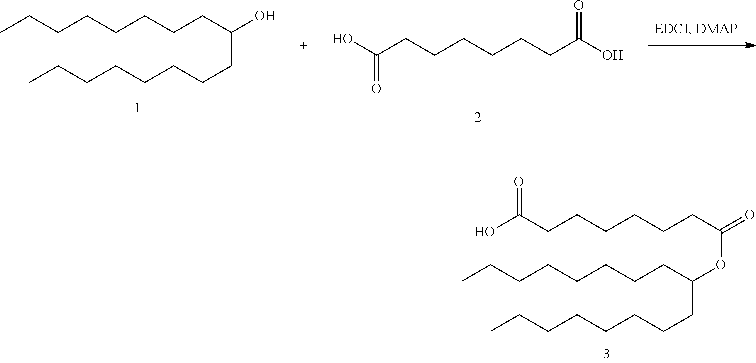 [SMILES: CCCCCCCCC(O)CCCCCCCC ; O=C(O)CCCCCCC(=O)O ; CCCCCCCCC(CCCCCCCC)OC(=O)CCCCCCC(=O)O]

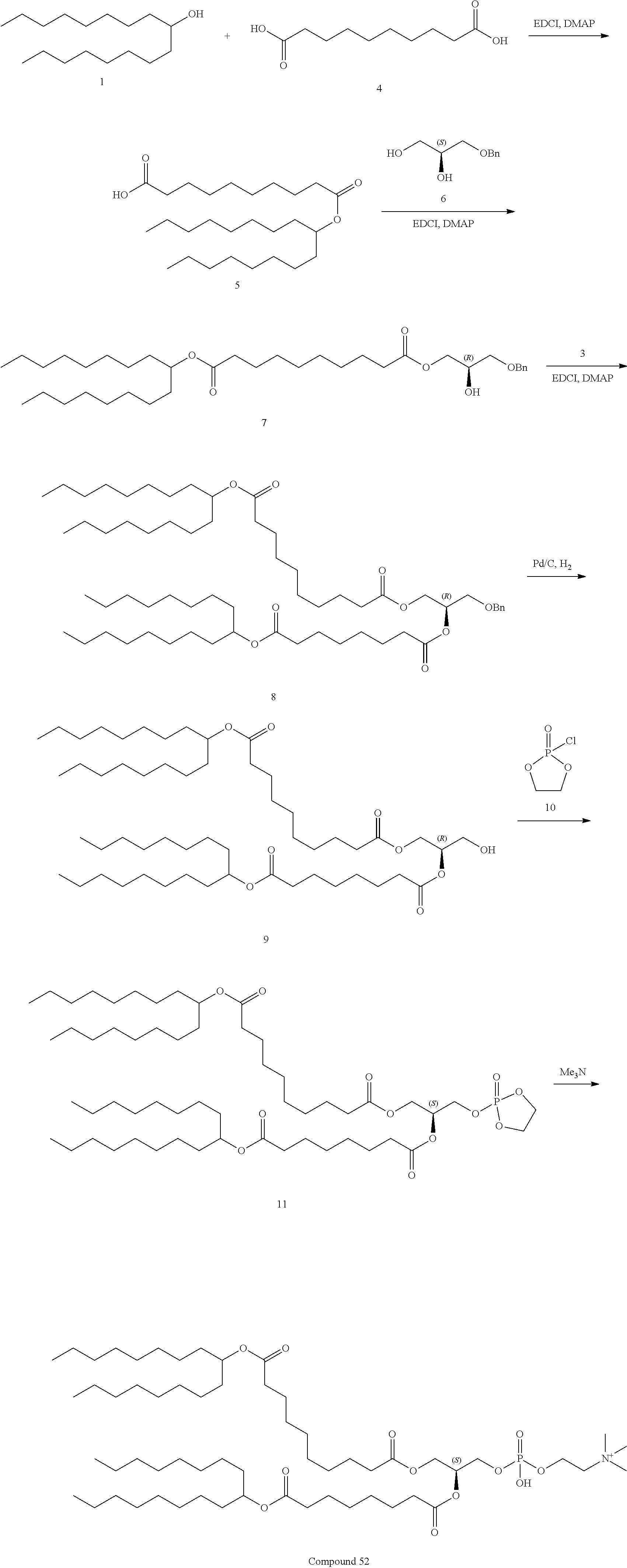

Synthetic Procedure for Compound 52:

### Step 1: Synthesis of 8-(Heptadecane-9-yloxy)-8-oxooctanoic acid (3)

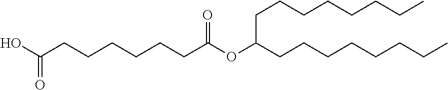 [SMILES: CCCCCCCCC(CCCCCCCC)OC(=O)CCCCCCC(=O)O]

To a stirred homogenous solution of suberic acid 2 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution 9-heptadecanol 1 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of 10-(Heptadecan-9-yloxy)-10-oxodecanoic acid (5)

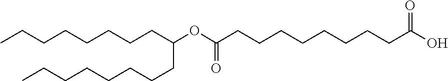 [SMILES: CCCCCCCCC(CCCCCCCC)OC(=O)CCCCCCCCC(=O)O]

To a stirred homogenous solution of sebacic acid 4 (26.3 g, 130 mmol) in 150 mL DMF: DCM (1:1, v/v), was added DMAP (5.2 g, 43.4 mmol) and EDCI (18 g, 95.5 mmol), and the mixture was stirred at room temperature for 10 minutes. Then a homogeneous solution of 9-heptadecanol 1 (11 g, 43.4 mmol) in 100 mL DMF: DCM (1:1, v/v) was added dropwise via addition funnel over a period of 8 hours. The mixture was stirred at room temperature for 10 h. After removing the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 10-(heptadecan-9-yloxy)-10-oxodecanoic acid as colorless oil (14.5 g, 76%).

### Step 3: Synthesis of (R)-1-(3-(Benzyloxy)-2-hydroxypropyl) 10-(heptadecan-9-yl) decanedioate (7)

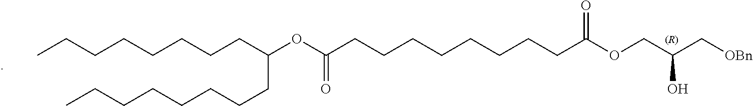

A mixture of (S)-3-(benzyloxy)propane-1,2-diol 6 (1.0 g, 6 mmol), 10-(heptadecan-9-yloxy)-10-oxodecanoic acid 5 (1.76 g, 4 mmol), EDCI (2.2 g, 12 mmol) and DMAP (0.48 g, 4 mmol) in 30 mL dichloromethane was stirred at room temperature for 15 h. After concentration, the crude was triturated with hexane, and the solvent was removed. The crude was purified by column chromatography eluted with 0-40% ethyl acetate in hexane to yield (R)-1-(3-(benzyloxy)-2-hydroxypropyl) 10-(heptadecan-9-yl) decanedioate as colorless oil (1.0 g, 41%).

### Step 4: Synthesis of (R)-1-(3-(Benzyloxy)-2-((8-(heptadecan-9-yloxy)-8-oxooctanoyl)oxy)propyl) 10-(heptadecan-9-yl) decanedioate (8)

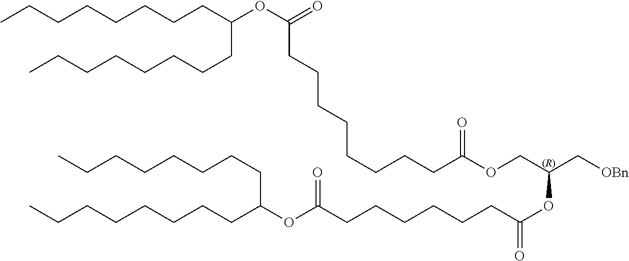

A mixture of (R)-1-(3-(benzyloxy)-2-hydroxypropyl) 10-(heptadecan-9-yl) decanedioate 7 (980 mg, 1.62 mmol), 8-(heptadecan-9-yloxy)-8-oxooctanoic acid 3 (1.25 g, 3.0 mmol), EDCI (0.6 g, 3 mmol) and DMAP (183 mg, 1.5 mmol) in 40 mL dichloromethane was stirred at room temperature overnight. TLC showed complete reaction. After concentration, the crude was triturated with hexane, and the solvent was removed. The crude was purified by column chromatography eluted with 0-15% ethyl acetate in hexane to yield (R)-1-(3-(benzyloxy)-2-((8-(heptadecan-9-yloxy)-8-oxooctanoyl)oxy)propyl) 10-(heptadecan-9-yl) decanedioate as colorless oil (1.55 g, 90%).

### Step 5: Synthesis of (R)-1-(Heptadecan-9-yl) 10-(2-((8-(heptadecan-9-yloxy)-8-oxooctanoyl)oxy)-3-hydroxypropyl) decanedioate (9)

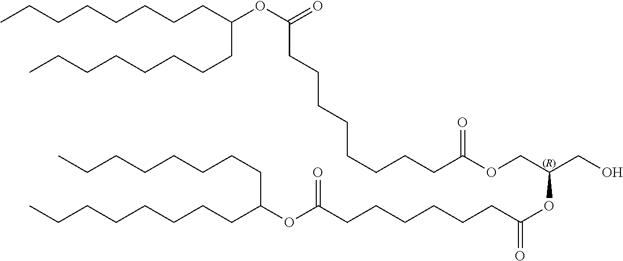

A mixture of (R)-1-(3-(benzyloxy)-2-((8-(heptadecan-9-yloxy)-8-oxooctanoyl)oxy)propyl) 10-(heptadecan-9-yl) decanedioate 8 (1.55 g, 1.54 mmol) and 10% palladium on carbon (500 mg) in 30 mL ethyl acetate was hydrogenated using balloon for 12 h. After filtration through silica gel and concentration, the product was used for the next step without further purification (1.4 g, quant.).

### Step 6: Synthesis of (S)-1-(Heptadecan-9-yl) 10-(2-((8-(heptadecan-9-yloxy)-8-oxooctanoyl)oxy)-3-((2-oxido-1,3,2-dioxaphospholan-2-yl)oxy)propyl) decanedioate (11)

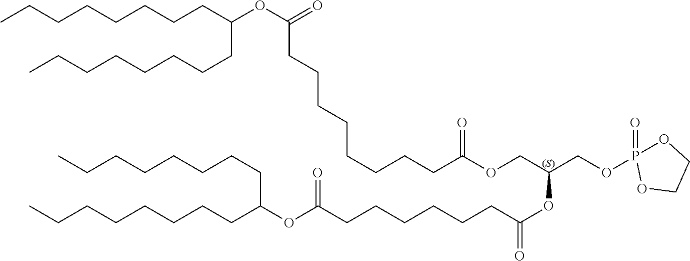

A solution of (R)-1-(heptadecan-9-yl) 10-(2-((8-(heptadecan-9-yloxy)-8-oxooctanoyl)oxy)-3-hydroxypropyl) decanedioate 9 (1.36 g, 1.5 mmol) and triethylamine (0.42 mL, 3 mmol) in 15 mL THF was cooled to 0° C., and then a solution of 2-chloro-1,3,2-dioxaphospholane 2-oxide 10 (428 mg, 3 mmol) in 1 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 7: Synthesis of (S)-3-((10-(Heptadecan-9-yloxy)-10-oxodecanoyl)oxy)-2-((8-(heptadecan-9-yloxy)-8-oxooctanoyl)oxy)propyl (2-(trimethylammonio)ethyl) phosphate (Compound 52)

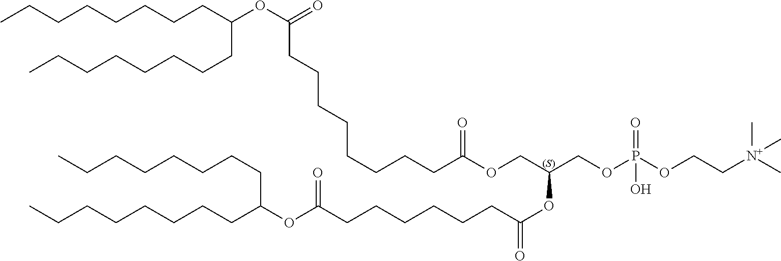

In a seal tube, a solution of (S)-1-(heptadecan-9-yl) 10-(2-((8-(heptadecan-9-yloxy)-8-oxooctanoyl)oxy)-3-((2-oxido-1,3,2-dioxaphospholan-2-yl)oxy)propyl) decanedioate 11 (crude) in 15 mL acetonitrile was cooled in ice bath, and a solution of trimethylamine (2 M in THF, 7.0 mL, 14.0 mmol) was added. The bottle was sealed and heated at 95° C. for 3 days. After cooling to room temperature, the volatiles were evaporated under vacuum, and the crude was purified by column chromatography (SiO₂: CHCl₃/MeOH/H₂O 32:13:0.4) to give the desired product as pale yellow solid (765 mg, 49%).

¹H NMR (400 MHz, CDCl₃) δ 5.26-5.12 (m, 1H), 4.90-4.76 (m, 2H), 4.43-4.26 (m, 3H), 4.18-4.04 (m, 1H), 4.02-3.88 (m, 2H), 3.87-3.76 (m, 2H), 3.36 (s, 9H), 2.35-2.17 (m, 8H), 1.67-1.39 (m, 16H), 1.36-1.10 (m, 60H), 0.86 (t, 12H).

ESI-MS analysis: Calculated C60H116NO12P [M+H]=1074.8, Observed=1074.7.

HPLC-ELSD: t_{R}=7.496 min (method 1).

### Synthetic Scheme for Compound 54

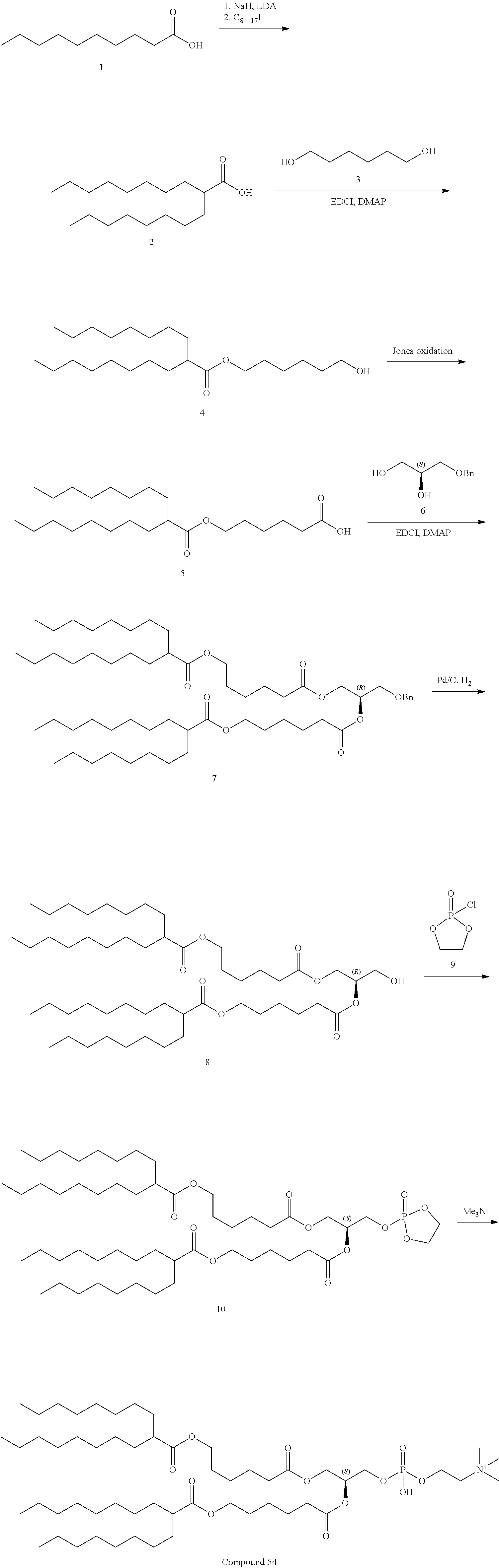

### Synthetic Procedure for Compound 54

### Step 1: Synthesis of 2-Octyldecanoic acid (2)

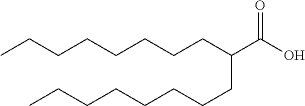 [SMILES: CCCCCCCCC(CCCCCCCC)C(=O)O]

A suspension of sodium hydride (60% in mineral oil, 2.56 g, 64 mmol) in 300 mL THF was cooled to 0° C., and a solution of decanoic acid 1 (10 g, 58 mmol) in 100 mL THF was added slowly, then a solution of lithium diisopropylamide (2 M in THF, 34.8 mL, 69.6 mmol) was slowly added. The reaction mixture was warmed up to room temperature for 30 min. After addition of 1-octyl iodide (16.7 g, 69.6 mmol), the resulting mixture was heated to reflux overnight. The reaction was cooled to room temperature and adjusted to pH 2 by 4 N HCl, and then extracted with ethyl acetate. The combined organic layer was concentrated and purified by column chromatography (SiO₂: 0 to 40% ethyl acetate in hexane) to give 2-octyldecanoic acid as white solid (7.4 g, 45%).

### Step 2: Synthetic of 6-Hydroxyhexyl 2-octyldecanoate (4)

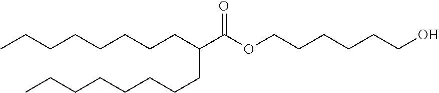 [SMILES: CCCCCCCCC(CCCCCCCC)C(=O)OCCCCCCO]

A mixture of 2-octyldecanoic acid 2 (3.0 g, 10.5 mmol), hexane-1,6-diol 3 (2.5 g, 21 mmol), EDCI (4.0 g, 21 mmol) and DMAP (1.28 g, 10.5 mmol) in 150 mL dichloromethane was stirred at room temperature for 3 days. The clear solution was diluted with water and extracted with dichloromethane. The combined organic layer was concentrated under vacuum, and the crude was purified by flash column chromatography (SiO₂: 0 to 40% ethyl acetate in hexane) to give 6-hydroxyhexyl 2-octyldecanoate as colorless oil (3.4 g, 68%).

### Step 3: Synthetic of 6-((2-Octyldecanoyl)oxy)hexanoic acid (5)

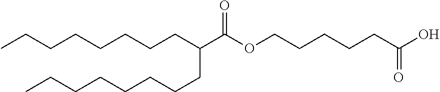 [SMILES: CCCCCCCCC(CCCCCCCC)C(=O)OCCCCCC(=O)O]

To a solution of 6-hydroxyhexyl 2-octyldecanoate 4 (3.7 g, 9.6 mmol) in 50 mL acetone, was added Jones reagent until the orange color persisted, and the resulting mixture was stirred for 30 min. The excess Jones reagent was consumed by adding few drops of 2-propanol, then the blue solution was diluted with water (100 mL) and extracted with ethyl acetate (3×100 mL). The combined organic layers were washed with brine, dried and concentrated to give the desired product (3.77 g, quant.), which was used for the next step without further purification.

### Step 4: Synthesis of (R)-((3-(Benzyloxy)propane-1,2-diyl)bis(oxy))bis(6-oxohexane-6,1-diyl) bis(2-octyldecanoate) (7)

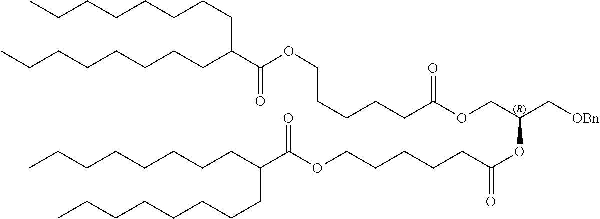

A solution of 6-((2-octyldecanoyl)oxy)hexanoic acid 5 (3.6 g, 9 mmol), (S)-3-(benzyloxy)propane-1,2-diol 6 (730 mg, 4 mmol), EDCI (4.0 g, 21 mmol) and DMAP (1.0 g, 9 mmol) in 60 mL dichloromethane was stirred at room temperature for 48 h. After concentration, the residue was partitioned with saturated ammonium chloride solution and ethyl acetate. The combined organic layer was concentrated under vacuum, and the crude was purified by flash column chromatography (SiO₂: 0 to 20% ethyl acetate in hexane) to give (R)-((3-(benzyloxy)propane-1,2-diyl)bis(oxy))bis(6-oxohexane-6,1-diyl) bis(2-octyldecanoate) as colorless oil (3.12 g, 79%).

### Step 5: Synthesis of (R)-((3-Hydroxypropane-1,2-diyl)bis(oxy))bis(6-oxohexane-6,1-diyl) bis(2-octyldecanoate) (8)

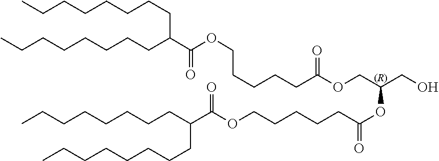

In a Parr reactor, a mixture of (R)-((3-(benzyloxy)propane-1,2-diyl)bis(oxy))bis(6-oxohexane-6,1-diyl) bis(2-octyldecanoate) 7 (1.0 g, 1.06 mmol) and 10% palladium on carbon (700 mg) in 60 mL ethyl acetate was hydrogenated under 40 psi for 16 h. After filtration and concentration, (R)-9-((3-hydroxypropane-1,2-diyl)bis(oxy))bis(6-oxohexane-6,1-diyl) bis(2-octyldecanoate) was obtained as colorless oil (937 mg, quant.).

### Step 6: Synthesis of (S)-((3-((2-Oxido-1,3,2-dioxaphospholan-2-yl)oxy)propane-1,2-diyl)bis(oxy))bis(6-oxohexane-6,1-diyl) bis(2-octyldecanoate) (10)

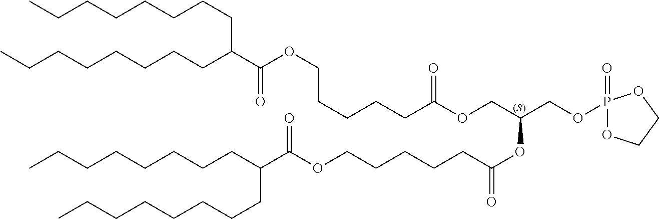

A solution of (R)-9-((3-hydroxypropane-1,2-diyl)bis(oxy))bis(6-oxohexane-6,1-diyl) bis(2-octyldecanoate) 8 (900 mg, 1.06 mmol) and triethylamine (0.29 mL, 2.12 mmol) in 10 mL THF was cooled to 0° C., and then a solution of 2-chloro-2-oxo-1,3,2-dioxaphospholane 9 (0.30 g, 2.12 mmol) in 2 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 7: Synthesis of (S)-2,3-Bis((6-((2-octyldecanoyl)oxy)hexanoyl)oxy)propyl (2-(trimethylammonio)ethyl) phosphate (Compound 54)

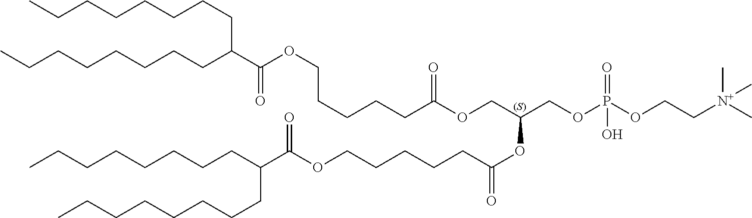

In a seal tube, a solution of (S)-((3-((2-oxido-1,3,2-dioxaphospholan-2-yl)oxy)propane-1,2-diyl)bis(oxy))bis(6-oxohexane-6,1-diyl) bis(2-octyldecanoate) 10 (crude) in 6 mL acetonitrile was cooled in ice bath, and a solution of trimethylamine (2 M in THF, 6.0 mL, 12 mmol) was added. The bottle was sealed and heated at 95° C. for 3 days. After cooling to room temperature, the volatiles were evaporated under vacuum, and the crude was purified by column chromatography (SiO₂: CHCl₃/MeOH/H₂O 32:13:0.4) to give the desired product as pale yellow solid (611 mg, 54%).

¹H NMR (400 MHz, CDCl₃) δ 5.30-5.05 (m, 1H), 4.45-4.28 (m, 3H), 4.15-4.08 (m, 1H), 4.03 (t, 4H), 3.99-3.90 (m, 2H), 3.86-3.77 (m, 2H), 3.36 (s, 9H), 2.42-2.18 (m, 6H), 1.70-1.49 (m, 12H), 1.47-1.32 (m, 8H), 1.31-1.12 (m, 48H), 0.86 (t, 12H).

³¹P NMR (162 MHz, CDCl₃) δ −0.24.

ESI-MS analysis: Calculated C50H108NO12P [M+H]=1018.8, Observed=1018.6.

HPLC-ELSD: t_{R}=6.851 min (method 1).

### Synthetic Scheme for Compound 56

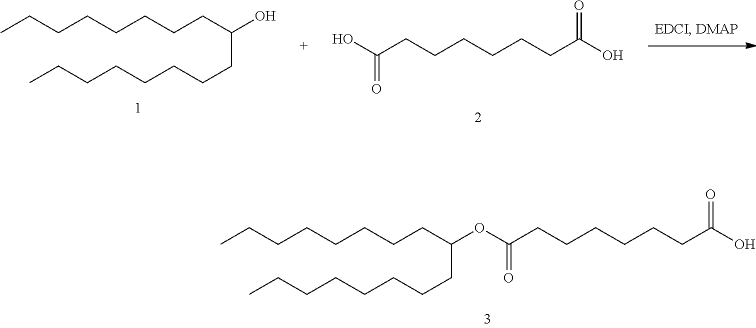 [SMILES: CCCCCCCCC(O)CCCCCCCC ; O=C(O)CCCCCCC(=O)O ; CCCCCCCCC(CCCCCCCC)OC(=O)CCCCCCC(=O)O]

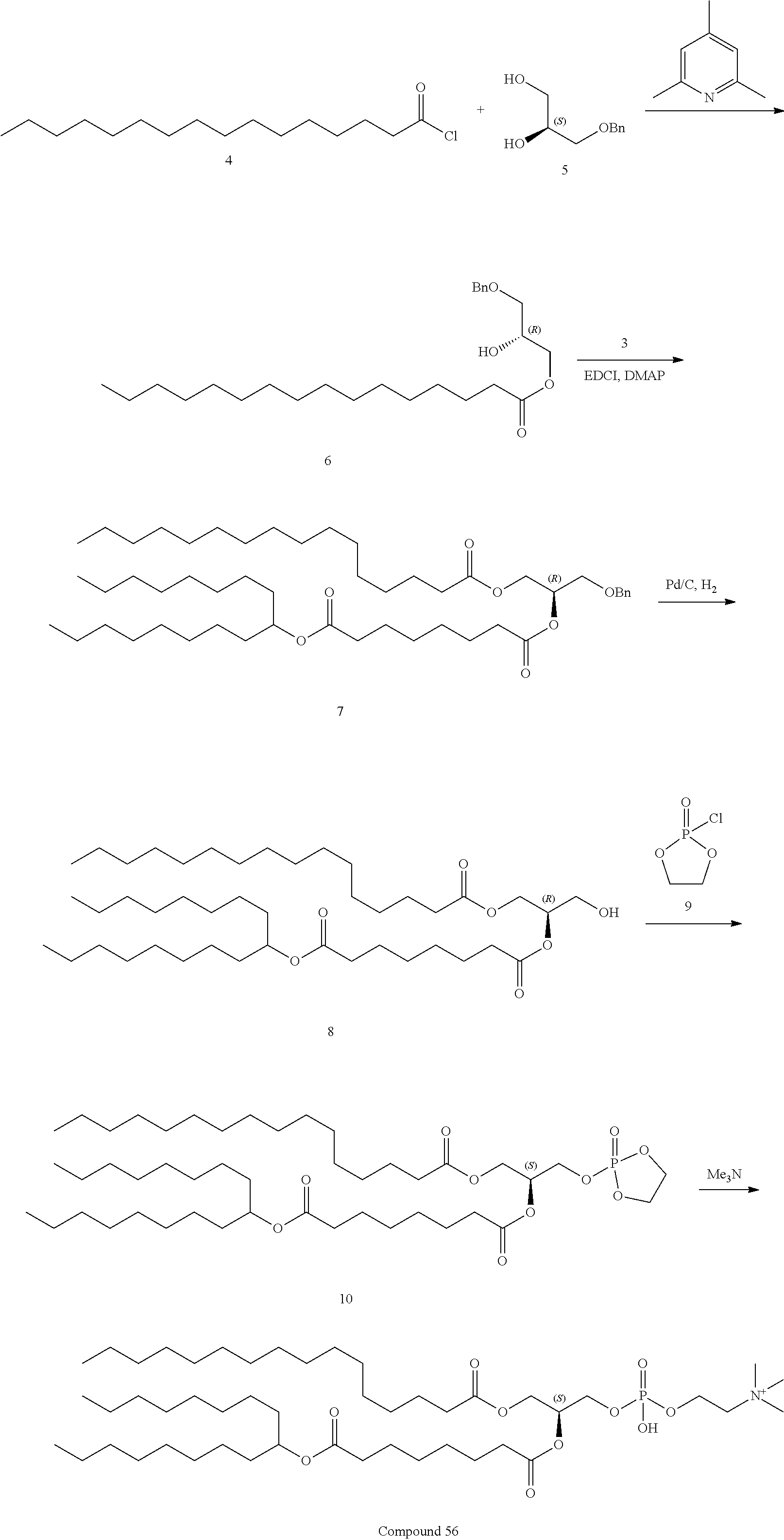

Synthetic Procedure for Compound 56:

### Step 1: Synthesis of 8-(Heptadecane-9-yloxy)-8-oxooctanoic acid (3)

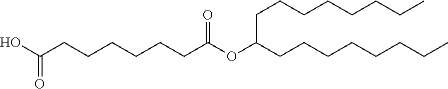 [SMILES: CCCCCCCCC(CCCCCCCC)OC(=O)CCCCCCC(=O)O]

To a stirred homogenous solution of suberic acid 4 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 1 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (R)-3-(Benzyloxy)-2-hydroxypropyl palmitate (6)

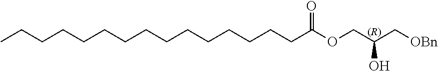

A mixture of (S)-3-(benzyloxy)propane-1,2-diol 5 (1.0 g, 6 mmol) and 2,4,6-trimethylpyridine (2.6 mL, 20 mmol) in 50 mL dichloromethane was cooled to 0° C., palmitoyl chloride 4 (1.2 mL, 4 mmol) was added dropwise, and then the reaction mixture was stirred at this temperature for 2 h. The mixture was diluted with dichloromethane and washed with 1 N HCl, water and brine. The combined organic layer was dried over sodium sulfate. After filtration and concentration, the crude was purified by column chromatography eluted with 0-20% ethyl acetate in hexane to yield (R)-3-(benzyloxy)-2-hydroxypropyl palmitate as colorless oil (1.38 g, 82%).

### Step 3: Synthesis of (R)-1-(1-(Benzyloxy)-3-(palmitoyloxy)propan-2-yl) 8-(heptadecan-9-yl) octanedioate (7)

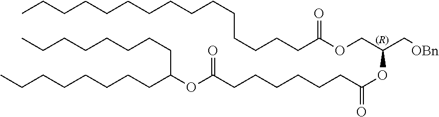

A mixture of (R)-3-(benzyloxy)-2-hydroxypropyl palmitate 6 (840 mg, 2.0 mmol), 8-(heptadecan-9-yloxy)-8-oxooctanoic acid 3 (1.25 g, 3.0 mmol), EDCI (0.6 g, 3 mmol) and DMAP (183 mg, 1.5 mmol) in 25 mL dichloromethane was stirred at room temperature overnight. TLC showed complete reaction. After concentration, the crude was triturated with hexane, and the solvent was removed. The crude was purified by column chromatography eluted with 0-15% ethyl acetate in hexane to yield (R)-1-(1-(benzyloxy)-3-(palmitoyloxy)propan-2-yl) 8-(heptadecan-9-yl) octanedioate as colorless oil (1.5 g, 93%).

### Step 4: Synthesis of (R)-1-(Heptadecan-9-yl) 8-(1-hydroxy-3-(palmitoyloxy)propan-2-yl) octanedioate (8)

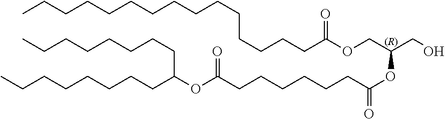

A mixture of (R)-1-(1-(benzyloxy)-3-(palmitoyloxy)propan-2-yl) 8-(heptadecan-9-yl) octanedioate 7 (1.5 g, 1.8 mmol) and 10% palladium on carbon (600 mg) in 25 mL ethyl acetate was hydrogenated using balloon for 12 h. After filtration through silica gel and concentration, the product was used for the next step without further purification (1.3 g, quant.).

### Step 5: Synthesis of (S)-1-(Heptadecan-9-yl) 8-(1-((2-oxido-1,3,2-dioxaphospholan-2-yl)oxy)-3-(palmitoyloxy)propan-2-yl) octanedioate (10)

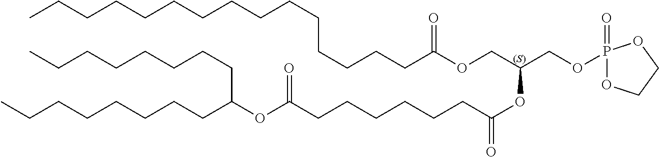

A solution of (R)-1-(heptadecan-9-yl) 8-(1-hydroxy-3-(palmitoyloxy)propan-2-yl) octanedioate 8 (1.3 g, 1.8 mmol) and triethylamine (0.42 mL, 3 mmol) in 15 mL THF was cooled to 0° C., and then a solution of 2-chloro-1,3,2-dioxaphospholane 2-oxide 9 (427 mg, 3 mmol) in 5 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 6: Synthesis of (S)-2-((8-(Heptadecan-9-yloxy)-8-oxooctanoyl)oxy)-3-(palmitoyloxy)propyl (2-(trimethylammonio)ethyl) phosphate (Compound 56)

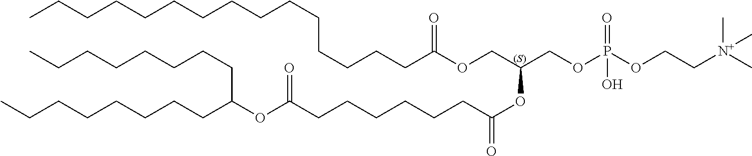

In a seal tube, a solution of (S)-1-(heptadecan-9-yl) 8-(1-((2-oxido-1,3,2-dioxaphospholan-2-yl)oxy)-3-(palmitoyloxy)propan-2-yl) octanedioate 10 (crude) in 15 mL acetonitrile was cooled in ice bath, and a solution of trimethylamine (2 M in THF, 10 mL, 20 mmol) was added. The bottle was sealed and heated at 95° C. overnight. After cooling to room temperature, the volatiles were evaporated under vacuum, and the crude was purified by column chromatography (SiO₂: CHCl₃/MeOH/H₂O 32:13:0.4) to give the desired product as pale yellow solid (1.13 g, 69%).

¹H NMR (400 MHz, CDCl₃) δ 5.24-5.12 (m, 1H), 4.88-4.77 (m, 1H), 4.45-4.22 (m, 3H), 4.16-4.04 (m, 1H), 3.98-3.76 (m, 4H), 3.37 (s, 9H), 2.37-2.18 (m, 6H), 1.69-1.42 (m, 8H), 1.36-1.14 (m, 54H), 0.86 (t, 9H).

³¹P NMR (162 MHz, CDCl₃) δ −0.24.

ESI-MS analysis: Calculated C49H96NO10P [M+H]=890.6, Observed=890.6, 912.5 (M+Na).

HPLC-ELSD: t_{R}=6.244 min (method 1).

### Synthetic Scheme for Compound 57

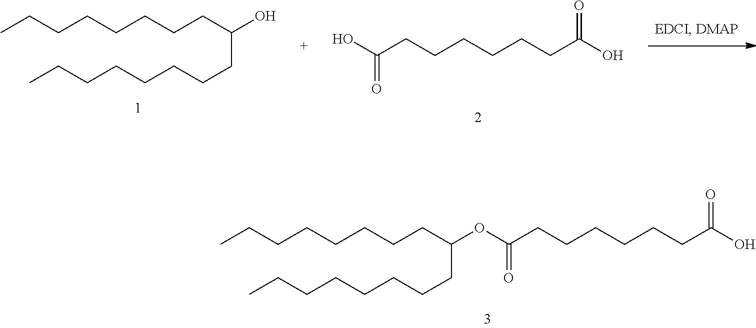 [SMILES: CCCCCCCCC(O)CCCCCCCC ; O=C(O)CCCCCCC(=O)O ; CCCCCCCCC(CCCCCCCC)OC(=O)CCCCCCC(=O)O]

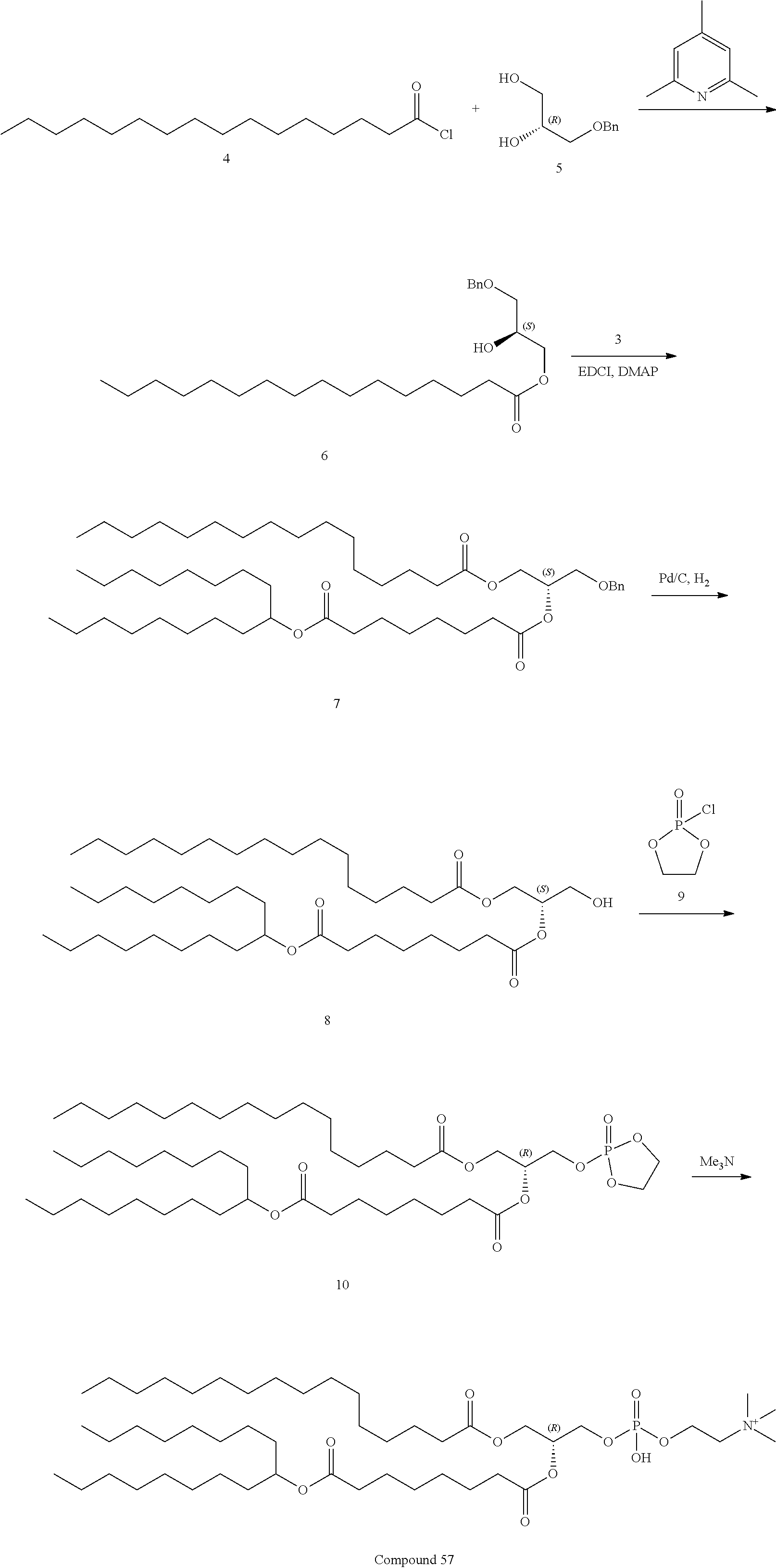

Synthetic Procedure for Compound 57:

### Step 1: Synthesis of 8-(Heptadecane-9-yloxy)-8-oxooctanoic acid (3)

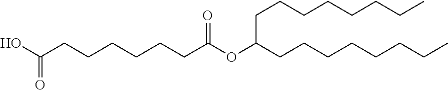 [SMILES: CCCCCCCCC(CCCCCCCC)OC(=O)CCCCCCC(=O)O]

To a stirred homogenous solution of suberic acid 2 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 1 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (S)-3-(Benzyloxy)-2-hydroxypropyl palmitate (6)

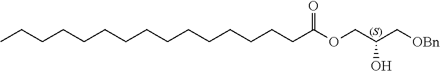

A mixture of (R)-3-(benzyloxy)propane-1,2-diol 5 (1.0 g, 6 mmol) and 2,4,6-trimethylpyridine (2.6 mL, 20 mmol) in 50 mL dichloromethane was cooled to 0° C., palmitoyl chloride 4 (1.2 mL, 4 mmol) was added dropwise, and then the reaction mixture was stirred at this temperature for 2 h. The mixture was diluted with dichloromethane and washed with 1 N HCl, water and brine. The combined organic layer was dried over sodium sulfate. After filtration and concentration, the crude was purified by column chromatography eluted with 0-30% ethyl acetate in hexane to yield (S)-3-(benzyloxy)-2-hydroxypropyl palmitate as colorless oil (1.35 g, 81%).

### Step 3: Synthesis of (S)-1-(1-(Benzyloxy)-3-(palmitoyloxy)propan-2-yl) 8-(heptadecan-9-yl) octanedioate (7)

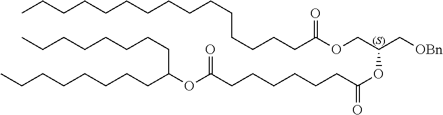

A mixture of (S)-3-(benzyloxy)-2-hydroxypropyl palmitate 6 (840 mg, 2.0 mmol), 8-(heptadecan-9-yloxy)-8-oxooctanoic acid 3 (1.25 g, 3.0 mmol), EDCI (0.6 g, 3 mmol) and DMAP (183 mg, 1.5 mmol) in 25 mL dichloromethane was stirred at room temperature overnight. TLC showed complete reaction. After concentration, the crude was triturated with hexane, and the solvent was removed. The crude was purified by column chromatography eluted with 0-15% ethyl acetate in hexane to yield (S)-1-(1-(benzyloxy)-3-(palmitoyloxy)propan-2-yl) 8-(heptadecan-9-yl) octanedioate as colorless oil (1.5 g, 93%).

### Step 4: Synthesis of (S)-1-(Heptadecan-9-yl) 8-(1-hydroxy-3-(palmitoyloxy)propan-2-yl) octanedioate (8)

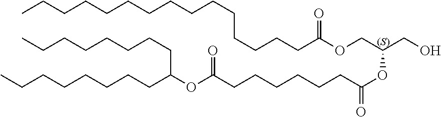

A mixture of (S)-1-(1-(benzyloxy)-3-(palmitoyloxy)propan-2-yl) 8-(heptadecan-9-yl) octanedioate 7 (1.5 g, 1.8 mmol) and 10% palladium on carbon (600 mg) in 25 mL ethyl acetate was hydrogenated under 40 psi for 26 h. After filtration through silica gel and concentration, the product was used for the next step without further purification (1.3 g, quant.).

### Step 5: Synthesis of (R)-1-(Heptadecan-9-yl) 8-(1-((2-oxido-1,3,2-dioxaphospholan-2-yl)oxy)-3-(palmitoyloxy)propan-2-yl) octanedioate (10)

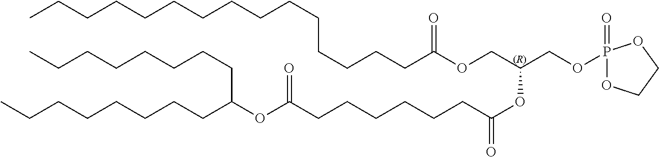

A solution of (S)-1-(heptadecan-9-yl) 8-(1-hydroxy-3-(palmitoyloxy)propan-2-yl) octanedioate 8 (1.3 g, 1.8 mmol) and triethylamine (0.50 mL, 3.6 mmol) in 15 mL THF was cooled to 0° C., and then a solution of 2-chloro-1,3,2-dioxaphospholane 2-oxide 9 (513 mg, 3.6 mmol) in 5 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 6: Synthesis of (R)-2-((8-(Heptadecan-9-yloxy)-8-oxooctanoyl)oxy)-3-(palmitoyloxy)propyl (2-(trimethylammonio)ethyl) phosphate (Compound 57)

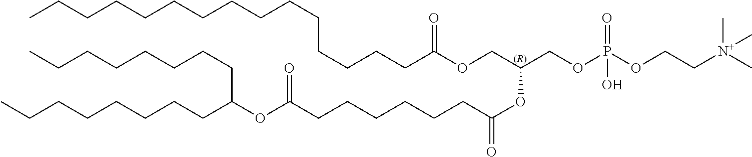

In a seal tube, a solution of (R)-1-(heptadecan-9-yl) 8-(1-((2-oxido-1,3,2-dioxaphospholan-2-yl)oxy)-3-(palmitoyloxy)propan-2-yl) octanedioate 10 (crude) in 15 mL acetonitrile was cooled in ice bath, and a solution of trimethylamine (2 M in THF, 10 mL, 20 mmol) was added. The bottle was sealed and heated at 95° C. overnight. After cooling to room temperature, the volatiles were evaporated under vacuum, and the crude was purified by column chromatography (SiO₂: CHCl₃/MeOH/H₂O 32:13:0.4) to give the desired product as pale yellow solid (0.95 g, 59%).

¹H NMR (400 MHz, CDCl₃) δ 5.23-5.13 (m, 1H), 4.89-4.77 (m, 1H), 4.47-4.26 (m, 3H), 4.16-4.04 (m, 1H), 4.00-3.79 (m, 4H), 3.37 (s, 9H), 2.37-2.16 (m, 6H), 1.68-1.42 (m, 8H), 1.37-1.08 (m, 54H), 0.86 (t, 9H).

³¹P NMR (162 MHz, CDCl₃) δ −0.27.

ESI-MS analysis: Calculated C49H96NO10P [M+H]=890.6, Observed=890.5, 912.5 (M+Na).

HPLC-ELSD: t_{R}=6.234 min (method 1).

### Synthetic Scheme for Compound 59

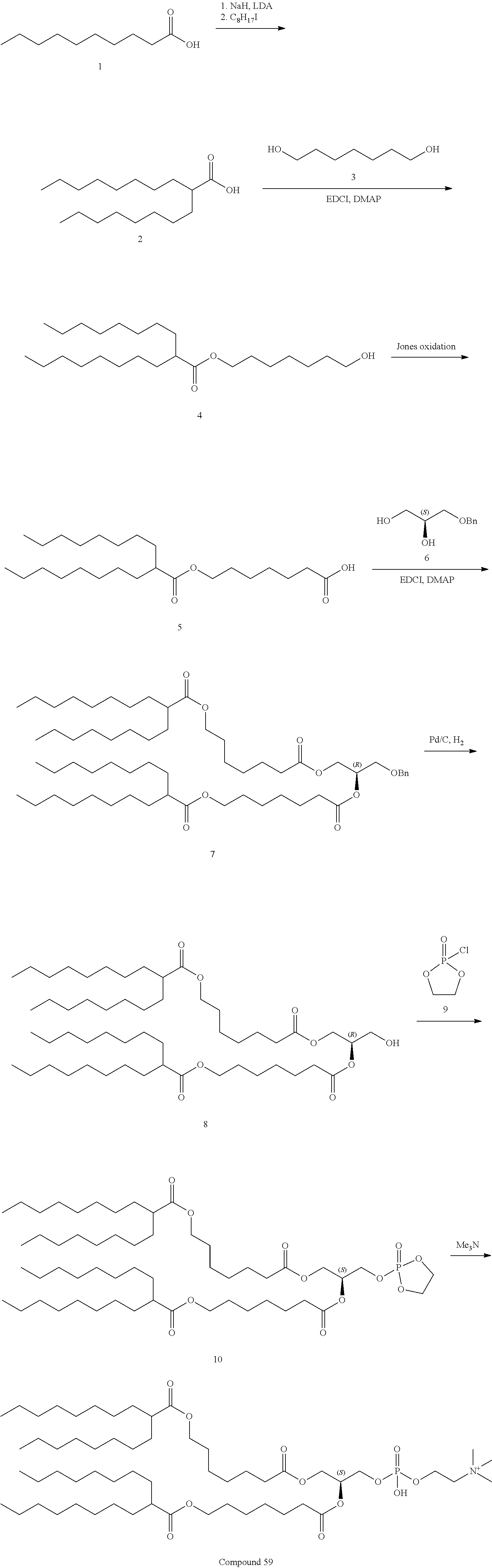

Synthetic Procedure for Compound 59:

### Step 1: Synthesis of 2-Octyldecanoic acid (2)

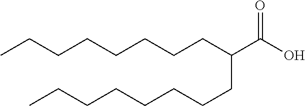 [SMILES: CCCCCCCCC(CCCCCCCC)C(=O)O]

A suspension of sodium hydride (60% in mineral oil, 2.56 g, 64 mmol) in 300 mL THF was cooled to 0° C., and a solution of decanoic acid 1 (10 g, 58 mmol) in 100 mL THF was added slowly, then a solution of lithium diisopropylamide (2 M in THF, 34.8 mL, 69.6 mmol) was slowly added. The reaction mixture was warmed up to room temperature for 30 min. After addition of 1-octyl iodide (16.7 g, 69.6 mmol), the resulting mixture was heated to reflux overnight. The reaction was cooled to room temperature and adjusted to pH 2 by 4 N HCl, and then extracted with ethyl acetate. The combined organic layer was concentrated and purified by column chromatography (SiO₂: 0 to 40% ethyl acetate in hexane) to give 2-octyldecanoic acid as white solid (7.4 g, 45%).

### Step 2: Synthesis of 7-Hydroxyheptyl 2-octyldecanoate (4)

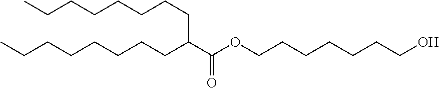 [SMILES: CCCCCCCCC(CCCCCCCC)C(=O)OCCCCCCCO]

A mixture of 2-octyldecanoic acid 2 (4.4 g, 15.5 mmol), heptane-1,7-diol 3 (4.0 g, 31 mmol), EDCI (6.0 g, 31 mmol) and DMAP (1.8 g, 15.5 mmol) in 150 mL dichloromethane was stirred at room temperature 3 days. The clear solution was diluted with water and extracted with dichloromethane. The combined organic layer was concentrated under vacuum, and the crude was purified by flash column chromatography (SiO₂: 0 to 40% ethyl acetate in hexane) to give 7-hydroxyheptyl 2-octyldecanoate as colorless oil (4.4 g, 72%).

### Step 3: Synthesis of 7-((2-Octyldecanoyl)oxy)heptanoic acid (5)

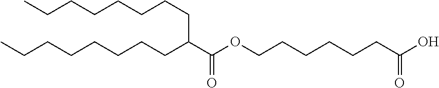 [SMILES: CCCCCCCCC(CCCCCCCC)C(=O)OCCCCCCC(=O)O]

To a solution of 7-hydroxyheptyl 2-octyldecanoate 4 (4.4 g, 11 mmol) in 50 mL acetone, was added Jones reagent until the orange color persisted, and the resulting mixture was stirred for 30 min. The excess Jones reagent was consumed by adding few drops of 2-propanol, then the blue solution was diluted with water (100 mL) and extracted with ethyl acetate (3×100 mL). The combined organic layers were washed with brine, dried and concentrated to give the desired product (4.53 g, quant.), which was used for the next step without further purification.

### Step 4: Synthesis of (R)-((3-(Benzyloxy)propane-1,2-diyl)bis(oxy))bis(7-oxoheptane-7,1-diyl) bis(2-octyldecanoate) (7)

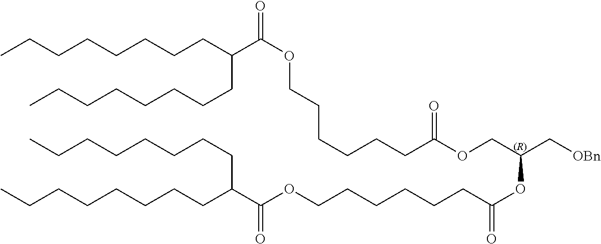

A solution of 7-((2-octyldecanoyl)oxy)heptanoic acid 5 (4.5 g, 11 mmol), (S)-3-(benzyloxy)propane-1,2-diol 6 (900 mg, 4.9 mmol), EDCI (4.8 g, 24.5 mmol) and DMAP (1.34 g, 11 mmol) in 60 mL dichloromethane was stirred at room temperature for 48 h. After concentration, the residue was partitioned with saturated ammonium chloride solution and ethyl acetate. The combined organic layer was concentrated under vacuum, and the crude was purified by flash column chromatography (SiO₂: 0 to 20% ethyl acetate in hexane) to give (R)-((3-(benzyloxy)propane-1,2-diyl)bis(oxy))bis(7-oxoheptane-7,1-diyl) bis(2-octyldecanoate) as colorless oil (3.45 g, 74%).

### Step 5: Synthesis of (R)-((3-Hydroxypropane-1,2-diyl)bis(oxy))bis(7-oxoheptane-7,1-diyl) bis(2-octyldecanoate) (8)

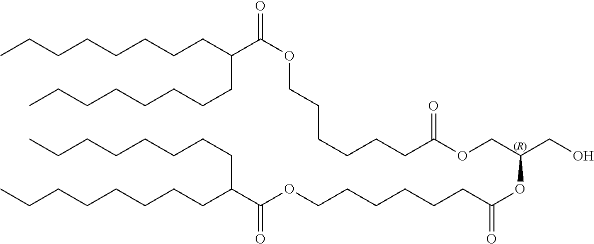

In a Parr reactor, a mixture of (R)-((3-(benzyloxy)propane-1,2-diyl)bis(oxy))bis(7-oxoheptane-7,1-diyl) bis(2-octyldecanoate) 7 (1.23 g, 1.27 mmol) and 10% palladium on carbon (600 mg) in 30 mL ethyl acetate was hydrogenated with balloon for 16 h. After filtration and concentration, (R)-((3-hydroxypropane-1,2-diyl)bis(oxy))bis(7-oxoheptane-7,1-diyl) bis(2-octyldecanoate) was obtained as colorless oil (1.15 g, quant.).

### Step 6: Synthesis of (S)-((3-((2-Oxido-1,3,2-dioxaphospholan-2-yl)oxy)propane-1,2-diyl)bis(oxy))bis(7-oxoheptane-7,1-diyl) bis(2-octyldecanoate) (10)

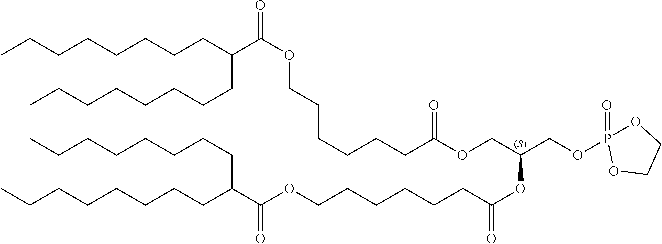

A solution of (R)-((3-hydroxypropane-1,2-diyl)bis(oxy))bis(7-oxoheptane-7,1-diyl) bis(2-octyldecanoate) 8 (1.12 g, 1.27 mmol) and triethylamine (0.45 mL, 3.2 mmol) in 10 mL THF was cooled to 0° C., and then a solution of 2-chloro-2-oxo-1,3,2-dioxaphospholane 9 (456 mg, 3.2 mmol) in 2 mL THF was added dropwise. The mixture was slowly warmed to room temperature and stirred for 15 h. TLC showed complete reaction. The triethylamine hydrochloride salt formed in the reaction was carefully filtered off under nitrogen, the filtrate was concentrated at 10° C. on a rotavapor, and the residual oil was dried under high vacuum for 2 h. The crude product was used for the next step without further purification.

### Step 7: Synthesis of (S)-2,3-Bis((7-((2-octyldecanoyl)oxy)heptanoyl)oxy)propyl (2-(trimethylammonio)ethyl) phosphate (Compound 59)

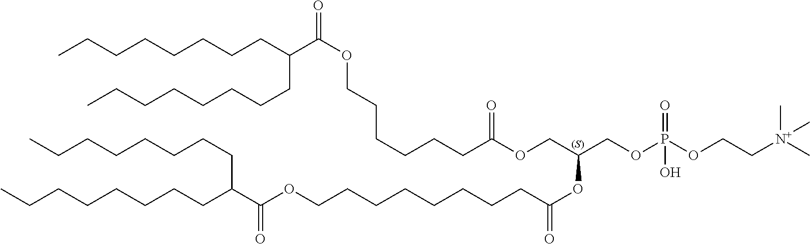

In a seal tube, a solution of (S)-((3-((2-oxido-1,3,2-dioxaphospholan-2-yl)oxy)propane-1,2-diyl)bis(oxy))bis(7-oxoheptane-7,1-diyl) bis(2-octyldecanoate) 10 (crude) in 16 mL acetonitrile was cooled in ice bath, and a solution of trimethylamine (2 M in THF, 8.0 mL, 16 mmol) was added. The bottle was sealed and heated at 95° C. overnight. After cooling to room temperature, the volatiles were evaporated under vacuum, and the crude was purified by column chromatography (SiO₂: CHCl₃/MeOH/H₂O 32:13:0.4) to give the desired product as white solid (550 mg, 42%).

¹H NMR (400 MHz, CDCl₃) δ 5.26-5.14 (m, 1H), 4.45-4.26 (m, 3H), 4.14-4.08 (m, 1H), 4.03 (t, 4H), 3.99-3.89 (m, 2H), 3.85-3.76 (m, 2H), 3.35 (s, 9H), 2.46-2.15 (m, 6H), 1.69-1.48 (m, 12H), 1.46-1.14 (m, 60H), 0.86 (t, 12H).

³¹P NMR (162 MHz, CDCl₃) δ −0.16.

ESI-MS analysis: Calculated C58H112NO12P [M+H]=1046.8, Observed=1046.8, 1068.6 (M+Na).

HPLC-ELSD: t_{R}=6.508 min (method 1).

### Synthetic Scheme for Compound 61

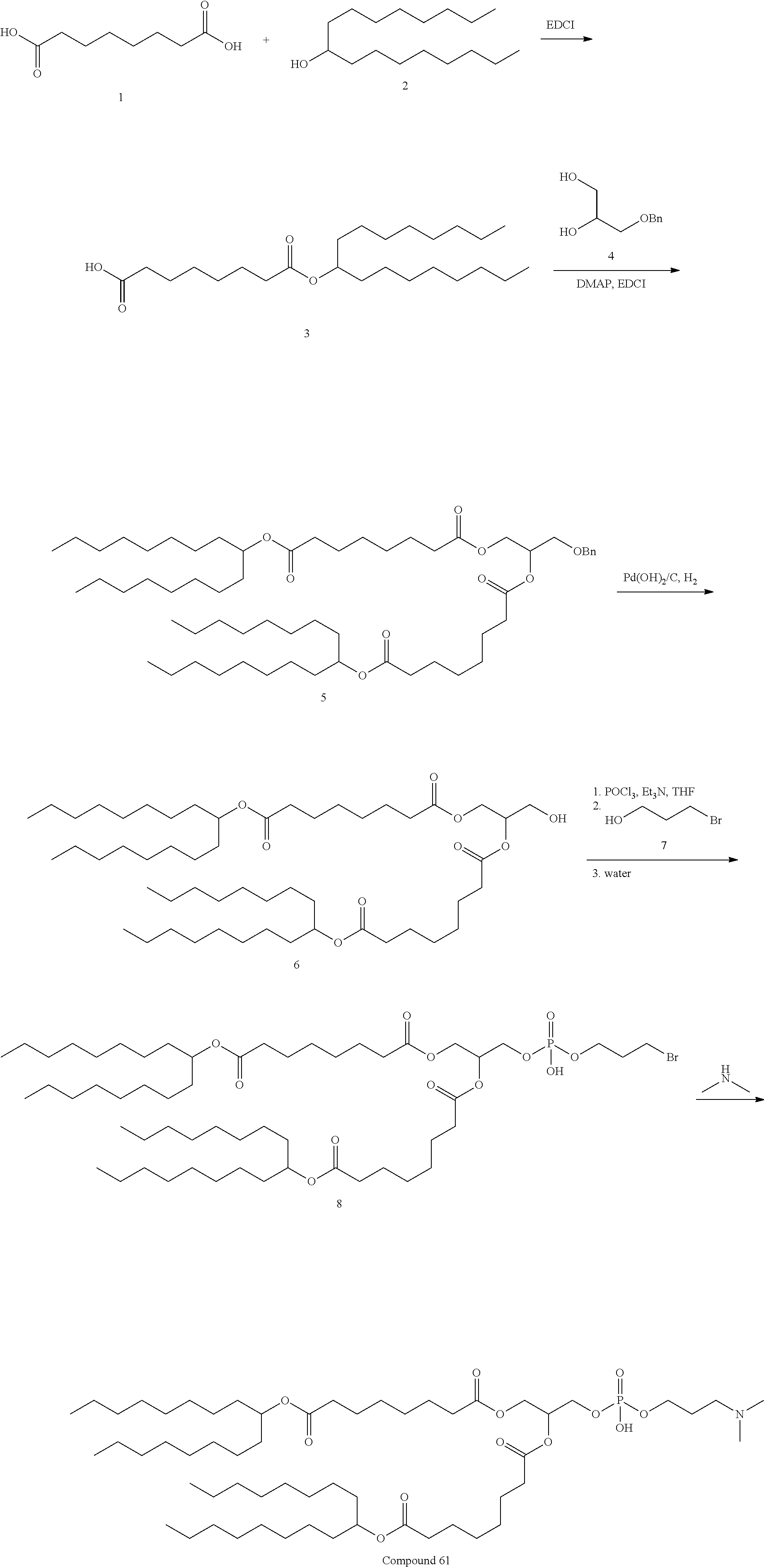

### Synthetic Procedure for Compound 61

### Step 1: Synthesis of 8-(Heptadecane-9-yloxy)-8-oxooctanoic acid (3)

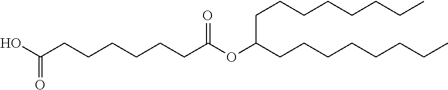 [SMILES: CCCCCCCCC(CCCCCCCC)OC(=O)CCCCCCC(=O)O]

A mixture of suberic acid 6 (22.0 g, 0.126 mole), 1-octylnonanol 14 (16.2 g, 63.1 mmol), EDCI (24.0 g, 0.126 mole) and DMAP (771 mg, 6.3 mmol) in 400 mL dichloromethane was stirred at room temperature overnight. TLC showed alcohol still present. The reaction mixture was diluted with water, extracted with dichloromethane, and the combined organic layer was washed with brine. After dried over sodium sulfate and concentrated, the residue was purified by flash column chromatography (SiO₂: 0 to 40% EtOAc in hexane) to give 8-(heptadecane-9-yloxy)-8-oxooctanoic acid as colorless oil (13.2 g, 50%).

### Step 2: Synthesis of O′¹,O¹-(3-(Benzyloxy)propane-1,2-diyl) 8-di(heptadecan-9-yl) di(octanedioate) (5)

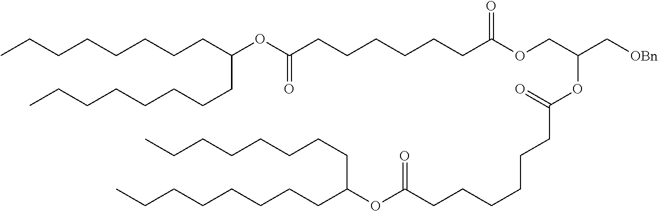 [SMILES: CCCCCCCCC(CCCCCCCC)OC(=O)CCCCCCC(=O)OCC(COCc1ccccc1)OC(=O)CCCCCCC(=O)OC(CCCCCCCC)CCCCCCCC]

A solution of 8-(heptadecane-9-yloxy)-8-oxooctanoic acid 3 (13.2 g, 32 mmol), 3-(benzyloxy)propane-1,2-diol 4 (2.65 g, 14.5 mmol), EDCI (6.17 g, 32 mmol) and DMAP (0.39 g, 3.2 mmol) in 200 mL dichloromethane was stirred at room temperature overnight. TLC showed the desired product along with monoester intermediate. The reaction mixture was diluted with water, extracted with dichloromethane, and the combined organic layer was washed with brine. After dried over sodium sulfate and concentrated, the crude was purified by flash column chromatography (SiO₂: 0 to 20% ethyl acetate in hexanes) to give O′¹,O¹-(3-(benzyloxy)propane-1,2-diyl) 8-di(heptadecan-9-yl) di(octanedioate) as colorless oil (9.55 g, 67%).

### Step 3: Synthesis of 8-Di(heptadecan-9-yl) O′¹,O¹-(3-hydroxypropane-1,2-diyl) di(octanedioate) (6)

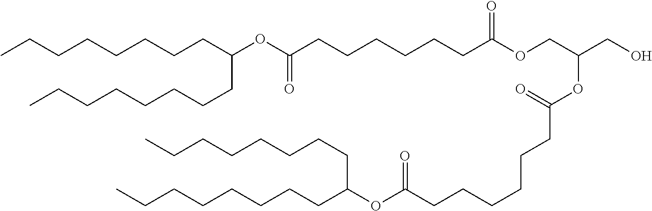 [SMILES: CCCCCCCCC(CCCCCCCC)OC(=O)CCCCCCC(=O)OCC(CO)OC(=O)CCCCCCC(=O)OC(CCCCCCCC)CCCCCCCC]

A mixture of O′¹,O¹-(3-(benzyloxy)propane-1,2-diyl) 8-di(heptadecan-9-yl) di(octanedioate) 5 (16 g, 16.4 mmol) and 10% Pd/C (1.6 g) in 100 mL ethyl acetate was purged with nitrogen and hydrogen 3 times respectively, and then the reaction was stirred at room temperature with hydrogen balloon for 16 h. After filtration and concentration, 8-di(heptadecan-9-yl) O′¹,O¹-(3-hydroxypropane-1,2-diyl) di(octanedioate) was obtained as colorless oil (13.67 g, 94%).

### Step 4: Synthesis of O′¹,O¹-(3-(((3-Bromopropoxy)(hydroxy)phosphoryl)oxy)propane-1,2-diyl) 8-di(heptadecan-9-yl) di(octanedioate) (8)

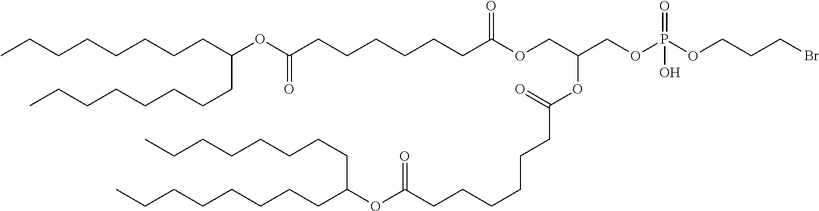 [SMILES: CCCCCCCCC(CCCCCCCC)OC(=O)CCCCCCC(=O)OCC(COP(=O)(O)OCCCBr)OC(=O)CCCCCCC(=O)OC(CCCCCCCC)CCCCCCCC]

To a solution of 8-di(heptadecan-9-yl) O,O′-(3-hydroxypropane-1,2-diyl) di(octanedioate) δ (500 mg, 0.51 mmol) and triethylamine (0.11 mL, 0.8 mmol) in 8 mL ether, was added POCl₃ (123 mg, 0.8 mmol) slowly at 0° C., and the resulting mixture was stirred at room temperature for 2 h. TLC showed the disappearance of alcohol. The volatiles were evaporated under vacuum, and the residue was redissolved in 5 mL toluene. After cooled to 0° C., a solution of 3-bromopropan-1-ol 7 (88 mg, 0.63 mmol) and triethylamine (0.11 mL, 0.8 mmol) in 2 mL toluene was added, and the mixture was stirred at room temperature overnight. 2 mL water was added, and the mixture was stirred for 2 h. After concentration, the crude was purified by column chromatography (SiO₂: 0-10% methanol in dichloromethane) to give the desired product with triethylamine, which was partitioned between water and dichloromethane to give pure product as wax (170 mg, 25%).

### Step 5: Synthesis of O′¹,O¹-(3-(((3-(Dimethylamino)propoxy)(hydroxy)phosphoryl)oxy)-propane-1,2-diyl) 8-di(heptadecan-9-yl) di(octanedioate) (Compound 61)

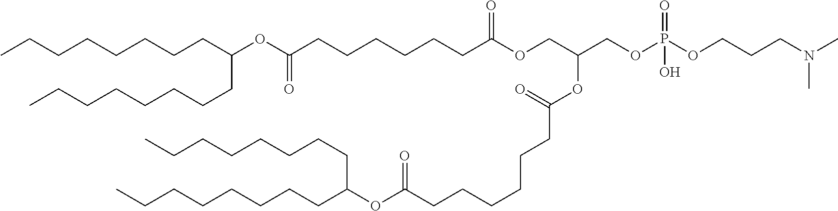 [SMILES: CCCCCCCCC(CCCCCCCC)OC(=O)CCCCCCC(=O)OCC(COP(=O)(O)OCCCN(C)C)OC(=O)CCCCCCC(=O)OC(CCCCCCCC)CCCCCCCC]

A solution of O′¹,O¹-(3-(((3-bromopropoxy)(hydroxy)phosphoryl)oxy)propane-1,2-diyl) 8-di(heptadecan-9-yl) di(octanedioate) 8 (170 mg, 0.13 mmol) in 5 mL 2 M dimethylamine solution in THF and 10 mL acetonitrile was heated at 85-90° C. in a seal tube for 3 h. MS showed the formation of the desired product. After cooling to room temperature and concentration, the crude was purified by column chromatography (SiO₂: CHCl₃/MeOH/H₂O 32:13:1) to give the desired product as pale yellow wax (127 mg, 77%).

¹H NMR (400 MHz, CDCl₃) δ 5.26-5.16 (m, 1H), 4.90-4.78 (m, 2H), 4.42-4.33 (m, 1H), 4.19-4.10 (m, 1H), 4.09-3.95 (m, 4H), 3.24-3.07 (m, 2H), 2.78 (s, 6H), 2.36-2.19 (m, 8H), 2.15-1.98 (m, 2H), 1.65-1.41 (m, 12H), 1.39-1.12 (m, 60H), 0.86 (t, 12H).

APCI-MS analysis: Calculated C58H112NO12P [M+H]=1046.8, Observed=1046.7.

HPLC-ELSD: t_{R}=7.874 min (method 1).

### Synthetic Scheme for Compound 63

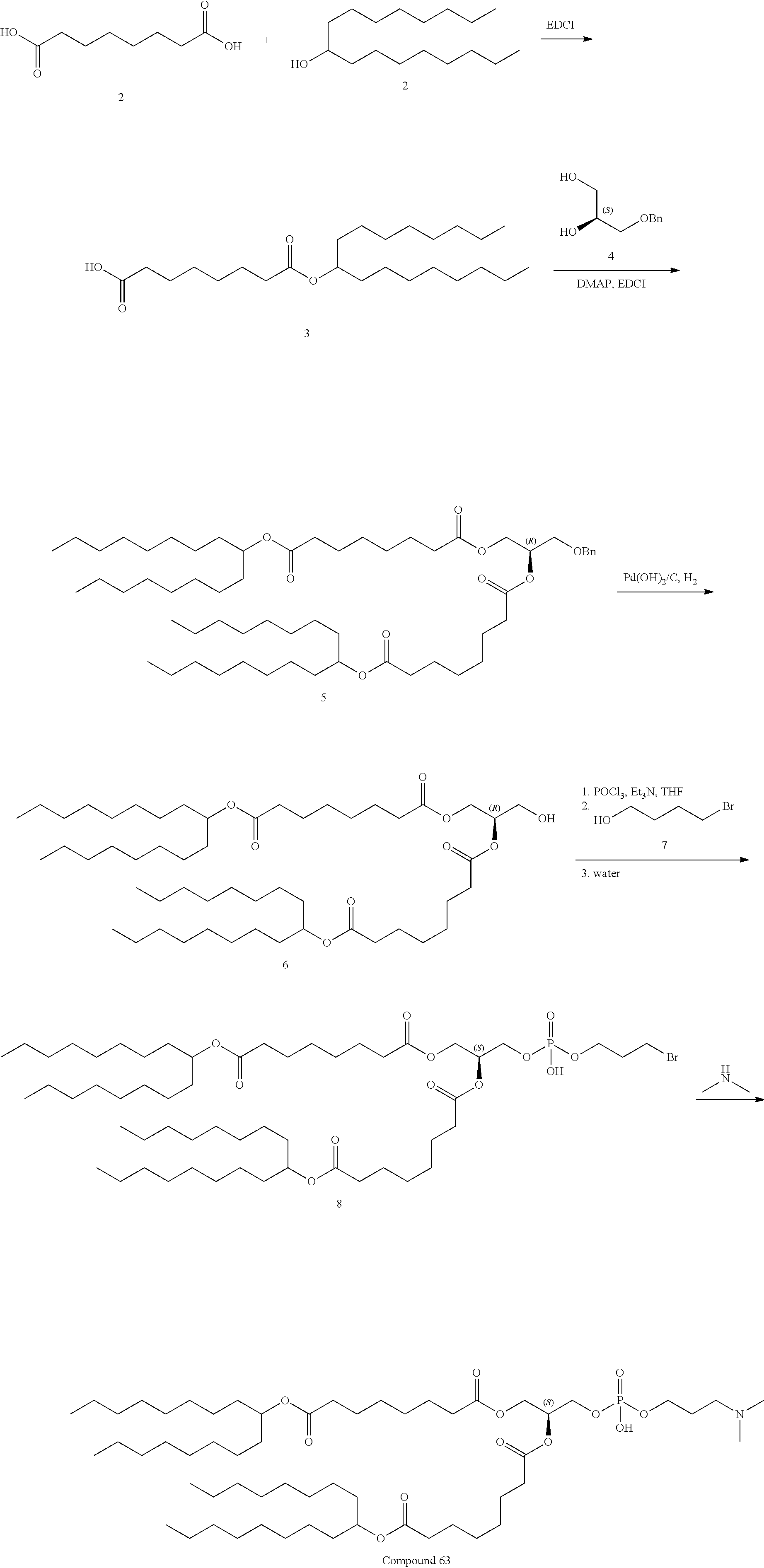

### Synthetic Procedure for Compound 63

### Step 1: Synthesis of 8-(Heptadecane-9-yloxy)-8-oxooctanoic acid (3)

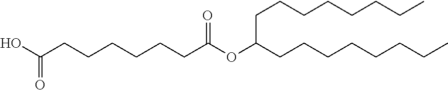 [SMILES: CCCCCCCCC(CCCCCCCC)OC(=O)CCCCCCC(=O)O]

To a stirred homogenous solution of suberic acid 1 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 2 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (R)—O′¹,O¹-(3-(Benzyloxy)propane-1,2-diyl) 8-di(heptadecan-9-yl) di(octanedioate) (5)

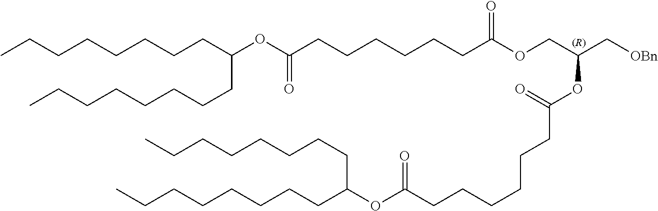

A mixture of (S)-3-(benzyloxy)propane-1,2-diol 4 (3.8 g, 20.9 mmol), 8-(heptadecane-9-yloxy)-8-oxooctanoic acid 3 (19.5 g, 47.3 mmol), EDCI (27.2 g, 142 mmol) and DMAP (5.8 g, 47.5 mmol) in 300 mL dichloromethane was stirred at room temperature for 15 h. TLC showed complete reaction. After concentration, the crude was triturated with hexane, and the solvent was removed. The crude was purified by column chromatography eluted with 0-15% ethyl acetate in hexane to yield (R)—O′¹,O¹-(3-(benzyloxy)propane-1,2-diyl) 8-di(heptadecan-9-yl) di(octanedioate) as colorless oil (18.2 g, 90%).

### Step 3: Synthesis of (R)-8-Di(heptadecan-9-yl) O′¹,O¹′-(3-hydroxypropane-1,2-diyl) di(octanedioate) (6)

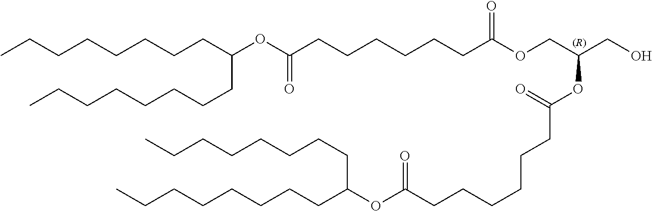

A mixture of (R)—O′¹,O¹-(3-(benzyloxy)propane-1,2-diyl) 8-di(heptadecan-9-yl) di(octanedioate) 5 (6.3 g, 6.5 mmol) and 20% Pd(OH)₂ on carbon (1 g) in 100 mL ethyl acetate was hydrogenated using Parr hydrogenator at 35-40 psi for 12 h. After filtration through silica gel and concentration, the product was used for the next step without further purification (5.6 g, 97%).

### Step 4: Synthesis of O′¹,O¹-((2S)-3-((((4-Bromobutyl)oxy)(hydroxy)phosphoryl)oxy)-propane-1,2-diyl) 8-di(heptadecan-9-yl) di(octanedioate) (8)

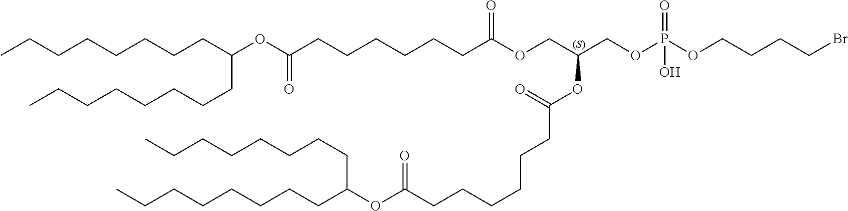

To a solution of (R)-8-di(heptadecan-9-yl) O′¹,O¹-(3-hydroxypropane-1,2-diyl) di(octanedioate) δ (750 mg, 0.85 mmol) and triethylamine (0.17 mL, 1.2 mmol) in 12 mL ether, was added POCl₃ (184 mg, 1.2 mmol) slowly at 0° C., and the resulting mixture was stirred at room temperature for 2 h. TLC showed the disappearance of alcohol. The volatiles were evaporated under vacuum, and the residue was redissolved in 8 mL toluene. After cooled to 0° C., a solution of 4-bromobutanol 7 (153 mg, 0.88 mmol) and triethylamine (0.17 mL, 1.2 mmol) in 2 mL toluene was added, and the mixture was stirred at room temperature overnight. 2 mL water was added, and the mixture was stirred for 2 h. After concentration, the crude was purified by column chromatography (SiO₂: 0-10% methanol in dichloromethane) to give the desired product with triethylamine, which was partitioned between water and dichloromethane to give slightly impure product as wax (200 mg, 21%).

### Step 5: Synthesis of O′¹,O¹-((2S)-3-((((4-(Dimethylamino)butoxy)(hydroxy)phosphoryl)-oxy)propane-1,2-diyl) 8-di(heptadecan-9-yl) di(octanedioate) (Compound 63)

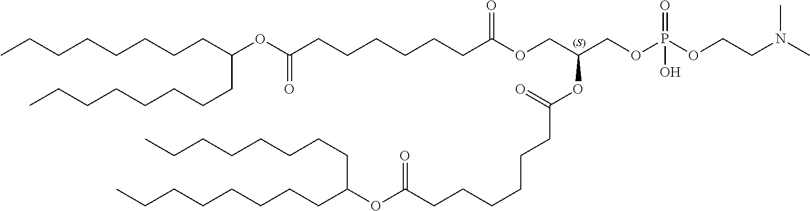

A solution of O′¹,O¹-((2S)-3-((((4-bromobutyl)oxy)(hydroxy)phosphoryl)oxy)-propane-1,2-diyl) 8-di(heptadecan-9-yl) di(octanedioate) 8 (200 mg, 0.18 mmol) in 6 mL 2 M dimethylamine solution in THF and 6 mL acetonitrile was heated at 85-90° C. in a seal tube for 8 h. MS showed the formation of the desired product. After cooling to room temperature and concentration, the crude was purified by column chromatography (SiO₂: CHCl₃/MeOH/H₂O 32:13:1) to give the desired product as pale yellow wax (60 mg, 32%).

¹H NMR (400 MHz, CDCl₃) δ 5.31-5.12 (m, 1H), 4.89-4.76 (m, 2H), 4.45-4.31 (m, 1H), 4.29-4.10 (m, 1H), 4.06-3.85 (m, 4H), 3.07 (m, 2H), 2.73 (s, 6H), 2.36-2.17 (m, 8H), 2.00-1.67 (m, 2H), 1.65-1.39 (m, 14H), 1.37-1.08 (m, 60H), 0.86 (t, 12H).

APCI-MS analysis: Calculated C59H114NO12P [M+H]=1060.8, Observed=1060.9.

HPLC-ELSD: t_{R}=7.370 min (method 1).

### Synthetic Scheme for Compound 65

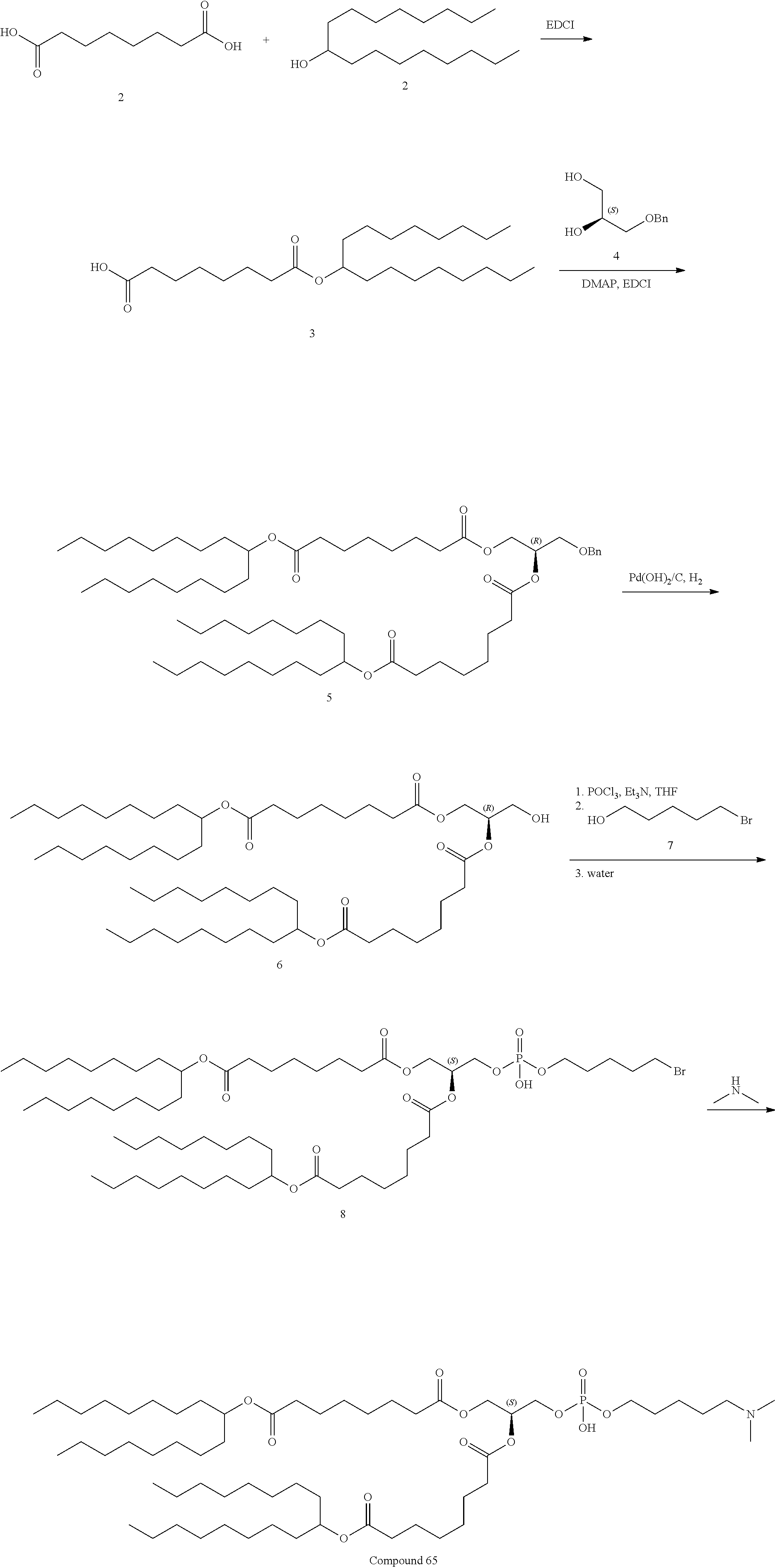

### Synthetic Procedure for Compound 65

### Step 1: Synthesis of 8-(Heptadecane-9-yloxy)-8-oxooctanoic acid (3)

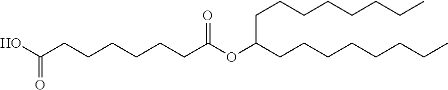 [SMILES: CCCCCCCCC(CCCCCCCC)OC(=O)CCCCCCC(=O)O]

To a stirred homogenous solution of suberic acid 1 (30 g, 172 mmol) in 200 mL DMF: DCM (1:1, v/v), was added DMAP (7.13 g, 59 mmol) and EDCI (25 g, 130 mmol), and the mixture was stirred at room temperature for 10 minutes. Then a homogeneous solution of 9-heptadecanol 2 (15 g, 59 mmol) in 100 mL DMF: DCM (1:1, v/v) was added dropwise via addition funnel over a period of 8 hours. The mixture was stirred at room temperature for 10 h. After removal of the volatile under vacuum, the residual DMF solution was partitioned with water and EtOAc. The combined organic layer was washed with brine and dried over sodium sulfate. After concentration, the crude was purified by column chromatography eluted with 0-20% EtOAc in hexane to give 8-(heptadecane-9-yloxy)-8-oxooctanoic acid as colorless oil (19.5 g, 81%).

### Step 2: Synthesis of (R)—O′¹,O¹-(3-(Benzyloxy)propane-1,2-diyl) 8-di(heptadecan-9-yl) di(octanedioate) (5)

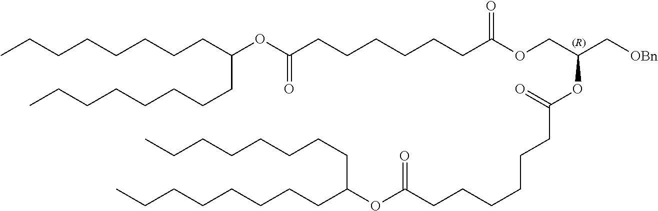

A mixture of (S)-3-(benzyloxy)propane-1,2-diol 4 (3.8 g, 20.9 mmol), 8-(heptadecane-9-yloxy)-8-oxooctanoic acid 3 (19.5 g, 47.3 mmol), EDCI (27.2 g, 142 mmol) and DMAP (5.8 g, 47.5 mmol) in 300 mL dichloromethane was stirred at room temperature for 15 h. TLC showed complete reaction. After concentration, the crude was triturated with hexane, and the solvent was removed. The crude was purified by column chromatography eluted with 0-15% ethyl acetate in hexane to yield (R)—O′¹,O¹-(3-(benzyloxy)propane-1,2-diyl) 8-di(heptadecan-9-yl) di(octanedioate) as colorless oil (18.2 g, 90%).

### Step 3: Synthesis of (R)-8-Di(heptadecan-9-yl) O′¹,O¹′-(3-hydroxypropane-1,2-diyl) di(octanedioate) (6)

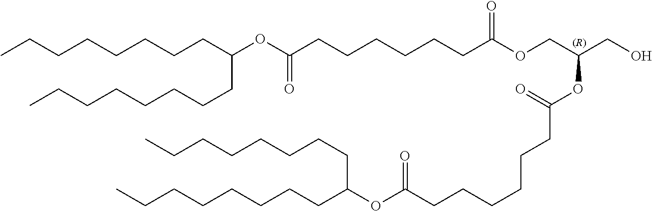

A mixture of (R)—O′¹,O¹-(3-(benzyloxy)propane-1,2-diyl) 8-di(heptadecan-9-yl) di(octanedioate) 5 (6.3 g, 6.5 mmol) and 20% Pd(OH)₂ on carbon (1 g) in 100 mL ethyl acetate was hydrogenated using Parr hydrogenator at 35-40 psi for 12 h. After filtration through silica gel and concentration, the product was used for the next step without further purification (5.6 g, 97%).

### Step 4: Synthesis of O′¹,O¹-((2S)-3-((((5-Bromopentyl)oxy)(hydroxy)phosphoryl)oxy)propane-1,2-diyl) 8-di(heptadecan-9-yl) di(octanedioate) (8)

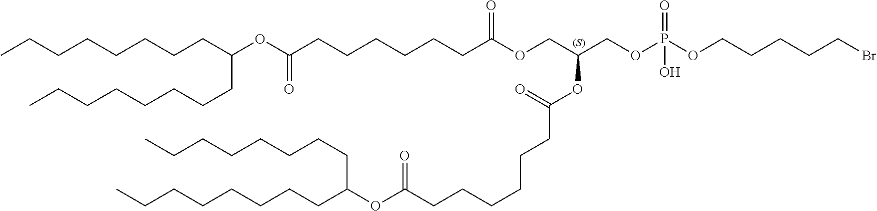

To a solution of (R)-8-di(heptadecan-9-yl) O′¹,O¹-(3-hydroxypropane-1,2-diyl) di(octanedioate) δ (1.23 g, 1.4 mmol) and triethylamine (0.28 mL, 2 mmol) in 12 mL ether, was added POCl₃ (300 mg, 1.96 mmol) slowly at 0° C., and the resulting mixture was stirred at room temperature for 2 h. TLC showed the disappearance of alcohol. The volatiles were evaporated under vacuum, and the residue was redissolved in 8 mL toluene. After cooled to 0° C., a solution of 5-bromopentanol 7 (334 mg, 2 mmol) and triethylamine (0.28 mL, 2 mmol) in 2 mL toluene was added, and the mixture was stirred at room temperature overnight. 2 mL water was added, and the mixture was stirred for 2 h. After concentration, the crude was purified by column chromatography (SiO₂: 0-10% methanol in dichloromethane) to give the desired product as wax (280 mg, 18%).

### Step 5: Synthesis of O′¹,O¹-((2S)-3-((((5-(Dimethylamino)pentyl)oxy)(hydroxy)phosphoryl)-oxy)propane-1,2-diyl) 8-di(heptadecan-9-yl) di(octanedioate) (Compound 65)

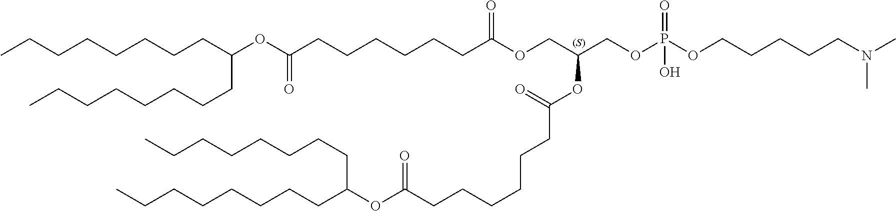

A solution of O′¹,O¹-((2S)-3-((((4-bromobutyl)oxy)(hydroxy)phosphoryl)oxy)-propane-1,2-diyl) 8-di(heptadecan-9-yl) di(octanedioate) 8 (280 mg, 0.25 mmol) in 6 mL 2 M dimethylamine solution in THF and 6 mL acetonitrile was heated at 85-90° C. in a seal tube for 17 h. MS showed the formation of the desired product. After cooling to room temperature and concentration, the crude was purified by column chromatography (SiO₂: CHCl₃/MeOH/H₂O 32:13:1) to give the desired product as pale yellow wax (150 mg, 56%).

¹H NMR (400 MHz, CDCl₃) δ 5.27-5.11 (m, 1H), 4.89-4.74 (m, 2H), 4.42-4.29 (m, 1H), 4.27-4.05 (m, 1H), 4.03-3.77 (m, 4H), 3.18-3.01 (m, 2H), 2.83 (s, 6H), 2.35-2.17 (m, 8H), 1.94-1.77 (m, 2H), 1.73-1.39 (m, 16H), 1.37-1.07 (m, 60H), 0.85 (t, 12H).

APCI-MS analysis: Calculated C60H116NO12P [M+H]=1074.8, Observed=1074.8.

HPLC-ELSD: t_{R}=6.683 min (method 1).

### HPLC Analytical Method 1:

HPLC: Agilent 1100

Column: Agela C18 column, 4.6×50 mm, 3 μm

Column temperature: 60° C.

Flow Rate: 0.5 mL/min

Detector: ELSD

Eluents: A, Isopropanol; B, water with 0.1% TFA.

Gradient:

| Time | A % | B % |
|---|---|---|
| 0 | 30% | 70% |
| 5 | 100% | 0% |
| 15 | 100% | 0% |
| 16 | 30% | 70% |

### HPLC Analytical Method 2:

HPLC: Agilent 1100

Column: Agela C4 column, 4.6×150 mm, 5 μm

Column temperature: 60° C.

Flow Rate: 0.5 mL/min

Detector: ELSD

Eluents: A, Isopropanol; B, water with 0.1% TFA.

Gradient:

| Time | A % | B % |
|---|---|---|
| 0 | 30% | 70% |
| 5 | 100% | 0% |
| 15 | 100% | 0% |
| 16 | 30% | 70% |

### Example 2—Formulations

The helper lipids described herein can be used in the preparation of lipid nanoparticles according to methods known in the art. For example, suitable methods include methods described in International Publication No. WO 2018/089801, which is hereby incorporated by reference in its entirety.

The lipid nanoparticles in the examples of the present invention were formulated using Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanolic solution of a mixture of lipids (cationic lipid, phosphatidylethanolamine, cholesterol, and polyethylene glycol-lipid) at a fixed lipid to mRNA ratio were combined with an aqueous buffered solution of target mRNA at an acidic pH under controlled conditions to yield a suspension of uniform LNPs. After ultrafiltration and diafiltration into a suitable diluent system, the resulting nanoparticle suspensions were diluted to final concentration, filtered, and stored frozen at −80° C. until use.

Lipid nanoparticle formulations 3 were prepared by Process A at a molar ratio of 40:1.5:30:28.5 (Cationic Lipid: DMG-PEG2000: Cholesterol: Helper Lipid) with the cationic lipids and helper lipids disclosed in the tables below. The Polydispersity Index (Pdl) of lipid nanoparticles can be determined by diluting the formulation in 10% trehalose at about 0.1 mg/ml mRNA concentration and then measuring the size on Malvern zetasizer. The lipid nanoparticle size can be obtained with Malvern Zetasizer Nano-ZS.

Dynamic light scattering (DLS) measurements were performed using a Malvern Instruments Zetasizer with a backscattering detector angle of 173° and a 4-mW, 633-nm He—Ne laser (Worcestershire, UK). The samples were analyzed by diluting in 10% Trehalose and measuring the size and Poly dispersity Index (PDI) in an optical grade polystyrene cuvette.

With few exceptions, size, PDI and encapsulation efficiency were measured within expected ranges.

Lipid nanoparticles were formulated containing the four lipid components shown below at a ratio of 40:30:28.5:1.5, complexed with a hEPO messenger RNA.
Cationic lipid, 40 mol %
Phospholipid (PEA) helper lipid, 30 mol %
Cholesterol, 28.5 mol %
DMG-PEG-2K (1,2-dimyristoyl-rac-glycero-3-methoxypolyethyleneglycol), 1.5 mol %
   The cationic lipid component of each formulation was selected from the compounds in the table below:

| Cationic | |
|---|---|
| lipid | Compound name and structure |
| CL-A | (3R,6S)-3,6-bis(4-(bis((9Z,12Z)-2-hydroxyoctadeca-9,12-dien-1- |
| | yl)amino)butyl)piperazine-2,5-dione |
| | 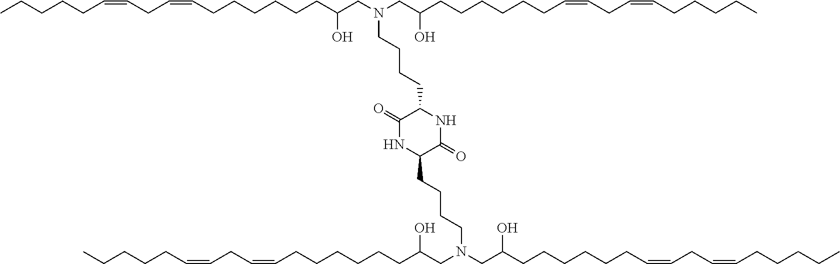 [SMILES: CCCCC/C=C\C/C=C\CCCCCCC(O)CN(CCCC[C@@H]1NC(=O)[C@@H](CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| CL-B | (3R,6S)-3,6-bis(2-hydroxydecyl)amino)butyl)piperazine-2,5-dione |
| | 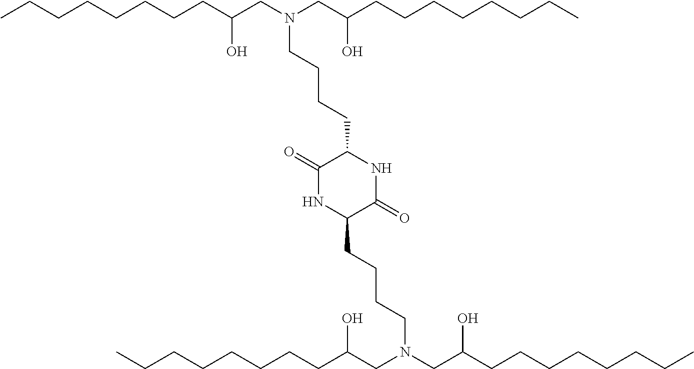 [SMILES: CCCCCCCCC(O)CN(CCCC[C@@H]1NC(=O)[C@@H](CCCCN(CC(O)CCCCCCCC)CC(O)CCCCCCCC)NC1=O)CC(O)CCCCCCCC] |
| CL-C | 2-(4-(2-((4-(bis(2-hydroxydecyl)amino)butyl)disulfaneyl)ethyl)piperazin-1-yl)ethyl 4- |
| | (bis(2-hydroxydodecyl)amino)butanoate |
| | 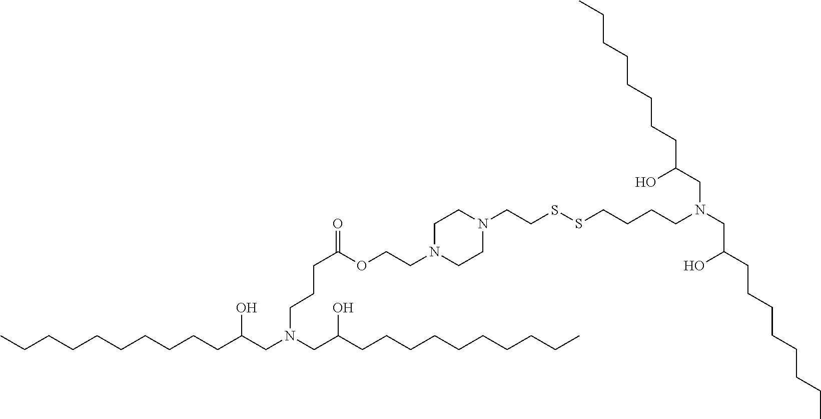 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC] |
| CL-D | 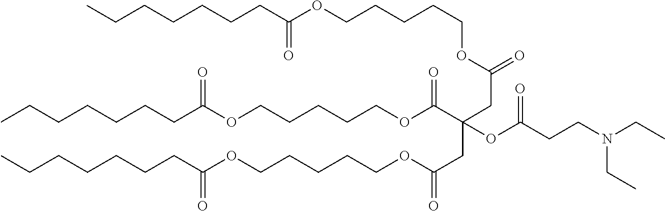 [SMILES: CCCCCCCC(=O)OCCCCCOC(=O)CC(CC(=O)OCCCCCOC(=O)CCCCCCC)(OC(=O)CCN(CC)CC)C(=O)OCCCCCOC(=O)CCCCCCC] |
| CL-E | 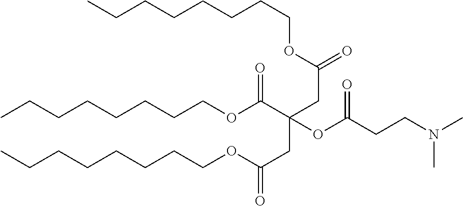 [SMILES: CCCCCCCCOC(=O)CC(CC(=O)OCCCCCCCC)(OC(=O)CCN(C)C)C(=O)OCCCCCCCC] |

TABLE 1
Analytics of novel helper lipid formulations with CL-A.
A total of 28 helper lipids were formulated with CL-A

| | | Poly- | Encapsulation |
|---|---|---|---|
| | Size | dispersity | efficiency (EE) |
| Compound | (nm) | Index (PDI) | % |
| DOPE | 129 | 0.06 | 91 |
| DOPE | 119 | 0.100 | 91 |
| 1 | 132 | 0.075 | 93 |
| 2 | 137 | 0.031 | 69 |
| 3 | 137 | 0.045 | 65 |
| 4 | 125 | 0.048 | 85 |
| 5 | 121 | 0.106 | 91 |
| 6 | 159 | 0.066 | 90 |
| 7 | 120 | 0.09 | 88 |
| 8 | 116 | 0.06 | 89 |
| 9 | 112 | 0.126 | 84 |
| 10 | 171 | 0.046 | 90 |
| 11 | 173 | 0.068 | 94 |
| 12 | 160 | 0.050 | 93 |
| 13 | 166 | 0.048 | 94 |
| 14 | 148 | 0.068 | 96 |
| 15 | 138 | 0.083 | 95 |
| 16 | 128 | 0.052 | 95 |
| 17 | 137 | 0.071 | 93 |
| 18 | 155 | 0.072 | 93 |
| 19 | 136 | 0.075 | 94 |
| 24 | 160 | 0.055 | 94 |
| 25 | 137 | 0.121 | 93 |
| 27 | 153 | 0.059 | 94 |

TABLE 2
Characterization of LNPs containing CL-B and CL-C.

| Com- | CL-B | | | CL-C | | |
|---|---|---|---|---|---|---|
| pound | Size(nm) | PDI | EE % | Size(nm) | PDI | EE % |
| 1 | 107.6 | 0.091 | 88 | 89.3 | 0.083 | 79 |
| 2 | 97.0 | 0.124 | 37 | 88.5 | 0.102 | 69 |
| 3 | 116.4 | 0.101 | 25 | 111.9 | 0.038 | 88 |
| 4 | 104.7 | 0.112 | 86 | 109.5 | 0.123 | 87 |
| 5 | 98.9 | 0.108 | 90 | 97.9 | 0.047 | 88 |
| 6 | 119.9 | 0.049 | 94 | 108.9 | 0.037 | 94 |
| 7 | 88.2 | 0.111 | 92 | 87.4 | 0.099 | 92 |
| 8 | 92.3 | 0.146 | 18 | 91.1 | 0.102 | 87 |
| 9 | 108.2 | 0.113 | 78 | 110.1 | 0.087 | 78 |
| 10 | 95.9 | 0.086 | 83 | 89.3 | 0.022 | 84 |
| 11 | 102.1 | 0.066 | 88 | 85.5 | 0.119 | 86 |
| 12 | 109.6 | 0.132 | 93 | 110.1 | 0.072 | 90 |
| 13 | 141.9 | 0.127 | 96 | 98.0 | 0.112 | 93 |
| 14 | 137.0 | 0.067 | 92 | 101.4 | 0.141 | 91 |
| 15 | 110.7 | 0.087 | 94 | 108.0 | 0.091 | 91 |
| 16 | 108.1 | 0.069 | 94 | 102.7 | 0.077 | 91 |
| 17 | 122.7 | 0.022 | 81 | 110.9 | 0.054 | 60 |
| 18 | 117.5 | 0.104 | 89 | 117.5 | 0.104 | 89 |
| 19 | 112.7 | 0.095 | 93 | 110.6 | 0.078 | 92 |
| 24 | 107.0 | 0.111 | 87 | 108.8 | 0.116 | 85 |
| 25 | 120.1 | 0.151 | 95 | 92.2 | 0.142 | 93 |
| 27 | 123.9 | 0.108 | 98 | 97.0 | 0.129 | 97 |

TABLE 3
Characterizations of LNPs containing additional lipids

| Com- | CL-D | | | CL-E | | |
|---|---|---|---|---|---|---|
| pound | Size(nm) | PDI | EE % | Size(nm) | PDI | EE % |
| 2 | 114.3 | 0.064 | 78 | ND | ND | ND |
| 12 | 113.4 | 0.094 | 92 | 112.6 | 0.072 | 91 |
| 7 | 121.9 | 0.089 | 85 | ND | ND | ND |
| 8 | 151.4 | 0.061 | 28 | ND | ND | ND |
| 1 | 100.8 | 0.098 | 92 | ND | ND | ND |
| 10 | 100.5 | 0.129 | 93 | ND | ND | ND |
| 6 | 131.5 | 0.063 | 94 | ND | ND | ND |
| 11 | 96.6 | 0.121 | 93 | ND | ND | ND |

### Example 3—IM EPO Expression of LNPS Containing New Helper Lipids

EPO expression studies were conducted with female BALB/cJ mice 6-8 weeks of age. Mice were dosed with 0.1 g EPO mRNA in 30 μL of LNPs by a single intramuscular (IM) injection into the gastrocnemius leg muscle. Blood samples were taken 6 and 24 hours post injection and hEPO levels were measured in the blood serum of the mice using an ELISA assay according to the manufacture's protocol. WO2022/099003 A1 also describes an in vivo assay for intramuscular administration (e.g. on page 46, paragraph [00206]).

Administration of LNPs including the new helper lipids consistently resulted in EPO expression, and in many cases the expression exceeded that of DOPE LNPs.

TABLE 4
EPO expression of helper lipid/cationic
lipid formulations

| | Cationic Lipid | | | | | |
|---|---|---|---|---|---|---|
| | CL-A | | CL-B | | CL-C | |
| | 6-h | 24-h | 6-h | 24-h | 6-h | 6-h |
| Com- | EPO/ | EPO/ | EPO/ | EPO/ | EPO/ | EPO/ |
| pound | ng/mL | ng/mL | ng/mL | ng/mL | ng/mL | ng/mL |
| DOPE | 26.23119 | 19.128 | 19.62 | 15.03 | 20.96 | 16.38 |
| 1 | ND | ND | 4.9 | 4.9 | 7.5 | 7.4 |
| 3 | ND | ND | ND | ND | 13.4 | 6.3 |
| 4 | ND | ND | 13.7 | 11.6 | 22.3 | 20.1 |
| 5 | ND | ND | 9.8 | 6.4 | 13.3 | 10.1 |
| 6 | ND | ND | 28.4 | 16.2 | 39.9 | 27.1 |
| 7 | ND | ND | 3.7 | 1.4 | 5.5 | 2.6 |
| 8 | ND | ND | ND | ND | 1.4 | 0.5 |
| 9 | ND | ND | 5.6 | 3.3 | 22.3 | 12.2 |
| 10 | 20.227 | 10.447 | 12.9 | 10.4 | 14.8 | 10.3 |
| 11 | 19.251 | 11.25 | 5.2 | 4.8 | 8.7 | 7.4 |
| 12 | 28.9209 | 19.526 | 39.6 | 24 | 38.4 | 26.2 |
| 14 | 0 | ND | 35.4 | ND | 34.6 | ND |
| 13 | 8.348 | ND | 53.8 | ND | 64.4 | ND |
| 15 | 25.3 | ND | 30 | ND | 15.2 | ND |
| 16 | 17.4 | ND | 32.1 | ND | 9.7 | ND |
| 17 | 16.3 | ND | 14.5 | ND | 31.7 | ND |
| 18 | 30.1 | ND | 23.7 | ND | 28.8 | ND |
| 19 | 11.7 | ND | 28.9 | ND | 12.9 | ND |
| 24 | 19.2 | ND | 29.4 | ND | 19.5 | ND |
| 25 | 16.5 | ND | 44.5 | ND | 56.5 | ND |
| 27 | 25.6 | ND | 53 | ND | 46.9 | ND |

TABLE 5
Normalized EPO expression of helper lipid CL-C formulations

| | Normalized | | |
|---|---|---|---|
| Compound | Mean | SD | N |
| DOPE | 1 | 0.353 | |
| Compound 60 | 1.39 | 0.567 | 4 |
| (racemic) | | | |
| Compound 65 | 0.825 | 0.405 | 4 |
| Compound 28 | 1.77 | 0.741 | 4 |

### Example 4—Flu HAl titer

Formulations with CL-B and DOPE or Compound 12 (see Example 2) were formulated with modified Tasmania FLU mRNAs. Groups of Balb/c mice (Mus musculus) as per the treatment group were immunized under isoflurane anesthesia with a dose of 0.4 ug per mouse in 0.05 mL of CL-B and DOPE/or CL-B and Compound 12/Modified Tasmania H3 mRNA-lipid nanoparticles via the IM route in the quadriceps, on day 0 in one hind leg and day 21 in the contralateral leg. Mice were evaluated for a minimum of 3 days post-administration and any animal that lost displayed severe clinical signs after the veterinarian's assessment was euthanized by administration of 5 mg/kg of meloxicam by subcutaneous injection.

Blood was collected via submandibular or orbital sinus bleeds (in-life bleeds were performed on day −1 and on day 20) and cardiac puncture (terminal bleed, day 35) from all animals under sedation. Mice were bled on pre-study to obtain a base-line pre-immune serum sample and for pre-screening purposes.

HAI assays were performed using the A/Tasmania/503/2020 (H3N2) virus stocks (BIOQUAL, Inc.). Sera were treated with receptor-destroying enzyme (RDE) by diluting one-part serum with three parts enzyme and incubated overnight in a 37° C. water bath. Enzyme was inactivated by a 30-minute incubation period at 56° C. followed by addition of six parts PBS for a final dilution of 1/10. HAI assays were performed in V-bottom 96-well plates using four hemagglutinating units (HAU) of virus and 0.5% turkey RBC. The reference serum for each strain was included as a positive control on every assay plate. Each plate also included a back-titration to confirm the antigen dose (4 HAU/25 pl) as well as a negative control sample (PBS or naive control serum). The HAI titer was determined as the highest dilution of serum resulting in complete inhibition of hemagglutination. Results were only valid for plates with the appropriate back-titration result (verifying 4 HAU/25 ul added) and a reference serum titer within 2-fold of the expected titer. CL-B/DOPE was shown to induce an HAI GMT of 113 whereas CL-B/Cmpd 12 was shown to induce an HAI GMT of 293, thereby demonstrating a significant improvement in HAI titers for a 0.4 ug dose following replacement of the helper lipid.

### Example 5—IM EPO expression of LNPs containing phosphatidylcholine helper lipids

Lipid nanoparticles were formulated containing the four lipid components shown below, complexed with a hEPO messenger RNA.
Cationic lipid CL-C, 40 mol %
Helper lipid, 13.5 mol %
Cholesterol, 44.8 mol %
DMG-PEG-2K (1,2-dimyristoyl-rac-glycero-3-methoxypolyethyleneglycol), 1.67 mol %

EPO expression studies were performed as described above in Example 3. Administration of LNPs including the new helper lipids consistently resulted in EPO expression, and in some cases the expression exceeded that of DOPE LNPs.

TABLE 6
Normalized EPO expression of PC/CL-C formulations

| | Normalized | | |
|---|---|---|---|
| Compound | Mean | SD | N |
| DOPE | 1 | 0.17 | 4 |
| Compound 41 | 1.3 | 0.43 | 4 |
| Compound 42 | 1.1 | 0.43 | 4 |
| Compound 48 | 1.1 | 0.29 | 4 |
| Compound 50 | 1 | 0.23 | 4 |
| Compound 52 | 1 | 0.32 | 4 |
| Compound 46 | 1.1 | 0.23 | 4 |
| Compound 44 | 1 | 0.24 | 4 |
| Compound 57 | 0.9 | 0.21 | 4 |
| Compound 56 | 1 | 0.26 | 4 |
| Compound 54 | 0.5 | 0.1 | 4 |

### Example 6. IV EPO Expression of LNPs

Lipid nanoparticles were formulated containing the four lipid components shown below at a ratio of 40:30:28.5:1.5, complexed with a hEPO messenger RNA.
Cationic lipid CL-0137, 40 mol %
Helper lipid, 30 mol %
Cholesterol, 28.5 mol %
DMG-PEG-2K (1,2-dimyristoyl-rac-glycero-3-methoxypolyethyleneglycol), 1.5 mol %

EPO expression studies were conducted with female BALB/cJ mice 6-8 weeks of age. Mice were dosed with 0.017 μg EPO mRNA LNPs in a volume of 5 ul by a single tail vein injection. Blood samples were taken 6 hours post injection and hEPO levels were measured in the blood serum of the mice using an ELISA assay according to the manufacture's protocol.

Administration of LNPs including the new helper lipids consistently resulted in EPO expression, and in many cases the expression exceeded that of DOPE LNPs.

TABLE 7
Normalized IV EPO expression

| | Normalized | | |
|---|---|---|---|
| Helper lipid | Mean | SD | N |
| DOPE | 1 | 0.1 | 4 |
| Compound 8 | 0.29 | 0.06 | 4 |
| Compound 7 | 0.52 | 0.06 | 4 |
| Compound 38 | 0.8 | 0.21 | 4 |
| Compound 14 | 0.95 | 0.15 | 4 |
| Compound 11 | 1.17 | 0.2 | 4 |
| Compound 25 | 1.24 | 0.23 | 4 |
| Compound 5 | 1.25 | 0.34 | 4 |
| Compound 13 | 1.47 | 0.19 | 4 |
| Compound 16 | 1.59 | 0.25 | 4 |
| Compound 27 | 1.7 | 0.28 | 4 |
| Compound 41 | 1.7 | 0.23 | 4 |
| Compound 10 | 1.72 | 0.45 | 4 |
| Compound 27 | 1.72 | 0.29 | 4 |
| Compound 24 | 1.82 | 0.25 | 4 |
| Compound 65 | 2.14 | 0.21 | 4 |
| Compound 12 | 2.23 | 0.6 | 4 |
| Compound 15 | 2.24 | 0.45 | 4 |
| Compound 4 | 2.38 | 0.19 | 4 |
| Compound 19 | 2.59 | 0.35 | 4 |
| Compound 6 | 2.67 | 0.21 | 4 |
| Compound 3 | 3.32 | 0.67 | 4 |
| Compound 9 | 3.68 | 0.59 | 4 |
| Compound 17 | 4.53 | 0.6 | 4 |
| Compound 18 | 4.68 | 0.96 | 4 |
| Compound 60 | 4.61 | 1 | 4 |

### Example 7. IV Delivery Using Phosphatidylcholine Helper Lipids

Lipid nanoparticles were formulated containing the four lipid components shown below, complexed with a hEPO messenger RNA.
Cationic lipid CL-0137, 40 mol %
Helper lipid, 13.5 mol %
Cholesterol, 44.8 mol %
DMG-PEG-2K (1,2-dimyristoyl-rac-glycero-3-methoxypolyethyleneglycol), 1.67 mol %

EPO expression studies were conducted with female BALB/cJ mice 6-8 weeks of age. Mice were dosed with 0.017 μg EPO mRNA LNPs in a volume of 5 ul by a single tail vein injection. Blood samples were taken 6 hours post injection and hEPO levels were measured in the blood serum of the mice using an ELISA assay according to the manufacture's protocol.

Administration of LNPs including the new phosphatidylcholine helper lipids consistently resulted in EPO expression, and in most cases the expression exceeded that of DOPE LNPs.

TABLE 7
Normalized IV EPO expression using PC LNPs

| | Normalized | | |
|---|---|---|---|
| Helper Lipid | mean | SD | N |
| DOPE | 1 | 0.1 | 4 |
| DSPC | 0.89 | 0.14 | 4 |
| Compound 42 | 1.16 | 0.09 | 4 |
| Compound 48 | 1.08 | 0.29 | 4 |
| Compound 50 | 1.6 | 0.32 | 4 |
| Compound 52 | 1.53 | 0.24 | 4 |
| Compound 46 | 1.34 | 0.18 | 4 |
| Compound 44 | 1.32 | 0.25 | 4 |
| Compound 56 | 1.52 | 0.19 | 4 |
| Compound 54 | 0.97 | 0.16 | 4 |

From the foregoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

### EMBODIMENTS

1. A compound having a structure according to Formula (I):

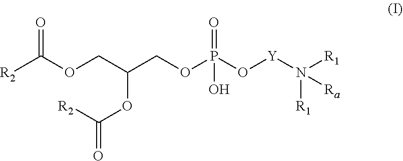

or a pharmaceutically acceptable salt thereof, wherein:
Y is selected from optionally substituted C₂-C₆ alkylene or optionally substituted C₄.C₆ alkenylene;
Rₐ is absent, or Rₐ is optionally substituted C₁-C₁₀ alkyl, wherein, when Rₐ is present, the nitrogen to which Rₐ is bonded bears a positive charge;
each R₁ is independently selected from hydrogen or optionally substituted C₁-C₁₀ alkyl;
each R₂ is independently selected from:
(i) optionally substituted C₄-C₂₄alkyl, optionally substituted C₄-C₂₄alkenyl, and optionally substituted C₄-C₂₄alkynyl;

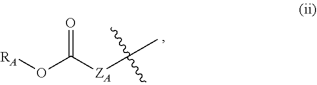

wherein each R_{A} is independently selected from optionally substituted C₁-C₃₁ alkyl, optionally substituted C₂-C₃₁ alkenyl, and optionally substituted C₂-C₃₁ alkynyl; and
   wherein each Z_{A} is independently selected from optionally substituted C₁-C₁₀ alkylene and optionally substituted C₂-C₁₀ alkenylene;

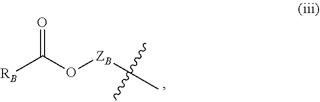

wherein each R_{B} is independently selected from optionally substituted C₁-C₃₁ alkyl, optionally substituted C₂-C₃₁alkenyl, and optionally substituted C₂-C₃₁ alkynyl; and
   wherein each Z_{B} is independently selected from optionally substituted C₁-C₁₀ alkylene and optionally substituted C₂-C₁₀ alkenylene.
2. The compound of embodiment 1, wherein the compound has a structure according to Formula (II):

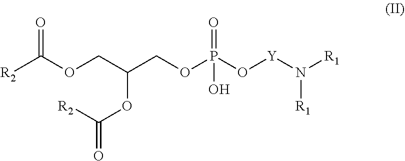

or a pharmaceutically acceptable salt thereof.
3. The compound of embodiment 2, wherein the compound has a structure according to Formula (IIa):

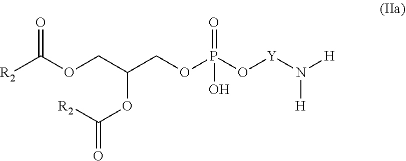

or a pharmaceutically acceptable salt thereof.
4. The compound of embodiment 2, wherein the compound has a structure according to Formula (IIIb):

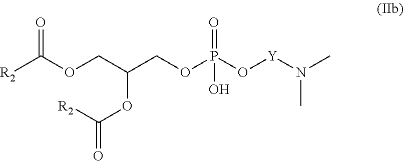

or a pharmaceutically acceptable salt thereof.
5. The compound of embodiment 2, wherein the compound has a structure according to Formula (IIc):

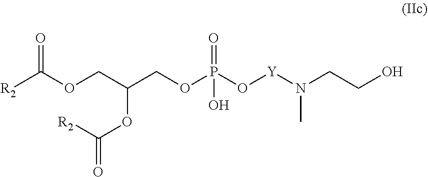

or a pharmaceutically acceptable salt thereof.
6. The compound of embodiment 2, wherein the compound has a structure according to Formula (IId):

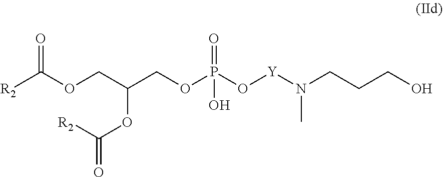

or a pharmaceutically acceptable salt thereof.
7. The compound of embodiment 1, wherein the compound has a structure according to Formula (III):

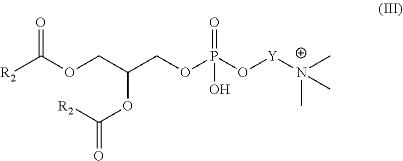

or a pharmaceutically acceptable salt thereof.
8. The compound of any one of embodiments 1-7, or a pharmaceutically acceptable salt thereof, wherein Y is selected from optionally substituted C₂₋₆ alkylene.
9. The compound of embodiment 8, or a pharmaceutically acceptable salt thereof, wherein Y is —CH₂CH₂—.
10. The compound of any one of embodiments 1-7, or a pharmaceutically acceptable salt thereof, wherein Y is selected from optionally substituted C₄₋₆ alkenylene.
11. The compound of embodiment 1, wherein the compound has a structure according to Formula (IV):

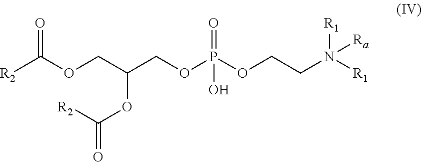

or a pharmaceutically acceptable salt thereof.
12. The compound of embodiment 11, wherein the compound has a structure according to Formula (V):

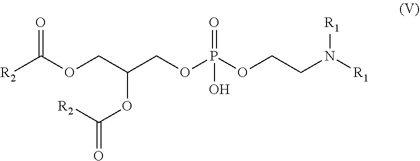

or a pharmaceutically acceptable salt thereof.
13. The compound of embodiment 12, wherein the compound has a structure according to Formula (Va):

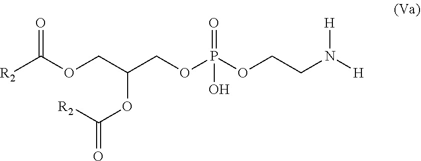

or a pharmaceutically acceptable salt thereof.
14. The compound of embodiment 12, wherein the compound has a structure according to Formula (Vb):

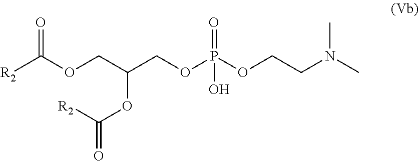

or a pharmaceutically acceptable salt thereof.
15. The compound of embodiment 12, wherein the compound has a structure according to Formula (Vc):

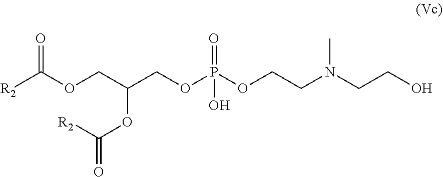

or a pharmaceutically acceptable salt thereof.
16. The compound of embodiment 12, wherein the compound has a structure according to Formula (Vd):

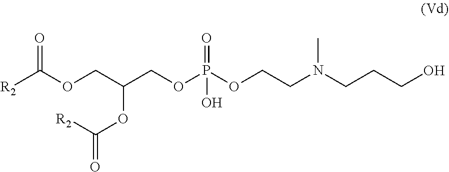

or a pharmaceutically acceptable salt thereof.
17. The compound of any one of embodiments 1-16, or a pharmaceutically acceptable salt thereof, wherein the stereochemistry of the carbon atom situated between R₂C(═O)O—CH₂— and —CH₂—OP(═O)(OH)—O— is as depicted in the following structure:

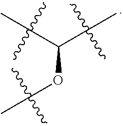

18. The compound of any one of embodiments 1-16, or a pharmaceutically acceptable salt thereof, wherein the stereochemistry of the carbon atom situated between R₂C(═O)O—CH₂— and —CH₂—OP(═O)(OH)—O— is as depicted in the following structure:

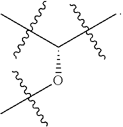

19. The compound of any one of embodiments 1-18, or a pharmaceutically acceptable salt thereof, wherein each R₂ is the same.
20. The compound of any one of embodiments 1-18, or a pharmaceutically acceptable salt thereof, wherein each R₂ is different.
21. The compound of any one of embodiments 1-20, or a pharmaceutically acceptable salt thereof, wherein the compound has a structure according to Formula (II) or (V), wherein each R₁ is independently selected from optionally substituted C₁-C₁₀ alkyl, for example wherein each R₁ is methyl, or wherein each R₁ is ethyl.
22. The compound of any one of embodiments 1-20, or a pharmaceutically acceptable salt thereof, wherein the compound has a structure according to Formula (I) or (IV), wherein each R₁ is independently selected from optionally substituted C₁-C₁₀ alkyl, and wherein Rₐ is present and is optionally substituted C₁-C₁₀ alkyl, for example wherein each R₁ is methyl and Rₐ is methyl, or wherein each R₁ is ethyl and Rₐ is ethyl.
23. The compound of any one of embodiments 1-22, or a pharmaceutically acceptable salt thereof, wherein each R₂ is independently selected from optionally substituted C₄-C₂₄alkyl, optionally substituted C₄-C₂₄alkenyl, and optionally substituted C₄-C₂₄alkynyl.
24. The compound of embodiment 23, or a pharmaceutically acceptable salt thereof, wherein each R₂ is independently selected from optionally substituted C₄-C₂₄alkyl, for example a branched C₄-C₂₄ alkyl.
25. The compound of embodiment 24, or a pharmaceutically acceptable salt thereof, wherein each R₂ is independently selected from

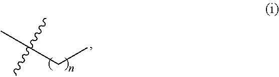

wherein each n is independently selected from 3, 4, 5, 6, 7, 8, 9, 10, 11, 12, 13, 14, 15, 16 17, 18, 19, 20, 21, 22, and 23,

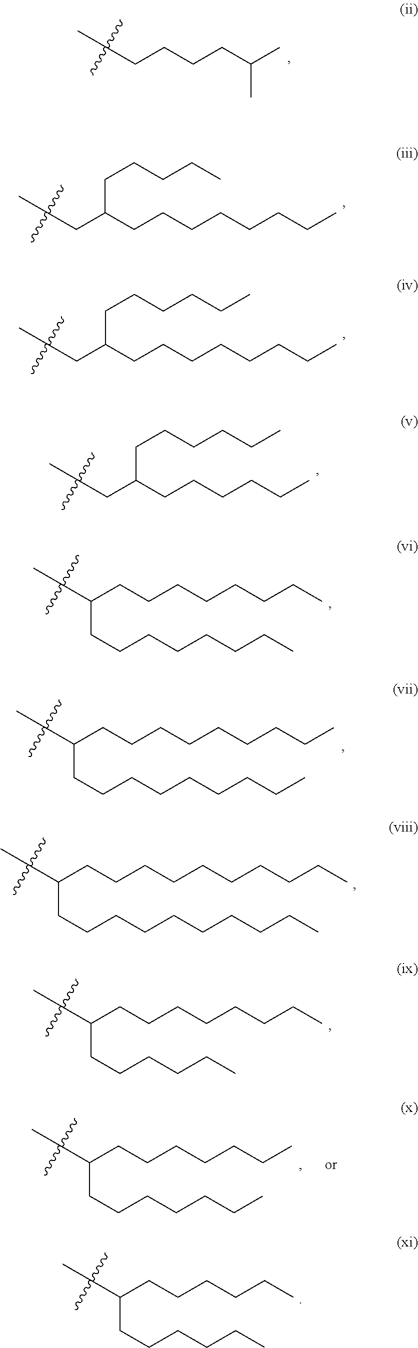

26. The compound of embodiment 25, or a pharmaceutically acceptable salt thereof, wherein each R₂ is independently selected from:

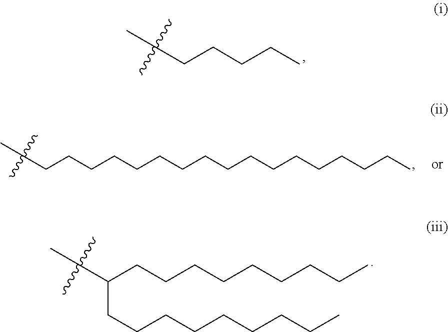

27. The compound of embodiment 23, or a pharmaceutically acceptable salt thereof, wherein each R₂ is independently selected from optionally substituted C₄-C₂₄ alkenyl.
28. The compound of embodiment 27, or a pharmaceutically acceptable salt thereof, wherein each R₂ is independently selected from:

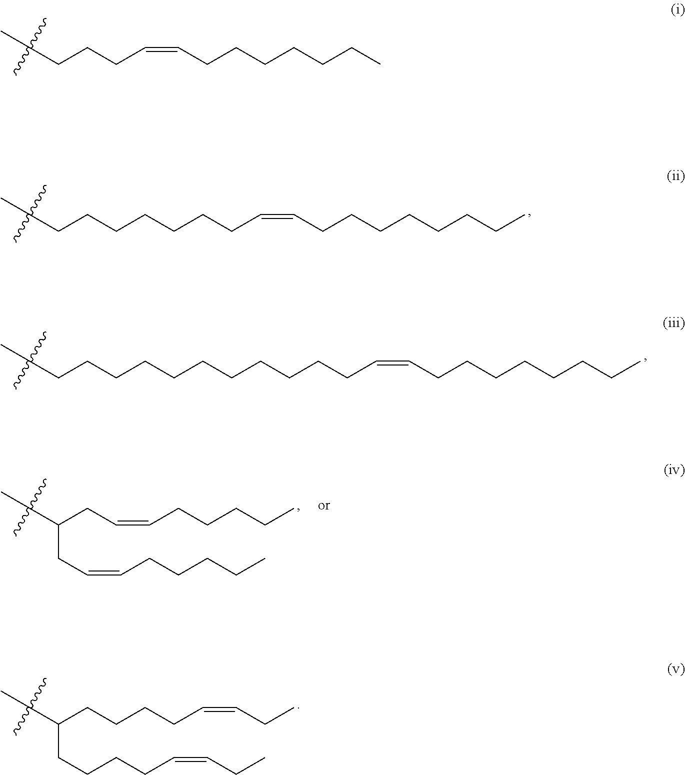 [SMILES: *CCC/C=C\CCCCCCC ; *CCCCCCC/C=C\CCCCCCCC ; *CCCCCCCCCCC/C=C\CCCCCCCC ; *C(C/C=C\CCCCC)C/C=C\CCCCC ; *C(CCCC/C=C\CC)CCCC/C=C\CC]

29. The compound of any one of embodiments 1-22, or a pharmaceutically acceptable salt thereof, wherein each R₂ is independently selected from

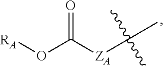

wherein each R_{A} is independently selected from optionally substituted C₁-C₃₁ alkyl, optionally substituted C₂-C₃₁alkenyl, and optionally substituted C₂-C₃₁ alkynyl; and wherein each Z_{A} is independently selected from optionally substituted C₁-C₁₀ alkylene and optionally substituted C₂-C₁₀ alkenylene.
30. The compound of embodiment 29, or a pharmaceutically acceptable salt thereof, wherein each R_{A} is independently selected from optionally substituted C₁-C₃₁alkyl, for example optionally substituted C₄-C₂₄ alkyl, for example a branched C₄-C₂₄ alkyl.
31. The compound of embodiment 30, or a pharmaceutically acceptable salt thereof, wherein each R_{A} is independently selected from a branched C₁₇ alkyl, for example

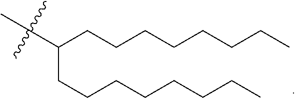 [SMILES: *C(CCCCCCCC)CCCCCCCC]

32. The compound of embodiment 30, or a pharmaceutically acceptable salt thereof, wherein each R_{A} is independently selected from a branched C₁₅ alkyl, for example

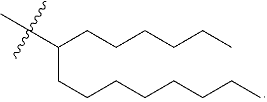 [SMILES: *C(CCCCCC)CCCCCCCC]

33. The compound of embodiment 30, or a pharmaceutically acceptable salt thereof, wherein each R_{A} is independently selected from a branched C₁₃ alkyl, for example

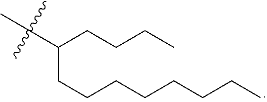 [SMILES: *C(CCCC)CCCCCCCC]

34. The compound of embodiment 30, or a pharmaceutically acceptable salt thereof, wherein each R_{A} is independently selected from:

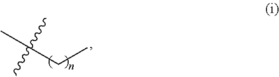

wherein each n is independently selected from 0, 1, 2, 3, 4, 5, 6, 7, 8, 9, 10, 11, 12, 13, 14, 15, 16 17, 18, 19, 20, 21, 22, 23, 24, 25, 26, 27, 28, 29, and 30,

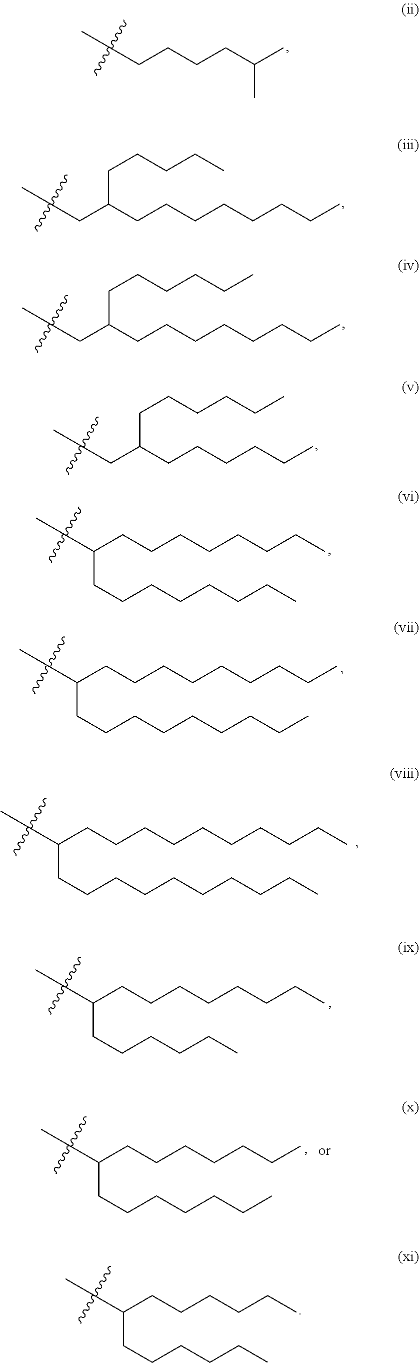

35. The compound of embodiment 29, or a pharmaceutically acceptable salt thereof, wherein each R_{A} is independently selected from optionally substituted C₂-C₃₁ alkenyl, for example optionally substituted C₄-C₂₄ alkenyl.
36. The compound of embodiment 35, or a pharmaceutically acceptable salt thereof, wherein each R_{A} is independently selected from:

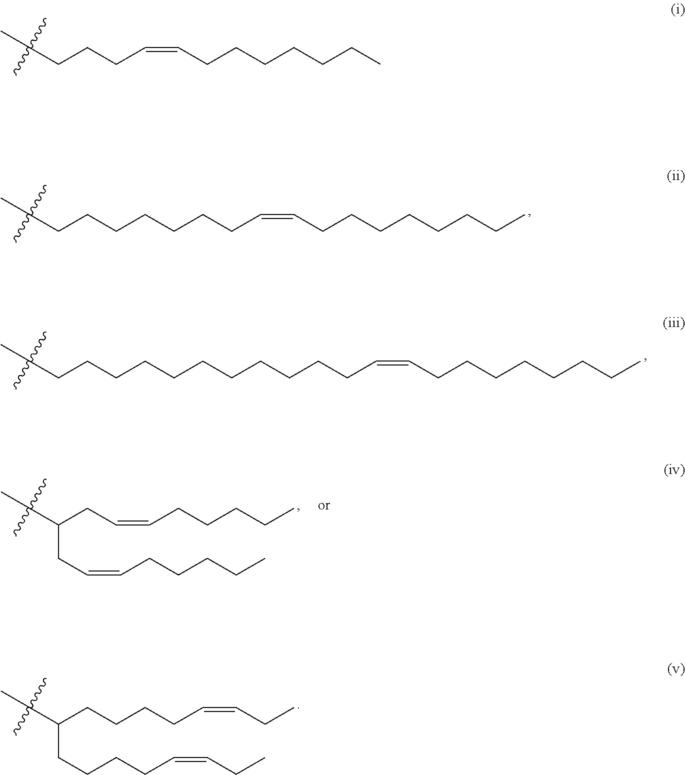 [SMILES: *CCC/C=C\CCCCCCC ; *CCCCCCC/C=C\CCCCCCCC ; *CCCCCCCCCCC/C=C\CCCCCCCC ; *C(C/C=C\CCCCC)C/C=C\CCCCC ; *C(CCCC/C=C\CC)CCCC/C=C\CC]

37. The compound of any one of embodiments 29-36, or a pharmaceutically acceptable salt thereof, wherein each Z_{A} is independently selected from optionally substituted C₁-C₁₀ alkylene, for example optionally substituted C₂-C₇ alkylene.
38. The compound of embodiment 37, or a pharmaceutically acceptable salt thereof, wherein each Z_{A} is independently selected from optionally substituted C₆ alkylene, for example unsubstituted C₆ alkylene, for example unsubstituted straight-chain C₆ alkylene.
39. The compound of any one of embodiments 29-36, or a pharmaceutically acceptable salt thereof, wherein each Z_{A} is independently selected from optionally substituted C₂-C₁₀ alkenylene, for example optionally substituted C₂-C₇ alkenylene.
40. The compound of embodiment 29, or a pharmaceutically acceptable salt thereof, wherein: Y if present is —CH₂CH₂—, each R_{A} is independently selected from optionally substituted C₁-C₃₁ alkyl, and each Z_{A} is independently selected from optionally substituted C₁-C₁₀ alkylene.
41. The compound of embodiment 40, or a pharmaceutically acceptable salt thereof, wherein each R_{A} is independently selected from optionally substituted C₄-C₂₄alkyl, and each Z_{A} is independently selected from optionally substituted C₂-C₇ alkylene.
42. The compound of embodiment 40 or 41, or a pharmaceutically acceptable salt thereof, wherein each R_{A} is independently selected from:

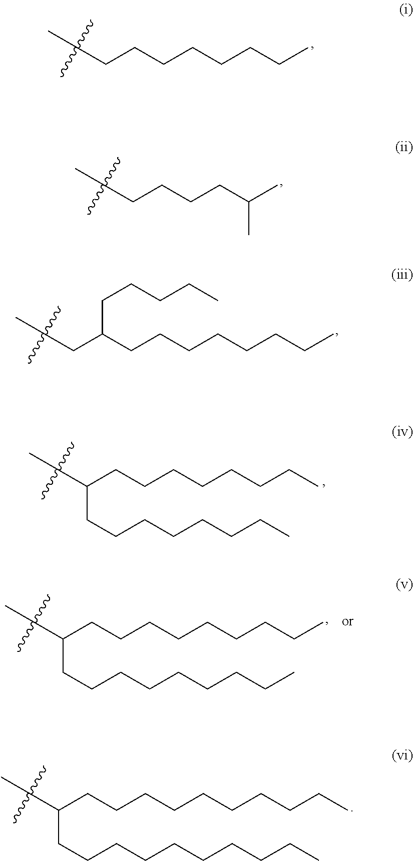

43. The compound of any one of embodiments 29-34 or 40 to 42, or a pharmaceutically acceptable salt thereof, wherein each R_{A} is

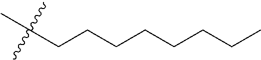 [SMILES: *CCCCCCCC]

and each Z_{A} is C₇ alkylene.
44. The compound of any one of embodiments 29-34 or 40 to 42, or a pharmaceutically acceptable salt thereof, wherein each R_{A} is

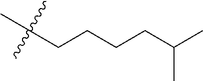 [SMILES: *CCCCC(C)C]

and each Z_{A} is C₆ alkylene.
45. The compound of any one of embodiments 29-34 or 40 to 42, or a pharmaceutically acceptable salt thereof, wherein each R_{A} is

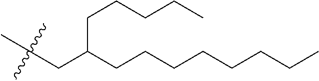 [SMILES: *CC(CCCCC)CCCCCCCC]

and each Z_{A} is C₂ alkylene.
46. The compound of any one of embodiments 29-34 or 40 to 42, or a pharmaceutically acceptable salt thereof, wherein each R_{A} is

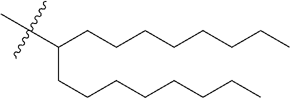 [SMILES: *C(CCCCCCCC)CCCCCCCC]

and each Z_{A} is independently selected from C₅-C₇ alkylene, for example wherein each Z_{A} is C₆ alkylene.
47. The compound of any one of embodiments 29-34 or 40 to 42, or a pharmaceutically acceptable salt thereof, wherein each R_{A} is

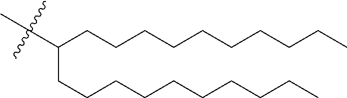 [SMILES: *C(CCCCCCCCCC)CCCCCCCCCC]

and each Z_{A} is C₆ alkylene.
48. The compound of embodiment 29, or a pharmaceutically acceptable salt thereof, wherein: Y if present is —CH₂CH₂—, each R_{A} is independently selected from optionally substituted C₁-C₃₁alkyl, and each Z_{A} is independently selected from optionally substituted C₂-C₁₀ alkenylene.
49. The compound of embodiment 48, or a pharmaceutically acceptable salt thereof, wherein each R_{A} is independently selected from optionally substituted C₄-C₂₄alkyl, and each Z_{A} is independently selected from optionally substituted C₂-C₇ alkenylene.
50. The compound of any one of embodiments 29-34 or 48-49, or a pharmaceutically acceptable salt thereof, wherein each R_{A} is

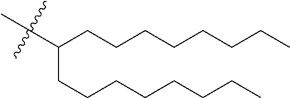 [SMILES: *C(CCCCCCCC)CCCCCCCC]

and each Z_{A} is C₆ alkenylene.
51. The compound of embodiment 29, or a pharmaceutically acceptable salt thereof, wherein: Y if present is —CH₂CH₂—, each R_{A} is independently selected from optionally substituted C₂-C₃₁alkenyl, and each Z_{A} is independently selected from optionally substituted C₁-C₁₀ alkylene.
52. The compound of embodiment 51, or a pharmaceutically acceptable salt thereof, wherein each R_{A} is independently selected from optionally substituted C₄-C₂₄alkenyl, and each Z_{A} is independently selected from optionally substituted C₂-C₇ alkylene.
53. The compound of any one of embodiments 29, 35-36, or 51-52, or a pharmaceutically acceptable salt thereof, wherein each R_{A} is

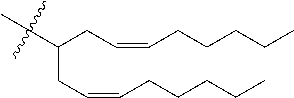 [SMILES: *C(C/C=C\CCCCC)C/C=C\CCCCC]

and each Z_{A} is C₆ alkylene.
54. The compound of any one of embodiments 29, 35-36, or 51-52, or a pharmaceutically acceptable salt thereof, wherein each R_{A} is

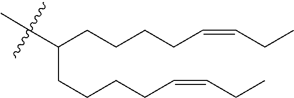 [SMILES: *C(CCCC/C=C\CC)CCCC/C=C\CC]

and each Z_{A} is C₆ alkylene.
55. The compound of embodiment 1, wherein the compound is selected from

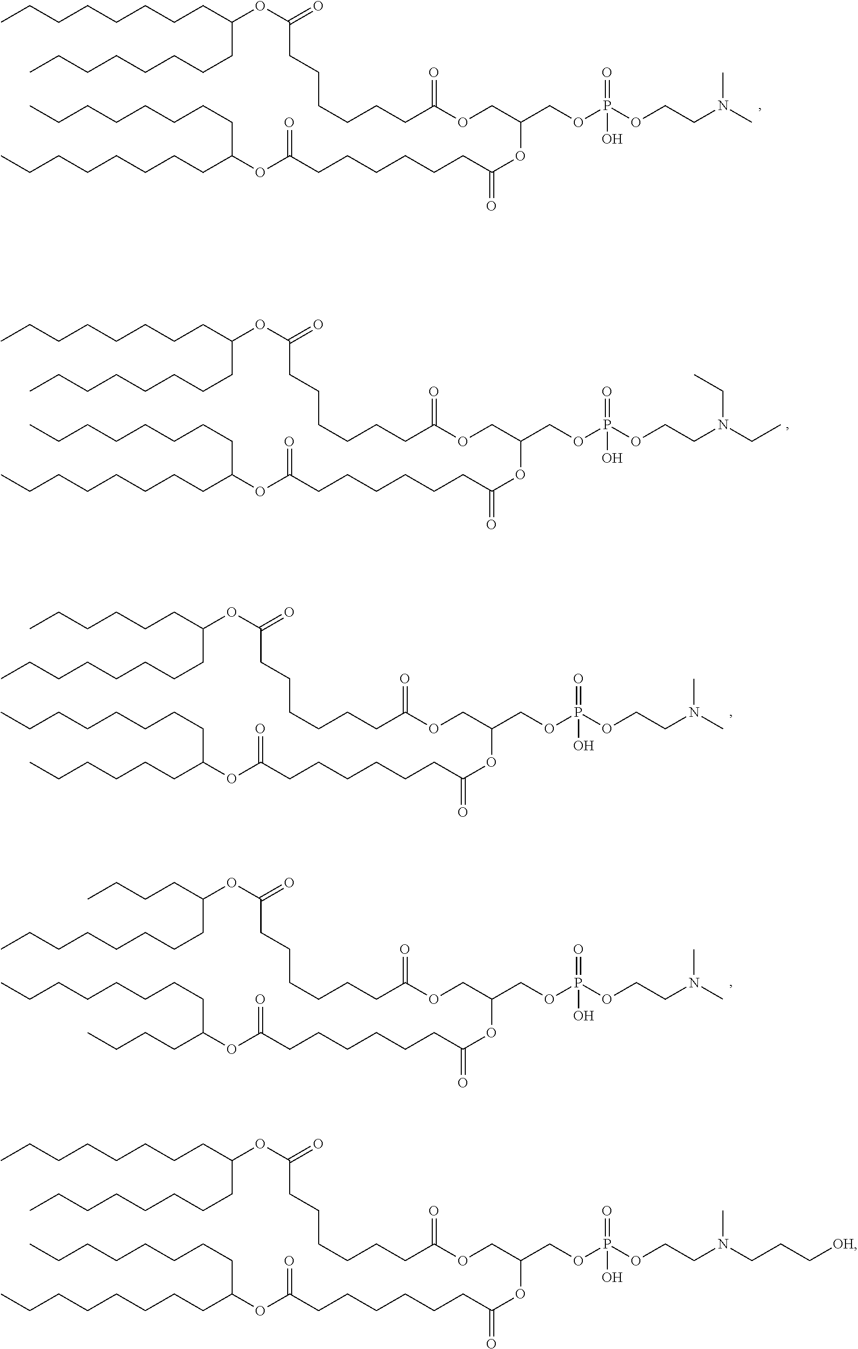

56. The compound of embodiment 1, wherein the compound is selected from

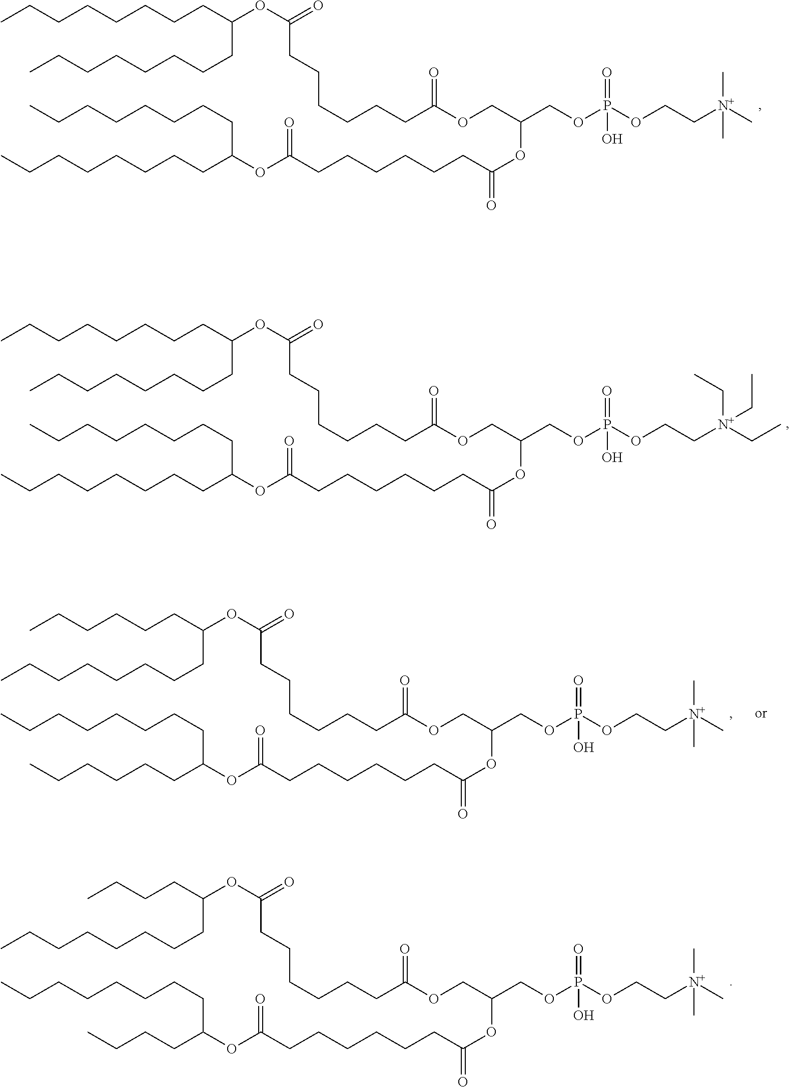

57. The compound of any one of embodiments 1-22, or a pharmaceutically acceptable salt thereof, wherein each R₂ is independently selected from

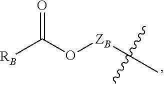

wherein each R_{B} is independently selected from optionally substituted C₁-C₃₁ alkyl, optionally substituted C₂-C₃₁alkenyl, and optionally substituted C₂-C₃₁alkynyl; and wherein each Z_{B} is independently selected from optionally substituted C₁-C₁₀ alkylene and optionally substituted C₂-C₁₀ alkenylene.
58. The compound of embodiment 57, or a pharmaceutically acceptable salt thereof, wherein each R_{B} is independently selected from optionally substituted C₁-C₃₁ alkyl, for example optionally substituted C₄-C₂₄ alkyl, for example a branched C₄-C₂₄ alkyl.
59. The compound of embodiment 58, or a pharmaceutically acceptable salt thereof, wherein each R_{B} is independently selected from a branched C₁₇ alkyl, for example

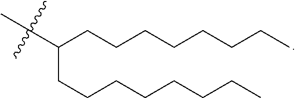 [SMILES: *C(CCCCCCCC)CCCCCCCC]

60. The compound of embodiment 58, or a pharmaceutically acceptable salt thereof, wherein each R_{B} is independently selected from branched C₄-C₂₄ alkyl, for example a branched C₁₅ alkyl, for example

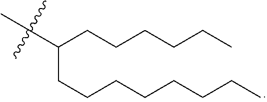 [SMILES: *C(CCCCCC)CCCCCCCC]

61. The compound of embodiment 58, or a pharmaceutically acceptable salt thereof, wherein each R_{B} is independently selected from branched C₄-C₂₄ alkyl, for example a branched C₁₃ alkyl, for example

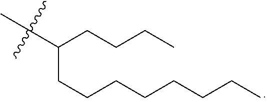 [SMILES: *C(CCCC)CCCCCCCC]

62. The compound of embodiment 58, or a pharmaceutically acceptable salt thereof, wherein each R_{B} is independently selected from:

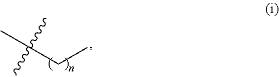

wherein each n is independently selected from 0, 1, 2, 3, 4, 5, 6, 7, 8, 9, 10, 11, 12, 13, 14, 15, 16 17, 18, 19, 20, 21, 22, 23, 24, 25, 26, 27, 28, 29, and 30,

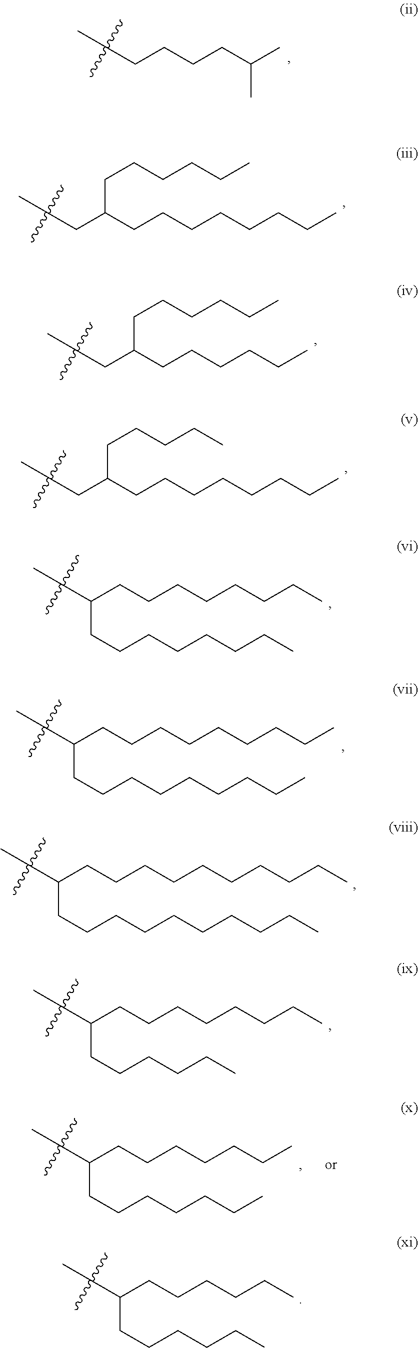

63. The compound of embodiment 57, or a pharmaceutically acceptable salt thereof, wherein each R_{B} is independently selected from optionally substituted C₂-C₃₁ alkenyl, for example optionally substituted C₄-C₂₄ alkenyl.
64. The compound of embodiment 63, or a pharmaceutically acceptable salt thereof, wherein each R_{B} is independently selected from:

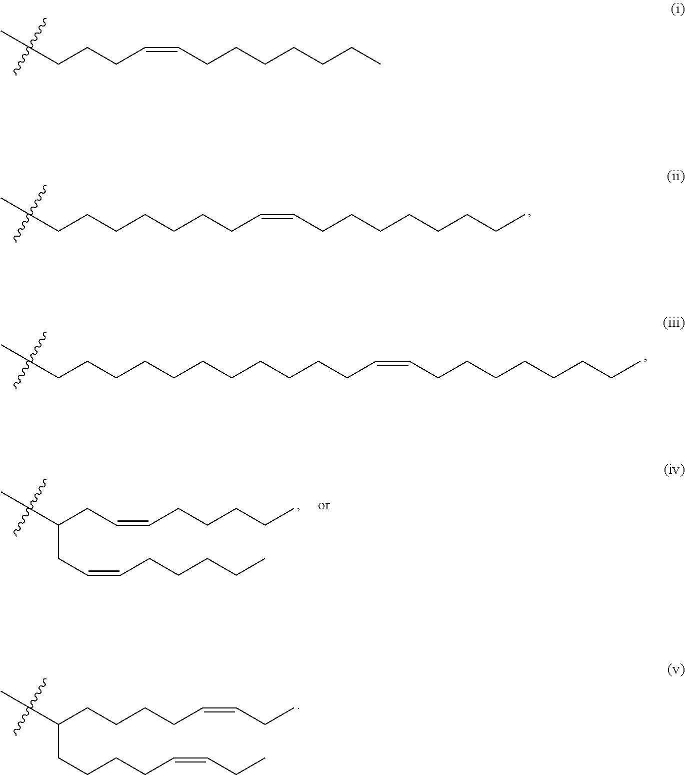 [SMILES: *CCC/C=C\CCCCCCC ; *CCCCCCC/C=C\CCCCCCCC ; *CCCCCCCCCCC/C=C\CCCCCCCC ; *C(C/C=C\CCCCC)C/C=C\CCCCC ; *C(CCCC/C=C\CC)CCCC/C=C\CC]

65. The compound of any one of embodiments 57-64, or a pharmaceutically acceptable salt thereof, wherein each Z_{B} is independently selected from optionally substituted C₁-C₁₀alkylene, for example optionally substituted C₂-C₇ alkylene.
66. The compound of embodiment 65, or a pharmaceutically acceptable salt thereof, wherein each Z_{B} is independently selected from optionally substituted C₆ alkylene, for example unsubstituted C₆ alkylene, for example unsubstituted straight-chain C₆ alkylene.
67. The compound of any one of embodiments 57-64, or a pharmaceutically acceptable salt thereof, wherein each Z_{B} is independently selected from optionally substituted C₂-C₁₀ alkenylene, for example optionally substituted C₂-C₇ alkenylene.
68. The compound of embodiment 57, or a pharmaceutically acceptable salt thereof, wherein: Y if present is —CH₂CH₂—, each R_{B} is independently selected from optionally substituted C₁-C₃₁ alkyl, and each Z_{B} is independently selected from optionally substituted C₁-C₁₀alkylene.
69. The compound of embodiment 68, or a pharmaceutically acceptable salt thereof, wherein each R_{B} is independently selected from optionally substituted C₄-C₂₄ alkyl, and each Z_{B} is independently selected from optionally substituted C₂-C₇ alkylene.
70. The compound of embodiment 68 or 69, or a pharmaceutically acceptable salt thereof, wherein each R_{B} is independently selected from:

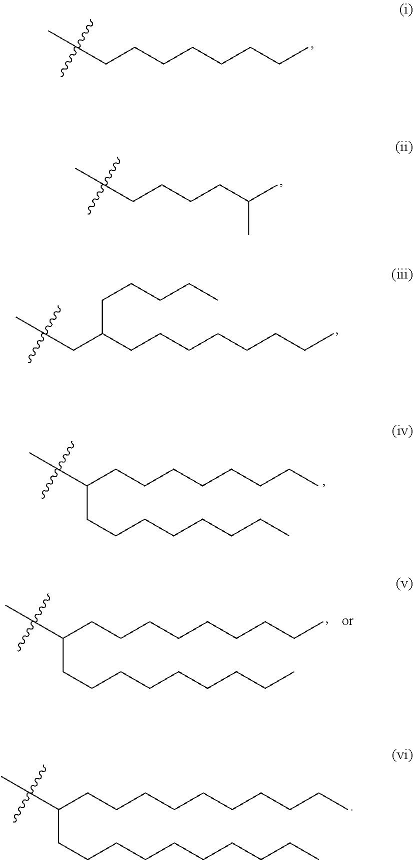

71. The compound of any one of embodiments 57-62 or 68-70, or a pharmaceutically acceptable salt thereof, wherein each R_{B} is

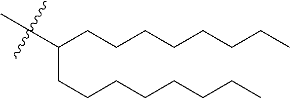 [SMILES: *C(CCCCCCCC)CCCCCCCC]

and each Z_{B} is C₅ alkylene.
72. The compound of any one of embodiments 57-62 or 68-70, or a pharmaceutically acceptable salt thereof, wherein each R_{B} is

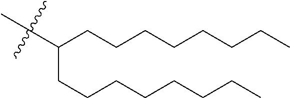 [SMILES: *C(CCCCCCCC)CCCCCCCC]

and each Z_{B} is C₆ alkylene.
73. A compound selected from those listed in Table A, or a pharmaceutically acceptable salt thereof.
74. A composition comprising one or more lipid(s) of any one of embodiments 1-73 or a pharmaceutically acceptable salt thereof, and further comprising:
   (i) one or more cationic lipids,
   (ii) one or more sterol-based lipids, and
   (iii) one or more PEG-modified lipids.
75. The composition of embodiment 74, wherein the one or more sterol-based lipids is a cholesterol-based lipid, for example cholesterol.
76. The composition of embodiment 74 or 75, wherein the composition is a lipid nanoparticle, optionally a liposome.
77. The composition of embodiment 76, wherein the one or more cationic lipid(s) constitute(s) about 20 mol % to about 60 mol % of the lipid nanoparticle.
78. The composition of any one of embodiments 76-77, wherein the one or more lipid(s) of any one of embodiments 1-73 constitute(s) about 10 mol % to about 50 mol % of the lipid nanoparticle.
79. The composition of any one of embodiments 76-78, wherein the one or more PEG-modified lipid(s) constitute(s) about 1 mol % to about 4 mol % of the lipid nanoparticle.
80. The composition of any one of embodiments 76-79, wherein the one or more sterol-based lipids constitute(s) about 10 mol % to about 50 mol % of the lipid nanoparticle.
81. The composition of any one of embodiments 76-80, wherein the lipid nanoparticle encapsulates a nucleic acid, optionally an mRNA encoding a peptide or protein.
82. The composition of any one of embodiments 76-81, wherein the lipid nanoparticle encapsulates an mRNA encoding a peptide or protein, optionally for use in a vaccine.
83. The composition of embodiment 82, wherein the lipid nanoparticles have an encapsulation percentage for mRNA of:
   (i) at least 50%;
   (ii) at least 55%;
   (iii) at least 60%;
   (iv) at least 65%;
   (v) at least 70%;
   (vi) at least 75%;
   (vii) at least 80%;
   (viii) at least 85%;
   (ix) at least 90%; or
   (x) at least 95%.
84. The composition of embodiment 83, wherein the lipid nanoparticles have an encapsulation percentage for mRNA of:
   (i) at least 85%;
   (ii) at least 90%; or
   (iii) at least 95%.
85. The composition of any one of embodiments 82-84 for use in therapy.
86. The composition of any one of embodiments 82-84 for use in a method of treating or preventing a disease amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.
87. The composition for use according to embodiment 85 or 86, wherein the composition is administered intravenously, intrathecally, or intramuscularly, or by pulmonary delivery, optionally through nebulization.
88. A method for treating or preventing a disease wherein said method comprises administering to a subject in need thereof the composition of any one of embodiments 82-84 and wherein the disease is amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.
89. The method of embodiment 88, wherein the composition is administered intravenously, intrathecally, or intramuscularly, or by pulmonary delivery, optionally through nebulization.

1. A compound having a structure according to Formula (I): 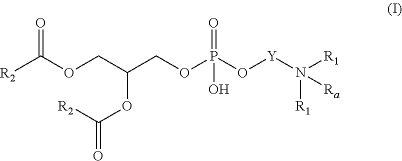
or a pharmaceutically acceptable salt thereof, wherein:
Y is selected from optionally substituted C₂-C₆ alkylene or optionally substituted C₄-C₆ alkenylene;
Rₐ is absent, or Rₐ is optionally substituted C₁-C₁₀ alkyl, wherein, when Rₐ is present, the nitrogen to which Rₐ is bonded bears a positive charge;
each R₁ is independently selected from hydrogen or optionally substituted C₁-C₁₀ alkyl;
each R₂ is independently selected from:
(i) optionally substituted C₄-C₂₄ alkyl, optionally substituted C₄-C₂₄ alkenyl, and optionally substituted C₄-C₂₄ alkynyl; 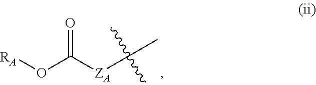
wherein each R_{A} is independently selected from optionally substituted C₁-C₃₁ alkyl, optionally substituted C₂-C₃₁ alkenyl, and optionally substituted C₂-C₃₁ alkynyl; and
wherein each Z_{A} is independently selected from optionally substituted C₁-C₁₀ alkylene and optionally substituted C₂-C₁₀ alkenylene; 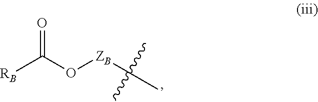
wherein each R_{B} is independently selected from optionally substituted C₁-C₃₁ alkyl, optionally substituted C₂-C₃₁ alkenyl, and optionally substituted C₂-C₃₁ alkynyl; and
wherein each Z_{B} is independently selected from optionally substituted C₁-C₁₀ alkylene and optionally substituted C₂-C₁₀ alkenylene.

2. The compound of claim 1, wherein the compound has a structure according to Formula (II), (IIa), (IIb), (IIc), (IId) or (III): 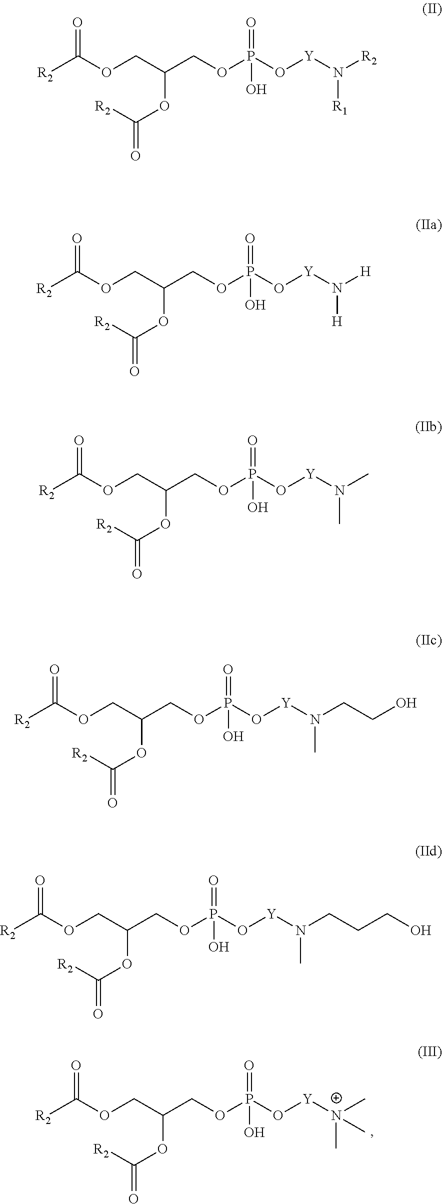
or a pharmaceutically acceptable salt thereof.

3. The compound of claim 1, or a pharmaceutically acceptable salt thereof, wherein Y is selected from (i) optionally substituted C₂₋₆ alkylene or (ii) optionally substituted C₄₋₆ alkenylene, for example wherein Y is —CH₂CH₂—.

4. The compound of claim 1, wherein the compound has a structure according to Formula (IV), (V), (Va), (Vb), (Vc), or (Vd): 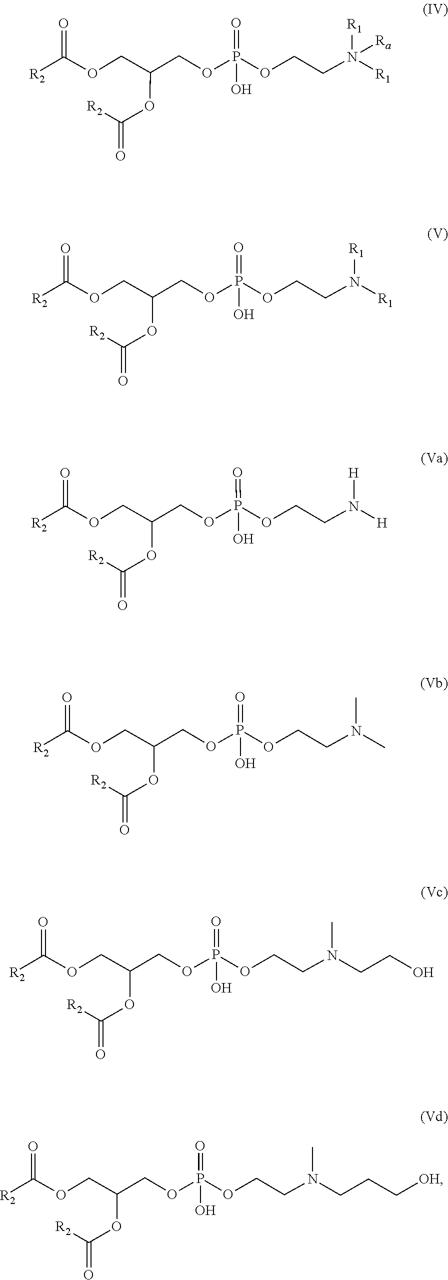
or a pharmaceutically acceptable salt thereof.

5. The compound of claim 1, or a pharmaceutically acceptable salt thereof, wherein
(i) the stereochemistry of the carbon atom situated between R₂C(═O)O—CH₂— and —CH₂—OP(═O)(OH)—O— is as depicted in the following structure:
or 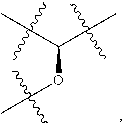
(ii) the stereochemistry of the carbon atom situated between R₂C(═O)O—CH₂— and —CH₂—OP(═O)(OH)—O— is as depicted in the following structure:

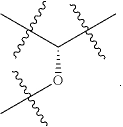

6. The compound of claim 1, or a pharmaceutically acceptable salt thereof, wherein each R₂ is independently selected from 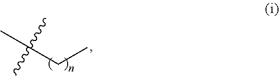
wherein each n is independently selected from 3, 4, 5, 6, 7, 8, 9, 10, 11, 12, 13, 14, 15, 16 17, 18, 19, 20, 21, 22, and 23,

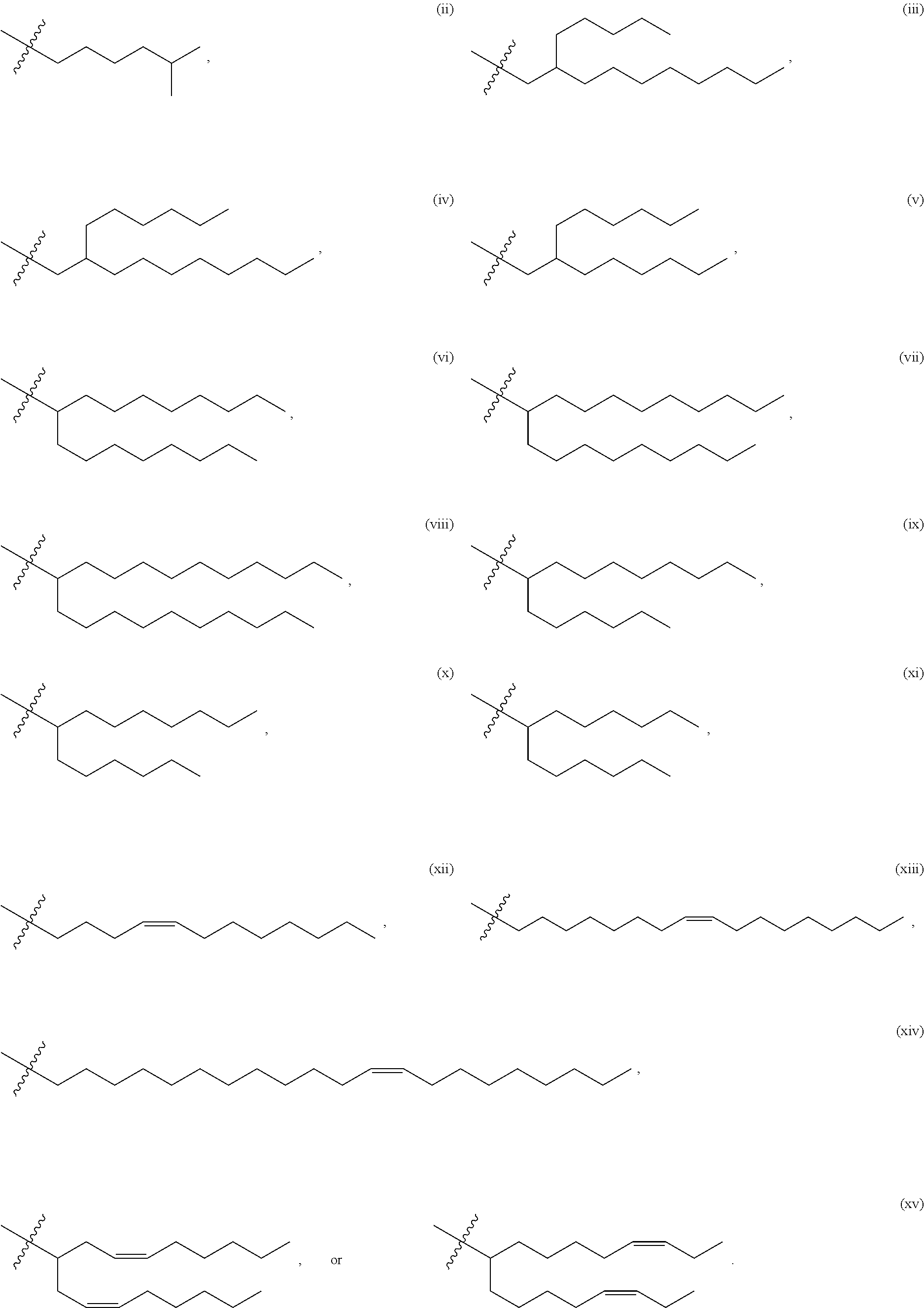

7. The compound of claim 1, or a pharmaceutically acceptable salt thereof, wherein each R_{A} or R_{B}, if present, is independently selected from: 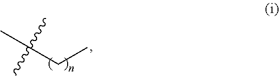
wherein each n is independently selected from 0, 1, 2, 3, 4, 5, 6, 7, 8, 9, 10, 11, 12, 13, 14, 15, 16 17, 18, 19, 20, 21, 22, 23, 24, 25, 26, 27, 28, 29, and 30,

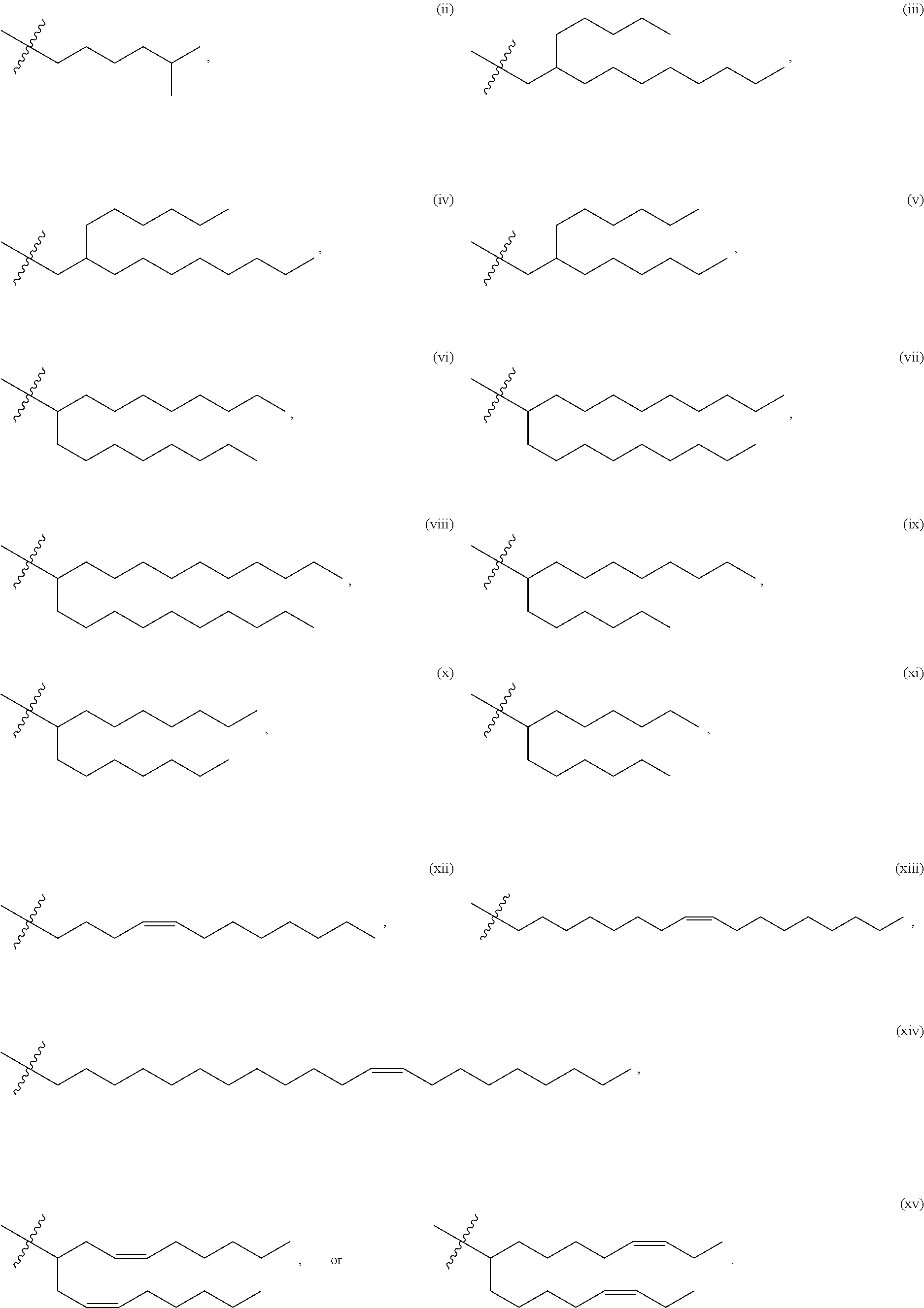

8. The compound of claim 1 wherein:
(i) each R_{A}, if present, is 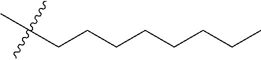 [SMILES: *CCCCCCCC]
and each Z_{A} is then C₇ alkylene;
(ii) each R_{A}, if present, is 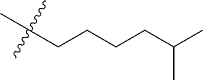 [SMILES: *CCCCC(C)C]
and each Z_{A} is then C₆ alkylene;
(iii) each R_{A}, if present, is 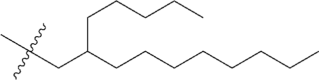 [SMILES: *CC(CCCCC)CCCCCCCC]
and each Z_{A} is then C₂ alkylene;
(iv) each R_{A}, if present, is 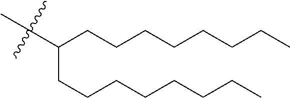 [SMILES: *C(CCCCCCCC)CCCCCCCC]
and each Z_{A} is then independently selected from C₅-C₇ alkylene;
(v) each R_{A}, if present, is 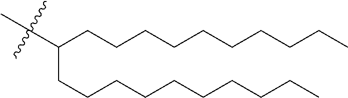 [SMILES: *C(CCCCCCCCCC)CCCCCCCCCC]
and each Z_{A} is then C₆ alkylene;
(vi) each R_{A}, if present, is 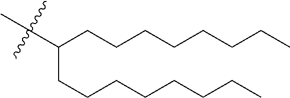 [SMILES: *C(CCCCCCCC)CCCCCCCC]
and each Z_{A} is then C₆ alkenylene;
(vii) each R_{A}, if present, is 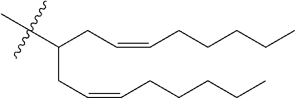 [SMILES: *C(C/C=C\CCCCC)C/C=C\CCCCC]
and each Z_{A} is then C₆ alkylene;
(viii) each R_{A}, if present, is 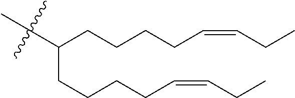 [SMILES: *C(CCCC/C=C\CC)CCCC/C=C\CC]
and each Z_{A} is then C₆ alkylene;
(ix) each R_{B}, if present, is 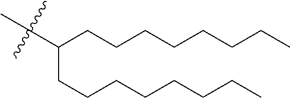 [SMILES: *C(CCCCCCCC)CCCCCCCC]
and each Z_{B} is then C₅ alkylene; or
(x) each R_{B}, if present, is 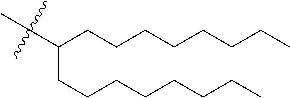 [SMILES: *C(CCCCCCCC)CCCCCCCC]
and each Z_{B} is then C₆ alkylene.

9. A compound selected from those listed in Table A, or a pharmaceutically acceptable salt thereof.

10. A composition comprising one or more lipid(s) of claim 1 or a pharmaceutically acceptable salt thereof, and further comprising:
(i) one or more cationic lipids,
(ii) one or more sterol-based lipids, and
(iii) one or more PEG-modified lipids.

11. The composition of claim 10, wherein the composition is a lipid nanoparticle.

12. The composition of claim 11, wherein the lipid nanoparticle encapsulates an mRNA encoding a peptide or protein.

13. (canceled)

14. (canceled)

15. A method for treating or preventing a disease wherein said method comprises administering to a subject in need thereof the composition of claim 12 and wherein the disease is amenable to treatment or prevention by the peptide or protein encoded by the mRNA.

16. The composition of claim 11, wherein:
(i) the one or more cationic lipid(s) constitute(s) about 20 mol % to about 60 mol % of the lipid nanoparticle;
(ii) the one or more lipid(s) of any one of claims 1-9 constitute(s) about 10 mol % to about 50 mol % of the lipid nanoparticle;
(iii) the one or more PEG-modified lipid(s) constitute(s) about 1 mol % to about 4 mol % of the lipid nanoparticle; and/or
(iv) the one or more sterol-based lipids constitute(s) about 10 mol % to about 50 mol % of the lipid nanoparticle.

17. A vaccine comprising the composition of claim 11, wherein the lipid nanoparticle encapsulates an mRNA encoding a peptide or protein.

18. The method of claim 15, wherein the mRNA encodes a peptide or protein that is an antigen.

19. The method of claim 15, wherein the disease is selected from (a) a protein deficiency, (b) an autoimmune disease, (c) an infectious disease, or (d) cancer.

20. The method of claim 15, wherein the composition is administered intravenously, intrathecally, intramuscularly, or by pulmonary delivery.
